(19) United States
(12) Patent Application Publication
(10) Pub. No.: US 2024/0228493 A1
(43) Pub. Date: Jul. 11, 2024

(54) LRRK2 INHIBITORS AND USES THEREOF

(71) Applicant: NEURON23, ICN., South San Francisco, CA (US)

(72) Inventors: Ming Yu, South San Francisco, CA (US); Gyanendra Kumar, South San Francisco, CA (US); Zhonghua Pei, South San Francisco, CA (US); Scott Savage, South San Francisco, CA (US); Kejia Wu, South San Francisco, CA (US); Jim X. Zheng, South San Francisco, CA (US)

(21) Appl. No.: 18/512,757

(22) Filed: Nov. 17, 2023

Related U.S. Application Data
(60) Provisional application No. 63/426,546, filed on Nov. 18, 2022.

(51) Int. Cl. C07D 471/14; C07D 519/00

(52) U.S. Cl. CPC C07D 471/14; C07D 519/00

(57) ABSTRACT

The invention provides compounds that modulate the activity of protein kinases that are associated with human diseases, disorders, and conditions. In particular, compounds of the invention inhibit LRRK2.

### FIELD OF INVENTION

The present invention relates to compounds that are capable of inhibiting one or more kinases, more particularly, LRRK2. The compounds find applications in the treatment of a variety of disorders, including cancer and neurodegenerative diseases such as Parkinson's disease.

### BACKGROUND

A variety of medical conditions that affect millions of people are caused or exacerbated by unregulated activity of protein kinases. For example, aberrant kinase activity is associated with autoimmune diseases, inflammatory diseases, bone diseases, metabolic diseases, neurological and neurodegenerative diseases, cancer, cardiovascular diseases, allergies, asthma, Alzheimer's disease, Parkinson's disease, skin disorders, eye diseases, infectious diseases and hormone-related diseases. For many such disorders, however, no effective inhibitor or activator exists for the particular kinase that causes the disorder or its symptoms. Consequently, patients continue to suffer from an array of disorders due to the lack of a suitable drug for their conditions.

Inflammatory bowel disease (IBD) is a term that describes disorders involving chronic inflammation and often destruction of tissues in the gastrointestinal tract (GI). The 2 most common types of IBD are Ulcerative colitis (UC) and Crohn's disease (CD). Both ulcerative colitis and Crohn's disease are typically characterized by diarrhea, rectal bleeding, abdominal pain, fatigue and weight loss. UC involves inflammation and ulcerations along the lining of your large intestine and rectum whereas CD is characterized by inflammation of the lining as well as deeper layers of the digestive tract. Although typically associated with inflammation in the small intestine, CD can also affect the large intestine and, less commonly, the upper gastrointestinal tract.

### SUMMARY

Inhibitors of leucine rich repeat kinase 2 (LRRK2), which may not to be CNS-penetrant, may be effective treatment of inflammation diseases such as IBD. The invention provides LRRK2 inhibitors for treatment of IBD and other inflammatory diseases. In one aspect, the invention provides a compound of Formula (I):

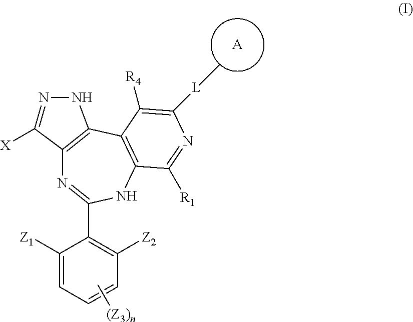

or an enantiomer, mixture of enantiomers, tautomer, or pharmaceutically acceptable salt thereof,
wherein:
n is 1, 2, or 3;
Y is N or CR₂;
Z₁, Z₂, and Z₃ are independently selected from H, —OH, halo, cyano, amino, C₁-C₆ alkyl, C₁-C₆ heteroalkyl, haloalkyl, alkoxy, haloalkxoxy, —CH(OH)-alkyl, hydroxyalkyl, or hydroxyalkoxy;
X is H, halo, cyano, cycloalkyl, C₁-C₆ alkyl, optionally deuterated C₁-C₆ alkyl, C₁-C₆ heteroalkyl, haloalkyl, alkoxy, haloalkxoxy, —CH(OH)-alkyl, hydroxyalkyl, or hydroxyalkoxy;
R₁, R₂, and R₄ are independently selected from H, halo, cyano, C₁-C₆ alkyl, optionally deuterated C₁-C₆ alkyl, C₁-C₆ heteroalkyl, haloalkyl, alkoxy, haloalkxoxy, —CH(OH)-alkyl, hydroxyalkyl, or hydroxyalkoxy;
L is a linker, wherein L is a single bond, substituted or unsubstituted C₁-C₄ alkyl, substituted or unsubstituted C₁-C₄ heteroalkyl, wherein the one or more heteroatoms are selected from O, S, or N;
A is 4-8 membered substituted or unsubstituted heterocycloalkyl, spiroheterocycloalkyl, heteroaryl, wherein one or more heteroatoms are selected from a group consisting of O, S, or N; and
wherein the substituents may be selected from a group consisting of substituted or unsubstituted 3-7 membered heterocycle, ═O, —CH₂-cycloalkyl, —CF₂-cycloalky, —C(═O)—O-alkyl, halo, deuterium, cyano, cyanoalkyl, —CF₃, mono-, di-, or tri-halo alkyl, CH(CH₃)-cycloalkyl, —CH₂-aryl, —CF₂-aryl, —CH(—CH₃)-aryl, C(═O)-alkyl, —C(═O)cycloalkyl, —C(═O)—NH-alkyl, —COOH (and esters and carboxamides thereof), —C(═O)-morpholine, —C(═O)-heterocycles, —C(—CH₃)₂—OH, —CH₂—C(═O)—NH₂; -hydroxy, alkylhydroxy, alkyl-COOH (and esters and carboxamides thereof), amino, NHC(═O)alkyl, —N(alkyl)C(═O)alkyl, —NHC(═O)aryl, —N(alkyl)C(═O)aryl, —N(H)S(═O)₂alkyl, —N(H)S(═O)₂aryl, —N(alkyl)S(═O)₂alkyl, cyanoalkyl, haloalkyl, ═S═(O)₂-alkyl, ═S═(O)₂-cycloalkyl, ═S═(O)₂-aryl, —S(═O)₂N(H)alkyl, substituted or unsubstituted morpholine, 3-7 membered heterocycle, any of which may have one or more substituents, 3-7 membered cycloalkyl or heterocycle, wherein the 3-7 membered cycloalkyl or heterocycle is optionally fused with another 3-7 cycloalkyl or heterocycle, wherein the rings are spiro, bridged bicyclic, or spiro, wherein the at least one heteroatom in the heterocycle rings are independently selected from O, S, and N; and
wherein one or more hydrogen atoms are optionally deuterium.

In another embodiment, Y is N.

In another embodiment, Y is CR₂.

In another embodiment, Z₁ and Z₂ are F.

In another embodiment, Z₁ and Z₂ are independently selected from Cl or F.

In another embodiment, Z₃ is H.

In another embodiment, Z₃ is F.

In another embodiment, Z₃ is —CH₂—OH.

In another embodiment, X is selected from a group consisting of methyl, H, Cl, or —CD₃.

In another embodiment, X is methyl.

In another embodiment, R₁ is selected from H or methyl.

In another embodiment, R₂ is H.

In another embodiment, R₄ is H.

In another embodiment, L is a single bond.

In another embodiment, L is alkyl.

In another embodiment, L is —C≡C—.

In another embodiment, A is not substituted or unsubstituted 1,2,3,6-Tetrahydropyridin.

In another embodiment, A is morpholine with optional substitutions

In another embodiment, A is selected from:

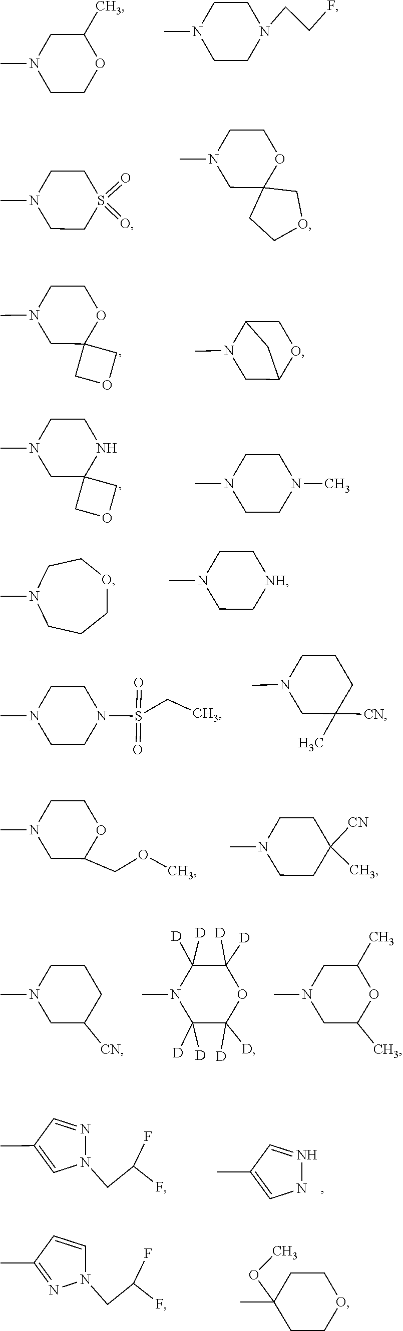

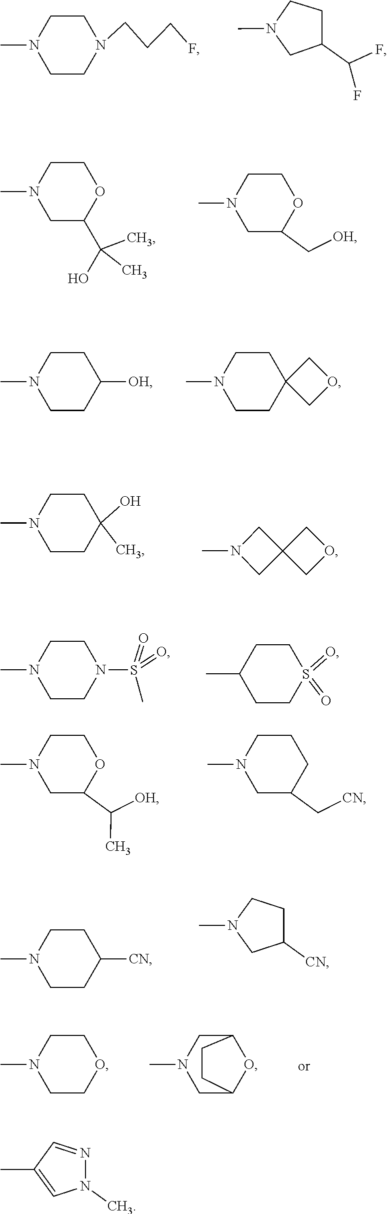

In another embodiment, the compound of Formula (I) is selected from:

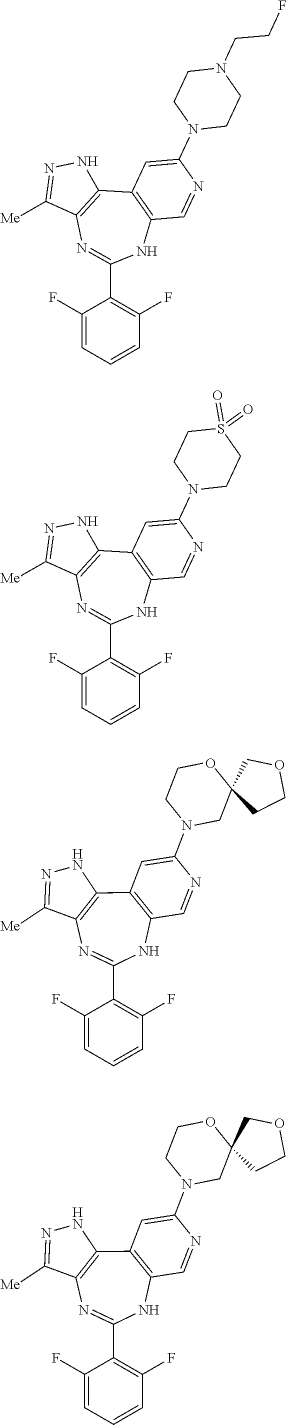 [SMILES: Cc1n[nH]c2c1N=C(c1c(F)cccc1F)Nc1cnc(N3CCN(CCF)CC3)cc1-2 ; Cc1n[nH]c2c1N=C(c1c(F)cccc1F)Nc1cnc(N3CCS(=O)(=O)CC3)cc1-2 ; Cc1n[nH]c2c1N=C(c1c(F)cccc1F)Nc1cnc(N3CCO[C@@]4(CCOC4)C3)cc1-2 ; Cc1n[nH]c2c1N=C(c1c(F)cccc1F)Nc1cnc(N3CCO[C@]4(CCOC4)C3)cc1-2]

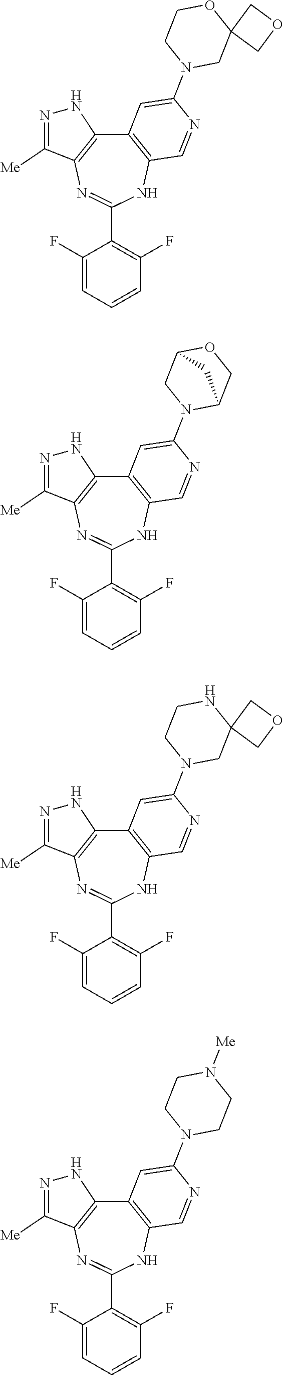 [SMILES: Cc1n[nH]c2c1N=C(c1c(F)cccc1F)Nc1cnc(N3CCOC4(COC4)C3)cc1-2 ; Cc1n[nH]c2c1N=C(c1c(F)cccc1F)Nc1cnc(N3C[C@@H]4C[C@H]3CO4)cc1-2 ; Cc1n[nH]c2c1N=C(c1c(F)cccc1F)Nc1cnc(N3CCNC4(COC4)C3)cc1-2 ; Cc1n[nH]c2c1N=C(c1c(F)cccc1F)Nc1cnc(N3CCN(C)CC3)cc1-2]

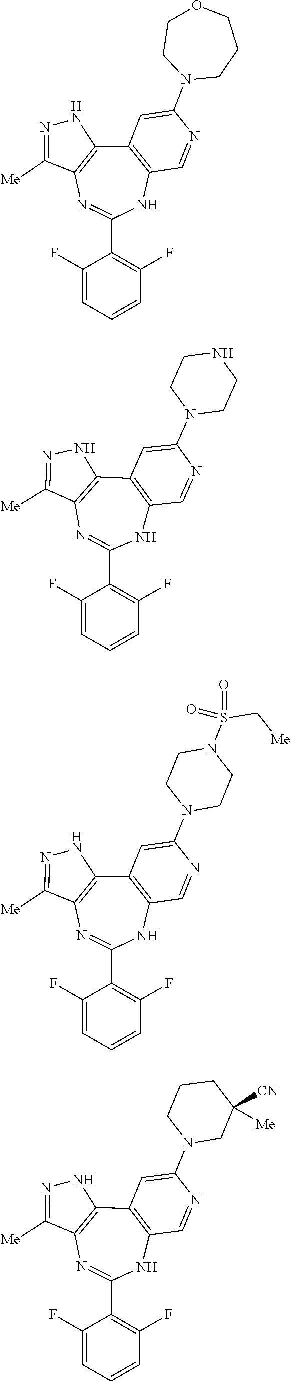 [SMILES: Cc1n[nH]c2c1N=C(c1c(F)cccc1F)Nc1cnc(N3CCCOCC3)cc1-2 ; Cc1n[nH]c2c1N=C(c1c(F)cccc1F)Nc1cnc(N3CCNCC3)cc1-2 ; CCS(=O)(=O)N1CCN(c2cc3c(cn2)NC(c2c(F)cccc2F)=Nc2c(C)n[nH]c2-3)CC1 ; Cc1n[nH]c2c1N=C(c1c(F)cccc1F)Nc1cnc(N3CCC[C@@](C)(C#N)C3)cc1-2]

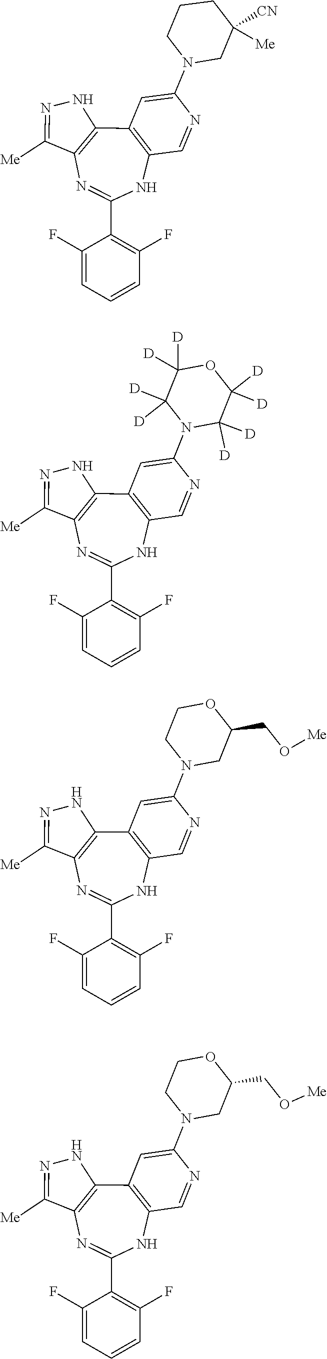 [SMILES: Cc1n[nH]c2c1N=C(c1c(F)cccc1F)Nc1cnc(N3CCC[C@](C)(C#N)C3)cc1-2 ; [2H]C1([2H])OC([2H])([2H])C([2H])([2H])N(c2cc3c(cn2)NC(c2c(F)cccc2F)=Nc2c(C)n[nH]c2-3)C1([2H])[2H] ; COC[C@H]1CN(c2cc3c(cn2)NC(c2c(F)cccc2F)=Nc2c(C)n[nH]c2-3)CCO1 ; COC[C@@H]1CN(c2cc3c(cn2)NC(c2c(F)cccc2F)=Nc2c(C)n[nH]c2-3)CCO1]

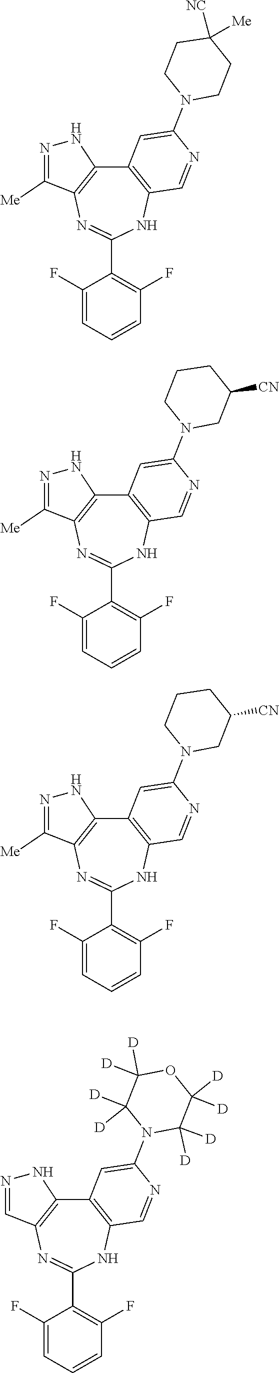 [SMILES: Cc1n[nH]c2c1N=C(c1c(F)cccc1F)Nc1cnc(N3CCC(C)(C#N)CC3)cc1-2 ; Cc1n[nH]c2c1N=C(c1c(F)cccc1F)Nc1cnc(N3CCC[C@@H](C#N)C3)cc1-2 ; Cc1n[nH]c2c1N=C(c1c(F)cccc1F)Nc1cnc(N3CCC[C@H](C#N)C3)cc1-2 ; [2H]C1([2H])OC([2H])([2H])C([2H])([2H])N(c2cc3c(cn2)NC(c2c(F)cccc2F)=Nc2cn[nH]c2-3)C1([2H])[2H]]

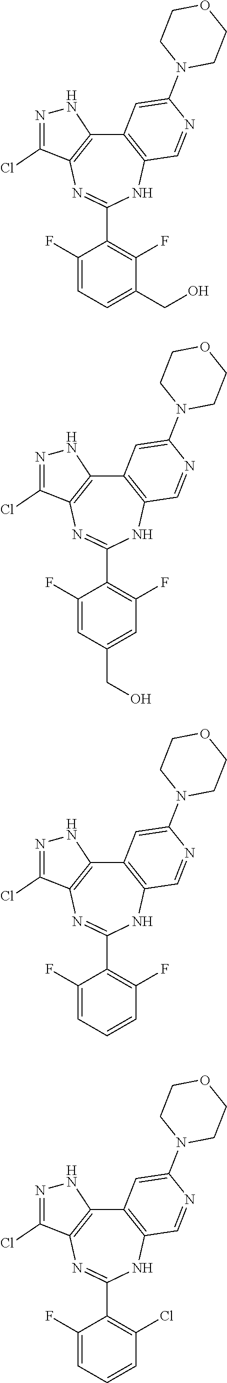

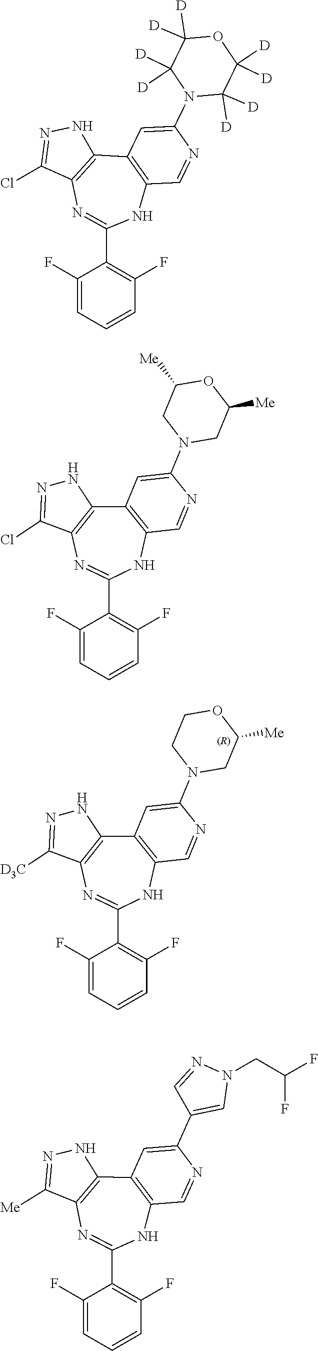

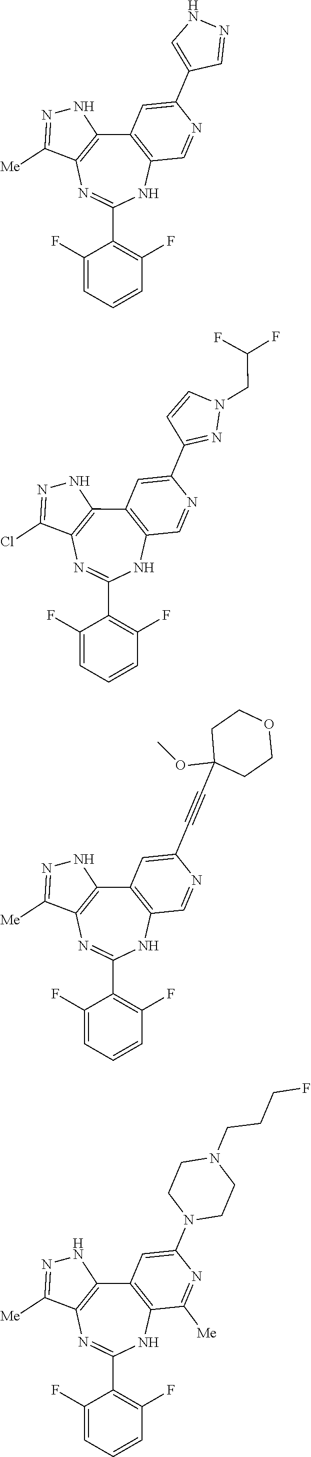 [SMILES: Cc1n[nH]c2c1N=C(c1c(F)cccc1F)Nc1cnc(-c3cn[nH]c3)cc1-2 ; Fc1cccc(F)c1C1=Nc2c(Cl)n[nH]c2-c2cc(-c3ccn(CC(F)F)n3)ncc2N1 ; COC1(C#Cc2cc3c(cn2)NC(c2c(F)cccc2F)=Nc2c(C)n[nH]c2-3)CCOCC1 ; Cc1n[nH]c2c1N=C(c1c(F)cccc1F)Nc1c-2cc(N2CCN(CCCF)CC2)nc1C]

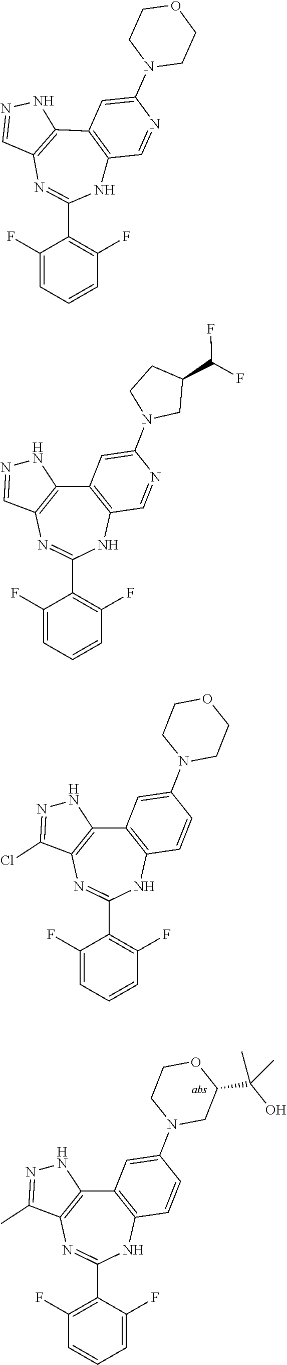 [SMILES: Fc1cccc(F)c1C1=Nc2cn[nH]c2-c2cc(N3CCOCC3)ncc2N1 ; Fc1cccc(F)c1C1=Nc2cn[nH]c2-c2cc(N3CC[C@@H](C(F)F)C3)ncc2N1 ; Fc1cccc(F)c1C1=Nc2c(Cl)n[nH]c2-c2cc(N3CCOCC3)ccc2N1 ; Cc1n[nH]c2c1N=C(c1c(F)cccc1F)Nc1ccc(N3CCO[C@H](C(C)(C)O)C3)cc1-2]

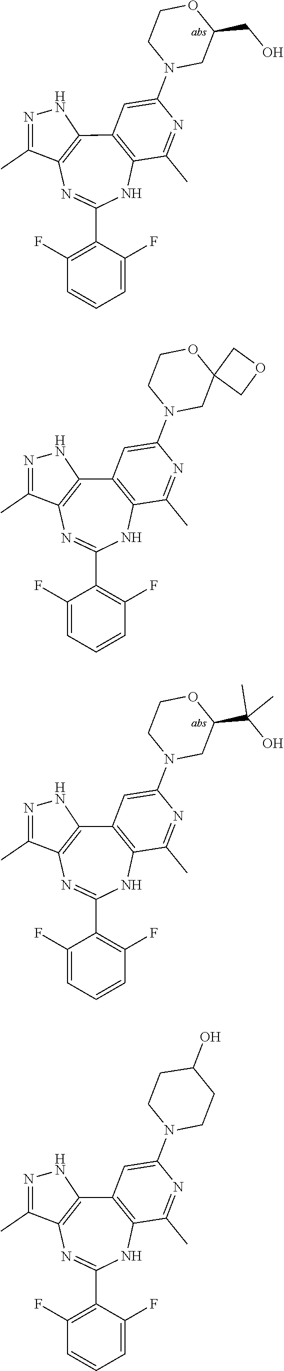

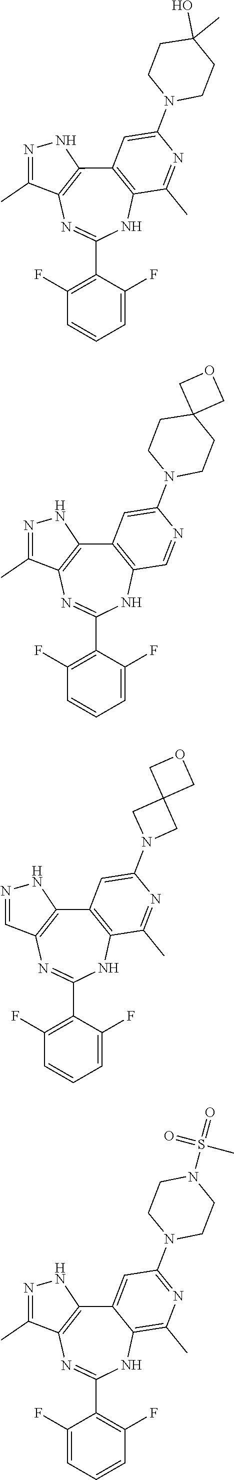

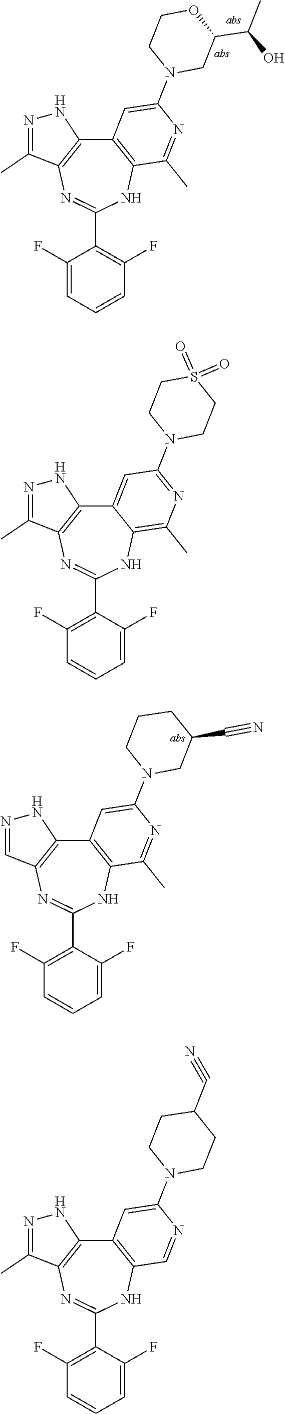 [SMILES: Cc1n[nH]c2c1N=C(c1c(F)cccc1F)Nc1c-2cc(N2CCO[C@H]([C@@H](C)O)C2)nc1C ; Cc1n[nH]c2c1N=C(c1c(F)cccc1F)Nc1c-2cc(N2CCS(=O)(=O)CC2)nc1C ; Cc1nc(N2CCC[C@@H](C#N)C2)cc2c1NC(c1c(F)cccc1F)=Nc1cn[nH]c1-2 ; Cc1n[nH]c2c1N=C(c1c(F)cccc1F)Nc1cnc(N3CCC(C#N)CC3)cc1-2]

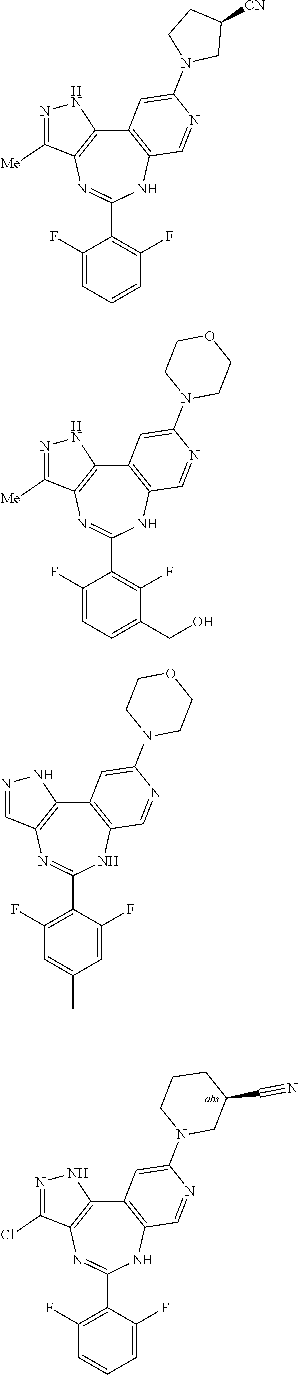

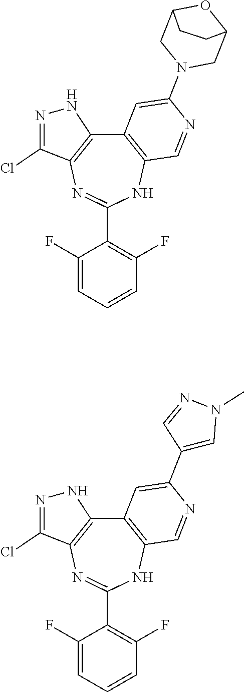 [SMILES: Fc1cccc(F)c1C1=Nc2c(Cl)n[nH]c2-c2cc(N3CC4CCC(C3)O4)ncc2N1 ; Cn1cc(-c2cc3c(cn2)NC(c2c(F)cccc2F)=Nc2c(Cl)n[nH]c2-3)cn1]

In one embodiment, the invention provides a pharmaceutical composition comprising a compound of the above Formula (I) and pharmaceutically acceptable salts thereof together with a pharmaceutically acceptable excipient.

In one embodiment, the invention provides compounds of the above Formula (I) and pharmaceutically acceptable salts thereof for use in therapy.

In one embodiment, the invention provides compounds of the above Formula (I) and pharmaceutically acceptable salts thereof for use in a method for the treatment of a disease associated with LRRK2.

In one embodiment, the invention relates to the use of a compound of the above Formula (I) and pharmaceutically acceptable salts thereof in the manufacture of a medicament for use in the treatment of a disease associated with LRRK2.

In one embodiment, the invention relates to a method for the treatment of a disease associated with LRRK2, including PD, the method comprising the administration of a therapeutically effective amount of a compound of the above formula A and pharmaceutically acceptable salts thereof to a patient in need thereof.

In another aspect, the invention provides methods of modulating the activity of a kinase by contacting cells containing a kinase with one or more compounds of the invention, such as any of those described above. The compound may inhibit activity of the kinase. The compound may increase the activity of the kinase. The kinase may be LRRK2.

In embodiments of the use, the condition treated by the compounds of the invention is an autoimmune disease, inflammatory disease, bone disease, metabolic disease, neurological or neurodegenerative disease, cancer, cardiovascular disease, allergies, asthma, Alzheimer's disease, Parkinson's disease, skin disorder, eye disease, infectious disease, or hormone-related disease.

In certain preferred embodiments, the compounds of the invention may be used for treatment of inflammatory bowel disease (IBD).

In another embodiment, the compounds of the invention are not effective in crossing the blood brain barrier. Thus, in the embodiment, the compounds of the invention may be effective through the systemic exposure of the compounds in the body.

### DETAILED DESCRIPTION

### LRRK2 and IBD

The leucine-rich repeat kinase 2 (LRRK2) gene encodes a 286 kDa multidomain protein with cellular functions that include neurite outgrowth, cytoskeletal maintenance, vesicle trafficking, regulation of autophagy, and immune cell functioning (Cookson M R. LRRK2 pathways leading to neurodegeneration. Curr Neurol Neurosci Rep. 2015; 15(7):42.; Wallings R, and Tansey, M. Biochem Soc Trans. 2019 Dec. 20; 47(6): 1581-1595). While mutations in LRRK2 are more commonly known for their association with Parkinson's disease (PD), genome-wide analysis has identified 17 loci (including known PD loci adjacent to LRRK2) that overlap between PD and autoimmune diseases such as rheumatoid arthritis and IBD (Witoelar A., Jansen I. E. Wang Y., Desikan R. S., Gibbs J. R., Blauwendraat C. et al. (2017) Genome-wide pleiotropy between Parkinson disease and autoimmune diseases. JAMA Neurol. 74, 780.). Independent research has shown that disease-linked mutations in LRRK2 confer a toxic gain in function in its kinase activity (West A B. Achieving neuroprotection with LRRK2 kinase inhibitors in Parkinson disease. Exp Neurol. 2017; 298(Pt B):236-45.) resulting in increased LRRK2 autophosphorylation (Sheng Z, Zhang S, Bustos D, Kleinheinz T, Le Pichon C E, Dominguez S L, et al. Ser1292 autophosphorylation is an indicator of LRRK2 kinase activity and contributes to the cellular effects of PD mutations. Sci Transl Med. 2012; 4(164):164ra161.) and phosphorylation of downstream substrates such as Ras-family Rab GTPases (Steger M, Tonelli F, Ito G, Davies P, Trost M, Vetter M, et al. Phosphoproteomics reveals that Parkinson's disease kinase LRRK2 regulates a subset of Rab GTPases. Elife. 2016; 5:e12813. doi: 10.7554/eLife.12813.). LRRK2 is a ubiquitously expressed protein with high expression observed in the brain, kidney and lungs. However, increased expression in immune cells, specifically in response to pro-inflammatory signals, has been observed in many immune cell types, strongly implicating LRRK2 as a regulator of the immune response. (Wallings R, and Tansey, M. Biochem Soc Trans. 2019 Dec. 20; 47(6): 1581-1595). Together these findings, LRRK2 inhibitors may be useful for the treatment of LRRK2-associated IBD.

### Chemical Definitions

The expression alkyl refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”, also referred to herein as “lower alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ 14alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), isobutyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Unless otherwise specified, each instance of an alkyl group is independently optionally substituted, i.e., unsubstituted (an “unsubstituted alkyl”) or substituted (a “substituted alkyl”) with one or more substituents; e.g., from 1 to 5 substituents, 1 to 3 substituents, or 1 substituent. In certain embodiments, the alkyl group is unsubstituted C₁₋₁₀ alkyl (e.g., —CH₃). In certain embodiments, the alkyl group is substituted C₁₋₁₀ alkyl. Common alkyl abbreviations include Me (—CH₃), Et (—CH₂CH₃), iPr (—CH(CH₃)₂), nPr (—CH₂CH₂CH₃), n-Bu (—CH₂CH₂CH₂CH₃), or i-Bu (—CH₂CH(CH₃)₂).

The expression heteroalkyl refers to an alkyl group, as defined herein, which further comprises 1 or more (e.g., 1, 2, 3, or 4) heteroatoms (e.g., oxygen, sulfur, nitrogen, boron, silicon, phosphorus) within the parent chain, wherein the one or more heteroatoms is inserted between adjacent carbon atoms within the parent carbon chain and/or one or more heteroatoms is inserted between a carbon atom and the parent molecule, i.e., between the point of attachment. In certain embodiments, a heteroalkyl group refers to a saturated group having from 1 to 10 carbon atoms and 1, 2, 3, or 4 heteroatoms (“heteroC₁₋₁₀ alkyl”). In some embodiments, a heteroalkyl group is a saturated group having 1 to 9 carbon atoms and 1, 2, 3, or 4 heteroatoms (“heteroC₁₋₉ alkyl”). In some embodiments, a heteroalkyl group is a saturated group having 1 to 8 carbon atoms and 1, 2, 3, or 4 heteroatoms (“heteroC₁₋₈ alkyl”). In some embodiments, a heteroalkyl group is a saturated group having 1 to 7 carbon atoms and 1, 2, 3, or 4 heteroatoms (“heteroC₁₋₇ alkyl”). In some embodiments, a heteroalkyl group is a group having 1 to 6 carbon atoms and 1, 2, or 3 heteroatoms (“heteroC₁₋₆ alkyl”). In some embodiments, a heteroalkyl group is a saturated group having 1 to 5 carbon atoms and 1 or 2 heteroatoms (“heteroC₁₋₁₀ alkyl”). In some embodiments, a heteroalkyl group is a saturated group having 1 to 4 carbon atoms and 1 or 2 heteroatoms (“heteroC₁₋₄ alkyl”). In some embodiments, a heteroalkyl group is a saturated group having 1 to 3 carbon atoms and 1 heteroatom (“heteroC₁₋₃ alkyl”). In some embodiments, a heteroalkyl group is a saturated group having 1 to 2 carbon atoms and 1 heteroatom (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms (“heteroC₂₋₆ alkyl”).

The expression alkenyl refers to a radical of a straight-chain or branched hydrocarbon group having from 2 to 20 carbon atoms, one or more carbon-carbon double bonds (e.g., 1, 2, 3, or 4 carbon-carbon double bonds) (“C₂₋₂₀ alkenyl”). In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently optionally substituted, i.e., unsubstituted (an “unsubstituted alkenyl”) or substituted (a “substituted alkenyl”) with one or more substituents e.g., from 1 to 5 substituents, 1 to 3 substituents, or 1 substituent. In certain embodiments, the alkenyl group is unsubstituted C₂₋₁₀ alkenyl. In certain embodiments, the alkenyl group is substituted C₂₋₁₀ alkenyl.

The term “heteroalkenyl,” as used herein, refers to an alkenyl group, as defined herein, which further comprises one or more (e.g., 1, 2, 3, or 4) heteroatoms (e.g., oxygen, sulfur, nitrogen, boron, silicon, phosphorus) wherein the one or more heteroatoms is inserted between adjacent carbon atoms within the parent carbon chain and/or one or more heteroatoms is inserted between a carbon atom and the parent molecule, i.e., between the point of attachment. In certain embodiments, a heteroalkenyl group refers to a group having from 2 to 10 carbon atoms, at least one double bond, and 1, 2, 3, or 4 heteroatoms (“heteroC₂₋₁₀ alkenyl”). In some embodiments, a heteroalkenyl group has 2 to 9 carbon atoms at least one double bond, and 1, 2, 3, or 4 heteroatoms (“heteroC₂₋₉ alkenyl”). In some embodiments, a heteroalkenyl group has 2 to 8 carbon atoms, at least one double bond, and 1, 2, 3, or 4 heteroatoms (“heteroC₂₋₈ alkenyl”). In some embodiments, a heteroalkenyl group has 2 to 7 carbon atoms, at least one double bond, and 1, 2, 3, or 4 heteroatoms (“heteroC₂₋₇ alkenyl”). In some embodiments, a heteroalkenyl group has 2 to 6 carbon atoms, at least one double bond, and 1, 2, or 3 heteroatoms (“heteroC₂₋₆ alkenyl”). In some embodiments, a heteroalkenyl group has 2 to 5 carbon atoms, at least one double bond, and 1 or 2 heteroatoms (“heteroC₂₋₅ alkenyl”). In some embodiments, a heteroalkenyl group has 2 to 4 carbon atoms, at least one double bond, and 1 or 2 heteroatoms (“heteroC₂₋₄ alkenyl”). In some embodiments, a heteroalkenyl group has 2 to 3 carbon atoms, at least one double bond, and 1 heteroatom (“heteroC₂₋₃ alkenyl”). In some embodiments, a heteroalkenyl group has 2 to 6 carbon atoms, at least one double bond, and 1 or 2 heteroatoms (“heteroCC₂₋₆ alkenyl”).

The expression cycloalkyl refers to a saturated or partially unsaturated (for example, a cycloalkenyl group) cyclic group that contains one or more rings, e.g., 2 or 3 rings, and contains from 3 to 14 ring carbon atoms, such as from 3 to 10 (e.g., 3, 4, 5, 6 or 7) ring carbon atoms. The expression cycloalkyl refers furthermore to groups in which one or more hydrogen atoms have been replaced by fluorine, chlorine, bromine or iodine atoms or by OH, ═O, SH, ═S, NH₂, ═NH, N₃ or NO₂ groups, thus, for example, cyclic ketones such as, for example, cyclohexanone, 2-cyclohexenone or cyclopentanone. Further specific examples of cycloalkyl groups are a cyclopropyl, cyclobutyl, cyclopentyl, spiro[4,5]decanyl, norbornyl, cyclohexyl, cyclopentenyl, cyclohexadienyl, decalinyl, bicyclo[4.3.0]nonyl, tetraline, cyclopentylcyclohexyl, fluorocyclohexyl or cyclohex-2-enyl group.

The expression cycloheteroalkyl or heterocycle refers to a cycloalkyl group as defined above in which one or more (e.g., 1, 2, or 3) ring carbon atoms have been replaced by an oxygen, nitrogen, silicon, selenium, phosphorus or sulfur atom or a SO group or a SO₂ group. A cycloheteroalkyl or heterocycle group may have 1 or 2 rings containing from 3 to 10 (e.g., 3, 4, 5, 6 or 7) ring atoms (e.g., C, O, N or S). Cycloheteroalkyl or heterocycle groups include cycloheteroalkenyl or heterocycloalkenyl groups. The expression cycloheteroalkyl or heterocycle refers furthermore to groups that are substituted by fluorine, chlorine, bromine or iodine atoms or by OH, ═O, SH, ═S, NH₂, ═NH, N₃ or NO₂ groups. Examples are a piperidinyl, prolinyl, imidazolidinyl, piperazinyl, morpholinyl, urotro pinyl, pyrrolidinyl, tetrahydrothiophenyl, tetrahydropyranyl, tetrahydrofuryl or 2-pyrazolinyl group and also lactams, lactones, cyclic imides and cyclic anhydrides.

The expression alkylcycloalkyl refers to groups that contain both cycloalkyl and also alkyl, alkenyl or alkynyl groups in accordance with the above definitions, for example alkylcycloalkyl, cycloalkylalkyl, alkylcycloalkenyl, alkenylcycloalkyl and alkynylcycloalkyl groups. An alkylcycloalkyl group preferably contains a cycloalkyl group that contains one or two rings having from 3 to 10 (e.g., 3, 4, 5, 6 or 7) ring carbon atoms, and one or two alkyl or alkynyl groups having 1 or 2 to 6 carbon atoms.

The expression heteroalkylcycloalkyl refers to alkylcycloalkyl groups as defined above in which one or more (e.g., 1, 2 or 3) carbon atoms have been replaced by an oxygen, nitrogen, silicon, selenium, phosphorus or sulfur atom or a SO group or a SO₂ group. A heteroalkylcycloalkyl group preferably contains 1 or 2 rings having from 3 to 10 (e.g., 3, 4, 5, 6 or 7) ring atoms, and one or two alkyl, alkenyl, alkynyl or heteroalkyl groups having from 1 or 2 to 6 carbon atoms. Examples of such groups are alkylheterocycle, alkylheterocycloalkenyl, alkenylheterocycle, alkynylheterocycle, heteroalkylcycloalkyl, heteroalkylheterocycle and heteroalkylheterocycloalkenyl, the cyclic groups being saturated or mono-, di- or tri-unsaturated.

The expression aryl refers to an aromatic group that contains one or more rings, e.g., 2 or 3 rings, containing from 6 to 14 ring carbon atoms, such as from 6 to 10 ring carbon atoms. The expression aryl refers furthermore to groups that are substituted by fluorine, chlorine, bromine or iodine atoms or by CH₃, OH, SH, NH₂, N₃ or NO₂ groups. Examples are the phenyl, naphthyl, biphenyl, 2-fluorophenyl, anilinyl, 3-nitrophenyl or 4-hydroxyphenyl group.

The expression heteroaryl refers to an aromatic group that contains one or more rings, e.g., 2 or 3 rings, containing from 5 to 14 ring atoms, such as from 5 to 10 ring atoms, and contains one or more (e.g., 1, 2, 3 or 4) oxygen, nitrogen, phosphorus or sulfur ring atoms. The expression heteroaryl refers furthermore to groups that are substituted by fluorine, chlorine, bromine or iodine atoms or by CH₃, OH, SH, N₃, NH₂ or NO₂ groups. Examples are pyridyl (e.g. 4-pyridyl), imidazolyl (e.g. 2-imidazolyl), phenylpyrrolyl (e.g. 3-phenylpyrrolyl), thiazolyl, isothiazolyl, 1,2,3-triazolyl, 1,2,4-triazolyl, oxadiazolyl, thiadiazolyl, indolyl, indazolyl, tetrazolyl, pyrazinyl, pyrimidinyl, pyridazinyl, oxazolyl, isoxazolyl, triazolyl, tetrazolyl, isoxazolyl, indazolyl, indolyl, benzimidazolyl, benzoxazolyl, benzisoxazolyl, benzthiazolyl, pyridazinyl, quinolinyl, isoquinolinyl, pyrrolyl, purinyl, carbazolyl, acridinyl, pyrimidyl, 2,3′-bifuryl, pyrazolyl (e.g. 3-pyrazolyl) and isoquinolinyl groups.

The expression aralkyl refers to groups containing both aryl and also alkyl, alkenyl, alkynyl and/or cycloalkyl groups in accordance with the above definitions, such as, for example, aryl-alkyl, arylalkenyl, arylalkynyl, arylcycloalkyl, arylcycloalkenyl, alkylarylcycloalkyl and alkylarylcycloalkenyl groups. Specific examples of aralkyls are toluene, xylene, mesitylene, styrene, benzyl chloride, o-fluorotoluene, 1H-indene, tetraline, dihydronaphthalene, indanone, phenylcyclopentyl, cumene, cyclohexylphenyl, fluorene and indane. An aralkyl group preferably contains one or two aromatic ring systems containing from 6 to 10 carbon atoms and one or two alkyl, alkenyl and/or alkynyl groups containing from 1 or 2 to 6 carbon atoms and/or a cycloalkyl group containing 5 or 6 ring carbon atoms.

The expression heteroaralkyl refers to an aralkyl group as defined above in which one or more (e.g., 1, 2, 3 or 4) carbon atoms have been replaced by an oxygen, nitrogen, silicon, selenium, phosphorus, boron or sulfur atom, that is to say to groups containing both aryl or heteroaryl, respectively, and also alkyl, alkenyl, alkynyl and/or heteroalkyl and/or cycloalkyl and/or heterocycle groups in accordance with the above definitions. A heteroaralkyl group preferably contains one or two aromatic ring systems containing from 5 or 6 to 10 ring carbon atoms and one or two alkyl, alkenyl and/or alkynyl groups containing 1 or 2 to 6 carbon atoms and/or a cycloalkyl group containing 5 or 6 ring carbon atoms, wherein 1, 2, 3 or 4 of these carbon atoms have been replaced by oxygen, sulfur or nitrogen atoms.

Examples are arylheteroalkyl, arylheterocycle, arylheterocycloalkenyl, arylalkyl heterocycle, arylalkenylheterocycle, arylalkynylheterocycle, arylalkylhetero cycloalkenyl, heteroarylalkyl, heteroarylalkenyl, heteroarylalkynyl, heteroarylheteroalkyl, heteroarylcycloalkyl, heteroarylcycloalkenyl, heteroarylheterocycle, hetero arylheterocycloalkenyl, heteroarylalkylcycloalkyl, heteroarylalkylheterocycloalkenyl, hetero arylheteroalkylcycloalkyl, heteroarylheteroalkylcycloalkenyl and heteroarylheteroalkylhetero cycloalkyl groups, the cyclic groups being saturated or mono-, di- or tri-unsaturated. Specific examples are a tetrahydroisoquinolinyl, benzoyl, 2- or 3-ethylindolyl, 4-methylpyridino, 2-, 3- or 4-methoxyphenyl, 4-ethoxyphenyl, 2-, 3- or 4-carboxyphenylalkyl group.

As stated above, the expressions cycloalkyl, cycloheteroalkyl, heterocycle, alkylcycloalkyl, heteroalkylcycloalkyl, aryl, heteroaryl, aralkyl and heteroaralkyl also refer to groups that are substituted by fluorine, chlorine, bromine or iodine atoms or by CH₃, OH, ═O, SH, ═S, NH₂, ═NH, N₃ or NO₂ groups.

The expression carbocyclyl or carbocyclic refers to a radical of a non-aromatic cyclic hydrocarbon group having from 3 to 10 ring carbon atoms (“C₃₋₁₀ carbocyclyl”) and zero heteroatoms in the nonaromatic ring system. In some embodiments, a carbocyclyl group has 3 to 8 ring carbon atoms 10 (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (G), cyclooctenyl (G), bicyclo[2.2.1]heptanyl (C₇), bicyclo[2.2.2]octanyl (G), and the like. Exemplary C₃₋₁₀ carbocyclyl groups include, without 20 limitation, the aforementioned G-s carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or contain a fused, bridged or spiro ring system such as a bicyclic system (“bicyclic carbocyclyl”) and can be saturated or can be partially unsaturated. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently optionally substituted, i.e., unsubstituted (an “unsubstituted carbocyclyl”) or substituted (a “substituted carbocyclyl”) with one or more substituents. In certain embodiments, the carbocyclyl group is unsubstituted C₃₋₁₀ carbocyclyl. In certain embodiments, the carbocyclyl group is a substituted C₃₋₁₀ carbocyclyl.

In some embodiments, “carbocyclyl” is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C₃₋₁₀ cycloalkyl. In certain embodiments, the cycloalkyl group is substituted C₃₋₁₀ cycloalkyl.

The expression heterocyclyl or heterocyclic refers to a radical of a 3- to 14-membered non-aromatic ring system having ring carbon atoms and 1 to 4 ring heteroatoms, wherein each heteroatom is independently selected from nitrogen, oxygen, sulfur, boron, phosphorus, and silicon (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, sulfur, boron, phosphorus, and silicon (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1¬4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups 5 containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

The expression optionally substituted means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction.

Heteroatoms, such as nitrogen, may have substituents, such as any suitable substituent described herein which satisfies the valencies of the heteroatoms and results in the formation of a stable moiety.

For example and without limitation, optional substituents include fluorine, chlorine, bromine, and iodine atoms and CF₃, CN, OH, ═O, SH, ═S, NH₂, ═NH, N₃ and NO₂ groups. Optional substituents also include C₁-C₁₀ alkyl, C₂-C₁₀ alkenyl, C₁-C₁₀ heteroalkyl, C₃-C₁₆ cycloalkyl, C₂-C₁₇ heterocycle, C₄-C₂₀ alkylcycloalkyl, C₂-C₁₉ heteroalkylcycloalkyl, C₆-C₁₈ aryl, C₁₋₁₇ heteroaryl, C₇-C₂₀ aralkyl or C₂-C₁₉ heteroaralkyl, C₁-C₆ alkyl, C₂-C₆ alkenyl, C₁-C₆ heteroalkyl, C₃-C₁₀ cycloalkyl, C₂-C₉ heterocycle, C₇-C₁₂ alkylcycloalkyl, C₂-C₁₁ heteroalkylcycloalkyl, C₆-C₁₀ aryl, C₁-C₉ heteroaryl, C₇-C₁₂ aralkyl, C₂-C₁₁ heteroaralkyl, and C₁-C₁₀ haloalkyl groups.

Exemplary substituents are F, Cl, Br, OH, SH, ═O, NH₂, amino, C₁₋₄ alkyl, C₁₋₄ heteroalkyl cyclopropyl, SF₅, NO, NO₂.

Other exemplary substituents are F, Cl, Br, OH, SH, ═O, NH₂, C₁₋₄ alkyl (e.g. methyl, ethyl, t-butyl), NMe₂, CONH₂, CH₂NMe₂, NHSO₂Me, C(CH₃)₂CN, COMe, OMe, SMe, COOMe, COOEt, CH₂COOH, OCH₂COOH, COOH, SOMe, SO₂Me, cyclopropyl, SO₂NH₂, SO₂NHMe, SO₂CH₂CH₂OH, NHCH₂CH₂OH, CH₂CH₂OCH₃, SF₅, SO₂NMe₂, NO, NO₂, OCF₃, SO₂CF₃, CN or CF₃.

Other exemplary substituents are F, Cl, Br, Me, OMe, CN or CF₃.

The term halogen preferably refers to F, Cl, Br or I.

When an aryl, heteroaryl, cycloalkyl, alkylcycloalkyl, heteroalkylcycloalkyl, heterocycle, aralkyl or heteroaralkyl group contains more than one ring, these rings may be bonded to each other via a single or double bond or these rings may be annulated.

According to certain embodiments, all alkyl, alkenyl, alkynyl, heteroalkyl, aryl, heteroaryl, cycloalkyl, heterocycle, alkylcycloalkyl, heteroalkylcycloalkyl, aralkyl and heteroaralkyl groups described herein may optionally be substituted.

Other optional substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, ═SR^{aa}, —SSR^{cc}, —C(O)R^{aa}, —CO₂H, —CHO, —C(OR^{cc})₂, —CO₂R^{aa}, —OC(O)R^{aa}, —OCO₂R^{aa}, —C(O)N(R^{bb})₂, —C(O)N(R^{aa})(R^{bb}), —OC(O)N(R^{bb})₂, —NR^{bb}C(O)R^{aa}, NR^{bb}CO₂R^{aa}, —NR^{bb}C(O)N(R^{bb})₂—C(NR^{bb})R^{aa}, —C(NR^{bb})OR^{aa}, —OC(NR^{bb})R^{aa}, —OC(NR^{bb})OR^{aa}, —C(NR^{bb})N(R^{bb})₂, —OC(NR^{bb})N(R^{bb})₂, —NR^{bb}C(NR^{bb})N(R^{bb})₂, —C(O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(O)R^{aa}, e.g., —S(O)R^{aa}, —OS(O)R^{aa}, ═Si(R^{aa})₃, —OSi(R^{aa})₃—C(S)N(R^{bb})₂, —C(O)SR^{aa}, —C(S)SR^{aa}, —SC(S)SR^{aa}, —SC(O)SR^{aa}, —OC(O)SR^{aa}, —SC(O)OR^{aa}, —SC(O)R^{aa}, —P(O)₂R^{aa}, —OP(O)₂R^{aa}, —P(O)(R^{aa})₂, —OP(O)(R^{aa})₂, —OP(O)(OR^{cc})₂, —P(O)₂N(R^{bb})₂, —OP(O)₂N(R^{bb})₂, —P(O)(NR^{bb})₂, —OP(O)(NR^{bb})₂, —NR^{bb}P(O)(OR)₂, —NR^{bb}P(O)(NR^{bb})₂, —P(R^{cc})₂, —P(R^{cc})₃, —OP(R^{cc})₂, —OP(R^{cc})₃, —B(R^{aa})₂, —B(OR^{cc})₂, —BR^{aa}(OR^{cc}), C₁₋₁₀ alkyl, C₁₋₁₀ haloalkyl, C₂₋₁₀ alkenyl, C₃₋₁₀ carbocyclyl, 3-14 membered heterocyclyl, C₆₋₁₄ aryl, and 5-14 membered heteroaryl, wherein each alkyl, alkenyl, carbocyclyl, heterocyclyl, aryl, and heteroaryl is independently substituted with 0, 1, 2, 3, 4, or 5 R^{dd} groups; or two geminal hydrogens on a carbon atom are replaced with the group ═O, ═S, ═NN(R^{bb})₂, —NNR^{bb}C(O)R^{aa}, —NNR^{bb}C(O)OR^{aa}, —NNR^{bb}S(O)₂R^{aa}, ═NR^{bb}, or ═NOR^{cc}; in which: each instance of R^{aa} is, independently, selected from C₁₋₁₀ alkyl, C₁₋₁₀ heteroalkyl, C₁₋₁₀ haloalkyl, C₂₋₁₀ alkenyl, C₃₋₁₀ cycloalkyl, C₃₋₁₀ cycloheteroalkyl, C₃₋₁₀ cycloalkenyl, C₃₋₁₀ cycloheteroalkenyl, C₃₋₁₀ carbocyclyl, 3-14 membered heterocyclyl, C₆₋₁₄ aryl, and 5-14 membered heteroaryl, or two R^{aa} groups are joined to form a 3-14 membered cycloalkyl, 3-14 membered cycloheteroalkyl, 3-14 membered heterocyclyl or 5-14 membered heteroaryl ring, wherein each alkyl, heteroalkyl, alkenyl, cycloalkyl, cycloheteroalkyl, cycloalkenyl, cycloheteroalkenyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{cc})², —CN, —C(O)R^{aa}, —C(O)N(R^{cc})₂, —CO₂R^{aa}, —SO₂R^{aa}, —C(NR^{cc})OR^{aa}, —C(NR^{cc})N(R^{cc})₂, —SO₂N(R^{cc})₂, —SO₂R^{cc}, —SO₂OR^{cc}, ═SOR^{aa}, —C(S)N(R^{cc})₂, —C(O)SR^{cc}, —C(S)SR^{cc}, —P(O)₂R^{aa}, —P(O)(R^{aa})₂, —P(O)₂N(R^{cc})₂, —P(O)(NR^{cc})₂, C₁₋₁₀ alkyl, C₁₋₁₀ heteroalkyl, C₁₋₁₀ haloalkyl, C₂₋₁₀ alkenyl, C₃₋₁₀ cycloalkyl, C₃₋₁₀ cycloheteroalkyl, C₃₋₁₀ cycloalkenyl, C₃₋₁₀ cycloheteroalkenyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, heteroalkyl, alkenyl, cycloalkyl, cycloheteroalkyl, cycloalkenyl, cycloheteroalkenyl, carbocyclyl, heterocyclyl, aryl, and heteroaryl is independently substituted with 0, 1, 2, 3, 4, or 5 R^{dd} groups;
each instance of RC is, independently, selected from hydrogen, C₁₋₁₀ alkyl, C₁₋₁₀ haloalkyl, C₂₋₁₀ alkenyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(Rn); CX˜, —N(OR^{ee})R^{ff}, —SH, ═SR^{ee}, ═SSR^{ee}, —C(O)R^{ee}, —CO₂H, —CO₂R^{ee}, —OC(O)R^{ee}, —OCO₂R^{ee}, —C(O)N(R^{ff})₂, —OC(O)N(R^{ff})₂, —NR^{ff}C(O)R^{ee}, —NR^{ff}CO₂R^{ee}, —NR^{ff}C(O)N(RN)₂, —C(NR^{ff})OR^{ee}, —OC(NR^{ff})R^{ee}, —OC(NR)OR^{ee}, —C(NR^{ff})N(R^{ff})₂, —OC(NR^{ff})N(R^{ff})₂, —NR^{ff}C(NR^{ff})N(RN)₂, —NR^{ff}SO₂R^{ee}, —SO₂N(R^{ee})₂, —SO₂R^{ee}, —SO₂OR^{ee}, —OSO₂R^{ee}, —S(O)R^{ee}, e.g., —S(O)R^{cc}, —Si(R^{ee})₃, —OSi(R^{ee})₃, —C(S)N(R^{ff})₂, —C(O)SR^{ee}, —C(S)SR^{ee}, —SC(S)SR^{ee}, —P(O)₂R^{ee}, —P(O)(R^{ee})₂, —OP(O)(R^{ee})₂, —OP(O)(OR^{ee})₂, C₁₋₆ alkyl, C₁₋₆ heteroalkyl, C₁₋₆ haloalkyl, C₂₋₆ alkenyl, C₃₋₁₀ carbocyclyl, 3-10 membered heterocyclyl, C₆₋₁₀ aryl, 5-10 membered heteroaryl, wherein each alkyl, alkenyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S;
each instance of R^{ee} is, independently, selected from C₁₋₆ alkyl, C₁₋₆ haloalkyl, C₂₋₆ alkenyl, C₃₋₁₀ carbocyclyl, 3-10 membered heterocyclyl, C₆₋₁₀ aryl, 5-10 membered heteroaryl, wherein each alkyl, alkenyl, carbocyclyl, heterocyclyl, aryl, and heteroaryl is independently substituted with 0, 1, 2, 3, 4, or 5 R^{gg} groups;
each instance of R^{ff} is, independently, selected from hydrogen, C₁₋₆ alkyl, C₁₋₆ haloalkyl, C₂₋₆ alkenyl, C₃₋₁₀ carbocyclyl, 3-10 membered heterocyclyl, C₆₋₁₀ aryl, 5-10 membered heteroaryl, or two R^{ff} groups are joined to form a 3-14 membered heterocyclyl or 5-14 membered heteroaryl ring, wherein each alkyl, alkenyl, carbocyclyl, heterocyclyl, aryl, and heteroaryl is independently substituted with 0, 1, 2, 3, 4, or 5 R^{gg} groups; and
each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻-MR⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(O)(C₁₋₆ alkyl), —CO₂H, —CO₂(C₁₋₆ alkyl), —OC(O)(C₁₋₆ alkyl), —OCO₂(C₁₋₆ alkyl), —C(O)NH₂, —C(O)N(C₁₋₆ alkyl)₂, —OC(O)NH(C₁₋₆ alkyl), —NHC(O)(C₁₋₆ alkyl), —N(C₁₋₆ alkyl)C(O)(C₁₋₆ alkyl), —NHCO₂(C₁₋₆ alkyl), —NHC(O)N(C₁₋₆ alkyl)₂, —NHC(O)NH(C₁₋₆ alkyl), —NHC(O)NH₂, —C(NH)O(C₁₋₆ alkyl), —OC(NH)(C₁₋₆ alkyl), —OC(NH)OC₁₋₆ alkyl, —C(NH)N(C₁₋₆ alkyl)₂, —C(NH)NH(C₁₋₆ alkyl), —C(NH)NH₂, —OC(NH)N(C₁₋₆ alkyl)₂, —OC(NH)NH(C₁₋₆ alkyl), —OC(NH)NH₂, —NHC(NH)N(C₁₋₆ alkyl)₂, —NHC(NH)NH₂, —NHSO₂(C₁₋₆ alkyl), —SO₂N(C₁₋₆ alkyl)₂, ═SO₂NH(C₁₋₆ alkyl), ═SO₂NH₂, —SO₂C₁₋₆ alkyl, —SO₂OC₁₋₆ alkyl, —OSO₂C₁₋₆ alkyl, —SOC₁₋₆ alkyl, ═Si(C₁₋₆ alkyl)₃, —OSi(C₁₋₆ alkyl)₃-C(S)N(C₁₋₆ alkyl)₂, C(S)NH(C₁₋₆ alkyl), C(S)NH₂, —C(O)S(C₁₋₆ alkyl), —C(S)SC₁₋₆ alkyl, —SC(S)SC₁₋₆ alkyl, —P(O)₂(C₁₋₆ alkyl), —P(O)(C₁₋₆ alkyl)₂, —OP(O)(C₁₋₆ alkyl)₂, —OP(O)(OC₁₋₆ alkyl)₂, C₁₋₆ alkyl, C₁₋₆ haloalkyl, C₂₋₆ alkenyl, C₃₋₁₀ carbocyclyl, C₃₋₁₀ aryl, 3-10 membered heterocyclyl, 5-10 membered heteroaryl; or two geminal R^{gg} substituents can be joined to form ═O or ═S; wherein X⁻ is a counterion.

### Compounds

In one aspect, the invention provides a compound of Formula (I):

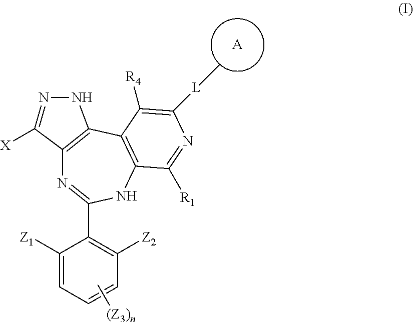

or an enantiomer, mixture of enantiomers, tautomer, or pharmaceutically acceptable salt thereof,
wherein:
n is 1, 2, or 3;
Y is N or CR₂;
Z₁, Z₂, and Z₃ are independently selected from H, —OH, halo, cyano, amino, C₁-C₆ alkyl, C₁-C₆ heteroalkyl, haloalkyl, alkoxy, haloalkxoxy, —CH(OH)-alkyl, hydroxyalkyl, or hydroxyalkoxy;
X is H, halo, cyano, cycloalkyl, C₁-C₆ alkyl, optionally deuterated C₁-C₆ alkyl, C₁-C₆ heteroalkyl, haloalkyl, alkoxy, haloalkxoxy, —CH(OH)-alkyl, hydroxyalkyl, or hydroxyalkoxy; R₁, R², and R⁴ are independently selected from H, halo, cyano, C₁-C₆ alkyl, optionally deuterated C₁-C₆ alkyl, C₁-C₆ heteroalkyl, haloalkyl, alkoxy, haloalkxoxy, —CH(OH)-alkyl, hydroxyalkyl, or hydroxyalkoxy;
L is a linker, wherein L is a single bond, substituted or unsubstituted C₁-C₄ alkyl, substituted or unsubstituted C₁-C₄ heteroalkyl, wherein the one or more heteroatoms are selected from O, S, or N;
A is 4-8 membered substituted or unsubstituted heterocycloalkyl, spiroheterocycloalkyl, heteroaryl, wherein one or more heteroatoms are selected from a group consisting of O, S, or N; and
wherein the substituents may be selected from a group consisting of substituted or unsubstituted 3-7 membered heterocycle, ═O, —CH₂-cycloalkyl, —CF₂-cycloalky, —C(═O)—O-alkyl, halo, deuterium, cyano, cyanoalkyl, —CF₃, mono-, di-, or tri-halo alkyl, CH(CH₃)-cycloalkyl, —CH₂-aryl, —CF₂-aryl, —CH(—CH₃)-aryl, C(═O)-alkyl, —C(═O)cycloalkyl, —C(═O)—NH-alkyl, —COOH (and esters and carboxamides thereof), —C(═O)-morpholine, —C(═O)-heterocycles, —C(—CH₃)₂—OH, —CH₂—C(═O)—NH₂; -hydroxy, alkylhydroxy, alkyl-COOH (and esters and carboxamides thereof), amino, NHC(═O)alkyl, —N(alkyl)C(═O)alkyl, —NHC(═O)aryl, —N(alkyl)C(═O)aryl, —N(H)S(═O)₂alkyl, —N(H)S(═O)₂aryl, —N(alkyl)S(═O)₂alkyl, cyanoalkyl, haloalkyl, ═S═(O)₂-alkyl, ═S═(O)₂-cycloalkyl, ═S═(O)₂-aryl, —S(═O)₂N(H)alkyl, substituted or unsubstituted morpholine, 3-7 membered heterocycle, any of which may have one or more substituents, 3-7 membered cycloalkyl or heterocycle, wherein the 3-7 membered cycloalkyl or heterocycle is optionally fused with another 3-7 cycloalkyl or heterocycle, wherein the rings are spiro, bridged bicyclic, or spiro, wherein the at least one heteroatom in the heterocycle rings are independently selected from O, S, and N; and
wherein one or more hydrogen atoms are optionally deuterium.

In another embodiment, Y is N.

In another embodiment, Y is CR₂.

In another embodiment, Z₁ and Z₂ are F.

In another embodiment, Z₁ and Z₂ are independently selected from Cl or F.

In another embodiment, Z₃ is H.

In another embodiment, Z₃ is F.

In another embodiment, Z₃ is —CH₂—OH.

In another embodiment, X is selected from a group consisting of methyl, H, Cl, or —CD₃.

In another embodiment, X is methyl.

In another embodiment, R₁ is selected from H or methyl.

In another embodiment, R₂ is H.

In another embodiment, R₄ is H.

In another embodiment, L is a single bond.

In another embodiment, L is alkyl.

In another embodiment, A is selected from:

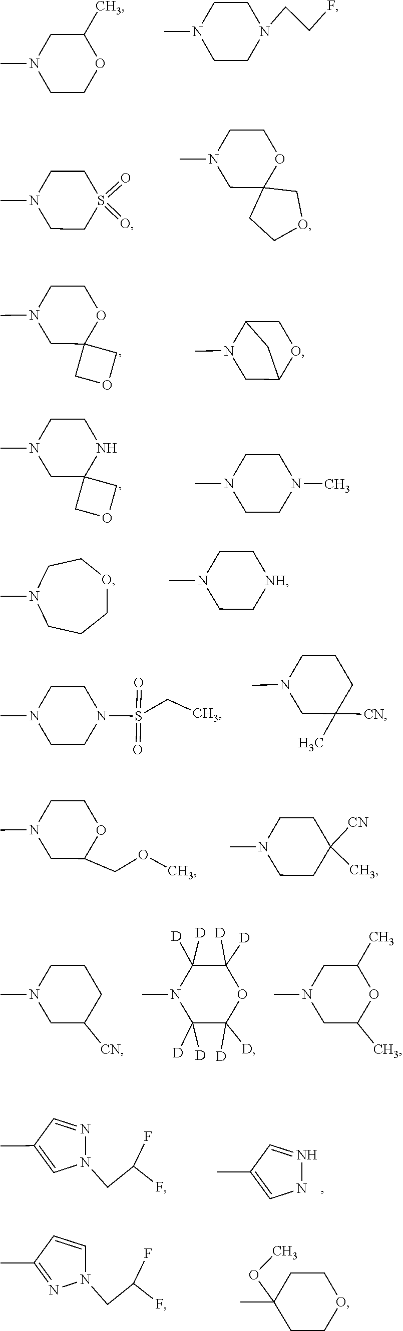

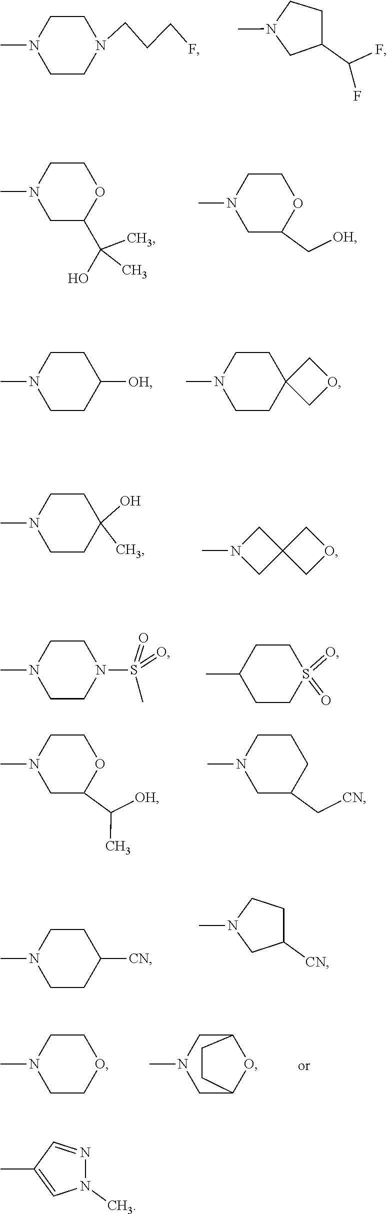

In another embodiment, the compound of Formula (I) is selected from:

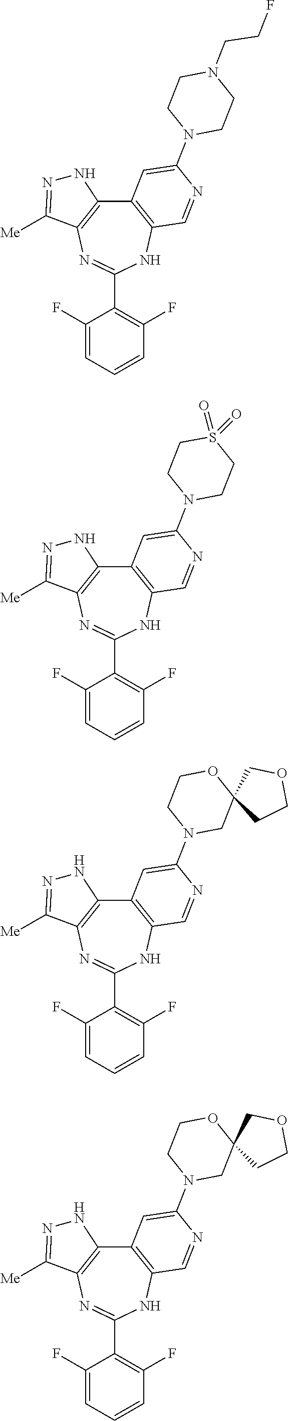 [SMILES: Cc1n[nH]c2c1N=C(c1c(F)cccc1F)Nc1cnc(N3CCN(CCF)CC3)cc1-2 ; Cc1n[nH]c2c1N=C(c1c(F)cccc1F)Nc1cnc(N3CCS(=O)(=O)CC3)cc1-2 ; Cc1n[nH]c2c1N=C(c1c(F)cccc1F)Nc1cnc(N3CCO[C@@]4(CCOC4)C3)cc1-2 ; Cc1n[nH]c2c1N=C(c1c(F)cccc1F)Nc1cnc(N3CCO[C@]4(CCOC4)C3)cc1-2]

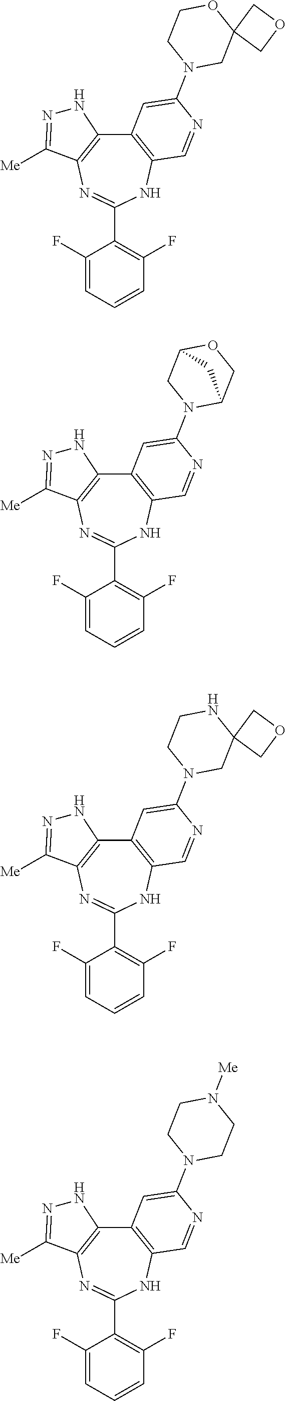 [SMILES: Cc1n[nH]c2c1N=C(c1c(F)cccc1F)Nc1cnc(N3CCOC4(COC4)C3)cc1-2 ; Cc1n[nH]c2c1N=C(c1c(F)cccc1F)Nc1cnc(N3C[C@@H]4C[C@H]3CO4)cc1-2 ; Cc1n[nH]c2c1N=C(c1c(F)cccc1F)Nc1cnc(N3CCNC4(COC4)C3)cc1-2 ; Cc1n[nH]c2c1N=C(c1c(F)cccc1F)Nc1cnc(N3CCN(C)CC3)cc1-2]

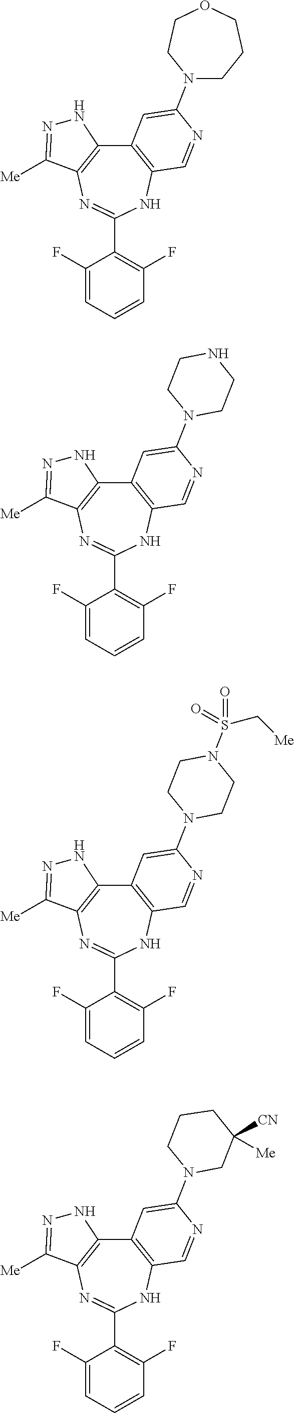 [SMILES: Cc1n[nH]c2c1N=C(c1c(F)cccc1F)Nc1cnc(N3CCCOCC3)cc1-2 ; Cc1n[nH]c2c1N=C(c1c(F)cccc1F)Nc1cnc(N3CCNCC3)cc1-2 ; CCS(=O)(=O)N1CCN(c2cc3c(cn2)NC(c2c(F)cccc2F)=Nc2c(C)n[nH]c2-3)CC1 ; Cc1n[nH]c2c1N=C(c1c(F)cccc1F)Nc1cnc(N3CCC[C@@](C)(C#N)C3)cc1-2]

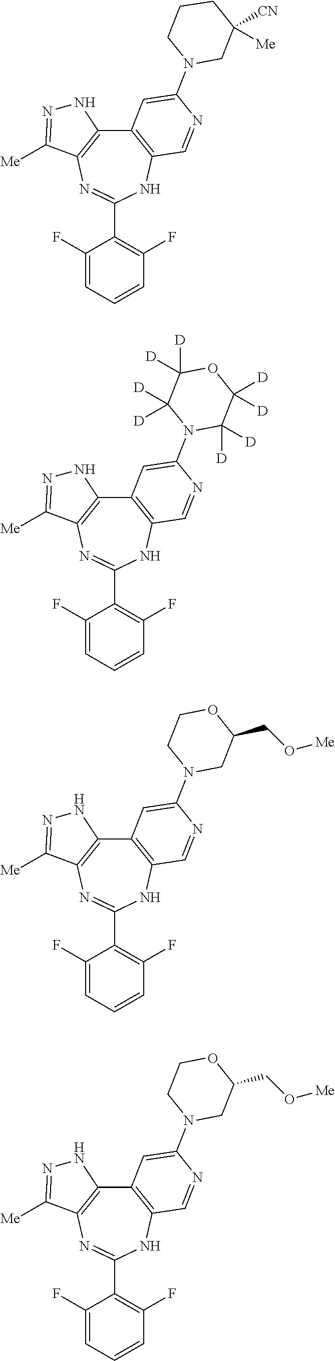 [SMILES: Cc1n[nH]c2c1N=C(c1c(F)cccc1F)Nc1cnc(N3CCC[C@](C)(C#N)C3)cc1-2 ; [2H]C1([2H])OC([2H])([2H])C([2H])([2H])N(c2cc3c(cn2)NC(c2c(F)cccc2F)=Nc2c(C)n[nH]c2-3)C1([2H])[2H] ; COC[C@H]1CN(c2cc3c(cn2)NC(c2c(F)cccc2F)=Nc2c(C)n[nH]c2-3)CCO1 ; COC[C@@H]1CN(c2cc3c(cn2)NC(c2c(F)cccc2F)=Nc2c(C)n[nH]c2-3)CCO1]

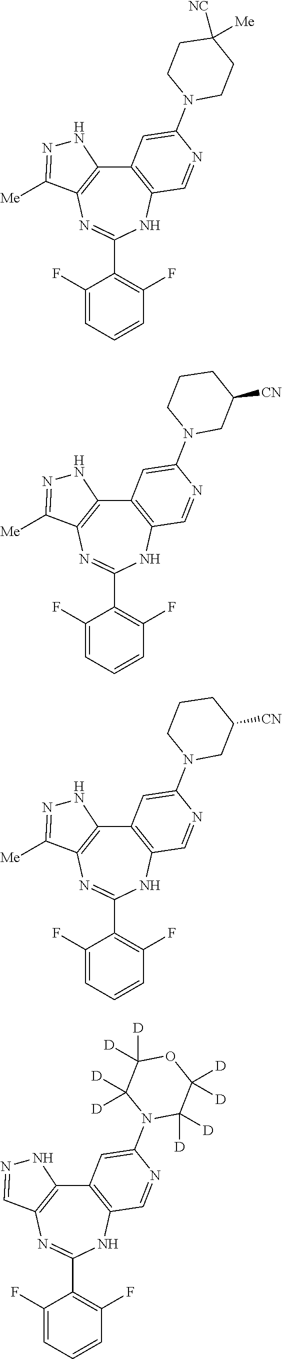

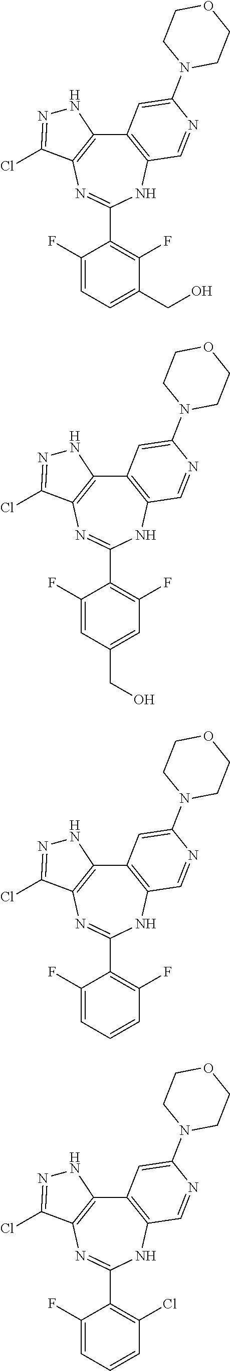

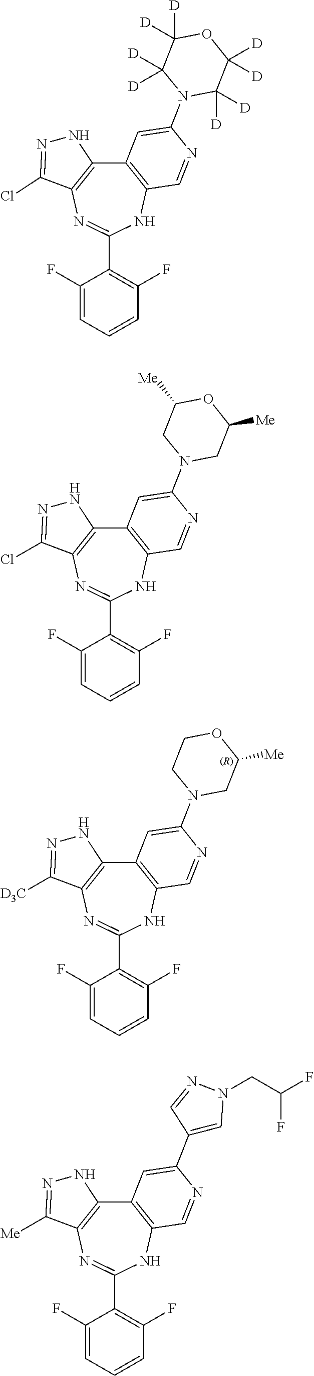

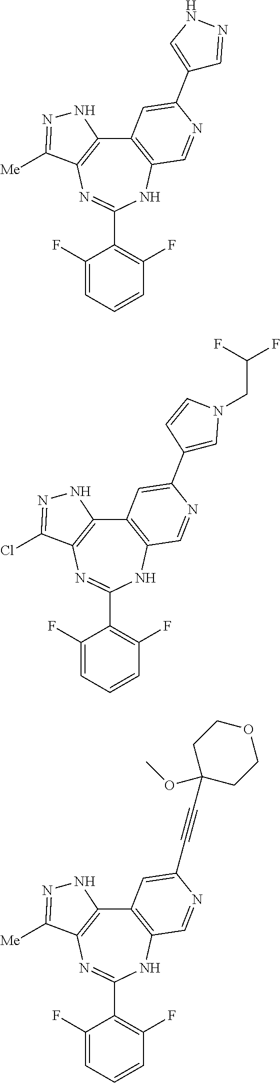 [SMILES: Cc1n[nH]c2c1N=C(c1c(F)cccc1F)Nc1cnc(-c3cn[nH]c3)cc1-2 ; Fc1cccc(F)c1C1=Nc2c(Cl)n[nH]c2-c2cc(-c3ccn(CC(F)F)c3)ncc2N1 ; COC1(C#Cc2cc3c(cn2)NC(c2c(F)cccc2F)=Nc2c(C)n[nH]c2-3)CCOCC1]

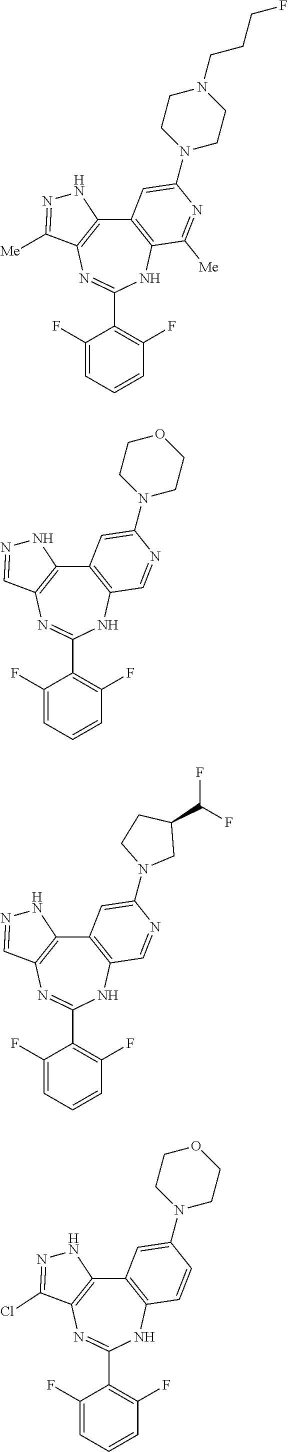 [SMILES: Cc1n[nH]c2c1N=C(c1c(F)cccc1F)Nc1c-2cc(N2CCN(CCCF)CC2)nc1C ; Fc1cccc(F)c1C1=Nc2cn[nH]c2-c2cc(N3CCOCC3)ncc2N1 ; Fc1cccc(F)c1C1=Nc2cn[nH]c2-c2cc(N3CC[C@@H](C(F)F)C3)ncc2N1 ; Fc1cccc(F)c1C1=Nc2c(Cl)n[nH]c2-c2cc(N3CCOCC3)ccc2N1]

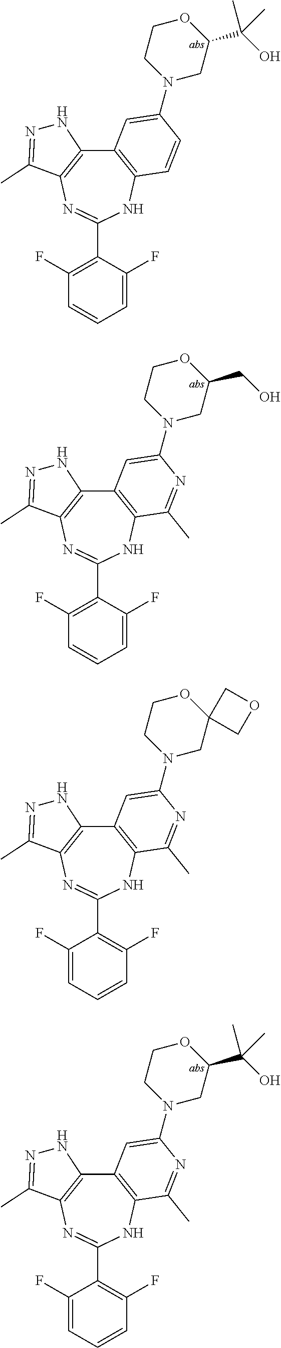

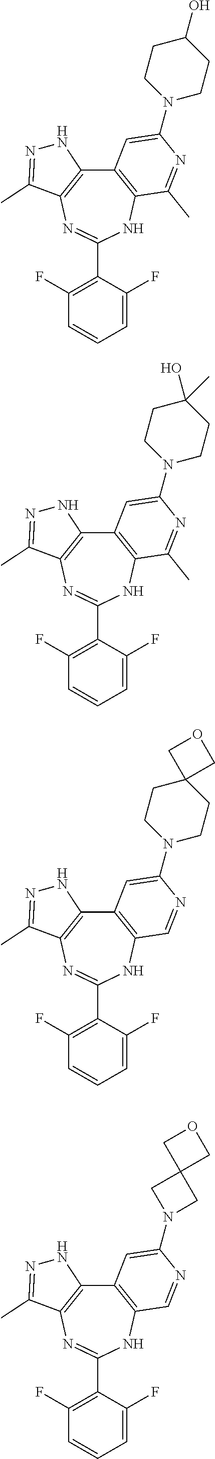

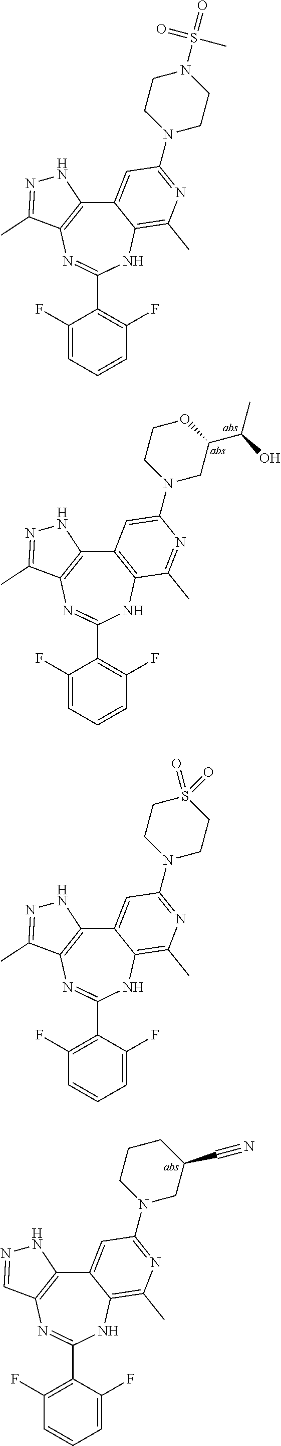

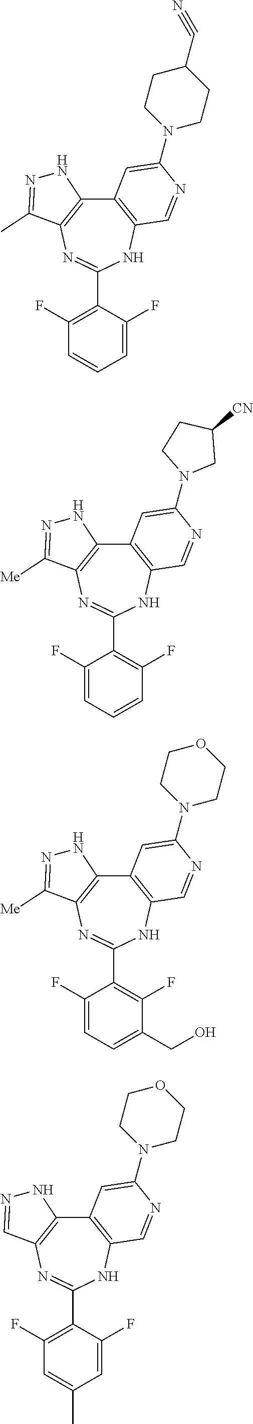

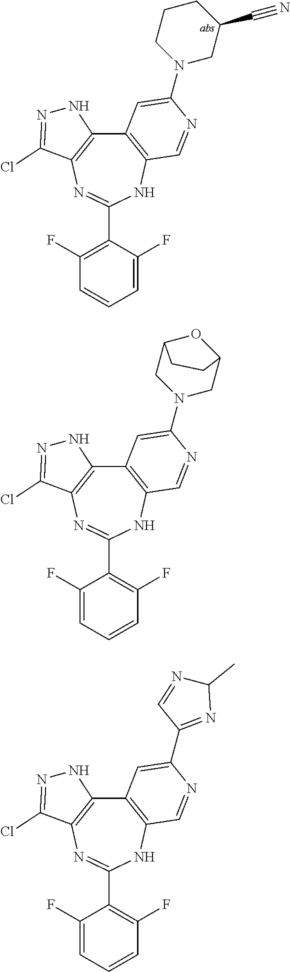 [SMILES: N#C[C@@H]1CCCN(c2cc3c(cn2)NC(c2c(F)cccc2F)=Nc2c(Cl)n[nH]c2-3)C1 ; Fc1cccc(F)c1C1=Nc2c(Cl)n[nH]c2-c2cc(N3CC4CCC(C3)O4)ncc2N1 ; CC1N=CC(c2cc3c(cn2)NC(c2c(F)cccc2F)=Nc2c(Cl)n[nH]c2-3)=N1]

### Pharmaceutical Compositions

The present invention provides pharmaceutical compositions containing one or more compounds described above, or a pharmaceutically acceptable ester, prodrug, hydrate, solvate or salt of such a compound, optionally in combination with a pharmaceutically acceptable carrier. The invention further provides such compounds for the preparation of a medicament for the treatment of one or more diseases mentioned herein.

A pharmaceutical composition may contain one or more compounds of the invention in a therapeutically effective amount. A therapeutically effective amount of a compound in accordance with this invention means an amount of compound that is effective to prevent, alleviate or ameliorate symptoms of disease or prolong the survival of the subject being treated. Determination of a therapeutically effective amount is within the skill in the art.

The therapeutically effective amount or dosage of a compound according to this invention can vary within wide limits and may be determined in a manner known in the art. Such dosage may be adjusted to the individual requirements in each particular case including the specific compound being administered, the route of administration, the condition being treated, as well as the patient being treated.

Compositions of the invention may include a vehicle for delivery of one or more compounds of the invention. For example, the composition may contain particles, such as nanoparticles, microparticles, liposomes, micelles, and virus particles.

Examples of pharmacologically acceptable salts of sufficiently basic compounds of the invention are salts of physiologically acceptable mineral acids like hydrochloric, hydrobromic, sulfuric and phosphoric acid; or salts of organic acids like methanesulfonic, p-toluenesulfonic, lactic, acetic, trifluoroacetic, citric, succinic, fumaric, maleic and salicylic acid. Further, a sufficiently acidic compound of the invention may form alkali or earth alkali metal salts, for example sodium, potassium, lithium, calcium or magnesium salts; ammonium salts; or organic base salts, for example methylamine, dimethylamine, trimethylamine, triethylamine, ethylenediamine, ethanolamine, choline hydroxide, meglumin, piperidine, morpholine, tris-(2-hydroxyethyl)amine, lysine or arginine salts; all of which are also further examples of salts of the invention. Compounds of the invention may be solvated, especially hydrated. The hydratization/hydration may occur during the process of production or as a consequence of the hygroscopic nature of the initially water free compounds of the invention. The solvates and/or hydrates may e.g. be present in solid or liquid form.

It should be appreciated that certain compounds of the invention may have tautomeric forms from which only one might be specifically mentioned or depicted in the following description, different geometrical isomers (which are usually denoted as cis/trans isomers or more generally as (E) and (Z) isomers) or different optical isomers as a result of one or more chiral carbon atoms (which are usually nomenclatured under the Cahn-Ingold-Prelog or R/S system). All these tautomeric forms, geometrical or optical isomers (as well as racemates and diastereomers) and polymorphous forms are included in the invention. Since the compounds of the invention may contain asymmetric C-atoms, they may be present either as achiral compounds, mixtures of diastereomers, mixtures of enantiomers or as optically pure compounds. The present invention comprises both all pure enantiomers and all pure diastereomers, and also the mixtures thereof in any mixing ratio.

According to a further embodiment of the present invention, one or more hydrogen atoms of the compounds of the present invention may be replaced by deuterium. Deuterium modification improves the metabolic properties of a drug with little or no change in its intrinsic pharmacology. Deuterium substitution at specific molecular positions improves metabolic stability, reduces formation of toxic metabolites and/or increases the formation of desired active metabolites. Accordingly, the present invention also encompasses the partially and fully deuterated compounds of the invention. The term hydrogen also encompasses deuterium.

The therapeutic use of compounds according to the invention, their pharmacologically acceptable salts, solvates and hydrates, respectively, as well as formulations and pharmaceutical compositions also lie within the scope of the present invention. The pharmaceutical compositions according to the present invention may comprise at least one compound of the invention as an active ingredient and, optionally, carrier substances and/or adjuvants.

The present invention also relates to prodrugs which are composed of a compound of the invention and at least one pharmacologically acceptable protective group which will be cleaved off under physiological conditions, such as an alkoxy-, arylalkyloxy-, acyl-, acyloxymethyl group (e.g. pivaloyloxymethyl), an 2-alkyl-, 2-aryl- or 2-arylalkyl oxycarbonyl-2-alkylidene ethyl group or an acyloxy group as defined herein, e.g. ethoxy, benzyloxy, acetyl or acetyloxy or, especially for a compound of the invention, carrying a hydroxy group (—OH): a sulfate, a phosphate (—OPO₃ or —OCH₂OPO₃) or an ester of an amino acid. For example, compositions may contain pro-drugs of the hydroxy group of a compound of the invention.

As used herein, the term pharmaceutically acceptable ester especially refers to esters which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include, but are not limited to, formates, acetates, propionates, butyrates, acrylates and ethylsuccinates.

The present invention also relates to a prodrug, a biohydrolyzable ester, a biohydrolyzable amide, a polymorph, tautomer, stereoisomer, metabolite, N-oxide, biohydrolyzable carbamate, biohydrolyzable ether, physiologically functional derivative, atropisomer, or in vivo-hydrolysable precursor, diastereomer or mixture of diastereomers, chemically protected form, affinity reagent, complex, chelate and a stereoisomer of the compounds of the invention.

As mentioned above, therapeutically useful agents that contain compounds of the invention, their solvates, salts or formulations are also comprised in the scope of the present invention. In general, compounds of the invention will be administered by using the known and acceptable modes known in the art, either alone or in combination with any other therapeutic agent.

For oral administration such therapeutically useful agents can be administered by one of the following routes: oral, e.g. as tablets, dragees, coated tablets, pills, semisolids, soft or hard capsules, for example soft and hard gelatin capsules, aqueous or oily solutions, emulsions, suspensions or syrups, parenteral including intravenous, intramuscular and subcutaneous injection, e.g. as an injectable solution or suspension, rectal as suppositories, by inhalation or insufflation, e.g. as a powder formulation, as microcrystals or as a spray (e.g. liquid aerosol), transdermal, for example via an transdermal delivery system (TDS) such as a plaster containing the active ingredient or intranasal. For the production of such tablets, pills, semisolids, coated tablets, dragees and hard, e.g. gelatin capsules, the therapeutically useful product may be mixed with pharmaceutically inert, inorganic or organic excipients as are e.g. lactose, sucrose, glucose, gelatine, malt, silica gel, starch or derivatives thereof, talc, stearinic acid or their salts, dried skim milk, and the like. For the production of soft capsules one may use excipients as are e.g. vegetable, petroleum, animal or synthetic oils, wax, fat, polyols. For the production of liquid solutions, emulsions or suspensions or syrups one may use as excipients e.g. water, alcohols, aqueous saline, aqueous dextrose, polyols, glycerin, lipids, phospholipids, cyclodextrins, vegetable, petroleum, animal or synthetic oils. Particularly useful are lipids, such as phospholipids (e.g., natural origin and/or with a particle size between 300 to 350 nm) in phosphate buffered saline (pH=7 to 8, e.g., 7.4). For suppositories one may use excipients as are e.g. vegetable, petroleum, animal or synthetic oils, wax, fat and polyols. For aerosol formulations one may use compressed gases suitable for this purpose, as are e.g. oxygen, nitrogen and carbon dioxide. The pharmaceutically useful agents may also contain additives for conservation, stabilization, e.g. UV stabilizers, emulsifiers, sweetener, aromatizers, salts to change the osmotic pressure, buffers, coating additives and antioxidants.

In general, in the case of oral or parenteral administration to adult humans weighing approximately 80 kg, a daily dosage of about 10 mg to about 10,000 mg, or from about 20 mg to about 1,000 mg, should be appropriate, although the upper limit may be exceeded when indicated. The daily dosage can be administered as a single dose or in divided doses, or for parenteral administration, it may be given as continuous infusion or subcutaneous injection.

### Methods of Making Compounds

The invention also provides methods of making compounds of the invention, such as those described above. Synthesis schemes for making specific compounds of Formula (I) are provided in the Examples below.

### Methods of Treating Conditions

The compounds and compositions of the invention modulate activity of one or more protein kinases. The compounds and compositions may inhibit, activate, or otherwise alter kinase activity. Consequently, the compounds and compositions may be used to diagnose, treat, or prevent a condition, such as a disease, disorder, or other condition for which modulation of kinase activity provides therapeutic benefit.

Diseases, disorders, and conditions that can be diagnosed and/or treated using compositions and methods of the invention include those associated with aberrant activity, e.g., increased activity or decreased activity, of one or more kinases. The disease, disorder, or condition may be associated with aberrant LRRK2 activity, such as Alzheimer's disease, Crohn's disease, inflammatory bowel disease, an inflammatory disease, leprosy, neurodegenerative diseases, a non-skin cancer, or Parkinson's disease, including familial Parkinson's disease, sporadic Parkinson's disease, late-onset Parkinson's disease (PD), and type 8 Parkinson's disease.

The disease, disorder, or condition may be or include a respiratory tract/obstructive airways disease or disorder, such as rhinorrhea, tracheal constriction, airway contraction, acute-, allergic, atrophic rhinitis or chronic rhinitis (such as rhinitis caseosa, hypertrophic rhinitis, rhinitis purulenta, rhinitis sicca), rhinitis medicamentosa, membranous rhinitis (including croupous, fibrinous and pseudomembranous rhinitis), scrofulous rhinitis, perennial allergic rhinitis, seasonal rhinitis (including rhinitis nervosa (hay fever) and vasomotor rhinitis), pollinosis, asthma (such as bronchial, atopic, allergic, intrinsic, extrinsic, exercise-induced, cold air-induced, occupational, bacterial infection-induced, and dust asthma particularly chronic or inveterate asthma (e.g. late asthma and airways hyper-responsiveness)), bronchitis (including chronic, acute, arachidic, catarrhal, croupus, phthinoid and eosinophilic bronchitis), cardiobronchitis, pneumoconiosis, chronic inflammatory disease of the lung which result in interstitial fibrosis, such as interstitial lung disease (ILD) (e.g., idiopathic pulmonary fibrosis, or ILD associated with rheumatoid arthritis, or other autoimmune conditions), acute lung injury (ALI), adult respiratory distress syndrome (ARDS), chronic obstructive pulmonary, airways or lung disease (CORD, COAD, COLD or COPD, such as irreversible COPD), chronic sinusitis, conjunctivitis (e.g. allergic conjunctivitis), cystic fibrosis, extrinsic allergic alveolitis (like farmer's lung and related diseases), fibroid lung, hypersensitivity lung diseases, hypersensitivity pneumonitis, idiopathic interstitial pneumonia, nasal congestion, nasal polyposis, otitis media, and cough (chronic cough associated with inflammation or iatrogenic induced), pleurisy, pulmonary congestion, emphysema, bronchiectasis, sarcoidosis, lung fibrosis, including cryptogenic fibrosing alveolitis, fibrosis complicating anti-neoplastic therapy and chronic infection, including tuberculosis and aspergillosis and other fungal infections, vasculitic and thrombotic disorders of the lung vasculature, and pulmonary hypertension, acute viral infection including the common cold, and infection due to respiratory syncytial virus, influenza, coronavirus (including SARS) and adenovirus, allergic bronchopulmonary mycosis, emphysema, diffuse panbronchiolitis, systemic anaphylaxis or hypersensitivity responses, drug allergies (e.g., to penicillin, cephalosporins), insect sting allergies, and food related allergies which may have effects remote from the gut (such as migraine, rhinitis and eczema), anaphylactic shock, or vascular spasms.

The disease, disorder, or condition may be or include a bone and joint related disease or disorder, such as osteoporosis, arthritis (including rheumatic, infectious, autoimmune, chronic, malignant), seronegative spondyloarthropathies (such as ankylosing spondylitis, rheumatoid spondylitis, psoriatic arthritis, enthesopathy, Bechet's disease, Marie-Strumpell arthritis, arthritis of inflammatory bowel disease, and Reiter's disease), systemic sclerosis, osteoarthritis, osteoarthrosis, both primary and secondary to e.g. congenital hip dysplasia, cervical and lumbar spondylitis, and low back and neck pain, Still's disease, reactive arthritis and undifferentiated spondarthropathy, septic arthritis and other infection-related arthropathies and bone disorders such as tuberculosis, including Pott's disease and Poncet's syndrome, acute and chronic crystal-induced synovitis including urate gout, calcium pyrophosphate deposition disease, and calcium apatite related tendon, bursar and synovial inflammation, primary and secondary Sjogren's syndrome, systemic sclerosis and limited scleroderma, mixed connective tissue disease, and undifferentiated connective tissue disease, inflammatory myopathies including, polymalgia rheumatica, juvenile arthritis including idiopathic inflammatory arthritides of whatever joint distribution and associated syndromes, other joint disease (such as intervertebral disc degeneration or temporomandibular joint degeneration), rheumatic fever and its systemic complications, vasculitides including giant cell arteritis, Takayasu's arteritis, polyarteritis nodosa, microscopic polyarteritis, and vasculitides to associated with viral infection, hypersensitivity reactions, cryoglobulins, paraproteins, low back pain, Familial Mediterranean fever, Muckle-Wells syndrome, and Familial Hibenian Fever, Kikuchi disease, drug-induced arthalgias, tendonititides, polychondritis, and myopathies, osteoporosis, osteomalacia like osteoporosis, osteopenia, osteogenesis imperfects, osteopetrosis, osteofibrosis, osteonecrosis, Paget's disease of bone, hypophosphatemia, Felty's syndrome, Still's disease, slack of artificial joint implant, sprain or strain of muscle or joint, tendinitis, fasciitis, periarthritis humeroscapularis, cervico-omo-brachial syndrome, or tenosynovitis.

The disease, disorder, or condition may be or include a skin or eye related disease or disorder, such as glaucoma, ocular hypertension, cataract, retinal detachment, psoriasis (including psoriasis vulgaris, pustular psoriasis, arthritic psoriasis, erythroderma psoriaticum), palmoplantar pustulosis, xerodoma, eczematous diseases (like atopic dermatitis, ultraviolet radiation dermatitis, contact dermatitis, and seborrheic dermatitis), phytodermatitis, photodermatitis, cutaneous eosinophilias, chronic skin ulcers, cutaneous lupus erythematosus, contact hypersensitivity/allergic contact dermatitis (including sensitivity to poison ivy, sumac, or oak), and eosinophilic folliculitis (Ofuji's disease), pruritus, drug eruptions, urticaria (acute or chronic, allergic or non-allergic), acne, erythema, dermatitis herpetiformis, scleroderma, vitiligo, lichen planus, lichen sclerosus et atrophica, pyodenna gangrenosum, skin sarcoid, pemphigus, ocular pemphigus, pemphigoid, epidermolysis bullosa, angioedema, vasculitides, toxic erythemas, cutaneous eosinophilias, alopecia areata, male-pattern baldness, Sweet's syndrome, Stevens-Johnson syndrome, Weber-Christian syndrome, erythema multiforme, cellulitis, both, infective and non infective, panniculitis, cutaneous Lymphomas, non, melanoma skin cancer and other dysplastic lesions, blepharitis, iritis, anterior and posterior uveitis, choroiditis, autoimmune, degenerative or inflammatory disorders affecting the retina, ophthalmitis including sympathetic ophthalmitis, sarcoidosis, xerosis infections including viral, fungal, and bacterial, allergic conjunctivitis, increased fibrosis, keloids, keloplasty, post surgical scars, epidermolysis bullosa, dry eye, ocular inflammation, allergic conjunctivitis, vernal conjunctivitis, vernal keratoconjunctivitis, and giant papillary conjunctivitis, ocular angiogenesis, cornea damage and scar, all forms of macular degeneration, macular edema, macular dystrophy, abnormal wound healing, scleritis, episcleritis, pachydermia, peripheral ulcerative keratitis, fungal keratitis, herpetic keratitis, invasive aspergillosis; conical cornea, dystorphia epithelialis comeae, or severe intraocular inflammation.

The disease, disorder, or condition may be or include a gastrointestinal tract and abdominal related disease or disorder, such as celiac/coeliac disease (e.g. celiac sprue), cholecystitis, enteritis (including infectious, ischemic, radiation, drug-induced, and eosinophilic gastroenteritis), eosinophilic esophagitis, eosinophilic gastrointestinal inflammation, allergen induced diarrhea, enteropathy associated with seronegative arthropathies, gastritis, autoimmune atrophic gastritis, ischemic bowel disease, inflammatory bowel disease (Crohn's disease and ulcerative colitis), colitis, Mooren's ulcer, irritable bowel syndrome, necrotizing enterocolitis, gut ischemia, glossitis, gingivitis, periodontitis, oesophagitis, including reflex, proctitis, fibrosis and cirrhosis of the liver, pancreatitis, both acute and chronic, pancreatic fibrosis, pancreatic sclerosis, pancreatolithiasis, hepatic cirrhosis, hepatitis (congestive, autoimmune, acute, fulminant, chronic, drug-induced, alcoholic, lupoid, steatohepatitis and chronic viral), fatty liver, primary biliary cirrhosis, hepatic porphyria, and gastrointestinal related allergic disorders, spastic colon, diverticulitis, gastroenteric bleeding, Behcet's disease; partial liver resection, acute liver necrosis (e.g. necrosis caused by toxins, viral hepatitis, shock or anoxia), or hemolytic uremic syndrome.

The disease, disorder, or condition may be or include a hematological disease or disorder, such as anemias, coagulation, myeloproliferative disorders, hemorrhagic disorders, leukopenia, eosinophilic disorders, leukemias (e.g. myelogenous, lymphomas, plasma cell dyscrasias, disorders of the spleen, Band's disease, hemophilia, purpura (including idiopathic thrombocytopenic purpura), or Wiskott-Aldrich syndrome.

The disease, disorder, or condition may be or include a metabolic disease or disorder, such as obesity, amyloidosis, disturbances of the amino and acid metabolism like branched chain disease, hyperaminoacidemia, hyperaminoaciduria, disturbances of the metabolism of urea, hyperammonemia, mucopolysaccharidoses e.g. Maroteaux-Lamy syndrome, storage disease like glycogen storage diseases and lipid storage diseases, glycogenosis I diseases like Cori's disease, malabsorption diseases like intestinal carbohydrate malabsorption, oligosaccharidase deficiency like maltase-, lactase-, sucrase-insufficiency, disorders of the metabolism of fructose, disorders of the metabolism of galactose, galactosaemia, disturbances of carbohydrate utilization like diabetes, hypoglycemia, disturbances of pyruvate metabolism, hypolipidemia, hypolipoproteinemia, hyperlipidemia, hyperlipoproteinemia, carnitine or carnitine acyltransferase deficiency, disturbances of the porphyrin metabolism, porphyrins, disturbances of the purine metabolism, lysosomal diseases, metabolic diseases of nerves and nervous systems like gangliosidoses, sphingolipidoses, sulfatidoses, leucodystrophies, or Lesch Nyhan syndrome.

The disease, disorder, or condition may be or include a cerebellar dysfunction or disturbance of brain metabolism, such as dementia, Alzheimer's disease, Huntington's chores, Parkinson's disease, Pick's disease, toxic encepha-lopathy, demyelinating neuropathies like inflammatory neuropathy, Guillain-Barre syndrome; Meniere's disease and radiculopathy, primary and secondary metabolic disorders associated with hormonal defects like any disorder stemming from either an hyperfunction or hypofunction of some hormone-secreting endocrine gland and any combination thereof. Sipple's syndrome, pituitary gland dysfunction and its effects on other endocrine glands, such as the thyroid, adrenals, ovaries, and testes, acromegaly, hyper- and hypothyroidism, euthyroid goiter, euthyroid sick syndrome, thyroiditis, and thyroid cancer, over or underproduction of the adrenal steroid hormones, adrenogenital syndrome, Cushing's syndrome, Addison's disease of the adrenal cortex, Addison's pernicious anemia, primary and secondary aldosteronism, diabetes insipidus, diabetes mellitus, carcinoid syndrome, disturbances caused by the dysfunction of the parathyroid glands, pancreatic islet cell dysfunction, diabetes, disturbances of the endocrine system of the female like estrogen deficiency, resistant ovary syndrome; muscle weakness, myotonia. Duchenne's and other muscular dystrophies, dystrophia myotonica of Steinert, mitochondrial myopathies like disturbances of the catabolic metabolism in the muscle, carbohydrate and lipid storage myopathies, glycogenoses, myoglobinuria, malignant hyperthermia, polymyalgia rheumatics, dermatomyositis, multiple myositis, primary myocardial disease, cardiomyopathy; disorders of the ectoderm, neurofibromatosis, scleroderma and polyar teritis, Louis-Bar syndrome, von Hippel-Lindau disease, Sturge-Weber syndrome, tuberous sclerosis, amyloidosis, porphyria; sexual dysfunction of the male and female; confused states and seizures due to inappropriate secretion of antidiuretic hormone from the pituitary gland, Liddle's syndrome, Bartter's syndrome, Fanconi's I syndrome, or renal electrolyte wasting.

The disease, disorder, or condition may be or include a transplant rejection related condition, such as acute and chronic allograft rejection following solid organ transplant, for example, transplantation of kidney, heart, liver, lung, and cornea, chronic graft versus host disease, skin graft rejection, and bone marrow transplant rejection, or immunosuppression.

The disease, disorder, or condition may be or include a genitourinary related condition, such as nephritis (interstitial, acute interstitial (allergic), and glomerulonephritis), nephrotic syndrome, cystitis including acute and chronic (interstitial) cystitis and Hunner's ulcer, acute and chronic urethritis, prostatitis, epididymitis, oophoritis, salpingitis, vulvo vaginitis, vulvovaginal candidiasis, Peyronie's disease, and erectile dysfunction, renal disease, renal fibrosis, nephropyelitis, secondary contracted kidney, steroid dependent and steroid-resistant nephrosis, or Goodpasture's syndrome.

The disease, disorder, or condition may be or include a CNS related disease or disorder, such as neurodegenerative diseases, Alzheimer's disease and other cementing disorders including CJD and nvCJD, amyloidosis, and other demyelinating syndromes, cerebral atherosclerosis and vasculitis, temporal arteritis, myasthenia gravis, acute and chronic so pain (acute, intermittent or persistent, whether of central or peripheral origin) including post-operative, visceral pain, headache, migraine, neuralgia (including trigeminal), atypical facial pain, joint and bone pain, pain arising from cancer and tumor invasion, neuropathic pain syndromes including diabetic, post-herpetic, and HIV-associated neuropathies, neurosarcoidosis, to brain injuries, cerebrovascular diseases and their consequences, Parkinson's disease, corticobasal degeneration, motor neuron disease, dementia, including ALS (Amyotrophic-lateral sclerosis), multiple sclerosis, traumatic brain injury, stroke, post-stroke, post-traumatic brain injury, and small-vessel cerebrovascular disease, dementias, vascular dementia, dementia with Lewy bodies, frontotemporal dementia and Parkinsonism linked 1 to chromosome 17, frontotemporal dementias, including Pick's disease, progressive supranuclear palsy, corticobasal degeneration, Huntington's disease, thalamic degeneration, HIV dementia, schizophrenia with dementia, and Korsakoffs psychosis, within the meaning of the definition are also considered to be CNS disorders central and peripheral nervous system complications of malignant, infectious or autoimmune processes, algesia, cerebral infarction, attack, cerebral ischemia, head injury, spinal cord injury, myelopathic muscular atrophy, Shy-Drager syndrome, Reye's syndrome, progressive multifocal leukoencephalopathy, normal pressure hydrocephalus, sclerosing panencephalitis, frontal lobe type dementia, acute anterior poliomyelitis (poliomyelitis), poliomyelitis neurosis, viral encephalitis, allergic encephalomyelitis, epileptic encephalopathies, Creutzfeldt-Jakob disease, Kuru disease, bovine spongiform encephalopathy (mad cow disease), scrapie, epilepsy, cerebral amyloid angiopathy, depression, mania, manic-depressive psychosis, hereditary cerebellar ataxia, peripheral neuropathy, Nasu-Hakola syndrome, or Machado-Joseph disease.

The disease, disorder, or condition may be or include an inflammatory or immunological disease or disorder, such as general inflammation (of the ocular, nasal, pulmonary, and gastrointestinal passages), mastocytosis/mast cell disorders (cutaneous, systemic, mast cell activation syndrome, and pediatric mast cell diseases), mastitis (mammary gland), vaginitis, vasculitis (e.g., necrotizing, cutaneous, and hypersensitivity vasculitis), Wegener granulamatosis, myyositis (including polymyositis, dermatomyositis), basophil related diseases including basophilic leukemia and basophilic leukocytosis, and eosinophil related diseases such as Churg-Strauss syndrome, eosinophilic granuloma, lupus erythematosus (such as, systemic lupus erythematosus, subacute cutaneous lupus erythematosus, and discoid lupus erythematosus), chronic thyroiditis, Hashimoto's thyroiditis, Grave's disease, type I diabetes, complications arising from diabetes mellitus, other immune disorders, eosinophilia fasciitis, hyper IgE syndrome, Addison's disease, antiphospholipid syndrome, immunodeficiency disease, acquired immune deficiency syndrome (AIDS), leprosy, Sezary syndrome, paraneoplastic syndromes, and other autoimmune disorders, fervescence, myositis, nervous diseases selected from multiple myositis, bursitis, Evans syndrome, leukotriene B4-mediated diseases, idiopathic hypoparathyroidism, nephrotic syndrome lupus, or immunosuppression.

The disease, disorder, or condition may be or include a cardiovascular disease or disorder, such as congestive heart failure, myocardial infarction, ischemic diseases of the heart, all kinds of atrial and ventricular arrhythmias, hypertension, cerebral trauma, occlusive vascular disease, stroke, cerebrovascular disorder, atherosclerosis, restenosis, affecting the coronary and peripheral is circulation, pericarditis, myocarditis, inflammatory and auto-immune cardiomyopathies including myocardial sarcoid, endocarditis, valvulitis, and aortitis including infective (e.g. syphilitic), hypertensive vascular diseases, peripheral vascular diseases, and atherosclerosis, vasculitides, disorders of the proximal and peripheral veins including phlebitis and thrombosis, including deep vein thrombosis and complications of varicose veins, aortic aneurism, periarteritis nodosa, cardiac fibrosis, post-myocardial infarction, idiopathic cardiomyopathy, or angioplasty.

The disease, disorder, or condition may be or include an oncological disease or disorder, such as common cancers (prostate, breast, lung, ovarian, pancreatic, bowel and colon, abdomen, stomach (and any other digestive system cancers), liver, pancreas, peritoneum, endocrine glands (adrenal, parathyroid, pituitary, testicles, ovary, thymus, thyroid), eye, head, neck, nervous system (central and peripheral), lymphatic system, blood, pelvic, skin, bone, soft tissue, spleen, thoracic, urogenital, and brain tumors), breast cancer, genitourinary cancer, lung cancer, gastrointestinal cancer, epidermoid cancer, melanoma, ovarian cancer, pancreas cancer, neuroblastoma, malignancies affecting the bone marrow (including the leukaemias) and lymphoproliferative systems, such as Hodgkin's and non-Hodgkin's lymphoma, B-cell lymphoma, follicular lymphoma, metastatic disease and tumor recurrences, and paraneoplastic syndromes, as well as hypergammaglobulinemia, lymphoproliferative diseases, disorders, and/or conditions, paraproteinemias, purpura (including idiopathic thrombocytopenic purpura), Waldenstron's Macroglobulinemia, Gaucher's Disease, histiocytosis, retinoblastoma and any other hyperproliferative disease, sarcomata, cachexia, tumor growth, tumor invasion, metastasis, AIDS-related lymphomas, malignant immunoproliferative diseases, multiple myeloma and malignant plasma cell neoplasms, lymphoid leukemia, acute or chronic myeloid leukemia, acute or chronic lymphocytic leukemia, monocytic leukemia, other leukemias of specified cell type, leukemia of unspecified cell type, other and unspecified malignant neoplasms of lymphoid, haematopoietic and related tissues, for example diffuse large cell lymphoma, T-cell lymphoma or cutaneous T-cell lymphoma). Myeloid cancer includes e.g. acute or chronic myeloid leukaemia, or keratoleukoma.

The disease, disorder, or condition may be or include another disease or disorder, such as pain, migraine, sleep disorders, fever, sepsis, idiopathic thrombocytopenia pupura, post-operative adhesions, flushing, ischemic/reperfusion injury in the heart, brain, peripheral limbs, bacterial infection, viral infection, fungal infection, thrombosis, endotoxin shock, septic shock, thermal regulation including fever, Raynaud's disease, gangrene, diseases requiring anti-coagulation therapy, congestive heart failure, mucus secretion disorders, pulmonary hypotension, prostanoid-induced smooth muscle contract associated with dysmenorrhea and premature labor, premature delivery, reperfusion injury, burn, thermal injury, hemorrhage or traumatic shock, menstrual pain, menstrual cramp, dysmenorrhea, periodontosis, rickettsial infectious disease, protozoal disease, reproduction disease, toothache, pain after tooth extraction, Herpes zoster, Herpes simplex, retroperitoneal fibrosis, or various radiation injuries.

In certain embodiments, the disease is selected from the group consisting of an inflammatory disease, an autoimmune disease, an allergic disorder, and an ocular disorder. In certain embodiments, the disease is selected from the group consisting of pruritus, eczema, asthma, rhinitis, dry eye, ocular inflammation, allergic conjunctivitis, vernal conjunctivitis, vernal keratoconjunctivitis, giant papillary conjunctivitis, fungal keratitis and uveitis.

The method may include modulating the activity of one or more kinases in a subject, such as any of the kinase described above. The method may include inhibiting a kinase. The method may include activating, e.g., stimulating or enhancing the activity of a kinase. The method may include modulating activity of a single kinase or preferentially modulating activity of a specific kinase over others. The method may include modulating activity of multiple kinases or preferentially modulating activity of two more specific kinases over others.

The method may include providing a compound of the invention. The method may include providing multiple compounds of the invention.

The method may include contacting cells containing a kinase with one or more compounds of the invention. For example and without limitation, contacting a cell with a compound may include exposing a cell to a compound, e.g., in a formulation, such as any of those described above; delivering a compound inside a cell; providing a compound to a subject and allowing a cell in the subject to become exposed to the compound. Contacting may be performed in vivo or in vitro. In vitro contact may include exposure of cells or tissue isolated from a subject. The method may include contacting cells with a single compound of the invention. The method may include contact cells with multiple compounds of the invention.

The compounds of the invention are particularly advantageous because they are useful in treatment of disorders related to central nervous system (CNS). In particular, the compounds of the invention cross the blood brain barrier. The blood-brain barrier (BBB) represents a major obstacle to the delivery of drugs to the central nervous system (CNS). Therefore, the BBB is a major impediment to the treatment of CNS diseases as many drugs are unable to reach this organ at therapeutic concentrations. The compounds of the invention transfer across the BBB and thus, they may be effective in treatment of disorders related to CNS.

In another embodiment, the compounds of the invention have a higher systemic exposure as compared to the exposure and have minimal permeation through the BBB. In these embodiments, the compounds of the invention may have a BBB permeation of less than about 10%, less than about 7.5%, less than about 5%, less than about 2.5%, or less than about 1%. In most preferred embodiments, the compounds of the invention have a BBB permeation of about 1% or lower. Thus, in certain embodiments, the compounds are beneficial because they do not cross the BBB which leads to reduced adverse effects as result of administration of compounds. In these embodiments, the compounds of the invention are beneficial for treatment of conditions that do not require the compounds to cross the BBB to have CNS exposure.

The method may include administration of a composition to a subject. The compositions may be provided by any suitable route of administration. For example and without limitation, the compositions may be administered buccally, by injection, dermally, enterally, intraarterially, intravenously, intranasally, e.g., by inhalation, intraocularly, orally, parenterally, pulmonarily, rectally, subcutaneously, systemically, topically, e.g., to the skin or eye, transdermally, or with or on an implantable medical device (e.g., stent or drug-eluting stent or balloon equivalents).

The method may include using a composition of the invention to diagnose a disease, disorder, or condition in a subject. For example, a radiolabeled form of a compound may be used a tracer in positron emission tomography (PET) to identify anatomical locations of aberrant kinase activity. PET is known in the art and described in, for example, Wadsak Wolfgang, Mitterhauser Markus (2010), “Basics and principles of radiopharmaceuticals for PET/CT”, European Journal of Radiology, 73 (3): 461-469. doi:10.1016/j.ejrad.2009.12.022; Bailey, D. L; D. W. Townsend; P. E. Valk; M. N. Maisey (2005), Positron Emission Tomography: Basic Sciences. Secaucus, NJ: Springer-Verlag, ISBN 1-85233-798-2; and Carlson, Neil (Jan. 22, 2012). Physiology of Behavior. Methods and Strategies of Research, 11th edition, Pearson, p. 151, ISBN 0205239390, the contents of each of which are incorporated herein by reference. The invention may include administering one or more compositions of the invention for both diagnostic and therapeutic purposes.

### EXAMPLES

### General Synthetic Scheme

Compounds of this disclosure can be made by the methods depicted in the reaction schemes shown below. The starting materials and reagents used in preparing these compounds are either available from commercial suppliers such as Sigma-Aldrich Chemical Co., (Milwaukee, Wis.), Acros Organics, Bachem (Torrance, Calif), Oakwood Chemicals, Matrix Chemicals, or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), March's Advanced Organic Chemistry, (John Wiley and Sons, 4th Edition) and Larock's Comprehensive Organic Transformations (VCH Publishers Inc., 1989).

Generic schemes are merely illustrative of some methods by which the compounds of this disclosure, and pharmaceutically acceptable salts thereof, can be synthesized, and various modifications to these schemes can be made and will be suggested to one skilled in the art reading this disclosure. The starting materials, the intermediates, and the final products of the reaction(s) may be isolated and purified if desired using conventional techniques, including but not limited to filtration, distillation, crystallization, chromatography and the like. Such materials may be characterized using conventional means, including physical constants and spectral data.

Unless specified to the contrary, the reactions described herein take place at atmospheric pressure over a temperature range from about −78° C. to about 200° C., such as from about 0° C. to about 125° C. and further such as at about room (or ambient) temperature, e.g., about 20° C. The routes shown and described herein are illustrative only and are not intended, nor are they to be construed, to limit the scope of the claims in any manner whatsoever. Those skilled in the art will be able to recognize modifications of the disclosed syntheses and to devise alternate routes based on the disclosures herein; all such modifications and alternate routes are within the scope of the claims.

Compounds of this invention can be synthesized by the methods depicted in the general reaction Schemes shown below.

The following General Scheme I represents a synthetic method for compounds of this invention, i.e., compounds of formula (A) (B) (C) (D) (E), wherein R¹, R², R³, R⁴, R⁵, R⁶, R⁷, R⁸, R⁹, R¹⁰, R¹¹, X and Y are as defined above.

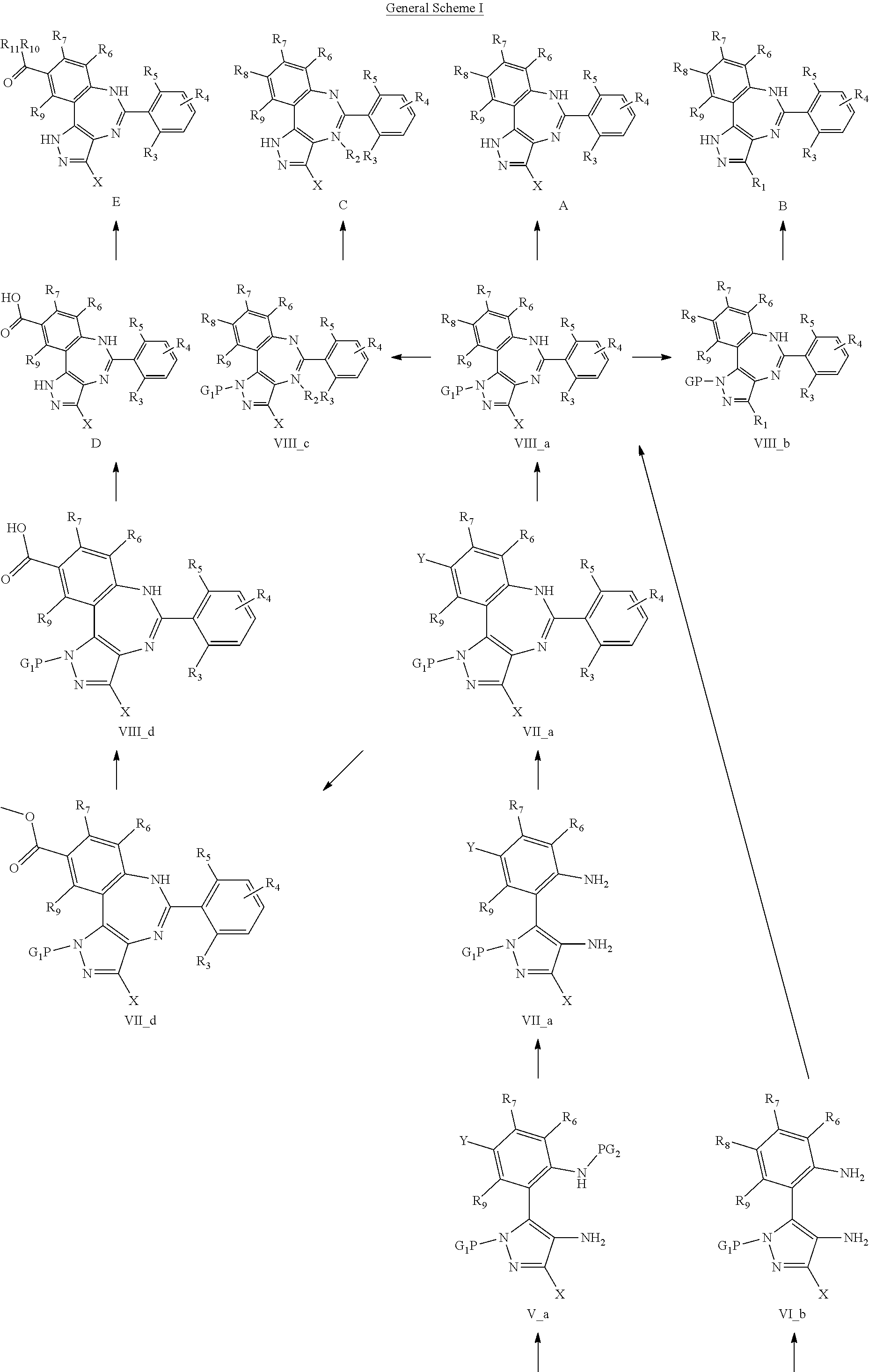

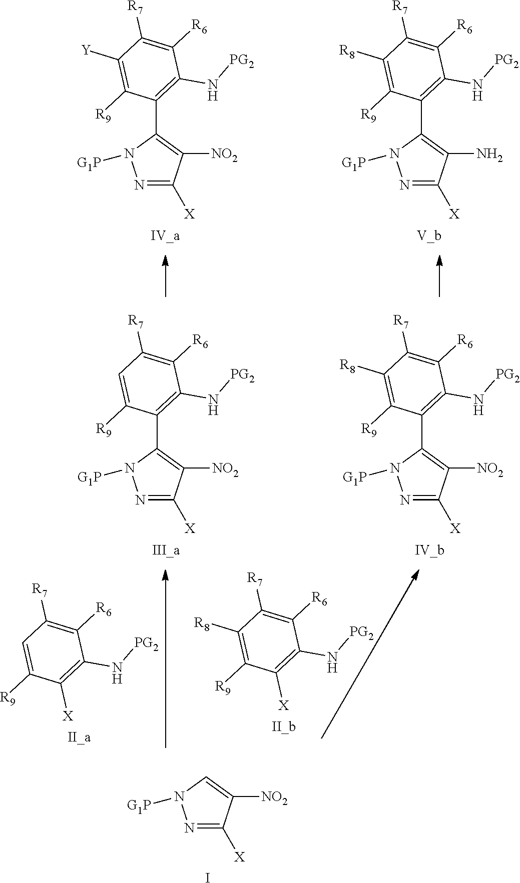

In General Scheme I, compound (I), compound (II_a) and compound (II_b) are purchased from commercial sources or prepared according to the literature methods.

The C—H activation between compound (I) and compound (II_a) affords compound (III_a), followed by a halogenation to afford compound (IV_a) which is reduced to compound (V_a) and then de-protected to give compound (VI_a). The ring-closure between compound (VI_a) and aldehyde gives compound (VII_a) which is converted into compound (VIII_a) by a cross coupling.

The de-deprotection of compound (VIII_a) affords the compound of formula (A). Compound (VIII_a) can be converted into compound (VIII b) by a cross coupling which is de-protected to obtain the compound of formula (B).

The C—H activation with compound (I) gives compound (IV_b), followed by a reduction to obtain compound (V_b). A direct de-protection affords compound (VI_b) which is used in a ring-closure reaction to give compound (VIII_a).

A substitution of compound (VII_a) gives compound (VII_c) which is de-protected to obtain the compound of formula (C).

The carbonylation of compound (VII_a) affords compound (VII_d) which is hydrolyzed to obtain compound (VIII_d). The de-protection of compound (VIII_d) gives the compound of formula (D) which is used in the acid-amine coupling to afford the compound of formula (E).

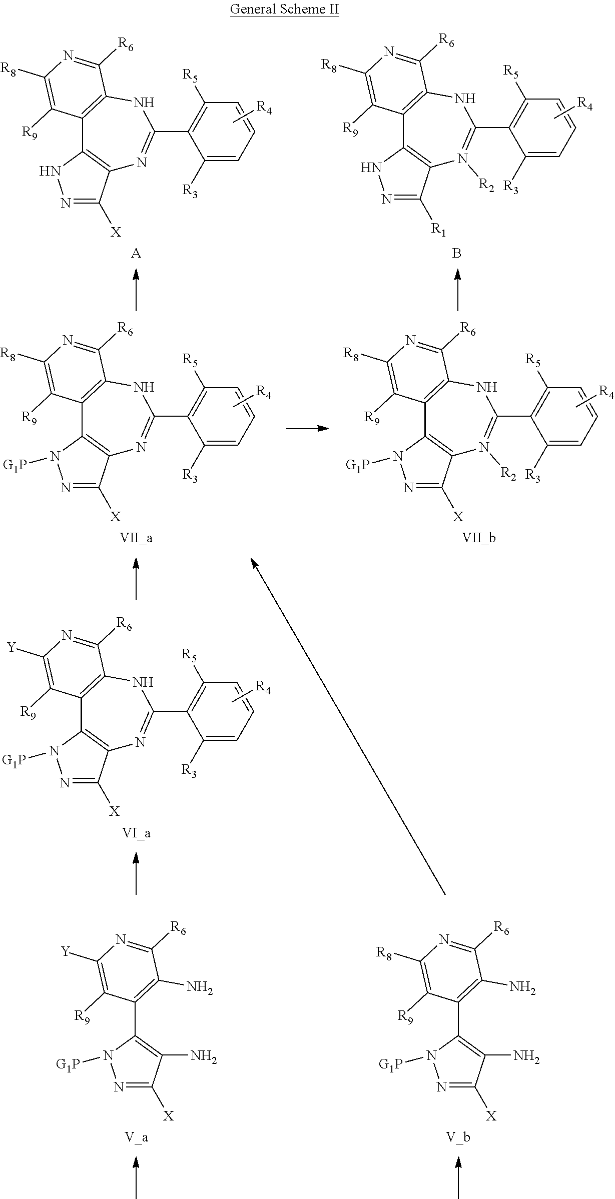

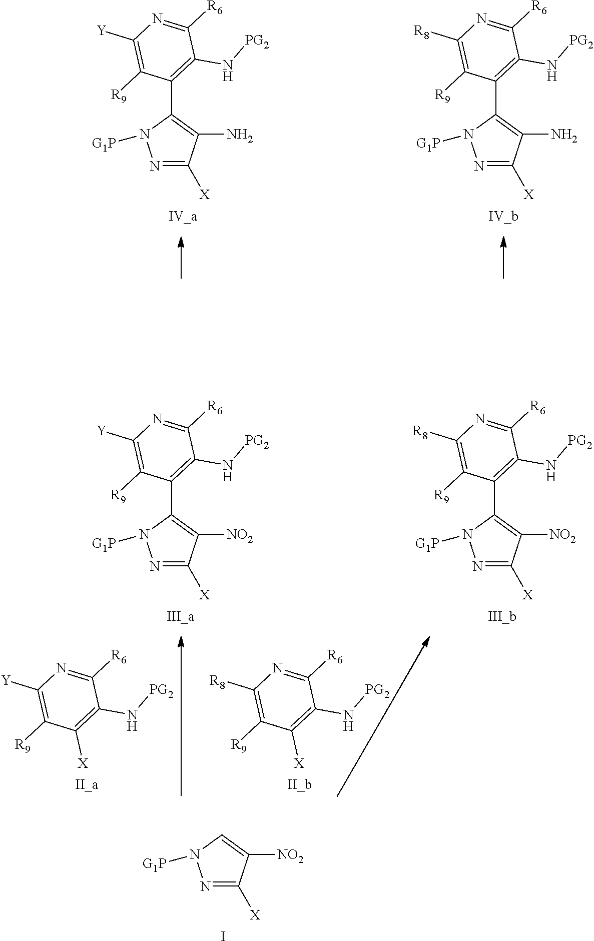

In General Scheme II, compound (I) (II_a) and (II_b) are purchased from commercial sources or prepared according to the literature methods.

The C—H activation between compound (I) and compound (II_a) affords compound (III_a), followed by a reduction to afford compound (IV_a) which is de-protected to give compound (V_a).

The ring-closure between compound (V_a) and aldehyde gives compound (VI_a) which is converted into compound (VII_a) by a cross coupling. The de-deprotection of compound (VII_a) affords the compound of formula (A). Compound (VII_a) can be converted into compound (VII_b) by a cross coupling which is de-protected to obtain the compound of formula (B).

The C—H activation with compound (I) gives compound (III_b), followed by a reduction to obtain compound (IV_b). A direct de-protection affords compound (V_b) which is used in a ring-closure reaction to give compound (VII_a).

### Synthetic Examples

The following preparations of intermediates, reference compounds, and/or compounds of Formula (I) are given to enable those skilled in the art to more clearly understand and to practice the present disclosure. They should not be considered as limiting the scope of the disclosure, but merely as being illustrative and representative thereof.

Representative compounds of Formula (I), or salts thereof, are disclosed in Table 1. Although Table 1 may show a specific salt of a compound of Formula (I), those skilled in the art will be able to recognize the parent compound (wherein the “parent compound” is a compound without a salt moiety present), and other salts, such as pharmaceutically acceptable salts, of those compounds in Table 1.

TABLE 1

| Ex. | | | MS Found |
|---|---|---|---|
| No. | Structure | IUPAC | [M + H]⁺ |
| 1. | 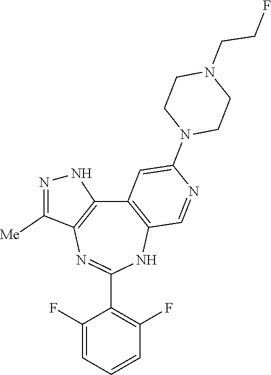 [SMILES: Cc1n[nH]c2c1N=C(c1c(F)cccc1F)Nc1cnc(N3CCN(CCF)CC3)cc1-2] | 8-(2,6-difluorophenyl)-13-[4-(2- fluoroethyl)piperazin-1-y1]-5-methyl- 3,4,7,9,12- pentazatricyclo[8.4.0.02,6]tetradeca- 1(10),2(6),4,7,11,13-hexaene | 442.2 |
| 2. | 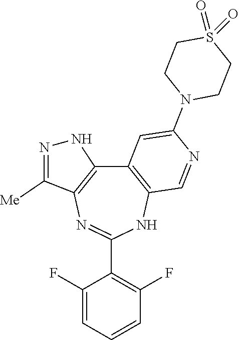 [SMILES: Cc1n[nH]c2c1N=C(c1c(F)cccc1F)Nc1cnc(N3CCS(=O)(=O)CC3)cc1-2] | 4-[8-(2,6-difluorophenyl)-5-methyl- 3,4,7,9,12- pentazatricyclo[8.4.0.02,6]tetradeca- 1(10),2(6),4,7,11,13-hexaen-13-y1]-1,4- thiazinane 1,1-dioxide | 444.4 |
| 3. | 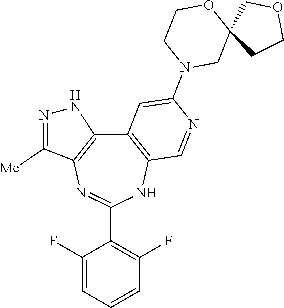 [SMILES: Cc1n[nH]c2c1N=C(c1c(F)cccc1F)Nc1cnc(N3CCO[C@@]4(CCOC4)C3)cc1-2] | (5S)-9-[8-(2,6-difluorophenyl)-5- methyl-3,4,7,9,12-pentazatricyclo [8.4.0.02,6]tetradeca- 1(10),2(6),4,7,11,13-hexaen-13-y1]-2,6- dioxa-9-azaspiro[4.5]decane | 453.3 |
| 4. | 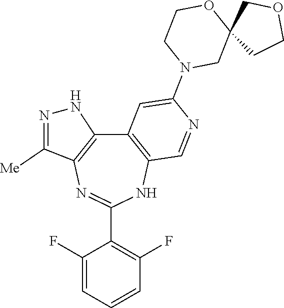 [SMILES: Cc1n[nH]c2c1N=C(c1c(F)cccc1F)Nc1cnc(N3CCO[C@]4(CCOC4)C3)cc1-2] | (5R)-9-[8-(2,6-difluorophenyl)-5- methyl-3,4,7,9,12- pentazatricyclo[8.4.0.02,6] tetradeca- 1(10),2(6),4,7,11,13-hexaen-13-yl]-2,6- dioxa-9-azaspiro[4.5]decane | 453.3 |
| 5. | 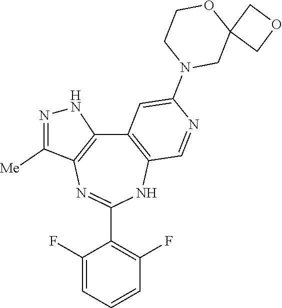 [SMILES: Cc1n[nH]c2c1N=C(c1c(F)cccc1F)Nc1cnc(N3CCOC4(COC4)C3)cc1-2] | 8-[8-(2,6-difluorophenyl)-5-methyl- 3,4,7,9,12- pentazatricyclo[8.4.0.02,6]tetradeca- 1(10),2(6),4,7,11,13-hexaen-13-y1]-2,5- dioxa-8-azaspiro[3.5]nonane | 438.4 |
| 6. | 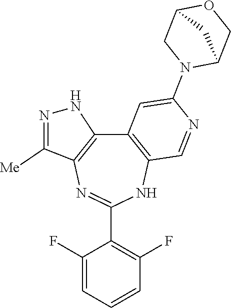 [SMILES: Cc1n[nH]c2c1N=C(c1c(F)cccc1F)Nc1cnc(N3C[C@@H]4C[C@H]3CO4)cc1-2] | (1S,4S)-5-[8-(2,6-difluorophenyl)-5- methyl-3,4,7,9,12- pentazatricyclo[8.4.0.02,6]tetradeca- 1(10),2(6),4,7,11,13-hexaen-13-y1]-2- oxa-5-azabicyclo[2.2.1]heptane | 409.2 |
| 7. | 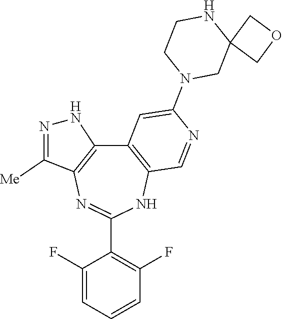 [SMILES: Cc1n[nH]c2c1N=C(c1c(F)cccc1F)Nc1cnc(N3CCNC4(COC4)C3)cc1-2] | 8-[8-(2,6-difluorophenyl)-5-methyl- 3,4,7,9,12- pentazatricyclo[8.4.0.02,6]tetradeca- 1(10),2(6),4,7,11,13-hexaen-13-y1]-2- oxa-5,8-diazaspiro[3.5]nonane | 437.5 |
| 8. | 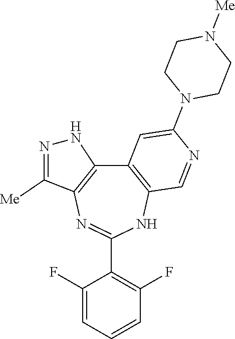 [SMILES: Cc1n[nH]c2c1N=C(c1c(F)cccc1F)Nc1cnc(N3CCN(C)CC3)cc1-2] | 8-(2,6-difluorophenyl)-5-methyl-13-(4- methylpiperazin-1-y1)-3,4,7,9,12- pentazatricyclo[8.4.0.02,6]tetradeca- 1(10),2(6),4,7,11,13-hexaene | 410.2 |
| 9. | 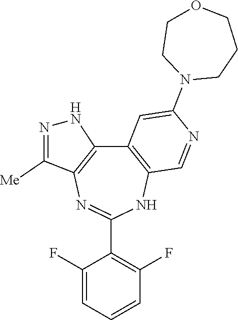 [SMILES: Cc1n[nH]c2c1N=C(c1c(F)cccc1F)Nc1cnc(N3CCCOCC3)cc1-2] | 4-[8-(2,6-difluorophenyl)-5-methyl- 3,4,7,9,12-pentazatricyclo[8.4.0.02,6] tetradeca-1(10),2(6),4,7,11,13-hexaen- 13-y1]-1,4-oxazepane | 411.1 |
| 10. | 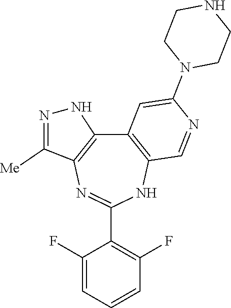 [SMILES: Cc1n[nH]c2c1N=C(c1c(F)cccc1F)Nc1cnc(N3CCNCC3)cc1-2] | 8-(2,6-difluorophenyl)-5-methyl-13- piperazin-1-yl-3,4,7,9,12- pentazatricyclo[8.4.0.02,6]tetradeca- 1(10),2(6),4,7,11,13-hexaene | 396.3 |
| 11. | 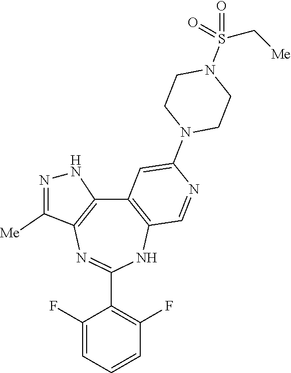 [SMILES: CCS(=O)(=O)N1CCN(c2cc3c(cn2)NC(c2c(F)cccc2F)=Nc2c(C)n[nH]c2-3)CC1] | 8-(2,6-difluorophenyl)-13-(4- ethylsulfonylpiperazin-1-y1)-5-methyl-3, 4,7,9,12-pentazatricyclo[8.4.0.02, 6]tetradeca-1(10),2(6),4,7,11,13- hexaene | 488.3 |
| 12. | 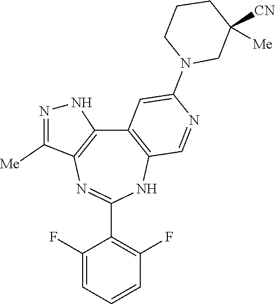 [SMILES: Cc1n[nH]c2c1N=C(c1c(F)cccc1F)Nc1cnc(N3CCC[C@@](C)(C#N)C3)cc1-2] | (R)-1-(5-(2,6-difluorophenyl)-3-methyl- 1,6-dihydropyrazolo[4,3-d]pyrido[4,3- f][1,3]diazepin-9-y1)-3- methylpiperidine-3-carbonitrile | 434.2 |
| 13. | 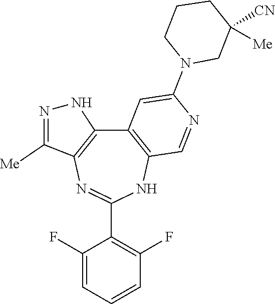 [SMILES: Cc1n[nH]c2c1N=C(c1c(F)cccc1F)Nc1cnc(N3CCC[C@](C)(C#N)C3)cc1-2] | (S)-1-(5-(2,6-difluorophenyl)-3-methyl- 1,6-dihydropyrazolo[4,3-d]pyrido[4,3- f][1,3]diazepin-9-yl)-3- methylpiperidine-3-carbonitrile | 434.2 |
| 14. | 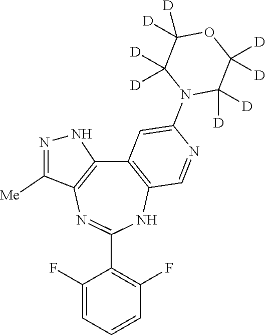 [SMILES: [2H]C1([2H])OC([2H])([2H])C([2H])([2H])N(c2cc3c(cn2)NC(c2c(F)cccc2F)=Nc2c(C)n[nH]c2-3)C1([2H])[2H]] | 2,2,3,3,5,5,6,6-octadeuterio-4-[8-(2,6- difluorophenyl)-5-methyl-3,4,7,9,12- pentazatricyclo[8.4.0.02,6]tetradeca- 1(10),2(6),4,7,11,13-hexaen-13- yl]morpholine | 405.0 |
| 15. | 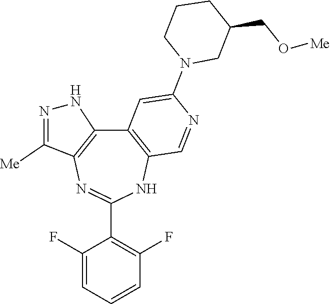 [SMILES: COC[C@@H]1CCCN(c2cc3c(cn2)NC(c2c(F)cccc2F)=Nc2c(C)n[nH]c2-3)C1] | (2R)-4-[8-(2,6-difluorophenyl)-5- methyl-3,4,7,9,12- pentazatricyclo[8.4.0.02,6]tetradeca- 1(10),2(6),4,7,11,13-hexaen-13-y1]-2- (methoxymethyl)morpholine | 441.2 |
| 16. | 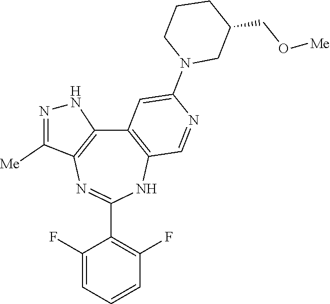 [SMILES: COC[C@H]1CCCN(c2cc3c(cn2)NC(c2c(F)cccc2F)=Nc2c(C)n[nH]c2-3)C1] | (2S)-4-[8-(2,6-difluorophenyl)-5- methyl-3,4,7,9,12- pentazatricyclo[8.4.0.02,6]tetradeca- 1(10),2(6),4,7,11,13-hexaen-13-y1]-2- (methoxymethyl)morpholine | 441.2 |
| 17. | 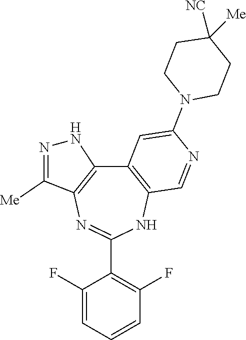 [SMILES: Cc1n[nH]c2c1N=C(c1c(F)cccc1F)Nc1cnc(N3CCC(C)(C#N)CC3)cc1-2] | 1-[8-(2,6-difluorophenyl)-5-methyl- 3,4,7,9,12- pentazatricyclo[8.4.0.02,6]tetradeca- 1(10),2(6),4,7,11,13-hexaen-13-y1]-4- methyl-piperidine-4-carbonitrile | 434.1 |
| 18. | 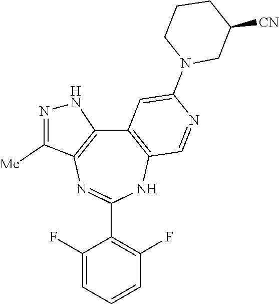 [SMILES: Cc1n[nH]c2c1N=C(c1c(F)cccc1F)Nc1cnc(N3CCC[C@@H](C#N)C3)cc1-2] | (3R)-1-[8-(2,6-difluorophenyl)-5- methyl-3,4,7,9,12- pentazatricyclo[8.4.0.02,6]tetradeca- 1(10),2(6),4,7,11,13-hexaen-13- yl]piperidine-3-carbonitrile | 420.2 |
| 19. | 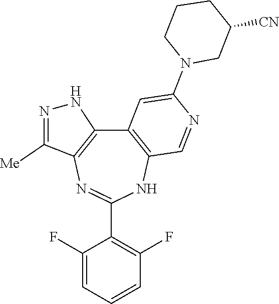 [SMILES: Cc1n[nH]c2c1N=C(c1c(F)cccc1F)Nc1cnc(N3CCC[C@H](C#N)C3)cc1-2] | (3S)-1-[8-(2,6-difluorophenyl)-5- methyl-3,4,7,9,12-pentazatricyclo [8.4.0.02,6]tetradeca- 1(10),2(6),4,7,11,13-hexaen-13- yl]piperidine-3-carbonitrile | 420.2 |
| 20 | 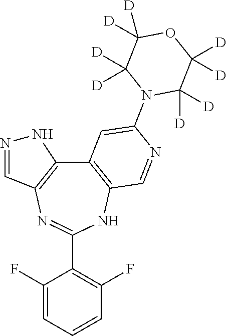 [SMILES: [2H]C1([2H])OC([2H])([2H])C([2H])([2H])N(c2cc3c(cn2)NC(c2c(F)cccc2F)=Nc2cn[nH]c2-3)C1([2H])[2H]] | 2,2,3,3,5,5,6,6-octadeuterio-4-[8-(2,6- difluorophenyl)-3,4,7,9,12- pentazatricyclo[8.4.0.02,6]tetradeca- 1(10),2(6),4,7,11,13-hexaen-13- yl]morpholine | 391.0 |
| 21 | 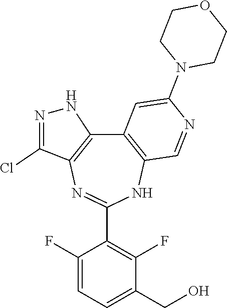 [SMILES: OCc1ccc(F)c(C2=Nc3c(Cl)n[nH]c3-c3cc(N4CCOCC4)ncc3N2)c1F] | (3-(3-chloro-9-morpholino-1,6- dihydropyrazolo[4,3-d]pyrido[4,3- f][1,3]diazepin-5-yl)-2,4- difluorophenyl)methanol | 447.0 |
| 22. | 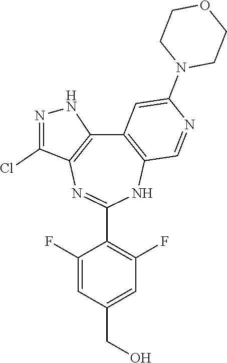 [SMILES: OCc1cc(F)c(C2=Nc3c(Cl)n[nH]c3-c3cc(N4CCOCC4)ncc3N2)c(F)c1] | (4-(3-chloro-9-morpholino-1,6- dihydropyrazolo[4,3-d]pyrido[4,3- f][1,3]diazepin-5-y1)-3,5- difluorophenyl)methanol | 447.0 |
| 23. | 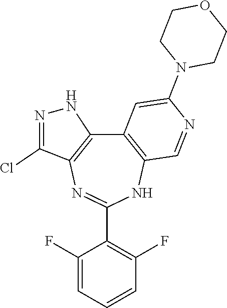 [SMILES: Fc1cccc(F)c1C1=Nc2c(Cl)n[nH]c2-c2cc(N3CCOCC3)ncc2N1] | 4-(3-chloro-5-(2,6-difluorophenyl)-1,6- dihydropyrazolo[4,3-d]pyrido[4,3- f][1,3]diazepin-9-yl)morpholine | 417.0 |
| 24 | 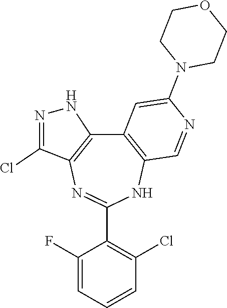 [SMILES: Fc1cccc(Cl)c1C1=Nc2c(Cl)n[nH]c2-c2cc(N3CCOCC3)ncc2N1] | (4-[5-chloro-8-(2-chloro-6-fluoro- phenyl)-3,4,7,9,12- pentazatricyclo[8.4.0.02,6]tetradeca- 1(10),2(6),4,7,11,13-hexaen-13- yl]morpholine) | 433.3 |
| 25. | 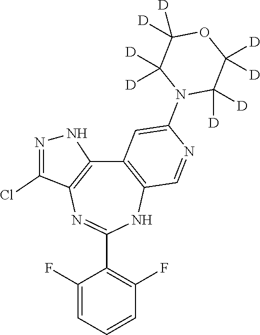 [SMILES: [2H]C1([2H])OC([2H])([2H])C([2H])([2H])N(c2cc3c(cn2)NC(c2c(F)cccc2F)=Nc2c(Cl)n[nH]c2-3)C1([2H])[2H]] | 4-[5-chloro-8-(2,6-difluorophenyl)- 3,4,7,9,12- pentazatricyclo[8.4.0.02,6]tetradeca- 1(10),2(6),4,7,11,13-hexaen-13-yl]- 2,2,3,3,5,5,6,6-octadeuterio-morpholine | 425.0 |
| 26. | 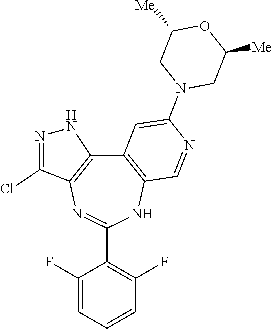 [SMILES: C[C@H]1CN(c2cc3c(cn2)NC(c2c(F)cccc2F)=Nc2c(Cl)n[nH]c2-3)C[C@H](C)O1] | (2S,6S)-4-(3-chloro-5-(2,6- difluorophenyl)-1,6- dihydropyrazolo[4,3-d]pyrido[4,3- f][1,3]diazepin-9-yl)-2,6- dimethylmorpholine | 445.1 |
| 27. | 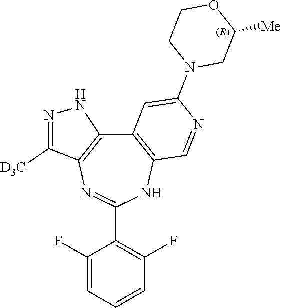 | (2R)-4-[8-(2,6-difluorophenyl)-5- (trideuteriomethyl)-3,4,7,9,12- pentazatricyclo[8.4.0.02,6]tetradeca- 1(10),2(6),4,7,11,13-hexaen-13-y1]-2- methyl-morpholine | 414.0 |
| 28. | 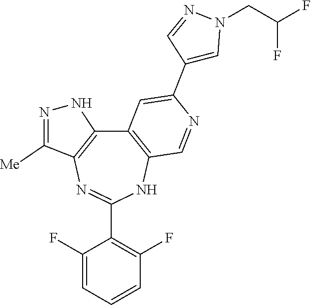 [SMILES: Cc1n[nH]c2c1N=C(c1c(F)cccc1F)Nc1cnc(-c3cnn(CC(F)F)c3)cc1-2] | 13-[1-(2,2-difluoroethyl)pyrazol-4-y1]- 8-(2,6-difluorophenyl)-5-methyl- 3,4,7,9,12- pentazatricyclo[8.4.0.02,6]tetradeca- 1(10),2(6),4,7,11,13-hexaene | 441.1 |
| 29. | 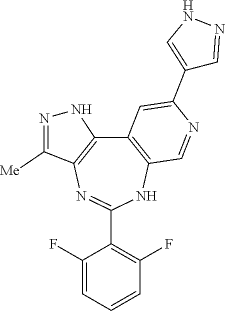 [SMILES: Cc1n[nH]c2c1N=C(c1c(F)cccc1F)Nc1cnc(-c3cn[nH]c3)cc1-2] | 8-(2,6-difluorophenyl)-5-methyl-13- (1H-pyrazol-4-y1)-3,4,7,9,12- pentazatricyclo[8.4.0.02,6]tetradeca- 1(10),2(6),4,7,11,13-hexaene | 378.1 |
| 30. | 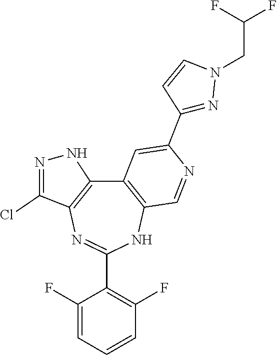 [SMILES: Fc1cccc(F)c1C1=Nc2c(Cl)n[nH]c2-c2cc(-c3ccn(CC(F)F)n3)ncc2N1] | (3-chloro-9-(1-(2,2-difluoroethyl)-1H- pyrazol-3-yl)-5-(2,6-difluorophenyl)- 1,6-dihydropyrazolo[4,3-d]pyrido[4,3- f][1,3]diazepine) | 461.0 |
| 31. | 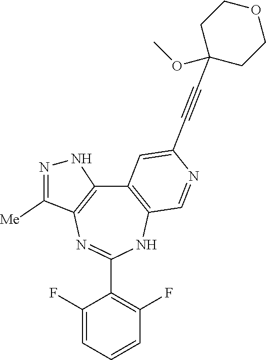 [SMILES: COC1(C#Cc2cc3c(cn2)NC(c2c(F)cccc2F)=Nc2c(C)n[nH]c2-3)CCOCC1] | 8-(2,6-difluorophenyl)-13-[2-(4- methoxytetrahydropyran-4-y1)ethynyl]- 5-methyl-3,4,7,9,12- pentazatricyclo[8.4.0.02,6]tetradeca- 1(10),2(6),4,7,11,13-hexaene | 450.1 |
| 32. | 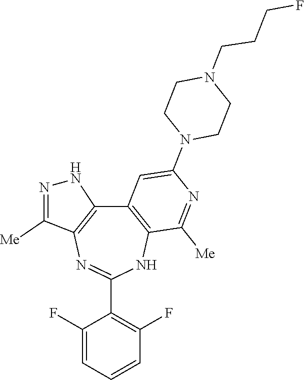 [SMILES: Cc1n[nH]c2c1N=C(c1c(F)cccc1F)Nc1c-2cc(N2CCN(CCCF)CC2)nc1C] | 8-(2,6-difluorophenyl)-13-[4-(3- fluoropropyl)piperazin-1-y1]-5,11- dimethyl-3,4,7,9,12- pentazatricyclo[8.4.0.02,6]tetradeca- 1(10),2(6),4,7,11,13-hexaene | 470.3 |
| 33. | 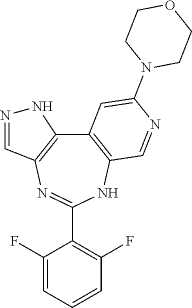 [SMILES: Fc1cccc(F)c1C1=Nc2cn[nH]c2-c2cc(N3CCOCC3)ncc2N1] | 4-[8-(2,6-difluorophenyl)-3,4,7,9,12- pentazatricyclo[8.4.0.02,6]tetradeca- 1(10),2(6),4,7,11,13-hexaen-13- yl]morpholine | 383.0 |
| 34. | 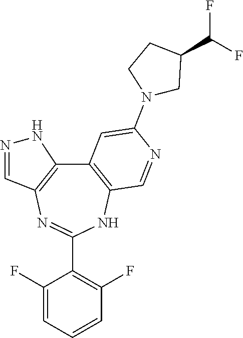 [SMILES: Fc1cccc(F)c1C1=Nc2cn[nH]c2-c2cc(N3CC[C@@H](C(F)F)C3)ncc2N1] | 13-[(3R)-3-(difluoromethyl)pyrrolidin- 1-yl]-8-(2,6-difluorophenyl)-3,4,7,9,12- pentazatricyclo[8.4.0.02,6]tetradeca- 1(10),2(6),4,7,11,13-hexaene | 417.2 |
| 35. | 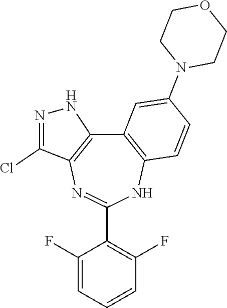 [SMILES: Fc1cccc(F)c1C1=Nc2c(Cl)n[nH]c2-c2cc(N3CCOCC3)ccc2N1] | 4-(3-chloro-5-(2,6-difluorophenyl)-1,6- dihydrobenzo[d]pyrazolo[3,4- f][1,3]diazepin-9-y1)morpholine | 416.0 |
| 36. | 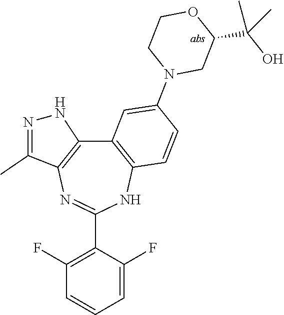 [SMILES: Cc1n[nH]c2c1N=C(c1c(F)cccc1F)Nc1ccc(N3CCO[C@H](C(C)(C)O)C3)cc1-2] | (S)-2-(4-(5-(2,6-difluorophenyl)-3- methyl-1,6- dihydrobenzo[d]pyrazolo[3,4- f][1,3]diazepin-9-yl)morpholin-2- y1)propan-2-ol | 453.48 |
| 37. | 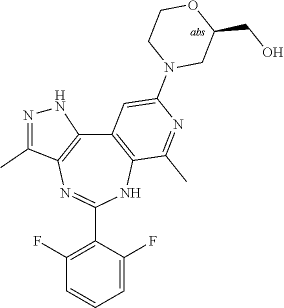 [SMILES: Cc1n[nH]c2c1N=C(c1c(F)cccc1F)Nc1c-2cc(N2CCO[C@@H](CO)C2)nc1C] | (R)-(4-(5-(2,6-difluorophenyl)-3,7- dimethyl-1,6-dihydropyrazolo[4,3- d]pyrido[4,3-f][1,3]diazepin-9- yl)morpholin-2-y1)methanol | 440.45 |
| 38. | 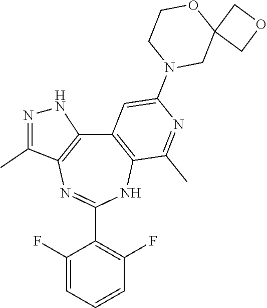 [SMILES: Cc1n[nH]c2c1N=C(c1c(F)cccc1F)Nc1c-2cc(N2CCOC3(COC3)C2)nc1C] | 8-(5-(2,6-difluorophenyl)-3,7-dimethyl- 1,6-dihydropyrazolo[4,3-d]pyrido[4,3- f][1,3]diazepin-9-yl)-2,5-dioxa-8- azaspiro[3.5]nonane | 452.46 |
| 39. | 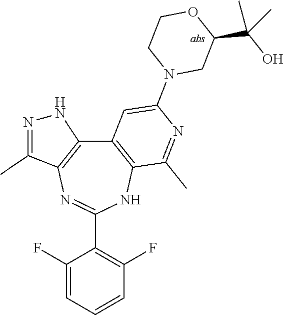 [SMILES: Cc1n[nH]c2c1N=C(c1c(F)cccc1F)Nc1c-2cc(N2CCO[C@@H](C(C)(C)O)C2)nc1C] | (R)-2-(4-(5-(2,6-difluorophenyl)-3,7- dimethyl-1,6-dihydropyrazolo[4,3- d]pyrido[4,3-f][1,3]diazepin-9- yl)morpholin-2-yl)propan-2-ol | 468.5 |
| 40. | 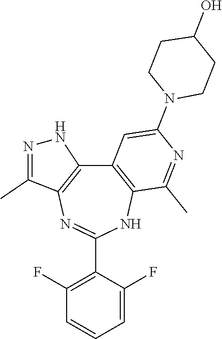 [SMILES: Cc1n[nH]c2c1N=C(c1c(F)cccc1F)Nc1c-2cc(N2CCC(O)CC2)nc1C] | 1-(5-(2,6-difluorophenyl)-3,7-dimethyl- 1,6-dihydropyrazolo[4,3-d]pyrido[4,3- f][1,3]diazepin-9-yl)piperidin-4-ol | 424.45 |
| 41. | 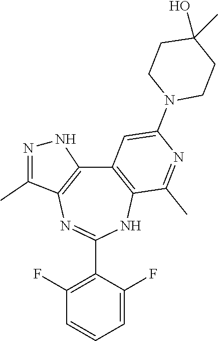 | 1-(5-(2,6-difluorophenyl)-3,7-dimethyl- 1,6-dihydropyrazolo[4,3-d]pyrido[4,3- f][1,3]diazepin-9-yl)-4-methylpiperidin- 4-ol | 438.47 |
| 42. | 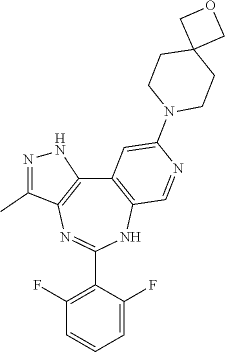 [SMILES: Cc1n[nH]c2c1N=C(c1c(F)cccc1F)Nc1cnc(N3CCC4(CC3)COC4)cc1-2] | 7-(5-(2,6-difluorophenyl)-3-methyl-1,6- dihydropyrazolo[4,3-d]pyrido[4,3- f][1,3]diazepin-9-yl)-2-oxa-7- azaspiro[3.5]nonane | 436.5 |
| 43. | 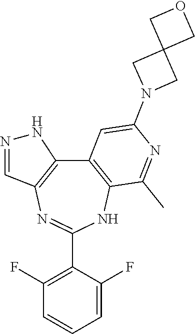 [SMILES: Cc1nc(N2CC3(COC3)C2)cc2c1NC(c1c(F)cccc1F)=Nc1cn[nH]c1-2] | 6-(5-(2,6-difluorophenyl)-7-methyl-1,6- dihydropyrazolo[4,3-d]pyrido[4,3- f][1,3]diazepin-9-y1)-2-oxa-6- azaspiro[3.3]heptane | 408.4 |
| 44. | 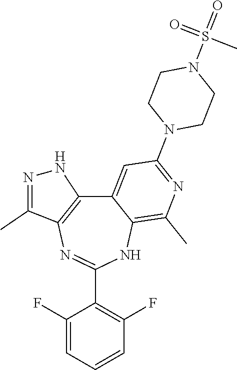 | 5-(2,6-difluorophenyl)-3,7-dimethyl-9- (4-(methylsulfonyl)piperazin-1-yl)-1,6- dihydropyrazolo[4,3-d]pyrido[4,3- f][1,3]diazepine | 487.5 |
| 45. | 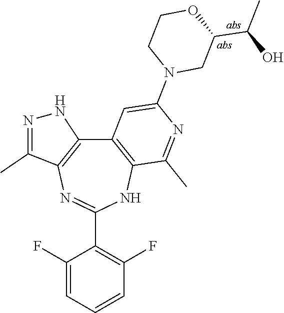 [SMILES: Cc1n[nH]c2c1N=C(c1c(F)cccc1F)Nc1c-2cc(N2CCO[C@H]([C@@H](C)O)C2)nc1C] | (R)-1-((S)-4-(5-(2,6-difluorophenyl)- 3,7-dimethyl-1,6-dihydropyrazolo[4,3- d]pyrido[4,3-f][1,3]diazepin-9- yl)morpholin-2-yl)ethan-1-ol | 454.5 |
| 46. | 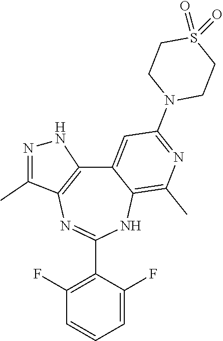 [SMILES: Cc1n[nH]c2c1N=C(c1c(F)cccc1F)Nc1c-2cc(N2CCS(=O)(=O)CC2)nc1C] | 4-(5-(2,6-difluorophenyl)-3,7-dimethyl- 1,6-dihydropyrazolo[4,3-d]pyrido[4,3- f][1,3]diazepin-9-yl)thiomorpholine 1,1- dioxide | 458.5 |
| 47. | 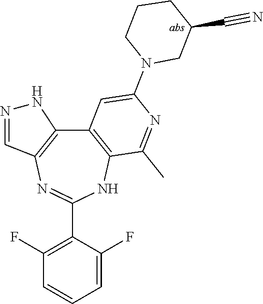 [SMILES: Cc1nc(N2CCC[C@@H](C#N)C2)cc2c1NC(c1c(F)cccc1F)=Nc1cn[nH]c1-2] | (R)-1-(5-(2,6-difluorophenyl)-7-methyl- 1,6-dihydropyrazolo[4,3-d]pyrido[4,3- f][1,3]diazepin-9-y1)piperidine-3- carbonitrile | 419.4 |
| 48. | 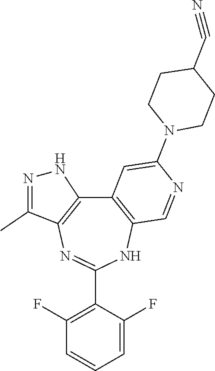 [SMILES: Cc1n[nH]c2c1N=C(c1c(F)cccc1F)Nc1cnc(N3CCC(C#N)CC3)cc1-2] | 1-(5-(2,6-difluorophenyl)-3-methyl-1,6- dihydropyrazolo[4,3-d]pyrido[4,3- f][1,3]diazepin-9-yl)piperidine-4- carbonitrile | 419.4 |
| 49. | 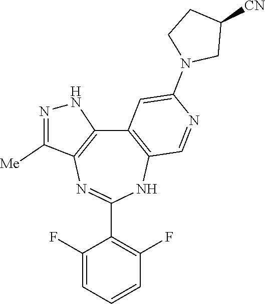 [SMILES: Cc1n[nH]c2c1N=C(c1c(F)cccc1F)Nc1cnc(N3CC[C@@H](C#N)C3)cc1-2] | (R)-1-(5-(2,6-difluorophenyl)-3-methyl- 1,6-dihydropyrazolo[4,3-d]pyrido[4,3- f][1,3]diazepin-9-y1)pyrrolidine-3- carbonitrile | 405.4 |
| 50. | 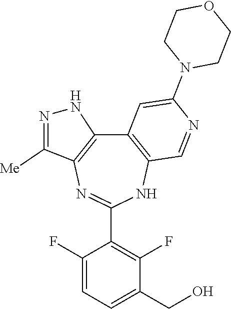 [SMILES: Cc1n[nH]c2c1N=C(c1c(F)ccc(CO)c1F)Nc1cnc(N3CCOCC3)cc1-2] | (2,4-difluoro-3-(3-methyl-9- morpholino-1,6-dihydropyrazolo[4,3- d]pyrido[4,3-f][1,3]diazepin-5- yl)phenyl)methanol | 426.4 |
| 51. | 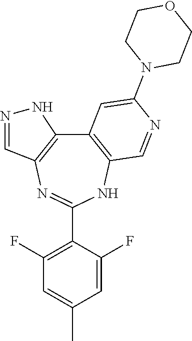 [SMILES: Cc1cc(F)c(C2=Nc3cn[nH]c3-c3cc(N4CCOCC4)ncc3N2)c(F)c1] | 4-(5-(2,6-difluoro-4-methylphenyl)-1,6- dihydropyrazolo[4,3-d]pyrido[4,3- f][1,3]diazepin-9-yl)morpholine | 396.4 |
| 52. | 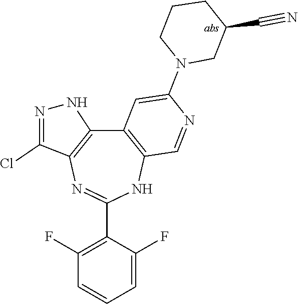 [SMILES: N#C[C@@H]1CCCN(c2cc3c(cn2)NC(c2c(F)cccc2F)=Nc2c(Cl)n[nH]c2-3)C1] | (R)-1-(3-chloro-5-(2,6-difluorophenyl)- 1,6-dihydropyrazolo[4,3-d]pyrido[4,3- f][1,3]diazepin-9-y1)piperidine-3- carbonitrile | 439.9 |
| 53. | 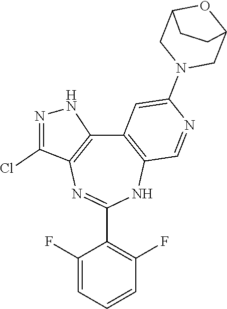 [SMILES: Fc1cccc(F)c1C1=Nc2c(Cl)n[nH]c2-c2cc(N3CC4CCC(C3)O4)ncc2N1] | 3-(3-chloro-5-(2,6-difluorophenyl)-1,6- dihydropyrazolo[4,3-d]pyrido[4,3- f][1,3]diazepin-9-yl)-8-oxa-3- azabicyclo[3.2.1]octane | 442.9 |
| 54. | 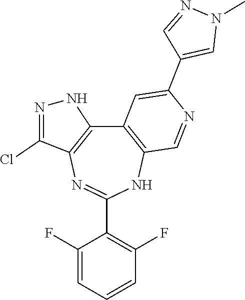 [SMILES: Cn1cc(-c2cc3c(cn2)NC(c2c(F)cccc2F)=Nc2c(Cl)n[nH]c2-3)cn1] | 3-chloro-5-(2,6-difluorophenyl)-9-(1- methyl-1H-pyrazol-4-yl)-1,6- dihydropyrazolo[4,3-d]pyrido[4,3- f][1,3]diazepine | 411.8 |

Major intermediates used in the synthetic preparations of the compounds described herein are provided below. These intermediates are given to enable those skilled in the art to more clearly understand and to practice the present disclosure. They should not be considered as limiting the scope of the disclosure, but merely as being illustrative and representative thereof.

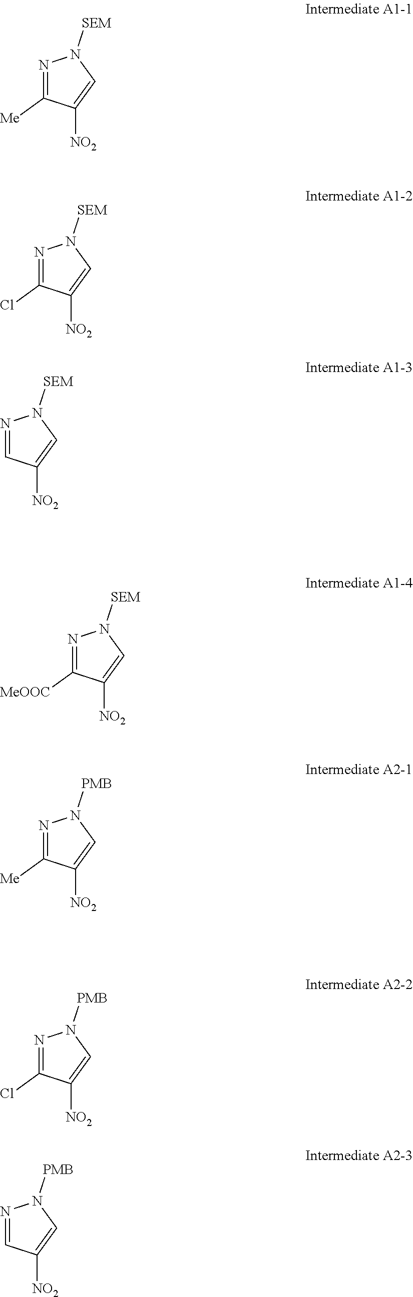

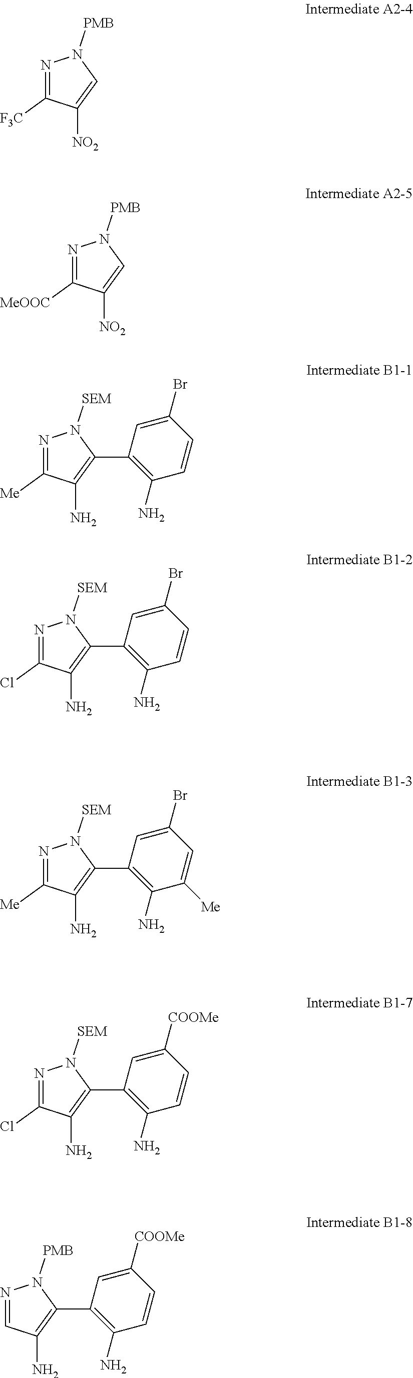

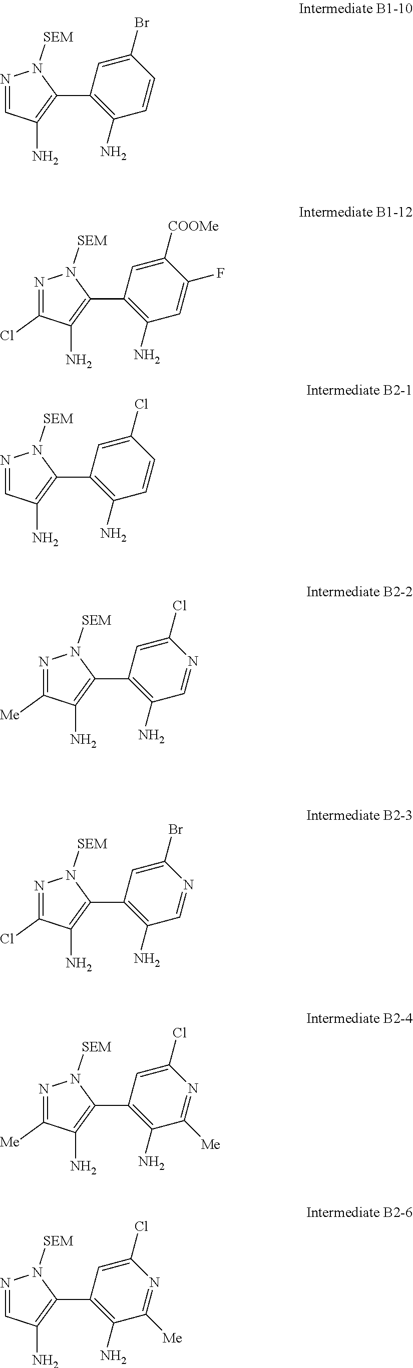

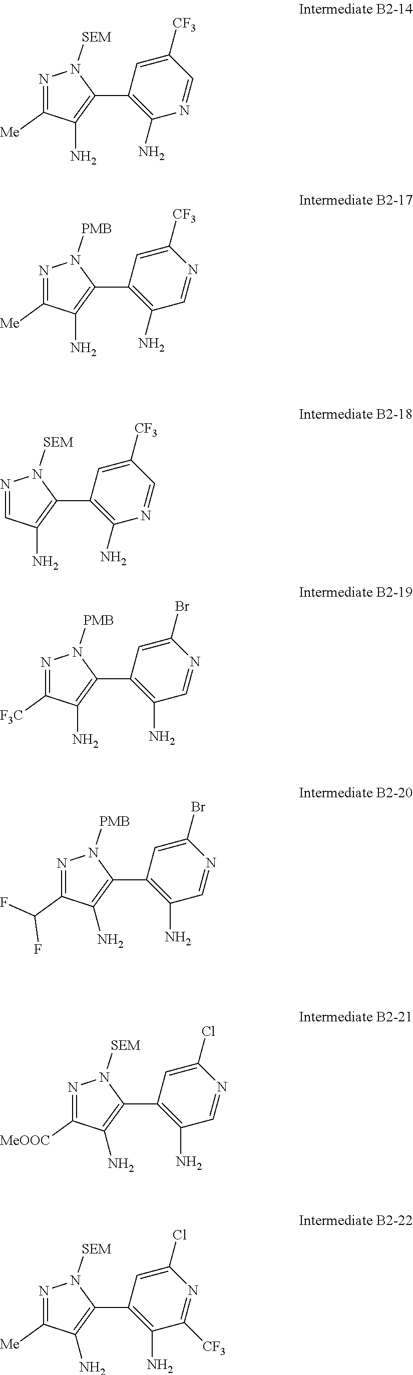

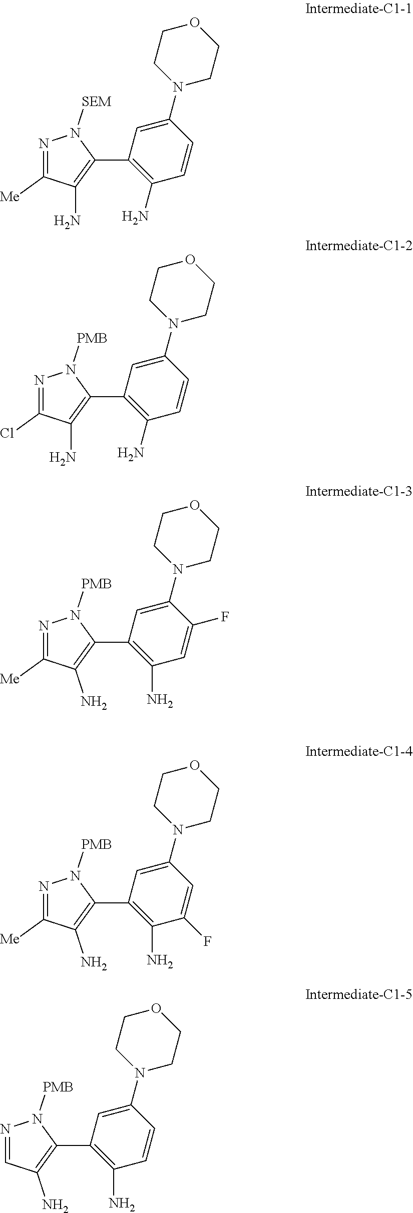

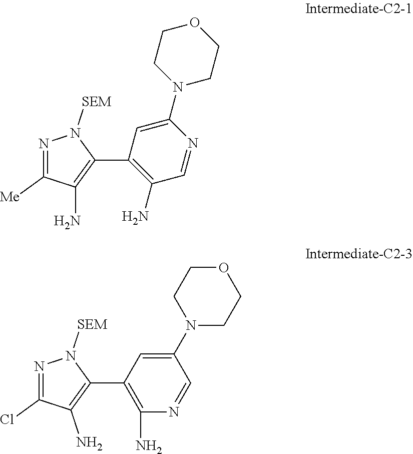 [SMILES: Cc1nn(COCC[Si](C)(C)C)c(-c2cc(N3CCOCC3)ncc2N)c1N ; C[Si](C)(C)CCOCn1nc(Cl)c(N)c1-c1cc(N2CCOCC2)cnc1N]

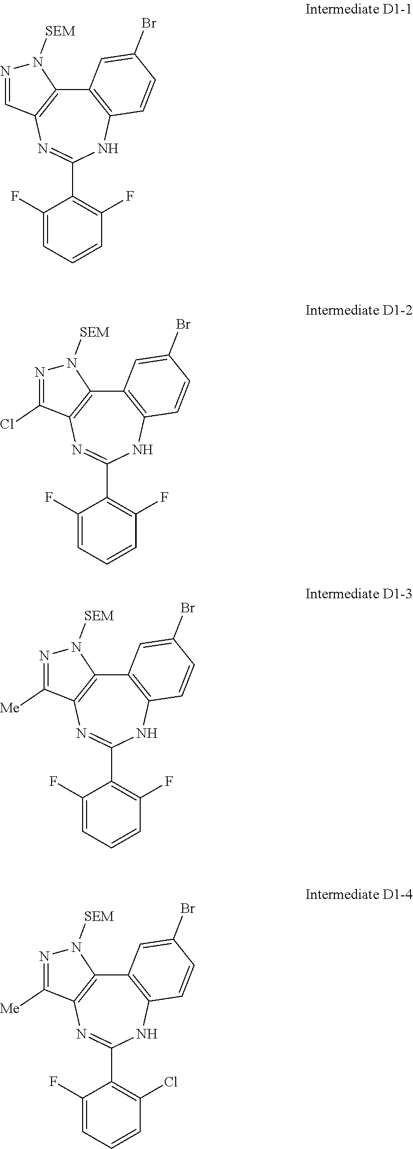 [SMILES: C[Si](C)(C)CCOCn1ncc2c1-c1cc(Br)ccc1NC(c1c(F)cccc1F)=N2 ; C[Si](C)(C)CCOCn1nc(Cl)c2c1-c1cc(Br)ccc1NC(c1c(F)cccc1F)=N2 ; Cc1nn(COCC[Si](C)(C)C)c2c1N=C(c1c(F)cccc1F)Nc1ccc(Br)cc1-2 ; Cc1nn(COCC[Si](C)(C)C)c2c1N=C(c1c(F)cccc1Cl)Nc1ccc(Br)cc1-2]

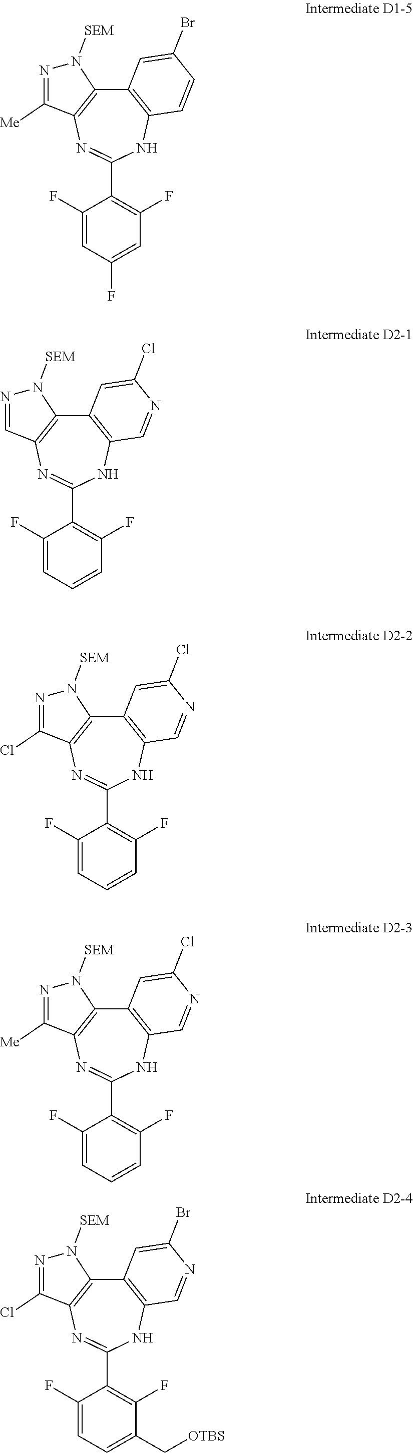 [SMILES: Cc1nn(COCC[Si](C)(C)C)c2c1N=C(c1c(F)cc(F)cc1F)Nc1ccc(Br)cc1-2 ; C[Si](C)(C)CCOCn1ncc2c1-c1cc(Cl)ncc1NC(c1c(F)cccc1F)=N2 ; C[Si](C)(C)CCOCn1nc(Cl)c2c1-c1cc(Cl)ncc1NC(c1c(F)cccc1F)=N2 ; Cc1nn(COCC[Si](C)(C)C)c2c1N=C(c1c(F)cccc1F)Nc1cnc(Cl)cc1-2 ; CC(C)(C)[Si](C)(C)OCc1ccc(F)c(C2=Nc3c(Cl)nn(COCC[Si](C)(C)C)c3-c3cc(Br)ncc3N2)c1F]

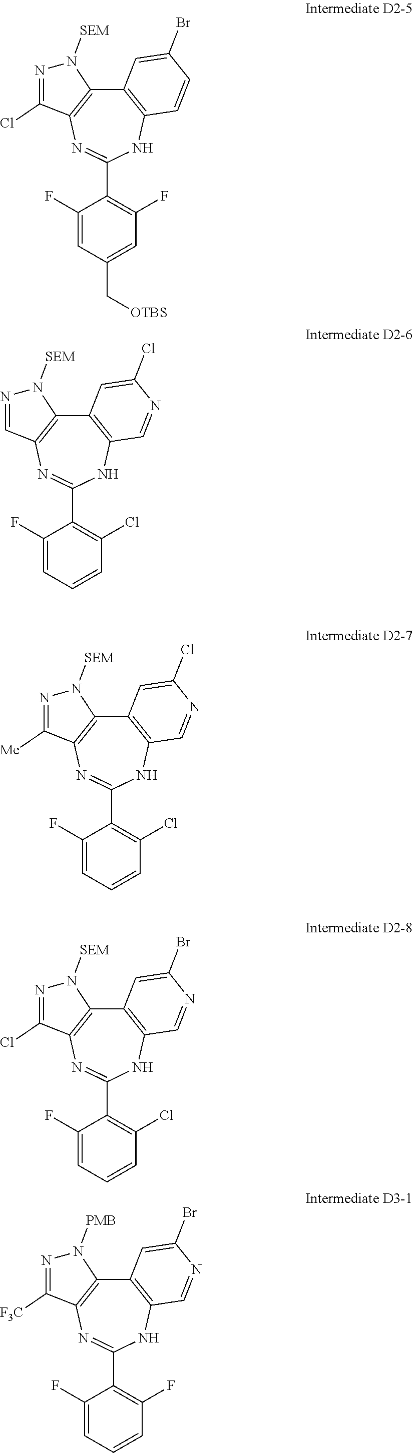

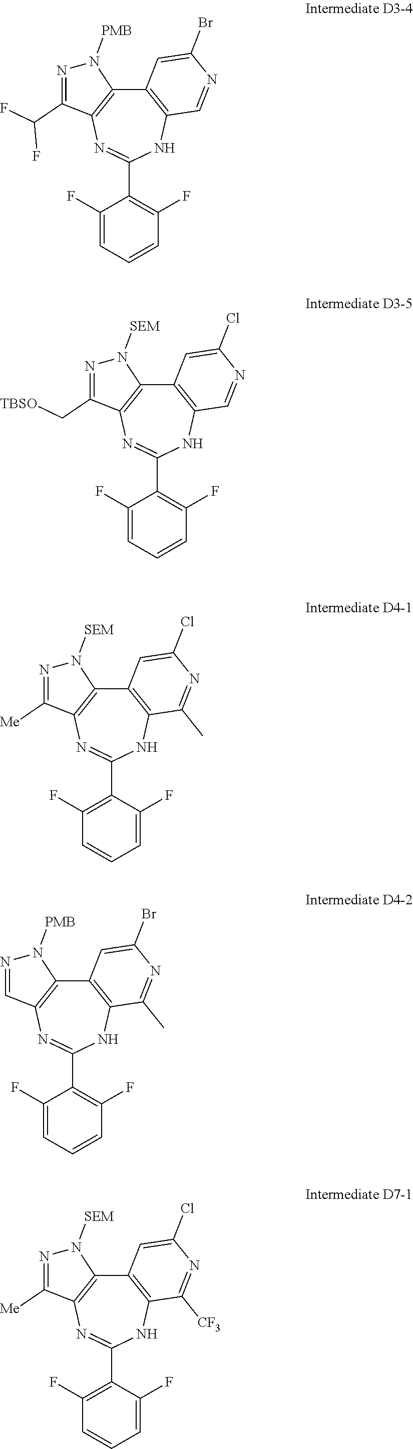

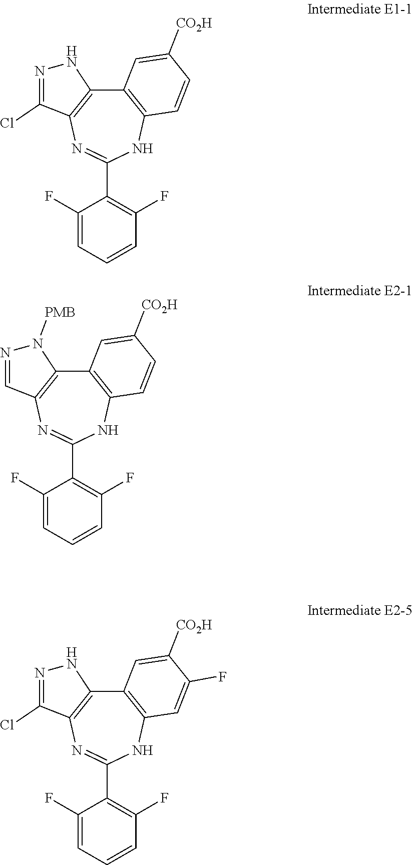

### Synthesis of 3-methyl-4-nitro-1-((2-(trimethylsilyl)ethoxy)methyl)-1H-pyrazole (Intermediate A1-1)

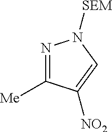 [SMILES: Cc1nn(COCC[Si](C)(C)C)cc1[N+](=O)[O-]]

To a solution of 3-methyl-4-nitro-1H-pyrazole (25 g, 0.197 mol, 1.0 eq) in THF (400 mL) was added NaH (11.8 g, 0.295 mol, 60% wt % in mineral oil, 1.5 eq) portion-wise at 0° C. under nitrogen atmosphere. The mixture was stirred at 0° C. for 30 min. SEM-Cl (42.0 mL, 0.237 mol, 1.2 eq) was added dropwise at 0° C. The mixture was warmed to 20° C. and stirred at 20° C. for 1 hr after the addition. The reaction was quenched with H₂O (100 mL) slowly under nitrogen atmosphere. The resulting mixture was diluted with water (100 mL), extracted with EtOAc (100 mL×3). The combined organic layers were washed with brine (100 mL×3) dried over anhydrous Na₂SO₄, filtered and concentrated under reduced pressure. The residue was purified by flash chromatography on silica gel column using 0-5% of EtOAc in petroleum ether as eluent), to provide the title compound as a yellow oil (41 g, 81%, containing some isomer). LCMS (ESI) m/z: 257.9 [M+H]⁺.

### Synthesis of 3-chloro-4-nitro-1-((2-(trimethylsilyl)ethoxy)methyl)-1H-pyrazole (Intermediate A1-2)

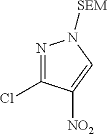 [SMILES: C[Si](C)(C)CCOCn1cc([N+](=O)[O-])c(Cl)n1]

### Step 1: Synthesis of 2-[(5-chloro-4-nitro-pyrazol-1-yl)methoxy]ethyl-trimethyl-silane

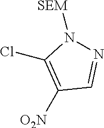 [SMILES: C[Si](C)(C)CCOCn1ncc([N+](=O)[O-])c1Cl]

To a mixture of 4-nitro-1-((2-(trimethylsilyl)ethoxy)methyl)-1H-pyrazole (12.5 g, 51.3 mmol, 1.0 eq) in THF (100 mL) was added 1M LiHMDS in THF (77.0 mL, 77 mmol, 1.5 eq) dropwise at −78° C. After addition, the resulting solution was stirred 0.5 hr at this temperature, followed by the addition of NCS (8.23 g, 61.6 mmol). The reaction mixture was stirred for 2 hr at −78° C. under N₂ atmosphere, then diluted with H₂O (30 mL) and extracted with EtOAc (30 mL×3). The combined organic layers were washed with brine (30 mL×2), dried with anhydrous Na₂SO₄, filtered and concentrated under reduced pressure. The residue was purified by flash chromatography (ISCO®; 330 g SepaFlash® Silica Flash Column, petroleum ether/EtOAc with EtOAc from 0˜2%, 100 mL/min, 254 mn), to provide the title compound as a yellow oil (12.2 g, 85%). LCMS (ESI) m/z: 278.1 [M+H]⁺.

### Step 2: Synthesis of 5-chloro-4-nitro-1H-pyrazole

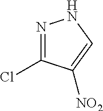

A mixture of 2-[(5-chloro-4-nitro-pyrazol-1-yl)methoxy]ethyl-trimethyl-silane (24 g, 86.4 mmol, 1.0 eq) in 4N HCl in MeOH (200 mL) was stirred at 20° C. for 2 hr. The reaction mixture was concentrated under reduced pressure to provide the crude product of 5-chloro-4-nitro-1H-pyrazole as a white solid (13 g).

### Step 3: Synthesis of 2-[(3-chloro-4-nitro-pyrazol-1-yl)methoxy]ethyl-trimethyl-silane

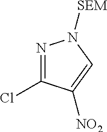 [SMILES: C[Si](C)(C)CCOCn1cc([N+](=O)[O-])c(Cl)n1]

A mixture of 3-chloro-4-nitro-1H-pyrazole (12 g, 81.3 mmol, 1.0 eq), SEM-Cl (14.9 g, 89.4 mmol, 1.1 eq) and Cs₂CO₃ (79.5 g, 0.244 mol, 3.0 eq) in THF (200 mL) was stirred at 20° C. for 2 hr. Then the reaction mixture was diluted with H₂O (100 mL) and extracted with EtOAc (100 mL×3). The combined organic layers were washed with brine (30 mL×2), dried over anhydrous Na₂SO₄, filtered and concentrated under reduced pressure. The crude product was purified by flash silica gel chromatography (ISCO®; 120 g SepaFlash® Silica Flash Column, Eluent of 0˜1.5% petroleum ether/EtOAc @ 35 mL/min) to provide 2-[(3-chloro-4-nitro-pyrazol-1-yl)methoxy]ethyl-trimethyl-silane as a white solid (14 g, 30.9%). LCMS (ESI) m/z: 278.1 [M+H]⁺.

### Synthesis of 4-nitro-1-((2-(trimethylsilyl)ethoxy)methyl)-1H-pyrazole (Intermediate A1-3)

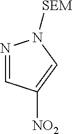 [SMILES: C[Si](C)(C)CCOCn1cc([N+](=O)[O-])cn1]

To a solution of 4-nitro-1H-pyrazole (10 g, 88.4 mmol) in THF (200 mL) was added NaH (5.31 g, 0.132 mol, 60% wt % in mineral oil, 1.5 eq) portion-wise at 0° C. under nitrogen atmosphere. The mixture was stirred at 0° C. for 30 min. SEM-Cl (16.2 g, 97.2 mmol, 1.2 eq) was added dropwise at 0° C. The mixture was warmed to 20° C. and stirred at 20° C. for 1 hr after the addition. The reaction was quenched with H₂O (100 mL) slowly under nitrogen atmosphere. The resulting mixture was diluted with water (100 mL), extracted with EtOAc (50 mL×3). The combined organic layers were washed with brine (50 mL×3) dried over anhydrous Na₂SO₄, filtered and concentrated under reduced pressure. The residue was purified by flash chromatography on silica gel column using 0-5% of EtOAc in petroleum ether as eluent), to provide the title compound as a yellow oil (20 g, 93%). LCMS (ESI) m/z: 244.1 [M+H]⁺.

### Synthesis of 4-nitro-1-((2-(trimethylsilyl)ethoxy)methyl)-1H-pyrazole (Intermediate A1-4)

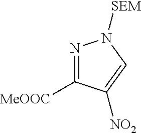 [SMILES: COC(=O)c1nn(COCC[Si](C)(C)C)cc1[N+](=O)[O-]]

To a solution of methyl 4-nitro-1H-pyrazole-3-carboxylate (10 g, 58.4 mmol, 1 eq) in THF (250 mL) was added NaH (3.51 g, 87.7 mmol, 60% purity, 1.5 eq) portion wise at 0° C. under nitrogen atmosphere. The mixture was stirred at 0° C. for 30 min. SEM-Cl (11.68 g, 70.1 mmol, 1.2 eq) was added dropwise at 0° C. The mixture was warmed to 20° C. and stirred at 20° C. for 1 hr after the addition. The reaction was quenched with H₂O (100 mL) slowly under nitrogen atmosphere. The mixture was extracted with EtOAc (100 mL×3). The combined organic layer was washed with brine (100 mL), dried over anhydrous Na₂SO₄, filtered and concentrated under reduced pressure. The residue was purified by flash chromatography (ISCO®; 220 g AgelaFlash® Silica Flash Column, DCM/MeOH with from MeOH 0˜10%, flow rate=100 mL/min, 254 nm) to afford methyl 4-nitro-1-(2-trimethylsilylethoxymethyl)pyrazole-3-carboxylate (15.37 g, 87.3%) as a yellow oil. LCMS (ESI) m/z: 302.1 [M+H]⁺.

### Synthesis of 1-[(4-methoxyphenyl)methyl]-4-nitro-pyrazole (Intermediate A2-1)

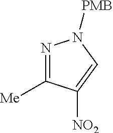

A mixture of 3-methyl-4-nitro-1H-pyrazole (50.6 g, 0.398 mmol, 1.0 eq), K₂CO₃ (82.5 g, 0.60 mol, 1.5 eq) and 1-(chloromethyl)-4-methoxybenzene (68.6 g, 0.438 mol, 1.1 eq) in MeCN (500 mL) was heated to 55° C. and stirred for 4 hr. The reaction mixture was cooled to room temperature, poured into H₂O (200 mL) and extracted with EtOAc (300 mL×2). The combined organic phase was washed with brine (200 mL), dried over anhydrous Na₂SO₄ and concentrated in vacuum. The residue was purified by flash chromatography on silica gel column using 0-30% of EtOAc in petroleum ether as eluent, to provide the title compound as a light-yellow solid (90 g, 91.5%). ¹H NMR (400 MHz, CDCl₃) δ ppm 3.84 (s, 3H), 5.26 (s, 2H), 6.93 (d, J=8.4 Hz, 2H), 6.93-6.98 (m, 1H), 7.27 (d, J 8.4 Hz, 2H), 8.02 (s, 1H) 8.10 (s, 1H).

### Synthesis of 3-chloro-1-(4-methoxybenzyl)-4-nitro-1H-pyrazole (Intermediate A2-2)

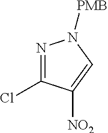

A mixture of 3-chloro-4-nitro-1H-pyrazole (10 g, 67.7 mmol, 1.0 eq), K₂CO₃ (28.1 g, 0.203 mol, 3.0 eq) and PMB-Cl (11.6 g, 74.5 mmol, 1.1 eq) in DMF (200 mL) was stirred at 25° C. for 16 hr. The reaction mixture was diluted with H₂O (300 mL) and extracted with EtOAc (200 mL×3). The combined organic layers were washed with brine (100 mL×2), dried over anhydrous Na₂SO₄, filtered and concentrated under reduced pressure. The crude product was further purified by flash silica gel chromatography (ISCO®; 20 g SepaFlash® Silica Flash Column, Eluent of 0˜20% petroleum ether/EtOAc @ 35 mL/min), to provide the title compound as a light-yellow solid (18 g, 50%). ¹H NMR (400 MHz, DMSO-d₆) δ ppm 3.74 (s, 3H), 5.28 (s, 2H), 6.93 (d, J=8.8 Hz, 2H), 7.33 (d, J=8.8 Hz, 2H), 9.06-9.17 (m, 1H).

### Synthesis of 3-chloro-1-(4-methoxybenzyl)-4-nitro-1H-pyrazole (Intermediate A2-3)

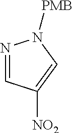

A mixture of 4-nitro-1H-pyrazole (45 g, 0.398 mol, 1.0 eq), K₂CO₃ (82.5 g, 0.60 mol) and 1-(chloromethyl)-4-methoxybenzene (68.56 g, 0.438 mol) in MeCN (500 mL) was stirred at 55° C. for 4 h. The reaction was cooled to room temperature and then poured into 200 mL of H₂O, extracted with EtOAc (200 mL×2). The combined extracts were washed with brine (200 mL), dried over anhydrous Na₂SO₄ and concentrated in vacuum. The residue was purified by flash chromatography on silica gel column using 0-30% of EtOAc in petroleum ether as eluent, to provide the title compound as a light-yellow solid (90 g, 97.0%). ¹H NMR (400 MHz, CDCl₃) δ ppm 3.84 (s, 3H), 5.26 (s, 2H), 6.95 (d, J 8.4 Hz, 2H), 6.93 (m, 1H), 7.27 (d, J 8.4 Hz, 2H), 8.02 (s, 1H), 8.10 (s, 1H).

### Synthesis of 1-(4-methoxybenzyl)-4-nitro-3-(trifluoromethyl)-1H-pyrazole (Intermediate A2-4)

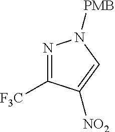

### Step 1: 4-nitro-3-(trifluoromethyl)-1H-pyrazole

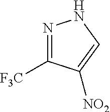 [SMILES: O=[N+]([O-])c1c[nH]nc1C(F)(F)F]

To a solution of 3-(trifluoromethyl)-1H-pyrazole (5 g, 36.74 mmol, 1.0 eq.) in H₂SO₄ (20 mL) was added dropwise the conc. HNO₃ (2.68 g, 40.4 mmol, 1.1 eq.) at 0° C. The mixture was stirred at 80° C. for 2 hr, then diluted with ice water (10 mL) and filtered to provide 4-nitro-3-(trifluoromethyl)-1H-pyrazole as a white solid (6.8 g, 51.1%). ¹H NMR (400 MHz, DMSO-d₆) δ ppm 9.15 (s, 1H).

### Step 2: 1-(4-methoxybenzyl)-4-nitro-3-(trifluoromethyl)-1H-pyrazole

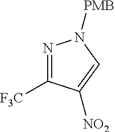

To a solution of 4-nitro-3-(trifluoromethyl)-1H-pyrazole (6.5 g, 35.9 mmol, 1.0 eq.) in MeCN (130 mL) was added K₂CO₃ (14.9 g, 0.108 mol, 3.0 eq.) and PMB-Cl (6.18 g, 39.5 mmol, 1.1 eq.). The mixture was stirred at 25° C. for 16 hr. The reaction mixture was filtered. The filter liquor was concentrated under reduced pressure. The residue was purified by flash chromatography on silica gel column using 0-10% of EtOAc in petroleum ether as eluent, to provide 1-[(4-methoxyphenyl)methyl]-4-nitro-3-(trifluoromethyl)pyrazole as a white solid (16.3 g, 75.4%).

### Synthesis of 4-[4-amino-5-(difluoromethyl)-2-[(4-methoxyphenyl)methyl]pyrazol-3-yl]-6-bromo-pyridin-3-amine (A-5)

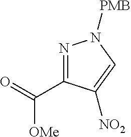

To a solution of methyl 4-nitro-1H-pyrazole-5-carboxylate (5 g, 29.2 mmol, 1 eq.) in DMF (200 mL) was added K₂CO₃ (8.08 g, 58.4 mmol, 2 eq.) and PMB-Cl (5.03 g, 32.1 mmol, 1.1 eq.). The mixture was stirred at 25° C. for 2 hr. The reaction mixture was added H₂O (50 mL) and extracted with EtOAc (50 mL×3). The combined organic layers were washed with brine (30 mL), dried with anhydrous Na₂SO₄, filtered and concentrated under reduced pressure. The residue was purified by flash chromatography on silica gel column using 0-30% of EtOAc in petroleum ether as eluent, to provide methyl 1-[(4-methoxyphenyl)methyl]-4-nitro-pyrazole-3-carboxylate as a yellow oil (6.5 g, 76.3%). ¹H NMR (400 MHz, DMSO-d₆) δ ppm 3.67-3.77 (m, 3H), 3.81-3.93 (m, 3H), 5.25-5.54 (m, 2H), 6.83-7.03 (m, 2H), 7.23-7.44 (m, 2H), 8.96-9.23 (m, 1H).

### Synthesis of 5-(2-amino-5-bromophenyl)-3-methyl-1-((2-(trimethylsilyl)ethoxy)methyl)-1H-pyrazol-4-amine (Intermediate B1-1)

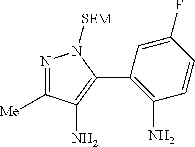 [SMILES: Cc1nn(COCC[Si](C)(C)C)c(-c2cc(F)ccc2N)c1N]

### Step 1: tert-butyl N-(2-bromophenyl)carbamate

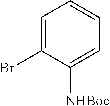 [SMILES: CC(C)(C)OC(=O)Nc1ccccc1Br]

A mixture of 2-bromoaniline (60.0 g, 0.349 mol, 1 eq.), Boc₂O (115 g, 0.527 mol, 1.5 eq.) was degassed and purged with N₂ for 3 times, and then the mixture was stirred at 100° C. for 48 hr under N₂ atmosphere until the starting material was completely consumed, cooled to room temperature. The resulting mixture was quenched by addition H₂O (100 mL) at 25° C., and then extracted with EtOAc (100 mL×3). The combined organic layers were washed with brine (100 mL×3), dried over anhydrous Na₂SO₄, filtered and concentrated under reduced pressure. The residue was purified by flash chromatography on silica gel column using 0-15% of EtOAc in petroleum ether as eluent, to provide tert-butyl N-(2-bromophenyl)carbamate as a yellow oil (87.0 g, 85.8%). LCMS (ESI) m/z: 271.7 [M+H]⁺.

### Step 2: tert-butyl (2-(3-methyl-4-nitro-1-((2-(trimethylsilyl)ethoxy)methyl)-1H-pyrazol-5-yl)phenyl)carbamate

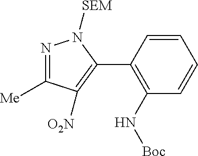 [SMILES: Cc1nn(COCC[Si](C)(C)C)c(-c2ccccc2NC(=O)OC(C)(C)C)c1[N+](=O)[O-]]

A mixture of tert-butyl N-(2-bromophenyl)carbamate (A1-1) (10 g, 36.8 mmol, 1 eq.), trimethyl-[2-[(3-methyl-4-nitro-pyrazol-1-yl)methoxy]ethyl]silane (19.0 g, 73.8 mmol, 2 eq.), Pd(OAc)₂ (825 mg, 3.67 mmol, 0.1 eq.), Xphos (5.26 g, 11.0 mmol, 0.3 eq.) and Cs₂CO₃ (24.0 g, 73.7 mmol, 2 eq.), 2,2-dimethylpropanoic acid (7.51 g, 73.5 mmol, 2 eq.) in toluene (200 mL) was degassed and purged with N₂ for 3 times, and then the mixture was stirred at 100° C. for 16 hr under N₂ atmosphere. The reaction mixture was quenched by addition H₂O (100 mL) at 25° C., and then extracted with EtOAc (100 mL×3). The combined organic layers were washed with brine (100 mL×3), dried over anhydrous Na₂SO₄, filtered and concentrated under reduced pressure to provide tert-butyl N-[2-[5-methyl-4-nitro-2-(2-trimethylsilylethoxymethyl)pyrazol-3-yl]phenyl]carbamate as a brown oil (22.5 g, 41.0%). LCMS (ESI) m/z: 449.1 [M+H]⁺.

### Step 3: tert-butyl (4-bromo-2-(3-methyl-4-nitro-1-((2-(trimethylsilyl)ethoxy)methyl)-1H-pyrazol-5-yl)phenyl)carbamate

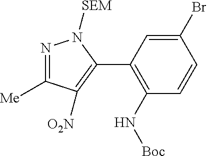 [SMILES: Cc1nn(COCC[Si](C)(C)C)c(-c2cc(Br)ccc2NC(=O)OC(C)(C)C)c1[N+](=O)[O-]]

A mixture of tert-butyl N-[2-[5-methyl-4-nitro-2-(2-trimethylsilylethoxymethyl)pyrazol-3-yl]phenyl]carbamate (30.0 g, 66.9 mmol, 1 eq.), NBS (12.0 g, 67.4 mmol, 1 eq.) in MeCN (150 mL) was degassed and purged with N₂ for 3 times, and then the mixture was stirred at 20° C. for 48 hr under N₂ atmosphere. The residue was filtered and concentrated under reduced pressure to provide the crude product of tert-butyl N-[4-bromo-2-[5-methyl-4-nitro-2-(2-trimethylsilylethoxymethyl)pyrazol-3-yl]phenyl]carbamate as a brown oil (45.0 g). LCMS (ESI) m/z: 529.1 [M+H]⁺.

### Step 4: tert-butyl (2-(4-amino-3-methyl-1-((2-(trimethylsilyl)ethoxy)methyl)-1H-pyrazol-5-yl)-4-bromophenyl)carbamate

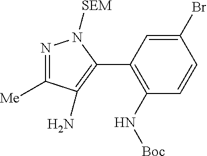 [SMILES: Cc1nn(COCC[Si](C)(C)C)c(-c2cc(Br)ccc2NC(=O)OC(C)(C)C)c1N]

A mixture of tert-butyl N-[4-bromo-2-[5-methyl-4-nitro-2-(2-trimethylsilylethoxymethyl) pyrazol-3-yl]phenyl]carbamate (40.0 g, 37.9 mmol, 1 eq.), Fe (10.6 g, 0.190 mol, 5 eq.), NH₄Cl (10.2 g, 0.190 mol, 5 eq.) in EtOH/H₂O (1:1, 200 mL) as degassed and purged with N₂ for 3 times, and then the mixture was stirred at 80° C. for 2 hr under N₂ atmosphere. The resulting mixture was quenched by addition H₂O (50 mL) at 25° C., and then extracted with EtOAc (50 mL×3). The combined organic layers were washed with brine (50 mL×3), dried over anhydrous Na₂SO₄, filtered and concentrated under reduced pressure. The residue was purified by flash chromatography on silica gel column using 0-15% of EtOAc in petroleum ether as eluent, to provide tert-butyl N-[2-[4-amino-5-methyl-2-(2-trimethylsilylethoxymethyl)pyrazol-3-yl]-4-bromo-phenyl]carbamate as a red oil (3.0 g, 15.9%). LCMS (ESI) m/z: 499.2 [M+H]⁺.

### Step 5: 5-(2-amino-5-bromophenyl)-3-methyl-1-((2-(trimethylsilyl)ethoxy)methyl)-1H-pyrazol-4-amine

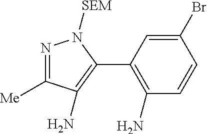 [SMILES: Cc1nn(COCC[Si](C)(C)C)c(-c2cc(Br)ccc2N)c1N]

To a rt solution of tert-butyl N-[2-[4-amino-5-methyl-2-(2-trimethylsilylethoxymethyl) pyrazol-3-yl]-4-bromo-phenyl]carbamate (7.86 g, 15.8 mmol, 1 eq.) in DCM (80 mL) was added ZnBr₂ (7.15 g, 31.7 mmol, 2 eq.) and the mixture was stirred at 40° C. for 12 hr under N₂ atmosphere. The reaction mixture was diluted with THF (20 mL) before the addition of anhydrous Na₂SO₄, filtered and concentrated under reduced pressure to provide the crude product of 5-(2-amino-5-bromo-phenyl)-3-methyl-1-(2-trimethylsilylethoxymethyl)pyrazol-4-amine as a brown oil (8 g). LCMS (ESI) m/z: 399.2 [M+H]⁺.

### Synthesis of 5-(2-amino-5-bromo-phenyl)-3-chloro-1-(2-trimethylsilylethoxymethyl) pyrazol-4-amine (Intermediate B1-2)

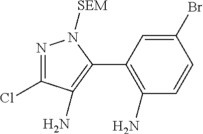 [SMILES: C[Si](C)(C)CCOCn1nc(Cl)c(N)c1-c1cc(Br)ccc1N]

### Step 1: 2-[5-chloro-4-nitro-2-(2-trimethylsilylethoxymethyl)pyrazol-3-yl]aniline

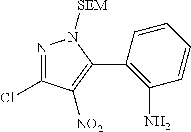 [SMILES: C[Si](C)(C)CCOCn1nc(Cl)c([N+](=O)[O-])c1-c1ccccc1N]

A mixture of 2-[(3-chloro-4-nitro-pyrazol-1-yl)methoxy]ethyl-trimethyl-silane (Intermediate A1-2) (10 g, 36 mmol, 1 eq), 2-iodoaniline (7.88 g, 36 mmol, 1 eq), Pd(PPh₃)₄ (4.16 g, 3.6 mmol, 0.1 eq), CuI (686 mg, 3.60 mmol, 0.1 eq) and Cs₂CO₃ (17.6 g, 54.0 mmol, 1.5 eq) in dioxane (100 mL) was degassed and purged with N₂ for 3 times, and then the mixture was stirred at 80° C. for 12 hr under N₂ atmosphere. The reaction mixture was filtered and concentrated under reduced pressure. The residue was purified by flash silica gel chromatography (ISCO®; 40 g SepaFlash® Silica Flash Column, Eluent of 0˜40% petroleum ether/EtOAc @ 45 mL/min) to give 2-[5-chloro-4-nitro-2-(2-trimethylsilylethoxymethyl)pyrazol-3-yl]aniline (6.6 g, 46.7%) was obtained as a dark green liquid. LCMS (ESI) m/z: 369.1 [M+H]⁺.

### Step 2: 4-bromo-2-[5-chloro-4-nitro-2-(2-trimethylsilylethoxymethyl)pyrazol-3-yl]aniline

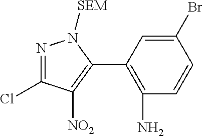 [SMILES: C[Si](C)(C)CCOCn1nc(Cl)c([N+](=O)[O-])c1-c1cc(Br)ccc1N]

A mixture of 2-[5-chloro-4-nitro-2-(2-trimethylsilylethoxymethyl)pyrazol-3-yl]aniline (8.1 g, 21.9 mmol, 1 eq), NBS (3.91 g, 21.9 mmol, 1 eq) in MeCN (80 mL) was degassed and purged with N₂ for 3 times, and then the mixture was stirred at 25° C. for 1 hr under N₂ atmosphere. The reaction mixture was concentrated under reduced pressure. The residue was purified by flash silica gel chromatography (ISCO®; 40 g SepaFlash® Silica Flash Column, Eluent of 0˜30% petroleum ether/EtOAc @ 35 mL/min) to give 4-bromo-2-[5-chloro-4-nitro-2-(2-trimethylsilylethoxymethyl)pyrazol-3-yl]aniline (6.30 g, 63.4%) was obtained as a yellow solid. LCMS (ESI) m/z: 449.1 [M+H]⁺.

### Step 3: 5-(2-amino-5-bromo-phenyl)-3-chloro-1-(2-trimethylsilylethoxymethyl) pyrazol-4-amine

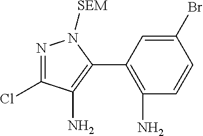 [SMILES: C[Si](C)(C)CCOCn1nc(Cl)c(N)c1-c1cc(Br)ccc1N]

To a solution of 4-bromo-2-[5-chloro-4-nitro-2-(2-trimethylsilylethoxymethyl)pyrazol-3-yl]aniline (5.8 g, 12.9 mmol, 1 eq) in EtOH (60 mL) and H₂O (20 mL) was added Fe (4.34 g, 77.7 mmol, 6 eq) and NH₄Cl (4.16 g, 77.7 mmol, 6 eq). The mixture was stirred at 80° C. for 2 hr and then concentrated under reduced pressure. The residue was purified by flash silica gel chromatography (ISCO®; 40 g SepaFlash® Silica Flash Column, Eluent of 0˜30% petroleum ether/EtOAc @ 35 mL/min) to give 5-(2-amino-5-bromo-phenyl)-3-chloro-1-(2-trimethylsilylethoxymethyl)pyrazol-4-amine (4.80 g, 81.6%) as a yellow solid. LCMS (ESI) m/z: 419.1 [M+H]⁺.

### Synthesis of 5-(2-amino-5-bromo-3-methyl-phenyl)-3-methyl-1-(2-trimethylsilylethoxymethyl)pyrazol-4-amine (Intermediate B1-3)

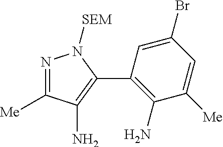 [SMILES: Cc1cc(Br)cc(-c2c(N)c(C)nn2COCC[Si](C)(C)C)c1N]

### Step 1: 2-methyl-6-[5-methyl-4-nitro-2-(2-trimethylsilylethoxymethyl)pyrazol-3-yl]aniline

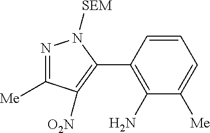 [SMILES: Cc1cccc(-c2c([N+](=O)[O-])c(C)nn2COCC[Si](C)(C)C)c1N]

To a solution of 2-bromo-6-methyl-aniline (5 g, 26.9 mmol, 1 eq.) in dioxane (150 mL) was added trimethyl-[2-[(3-methyl-4-nitro-pyrazol-1-yl)methoxy]ethyl]silane (Intermediate A1-1) (11.5 g, 26.9 mmol, 60% purity, 1 eq.), Cs₂CO₃ (13.1 g, 40.3 mmol, 1.5 eq.), Pd(OAc)₂ (603 mg, 2.69 mmol, 0.1 eq.), XPhos (2.56 g, 5.37 mmol, 0.2 eq.) and CsOPiv (6.92 g, 29.6 mmol, 1.1 eq.). The mixture was degassed and purged with N₂ for three times. The mixture was stirred at 100° C. for 12 hr under N₂ atmosphere. The reaction mixture was concentrated under reduced pressure. The residue was diluted with H₂O (300 mL) and extracted with EtOAc (400 mL×3). The combined organic layers were washed with brine (300 mL), dried over anhydrous Na₂SO₄, filtered and concentrated under reduced pressure. The residue was purified by flash chromatography on silica gel column using 0-15% of EtOAc in petroleum ether as eluent, to provide 2-methyl-6-[5-methyl-4-nitro-2-(2-trimethylsilylethoxymethyl)pyrazol-3-yl]aniline as a black oil (8.5 g, 29.1%). LCMS (ESI) m/z: 363.2 [M+H]⁺.

### Step 2: 4-bromo-2-methyl-6-[5-methyl-4-nitro-2-(2-trimethylsilylethoxymethyl)pyrazol-3-yl]aniline

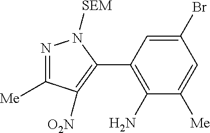 [SMILES: Cc1cc(Br)cc(-c2c([N+](=O)[O-])c(C)nn2COCC[Si](C)(C)C)c1N]

To a solution of 2-methyl-6-[5-methyl-4-nitro-2-(2-trimethylsilylethoxymethyl)pyrazol-3-yl]aniline (4.25 g, 11.7 mmol, 1 eq.) in MeCN (50 mL) was added NBS (2.30 g, 12.9 mmol, 1.1 eq) and the mixture was stirred at 25° C. for 12 hr. The reaction mixture was concentrated under reduced pressure, diluted with H₂O (100 mL) and extracted with EtOAc (150 mL×3). The combined organic layers were washed with brine (100 mL), dried over anhydrous Na₂SO₄, filtered and concentrated under reduced pressure to provide the crude product 4-bromo-2-methyl-6-[5-methyl-4-nitro-2-(2-trimethylsilylethoxymethyl)pyrazol-3-yl]aniline as a black oil (10.1 g). LCMS (ESI) m/z: 441.1 [M+H]⁺.

### Step 3: 2-methyl-6-[5-methyl-4-nitro-2-(2-trimethylsilylethoxymethyl)pyrazol-3-yl]aniline

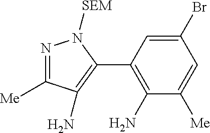 [SMILES: Cc1cc(Br)cc(-c2c(N)c(C)nn2COCC[Si](C)(C)C)c1N]

To a solution of 4-bromo-2-methyl-6-[5-methyl-4-nitro-2-(2-trimethylsilylethoxymethyl) pyrazol-3-yl]aniline (5 g, 11.3 mmol, 1 eq.) in EtOH/H₂O (3:1, 130 mL) was added NH₄Cl (3.03 g, 56.6 mmol, 5 eq.) and Fe (3.16 g, 56.6 mmol, 5 eq.). The mixture was stirred at 80° C. for 3 hr. The solution was filtered to collect the solution. The mixture was concentrated under reduced pressure. The residue was diluted with H₂O (100 mL) and extracted with EtOAc (200 mL×3). The combined organic layers were washed with brine (100 mL), dried over anhydrous Na₂SO₄, filtered and concentrated under reduced pressure to provide 5-(2-amino-5-bromo-3-methyl-phenyl)-3-methyl-1-(2-trimethylsilylethoxymethyl)pyrazol-4-amine as a black oil (8.1 g, 69.5%, 80% purity). LCMS (ESI) m/z: 412.9 [M+H]⁺.

### Synthesis of methyl 4-amino-3-[4-amino-5-chloro-2-(2-trimethylsilylethoxymethyl)pyrazol-3-yl]benzoate

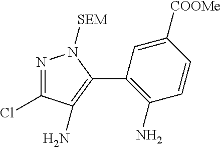 [SMILES: COC(=O)c1ccc(N)c(-c2c(N)c(Cl)nn2COCC[Si](C)(C)C)c1]

### Step 1: methyl 4-(tert-butoxycarbonylamino)-3-iodo-benzoate

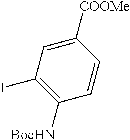 [SMILES: COC(=O)c1ccc(NC(=O)OC(C)(C)C)c(I)c1]

To a solution of methyl 4-amino-3-iodo-benzoate (20 g, 72.1 mmol, 1.0 eq) in THF (400 mL) was added 1M NaHMDS in THF (158 mL, 0.158 mol, 2.2 eq) dropwise over 30 mins at 0° C. under N₂ atmosphere. Then (Boc)₂O (16.5 g, 75.8 mmol, 1.05 eq) was added and the reaction mixture was warmed to 25° C. for 16 hr. The resulting mixture was poured into ice-water (200 mL) and extracted with EtOAc (200 mL×2). The combined organic phase was washed with brine (100 mL), dried over anhydrous Na₂SO₄, filtered and concentrated in vacuum. The residue was purified by flash chromatography (ISCO®; 220 g SepaFlash® Silica Flash Column, petroleum ether/EtOAc with EtOAc from 0˜20%, 100 mL/min, 254 mn), to provide 4-(tert-butoxycarbonylamino)-3-iodo-benzoate as a yellow solid (21.5 g, 78.9%). ¹H NMR (400 MHz, DMSO-d₆) δ ppm 1.41-1.56 (m, 9H), 3.79-3.91 (m, 3H), 7.67 (d, J=8.56 Hz, 1H), 7.93 (dd, J=8.44, 1.96 Hz, 1H), 8.34 (d, J=2.08 Hz, 1H), 8.45-8.57 (m, 1H); LCMS (ESI) m/z: 378.0 [M+H]⁺.

### Step 2: methyl 4-(tert-butoxycarbonylamino)-3-[5-chloro-4-nitro-2-(2-trimethylsilylethoxymethyl)pyrazol-3-yl]benzoate

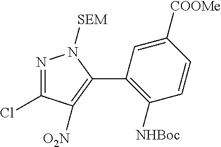 [SMILES: COC(=O)c1ccc(NC(=O)OC(C)(C)C)c(-c2c([N+](=O)[O-])c(Cl)nn2COCC[Si](C)(C)C)c1]

A mixture of methyl 4-(tert-butoxycarbonylamino)-3-iodo-benzoate (2.0 g, 5.30 mmol, 1.0 eq), 2-[(3-chloro-4-nitro-pyrazol-1-yl)methoxy]ethyl-trimethyl-silane (Intermediate A1-2) (1.47 g, 5.30 mmol, 1.0 eq), Pd(OAc)₂ (119 mg, 0.530 mmol, 0.1 eq), XPhos (1.26 g, 2.65 mmol, 0.5 eq) and Cs₂CO₃ (2.59 g, 7.95 mmol, 1.5 eq), cesium; 2,2-dimethylpropanoate (1.37 g, 5.83 mmol, 1.1 eq) in toluene (50 mL) was degassed and purged with N₂ for 3 times, and then the mixture was stirred at 100° C. for 16 hr under N₂ atmosphere. The reaction mixture was filtered and concentrated under reduced pressure. The crude product was purified by flash chromatography (ISCO®; 20 g SepaFlash® Silica Flash Column, petroleum ether/EtOAc with EtOAc from 0˜20%, 50 mL/min, 254 mn), to provide methyl 4-(tert-butoxycarbonylamino)-3-[5-chloro-4-nitro-2-(2-trimethylsilylethoxymethyl)pyrazol-3-yl]benzoate as a white solid (1.1 g, 39.3% yield). ¹H NMR (400 MHz, CD₃OD) δ ppm 0.06 (s, 9H), 0.87-1.05 (m, 2H), 1.26-1.29 (m, 9H), 2.13-2.38 (m, 2H), 2.53 (s, 3H), 3.60-3.92 (m, 3H), 4.13-4.21 (m, 1H), 4.28 (t, J=6.36 Hz, 2H), 5.33 (s, 2H), 7.67-7.87 (m, 1H); LCMS (ESI) m/z: 549.1 [M+H]⁺.

### Step 3: methyl 3-[4-amino-5-chloro-2-(2-trimethylsilylethoxymethyl)pyrazol-3-yl]-4-(tert-butoxycarbonylamino)benzoate

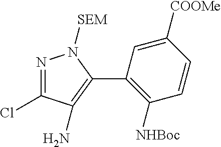 [SMILES: COC(=O)c1ccc(NC(=O)OC(C)(C)C)c(-c2c(N)c(Cl)nn2COCC[Si](C)(C)C)c1]

A mixture of methyl 4-(tert-butoxycarbonylamino)-3-[5-chloro-4-nitro-2-(2-trimethyl silylethoxymethyl)pyrazol-3-yl]benzoate (250 mg, 0.474 mmol, 1.0 eq), Zn (310 mg, 4.74 mmol, 10.0 eq), NH₄Cl (253 mg, 4.74 mmol, 10.0 eq) in EtOH/H₂O (3:1, 8 mL) was degassed and purged with N₂ for 3 times. The reaction mixture was stirred at 50° C. for 16 hr under N₂ atmosphere, filtered and concentrated under reduced pressure. The residue was extracted with EtOAc (10 mL×3). The combined organic layers were dried over Na₂SO₄ and concentrated to provide methyl 3-[4-amino-5-chloro-2-(2-trimethylsilylethoxymethyl)pyrazol-3-yl]-4-(tert-butoxycarbonylamino)benzoate as a white solid (200 mg, 84.8%). LCMS (ESI) m/z: 519.2 [M+H]⁺.

### Step 4: methyl 4-amino-3-[4-amino-5-chloro-2-(2-trimethylsilylethoxymethyl)pyrazol-3-yl]benzoate

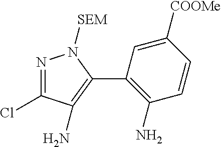 [SMILES: COC(=O)c1ccc(N)c(-c2c(N)c(Cl)nn2COCC[Si](C)(C)C)c1]

A mixture of methyl 3-[4-amino-5-chloro-2-(2-trimethylsilylethoxymethyl)pyrazol-3-yl]-4-(tert-butoxycarbonylamino)benzoate (300 mg, 0.603 mmol) in 4N HCl in MeOH (2 mL) was stirred at 25° C. for 1 hr. The reaction mixture was quenched by NaHCO₃/H₂O (20 mL) until the pH was adjusted to 8, and extracted with EtOAc (25 mL×2). The extracts were dried over Na₂SO₄ and concentrated to provide methyl 4-amino-3-[4-amino-5-chloro-2-(2-trimethylsilylethoxymethyl) pyrazol-3-yl]benzoate as a yellow solid (200 mg), which was used for the next step directly without any further purification. ¹H NMR (400 MHz, CDCl₃) δ ppm 0.04 (s, 9H), 0.83-0.95 (m, 2H), 3.51-3.66 (m, 2H), 3.87-3.90 (m, 3H), 5.22 (br d, J=17.4 Hz, 2H), 6.81 (d, J=8.5 Hz, 1H), 7.92-8.03 (m, 2H); LCMS (ESI) m/z: 419.1 [M+H]⁺.

### Synthesis of 5-(2,6-difluorophenyl)-1-(4-methoxybenzyl)-1,6-dihydrobenzo[d]pyrazolo[3,4-f][1,3]diazepine-9-carboxylic acid (Intermediate B1-8)

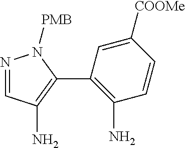

### Step 1: methyl 4-(tert-butoxycarbonylamino)-3-iodo-benzoate

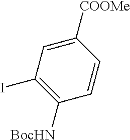 [SMILES: COC(=O)c1ccc(NC(=O)OC(C)(C)C)c(I)c1]

To a solution of methyl 4-amino-3-iodo-benzoate (50 g, 0.180 mol, 1.0 eq) in THF (500 mL) was added 1M NaHMDS in THF (397 mL, 0.397 mol, 2.2 eq) dropwise and the resulting mixture was stirred at 0° C. for 30 min. Then Boc₂O (41.4 g, 0.189 mol, 1.05 eq) was added and the reaction was stirred at 25° C. for another 2 hr. The reaction solution was concentrated and dissolved in EtOAc (1 L), washed with brine (200 mL×3). The organic layer was concentrated in a vacuum and the residue was purified by flash chromatography on silica gel column (using 0-20% of EtOAc in petroleum ether as eluent) to provide methyl 4-(tert-butoxycarbonylamino)-3-iodo-benzoate as a white solid (19.0 g, 27.9%). ¹H NMR (500 MHz, DMSO-d₆) δ ppm 1.48 (s, 9H), 3.84 (s, 3H), 7.67 (d, J=8.5 Hz, 1H), 7.92 (dd, J=8.5, 1.9 Hz, 1H), 8.33 (d, J=1.8 Hz, 1H), 8.51 (s, 1H); LCMS (ESI) m/z: 321.9 [M+H]⁺.

### Step 2: methyl 4-(tert-butoxycarbonylamino)-3-[2-[(4-methoxyphenyl)methyl]-4-nitro-pyrazol-3-yl]benzoate

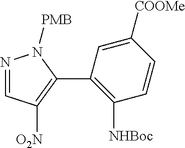

A mixture of methyl 4-(tert-butoxycarbonylamino)-3-iodo-benzoate (10.0 g, 26.5 mmol, 1.0 eq), 1-[(4-methoxyphenyl)methyl]-4-nitro-pyrazole (Intermediate A2-3) (6.18 g, 26.5 mmol, 1.0 eq), Pd(OAc)₂ (595 mg, 2.65 mmol, 0.2 eq), XPhos (6.32 g, 13.3 mmol, 0.5 eq) and Cs₂CO₃ (12.9 g, 39.7 mmol, 1.5 eq), 2,2-dimethylpropanoate (6.82 g, 29.1 mmol, 1.1 eq) in toluene (300 mL) was degassed and purged with N₂ for 3 times. The mixture was stirred at 100° C. for 16 hr under N₂ atmosphere, filtered and concentrated under reduced pressure. The crude product was purified by flash chromatography on silica gel column using 0-50% of EtOAc in petroleum ether as eluent, to provide methyl 4-(tert-butoxycarbonylamino)-3-[2-[(4-methoxyphenyl)methyl]-4-nitro-pyrazol-3-yl]benzoate as a white solid (6.30 g, 49.2%). ¹H NMR (400 MHz, DMSO-d₆) δ ppm 1.37 (s, 9H), 3.72-3.73 (m, 3H), 3.80 (s, 3H), 4.88 (d, J=15.13 Hz, 1H), 5.10 (d, J=15.26 Hz, 1H), 6.84 (d, J=8.76 Hz, 2H), 7.02 (d, J=8.63 Hz, 2H), 7.62 (d, J=2.00 Hz, 1H), 7.81 (d, J=8.63 Hz, 1H), 8.07 (d d, J=8.63, 2.00 Hz, 1H), 8.36-8.46 (m, 1H), 9.28 (s, 1H); LCMS (ESI) m/z: 505.2 [M+H]⁺.

### Step 3: methyl 3-[4-amino-2-[(4-methoxyphenyl)methyl]pyrazol-3-yl]-4-(tert-butoxycarbonylamino)benzoate

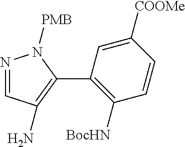

Methyl 4-(tert-butoxycarbonylamino)-3-[2-[(4-methoxyphenyl)methyl]-4-nitropyrazol-3-yl]benzoate (5.80 g, 12.0 mmol, 1.0 eq) in EtOH/H₂O (1:1, 70 mL) was added Zn (7.86 g, 0.120 mol, 10.0 eq) and NH₄Cl (6.43 g, 0.120 mol, 10.0 eq). The mixture was stirred at 50° C. for 2 hr, filtered and the filtration was extracted with EtOAc (100 mL×2) and saturated brine water (100 mL×2). The organic layer was concentrated in vacuum to remove solvent. The residue was purified by flash silica gel chromatography (ISCO®; 40 g SepaFlash® Silica Flash Column, Eluent of 0˜60% EtOAc/petroleum ether gradient @ 50 mL/min), to provide methyl 3-[4-amino-2-[(4-methoxyphenyl) methyl] pyrazol-3-yl]-4-(tert-butoxycarbonylamino) benzoate as a purple oil (1.90 g, 34.9%). ¹H NMR (400 MHz, DMSO-d₆) δ ppm 1.45 (s, 9H), 3.73 (s, 3H), 3.87 (s, 3H), 4.12 (q, J=7.13 Hz, 3H), 5.00 (d, J=13.63 Hz, 2H), 6.72 (d, J=8.63 Hz, 2H), 6.87 (d, J=8.63 Hz, 3H), 7.79 (d, J=2.00 Hz, 1H), 8.07 (dd, J=8.76, 2.00 Hz, 1H), 8.25 (d, J=8.88 Hz, 1H); LCMS (ESI) m/z: 475.2 [M+H]⁺.

### Step 4: methyl 4-amino-3-[4-amino-2-[(4-methoxyphenyl)methyl]pyrazol-3-yl]benzoate

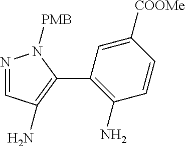

Methyl 3-[4-amino-2-[(4-methoxyphenyl)methyl]pyrazol-3-yl]-4-(tert-butoxycarbonyl amino) benzoate (300 mg, 663 umol, 1.0 eq) in 4N HCl in MeOH (5 mL) was stirred at 25° C. for 1 hr. The mixture was concentrated in vacuum to provide methyl 4-amino-3-[4-amino-2-[(4-methoxyphenyl)methyl]pyrazol-3-yl]benzoate as a yellow solid (300 mg). ¹H NMR (400 MHz, CDCl₃) δ ppm 3.83 (s, 5H), 4.59 (s, 2H), 6.91 (d, J=8.6 Hz, 3H), 7.34 (d, J=8.6 Hz, 3H); LCMS (ESI) m/z: 352.7 [M+H]⁺.

### Synthesis of 5-(2-amino-5-bromo-phenyl)-1-(2-trimethylsilylethoxymethyl)pyrazol-4-amine (Intermediate B1-10)

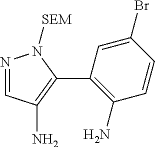 [SMILES: C[Si](C)(C)CCOCn1ncc(N)c1-c1cc(Br)ccc1N]

### Step 1: tert-butyl N-(2-bromophenyl)carbamate

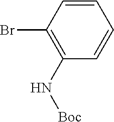 [SMILES: CC(C)(C)OC(=O)Nc1ccccc1Br]

A solution of 2-bromoaniline (10 g, 58.1 mmol, 1 eq.) in Boc₂O (38.1 g, 0.174 mol, 3 eq.) was stirred at 100° C. for 24 hr. The reaction mixture was concentrated under reduced pressure. The residue was diluted with H₂O (1000 mL) and extracted with EtOAc (1500 mL×3). The combined organic layers were washed with brine (1000 mL), dried over anhydrous Na₂SO₄, filtered and concentrated under reduced pressure to provide the crude product tert-butyl N-(2-bromophenyl)carbamate as a yellow oil (156 g). ¹H NMR (400 MHz, CDCl₃) δ ppm 1.52-1.57 (m, 9H), 6.82-6.94 (m, 1H), 7.01 (br s, 1H), 7.25-7.32 (m, 1H), 7.50 (dd, J=7.9, 1.1 Hz, 1H), 8.15 (d, J=8.3 Hz, 1H).

### Step 2: tert-butyl (2-(4-nitro-1-((2-(trimethylsilyl)ethoxy)methyl)-1H-pyrazol-5-yl)phenyl)carbamate

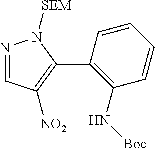 [SMILES: CC(C)(C)OC(=O)Nc1ccccc1-c1c([N+](=O)[O-])cnn1COCC[Si](C)(C)C]

To a solution of tert-butyl N-(2-bromophenyl)carbamate (5 g, 18.4 mmol, 1 eq.) in toluene (50 mL) was added trimethyl-[2-[(4-nitropyrazol-1-yl)methoxy]ethyl]silane (Intermediate A1-3) (4.69 g, 19.3 mmol, 1.05 eq.), Cs₂CO₃ (12.0 g, 36.8 mmol, 2 eq.), 2,2-dimethylpropanoic acid (3.75 g, 36.8 mmol, 2 eq.), Pd(OAc)₂ (412 mg, 1.84 mmol, 0.1 eq.) and XPhos (2.63 g, 5.51 mmol, 0.3 eq.). The mixture was degassed and purged with N₂ for three times. The mixture was stirred at 100° C. for 3 hrs under N₂ atmosphere. The reaction mixture was concentrated under reduced pressure. The residue was diluted with H₂O (500 mL) and extracted with EtOAc (500 mL×3). The combined organic layers were washed with brine (500 mL), dried over anhydrous Na₂SO₄, filtered and concentrated under reduced pressure, to provide crude product tert-butyl N-[2-[4-nitro-2-(2-trimethylsilylethoxymethyl)pyrazol-3-yl]phenyl]carbamate as a brown oil (40 g).

### Step 3: tert-butyl N-[4-bromo-2-[4-nitro-2-(2-trimethylsilylethoxymethyl)pyrazol-3-yl]phenyl]carbamate

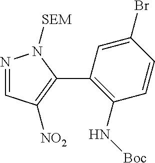 [SMILES: CC(C)(C)OC(=O)Nc1ccc(Br)cc1-c1c([N+](=O)[O-])cnn1COCC[Si](C)(C)C]

To a solution of tert-butyl N-[2-[4-nitro-2-(2-trimethylsilylethoxymethyl)pyrazol-3-yl]phenyl]carbamate (8 g, 18.4 mmol, 1 eq.) in MeCN (300 mL) was added NBS (3.60 g, 20.3 mmol, 1.1 eq.) and the mixture was stirred at 50° C. for 12 hrs. The reaction mixture was concentrated under reduced pressure. The residue was diluted with H₂O (100 mL) and extracted with EtOAc (150 mL×3). The combined organic layers were washed with brine (100 mL), dried over anhydrous Na₂SO₄, filtered and concentrated under reduced pressure, to provide tert-butyl N-[4-bromo-2-[4-nitro-2-(2-trimethylsilylethoxymethyl)pyrazol-3-yl]phenyl]carbamate as a brown oil (26 g, 55.0%).

### Step 4: tert-butyl N-[2-[4-amino-2-(2-trimethylsilylethoxymethyl)pyrazol-3-yl]-4-bromo-phenyl]carbamate

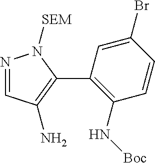 [SMILES: CC(C)(C)OC(=O)Nc1ccc(Br)cc1-c1c(N)cnn1COCC[Si](C)(C)C]

To a solution of tert-butyl N-[4-bromo-2-[4-nitro-2-(2-trimethylsilylethoxymethyl) pyrazol-3-yl]phenyl]carbamate (26 g, 50.6 mmol, 1 eq.) in EtOH/H₂O (1:1, 200 mL) was added NH₄Cl (19.0 g, 0.354 mol, 7 eq.) and Fe (19.8 g, 0.354 mol, 7 eq.). The mixture was stirred at 80° C. for 3 hrs and filtered. The filtrate was concentrated under reduced pressure. The residue was purified by flash chromatography on silica gel column using 0-50% of EtOAc in petroleum ether as eluent to provide tert-butyl N-[2-[4-amino-2-(2-trimethylsilylethoxymethyl)pyrazol-3-yl]-4-bromo-phenyl]carbamate as a red oil (10.1 g, 40.0%). LCMS (ESI) m/z: 483.1 [M+H]⁺.

### Step 5: 5-(2-amino-5-bromophenyl)-1-((2-(trimethylsilyl)ethoxy)methyl)-1H-pyrazol-4-amine

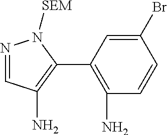 [SMILES: C[Si](C)(C)CCOCn1ncc(N)c1-c1cc(Br)ccc1N]

To a solution of tert-butyl N-[2-[4-amino-2-(2-trimethylsilylethoxymethyl)pyrazol-3-yl]-4-bromo-phenyl]carbamate (5 g, 10.3 mmol, 1 eq.) in DCM (40 mL) was added ZnBr₂ (4.66 g, 20.7 mmol, 2 eq.). The mixture was stirred at 40° C. for 12 hrs. The mixture was filtered to collect the solid. The solid was dissolved with EtOAc (200 mL). The combined organic layers were washed with brine (150 mL), dried over anhydrous Na₂SO₄, filtered and concentrated under reduced pressure to provide the crude product 5-(2-amino-5-bromo-phenyl)-1-(2-trimethylsilylethoxymethyl)pyrazol-4-amine as a red solid (7.35 g). LCMS (ESI) m/z: 383.0 [M+H]⁺.

### Synthesis of 3-chloro-5-(2,6-difluorophenyl)-8-fluoro-1,6-dihydrobenzo[d]pyrazolo[3,4-f][1,3]diazepine-9-carboxylic acid (Intermediate B1-12)

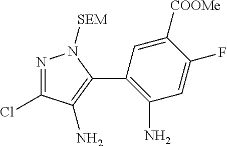 [SMILES: COC(=O)c1cc(-c2c(N)c(Cl)nn2COCC[Si](C)(C)C)c(N)cc1F]

### Step 1: methyl 4-amino-2-fluoro-5-iodo-benzoate

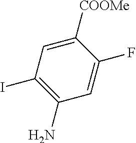 [SMILES: COC(=O)c1cc(I)c(N)cc1F]

A mixture of methyl 4-amino-2-fluoro-benzoate (5 g, 29.5 mmol, 1.0 eq), ICl (5.28 g, 32.5 mmol, 1.1 eq), CaCO₃ (5.92 g, 59.1 mmol) in DCM/MeOH (60 mL, v/v=1/1) was degassed and purged with N₂ for 3 times, and then the mixture was stirred at 25° C. for 2 hr. The reaction solution was filtrated and the filtrate was washed with saturated sodium thiosulfate solution (20 mL×2), dried over anhydrous sodium sulfate, and filtrated, the filtrate was concentrated under reduced pressure, the residue was purified by flash chromatography (ISCO®; 40 g SepaFlash® Silica Flash Column, petroleum ether/EtOAc with EtOAc from 0˜40%, 50 mL/min, 254 mn/I₂), to provide methyl 4-amino-2-fluoro-5-iodo-benzoate as a white solid (2.5 g, 28.6% yield). ¹H NMR (400 MHz, DMSO-d₆) δ ppm 3.71 (s, 3H), 6.29 (br s, 2H), 6.46 (d, J=13.94 Hz, 1H), 7.99 (d, J=8.07 Hz, 1H); LCMS (ESI) m/z: 296.1 [M+H]⁺.

### Step 2: methyl 4-(tert-butoxycarbonylamino)-2-fluoro-5-iodo-benzoate

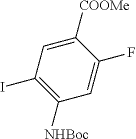 [SMILES: COC(=O)c1cc(I)c(NC(=O)OC(C)(C)C)cc1F]

To a solution of methyl 4-amino-2-fluoro-5-iodo-benzoate (2.5 g, 8.47 mmol, 1.0 eq) in THF (30 mL) was added dropwise 1M LiHMDS in THF (16.9 mL, 2.0 eq) at 0° C. for 30 min. The mixture was stirred at 0° C. for another 30 min, before Boc₂O (2.22 g, 2.34 mmol) was added dropwise at 0° C. The resulting mixture was stirred at 25° C. for 2 h, then quenched by the addition of H₂O (50 mL) at 0° C., and extracted with EtOAc (50 mL×3). The combined organic layers were concentrated under reduced pressure. The crude product was purified by flash chromatography (ISCO®; 20 g SepaFlash® Silica Flash Column, petroleum ether/EtOAc with EtOAc from 0˜20%, 50 mL/min, 254 mn/I₂), to provide methyl 4-(tert-butoxycarbonylamino)-2-fluoro-5-iodo-benzoate as a white solid (2.3 g, 68.6%). ¹H NMR (400 MHz, DMSO-d₆) δ ppm 1.59 (s, 9H), 3.79-3.93 (m, 3H), 7.44-7.71 (m, 1H), 8.13-8.34 (m, 1H), 8.43 (br d, J=9.66 Hz, 1H); LCMS (ESI) m/z: 396.1 [M+H]⁺.

### Step 3: methyl 4-amino-5-[5-chloro-4-nitro-2-(2-trimethylsilylethoxymethyl)pyrazol-3-yl]-2-fluoro-benzoate

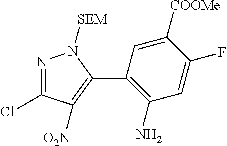 [SMILES: COC(=O)c1cc(-c2c([N+](=O)[O-])c(Cl)nn2COCC[Si](C)(C)C)c(N)cc1F]

A mixture of methyl 4-(tert-butoxycarbonylamino)-2-fluoro-5-iodo-benzoate (2.3 g, 5.82 mmol, 1.0 eq), 2-[(3-chloro-4-nitro-pyrazol-1-yl)methoxy]ethyl-trimethyl-silane (1.62 g, 5.82 mmol, 1.0 eq), Pd(OAc)₂ (392 mg, 1.75 mmol, 0.3 eq), XPhos (1.39 g, 2.91 mmol, 0.5 eq), Cs₂CO₃ (2.84 g, 8.73 mmol, 1.5 eq) and cesium(I) 2,2-dimethylpropanoate (1.50 g, 6.40 mmol, 1.1 eq) in toluene (50 mL) was degassed and purged with N₂ for 3 times. The mixture was stirred at 100° C. for 16 hr under N₂ atmosphere, filtered off the solid and concentrated under reduced pressure to remove solvent. The residue was purified by flash chromatography (ISCO®; 20 g SepaFlash® Silica Flash Column, petroleum ether/EtOAc with EtOAc from 0˜40%, 50 mL/min, 254 mn), to provide methyl 4-amino-5-[5-chloro-4-nitro-2-(2-trimethylsilylethoxymethyl)pyrazol-3-yl]-2-fluoro-benzoate as a white solid (1.2 g, 46.3%). ¹H NMR (400 MHz, DMSO-d₆) δ ppm 0.06 (s, 9H), 0.73-0.81 (m, 2H), 3.44 (td, J=8.3, 3.6 Hz, 2H), 3.73 (s, 3H), 5.09-5.31 (m, 2H), 6.44-6.50 (m, 2H), 6.50-6.57 (m, 1H), 7.73 (d, J=8.2 Hz, 1H).

### Step 4: methyl 4-amino-5-[4-amino-5-chloro-2-(2-trimethylsilylethoxymethyl)pyrazol-3-yl]-2-fluoro-benzoate

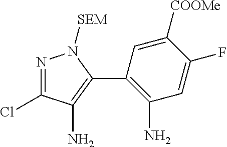 [SMILES: COC(=O)c1cc(-c2c(N)c(Cl)nn2COCC[Si](C)(C)C)c(N)cc1F]

A mixture of methyl 4-amino-5-[5-chloro-4-nitro-2-(2-trimethylsilylethoxymethyl) pyrazol-3-yl]-2-fluoro-benzoate (1.2 g, 2.70 mmol, 1.0 eq), Fe (1.51 g, 26.9 mmol, 10.0 eq) and NH₄Cl (1.44 g, 26.9 mmol, 10.0 eq) in EtOH/H₂O (v/v=5/1, 30 mL) was stirred at 50° C. for 2 hr. The reaction mixture was extracted with EtOAc (50 mL×2). The combined organic layers were concentrated under reduced pressure to remove the solvent. The residue was purified by flash chromatography (ISCO®; 20 g SepaFlash® Silica Flash Column, petroleum ether/EtOAc with EtOAc from 0˜40%, 50 mL/min, 254 mn), to provide methyl 4-amino-5-[4-amino-5-chloro-2-(2-trimethylsilylethoxymethyl) pyrazol-3-yl]-2-fluoro-benzoate as a white solid (600 mg, 53.6%). ¹H NMR (400 MHz, DMSO-d₆) δ ppm 0.07 (m, 9H), 0.74 (br dd, J=8.38, 5.32 Hz, 2H), 3.33 (br s, 2H), 3.74 (s, 3H), 3.85 (s, 2H), 4.81-5.27 (m, 2H), 6.09 (s, 2H), 6.54 (d, J=13.82 Hz, 1H), 7.63 (d, J=8.44 Hz, 1H).

### Synthesis of 4-[4-amino-2-(2-trimethylsilylethoxymethyl)pyrazol-3-yl]-6-chloro-pyridin-3-amine (Intermediate B2-1)

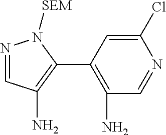 [SMILES: C[Si](C)(C)CCOCn1ncc(N)c1-c1cc(Cl)ncc1N]

### Step 1: 6-chloro-4-[4-nitro-2-(2-trimethylsilylethoxymethyl)pyrazol-3-yl]pyridin-3-amine

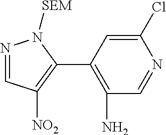 [SMILES: C[Si](C)(C)CCOCn1ncc([N+](=O)[O-])c1-c1cc(Cl)ncc1N]

A mixture of 6-chloro-4-iodo-pyridin-3-amine (2 g, 7.86 mmol, 1 eq.), trimethyl-[2-[(4-nitropyrazol-1-yl)methoxy]ethyl]silane (Intermediate A1-3) (2.11 g, 8.65 mmol, 1.1 eq.), Pd(PPh₃)₄ (0.91 g, 0.79 mmol, 0.1 eq.), Cs₂CO₃ (7.68 g, 23.6 mmol, 3.0 eq.) and CuI (299 mg, 2.36 mmol, 0.3 eq.) in dioxane (20 mL) was degassed and purged with N₂ for 3 times, and then the mixture was stirred at 80° C. for 16 hr under N₂ atmosphere. The reaction mixture was then filtered and concentrated under reduced pressure. The residue was purified by flash chromatography on silica gel column using 0-27% of EtOAc in petroleum ether as eluent to provide 6-chloro-4-[4-nitro-2-(2-trimethylsilylethoxymethyl)pyrazol-3-yl]pyridin-3-amine as a yellow solid (1.2 g, 37.2%). ¹H NMR (400 MHz, DMSO-d₆) δ ppm −0.04-0.09 (m, 9H), 0.80-0.72 (m, 2H), 3.47-3.41 (m, 2H), 5.19 (d, J=11.3 Hz, 1H), 5.35 (d, J=11.0 Hz, 1H), 5.73 (s, 2H), 7.27 (s, 1H), 7.94 (s, 1H), 8.48 (s, 1H); LCMS (ESI) m/z: 370.2 [M+H]⁺.

### Step 2: 4-[4-amino-2-(2-trimethylsilylethoxymethyl)pyrazol-3-yl]-6-chloro-pyridin-3-amine (B2-1)

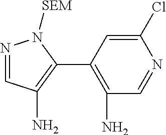 [SMILES: C[Si](C)(C)CCOCn1ncc(N)c1-c1cc(Cl)ncc1N]

A mixture of 6-chloro-4-[4-nitro-2-(2-trimethylsilylethoxymethyl)pyrazol-3-yl]pyridin-3-amine (1.2 g, 3.24 mmol, 1 eq.), Fe (1.45 g, 26.0 mmol, 8 eq.), NH₄Cl (1.39 g, 26.0 mmol, 8 eq.) in EtOH/H₂O (2:1, 12 mL) was degassed and purged with N₂ for 3 times, and then the mixture was stirred at 60° C. for 3 hr under N₂ atmosphere. The reaction mixture was filtered and extracted with EtOAc (20 mL×3). The combined organic layers were washed with brine (20 mL), dried over anhydrous Na₂SO₄, filtered and concentrated under reduced pressure. The residue was purified by flash chromatography on silica gel column using 0-56% of EtOAc in petroleum ether as eluent to provide 4-[4-amino-2-(2-trimethylsilylethoxymethyl)pyrazol-3-yl]-6-chloro-pyridin-3-amine as a yellow oil (1.0 g, 88.0%). LCMS (ESI) m/z: 340.1 [M+H]⁺.

### Synthesis of 4-[4-amino-5-methyl-2-(2-trimethylsilylethoxymethyl) pyrazol-3-yl]-6-chloro-pyridin-3-amine (Intermediate B2-2)

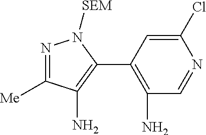 [SMILES: Cc1nn(COCC[Si](C)(C)C)c(-c2cc(Cl)ncc2N)c1N]

### Step 1: 6-chloro-4-[5-methyl-4-nitro-2-(2-trimethylsilylethoxymethyl) pyrazol-3-yl] pyridin-3-amine

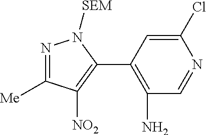 [SMILES: Cc1nn(COCC[Si](C)(C)C)c(-c2cc(Cl)ncc2N)c1[N+](=O)[O-]]

To a mixture of 6-chloro-4-iodo-pyridin-3-amine (30 g, 0.118 mol, 1 eq.), trimethyl-[2-[(3-methyl-4-nitro-pyrazol-1-yl) methoxy] ethyl] silane (Intermediate A1-1) (75.9 g, 0.295 mol, 2.5 eq.), CuI (26.9 g, 0.141 mol, 1.2 eq.), Cs₂CO₃ (96.0 g, 0.295 mol, 2.5 eq.) and 2,2-dimethylpropanoic acid (30.6 g, 0.299 mol, 2.5 eq.) in dioxane (50 mL) was added Pd(PPh₃)₄ (20.4 g, 17.7 mmol, 0.15 eq.). The system was purged with nitrogen atmosphere for 3 times. The mixture was heated to 100° C. and stirred for 12 hrs under nitrogen atmosphere. Then the reaction mixture was quenched with H₂O (100 mL) slowly under nitrogen atmosphere and a extracted with EtOAc (100 mL×3). The combined organic layer was washed with brine (100 mL), dried over anhydrous Na₂SO₄, filtered and concentrated under reduced pressure. The residue was purified by flash chromatography on silica gel column using 0-16% of EtOAc in petroleum ether as eluent to provide 6-chloro-4-[5-methyl-4-nitro-2-(2-trimethylsilylethoxymethyl) pyrazol-3-yl]pyridin-3-amine as a yellow solid (31.5 g, 64.0%). ¹H NMR (400 MHz, CDCl₃) δ ppm 0.00 (s, 9H), 0.84-1.00 (m, 2H), 2.62 (s, 3H), 3.59-3.67 (m, 2H), 3.83 (s, 2H), 5.12-5.31 (m, 2H), 7.05-7.16 (m, 1H), 8.05 (s, 1H); LCMS (ESI) m/z: 384.2 [M+H]⁺.

### Step 2: 4-[4-amino-5-methyl-2-(2-trimethylsilylethoxymethyl) pyrazol-3-yl]-6-chloro-pyridin-3-amine (B2-2)

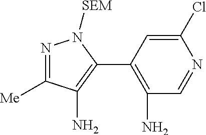 [SMILES: Cc1nn(COCC[Si](C)(C)C)c(-c2cc(Cl)ncc2N)c1N]

To a solution of 6-chloro-4-[5-methyl-4-nitro-2-(2-trimethylsilylethoxymethyl) pyrazol-3-yl] pyridin-3-amine (11.6 g, 30.2 mmol, 1 eq.) in EtOH/H₂O (3:1, 120 mL) was added Fe (10.1 g, 0.181 mol, 6 eq.) and NH₄Cl (9.70 g, 0.181 mol, 6 eq.) and the mixture was stirred at 80° C. for 6 hr. The mixture was filtered and the filtrate was concentrated under reduced pressure. The mixture was diluted with water (100 mL) and extracted with EtOAc (100 mL×3). The combined organic layer was washed with brine (100 mL), dried over anhydrous Na₂SO₄, filtered and concentrated under reduced pressure to provide crude product 4-[4-amino-5-methyl-2-(2-trimethylsilylethoxymethyl) pyrazol-3-yl]-6-chloro-pyridin-3-amine as a gray solid (9.6 g). ¹H NMR (400 MHz, CDCl₃) δ ppm −0.05-0.03 (m, 9H), 0.91 (dd, J=9.0, 7.6 Hz, 2H), 1.19-1.31 (m, 1H), 2.24 (s, 3H), 3.60 (br t, J=8.3 Hz, 2H), 4.12 (q, J=7.1 Hz, 1H), 5.19 (s, 2H), 6.94-7.24 (m, 1H), 7.35 (s, 1H), 7.66 (br s, 1H), 7.97 (s, 1H); LCMS (ESI) m/z: 354.3 [M+H]⁺.

### Synthesis of 4-[4-amino-5-chloro-2-(2-trimethylsilylethoxymethyl)pyrazol-3-yl]-6-bromo-pyridin-3-amine (Intermediate B2-3)

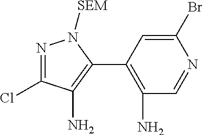 [SMILES: C[Si](C)(C)CCOCn1nc(Cl)c(N)c1-c1cc(Br)ncc1N]

### Step 1: 6-bromo-4-iodo-pyridine-3-carboxylic acid

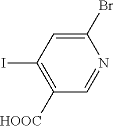 [SMILES: O=C(O)c1cnc(Br)cc1I]

To a solution of TMP (4.20 g, 29.7 mmol, 3 eq.) in THF (20 mL) was added dropwise 2.5M n-BuLi in n-hexane (11.9 mL, 29.7 mmol, 3 eq.) at −78° C. The mixture was stirred at −78° C. for 30 min. Then 6-bromopyridine-3-carboxylic acid (2.0 g, 9.9 mmol, 1 eq.) was added to the above solution and the mixture was stirred at −78° C. for 2 hr. I₂ (7.54 g, 29.7 mmol, 3 eq.) was added to the solution at −78° C. and the mixture was stirred at 25° C. for 12 hr. The reaction mixture was then concentrated under vacuum and the residue was suspended in water (50 mL) and washed with DCM (100 mL×2). The aqueous layer was acidified by addition of concentrated 12N HCl in H₂O until the pH was adjusted to 2. The precipitate was collected by filtration, washed with water (30 mL) and then dried under vacuum to provide 6-bromo-4-iodo-pyridine-3-carboxylic acid as a light-yellow solid (2.7 g, 83.2%). ¹H NMR (400 MHz, DMSO-d₆) δ ppm 8.35 (s, 1H), 8.62 (s, 1H), 13.83 (br s, 1H).

### Step 2: tert-butyl N-(6-bromo-4-iodo-3-pyridyl)carbamate

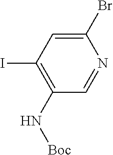 [SMILES: CC(C)(C)OC(=O)Nc1cnc(Br)cc1I]

To a solution of 6-bromo-4-iodo-pyridine-3-carboxylic acid (1.00 g, 3.05 mmol, 1 eq.) in ^{t}BuOH/toluene (1:1, 10 mL) was added DPPA (1.26 g, 4.57 mmol, 1.5 eq.) and TEA (926 mg, 9.15 mmol, 3 eq.). The mixture was stirred at 110° C. for 3 hr and concentrated under reduced pressure. The residue was purified by flash chromatography on silica gel column using 0-20% of EtOAc in petroleum ether as eluent to provide tert-butyl N-(6-bromo-4-iodo-3-pyridyl)carbamate as a white solid (250 mg, 20.3%). LCMS (ESI) m/z: 398.7 [M+H]⁺.

### Step 3: 6-bromo-4-iodo-pyridin-3-amine

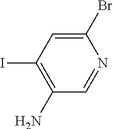

To a solution of tert-butyl N-(6-bromo-4-iodo-3-pyridyl)carbamate (6.50 g, 16.3 mmol, 1 eq.) in DCM (50 mL) was added TFA (36.8 g, 0.323 mol, 20 eq.). The mixture was stirred at 25° C. for 2 hr under N₂ atmosphere. The reaction mixture was concentrated under reduced pressure. The residue was diluted with saturated NaHCO₃ aqueous solution (10 mL) and extracted with EtOAc (15 mL×3). The combined organic layers were washed with brine (10 mL), dried over anhydrous Na₂SO₄, filtered and concentrated under reduced pressure. The residue was purified by flash chromatography on silica gel column using 0-30% of EtOAc in petroleum ether as eluent to provide 6-bromo-4-iodo-pyridin-3-amine as a white solid (4.2 g, 36.2%). LCMS (ESI) m/z: 298.7 [M+H]⁺.

### Step 4: 6-bromo-4-[5-chloro-4-nitro-2-(2-trimethylsilylethoxymethyl)pyrazol-3-yl]pyridin-3-amine

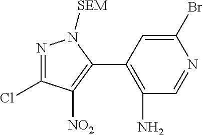 [SMILES: C[Si](C)(C)CCOCn1nc(Cl)c([N+](=O)[O-])c1-c1cc(Br)ncc1N]

To a solution of 6-bromo-4-iodo-pyridin-3-amine (3.0 g, 10.0 mmol, 1 eq.) in dioxane (30 mL) was added Pd(PPh₃)₄ (1.16 g, 1.00 mmol, 0.1 eq.), CuI (382 mg, 2.01 mmol, 0.2 eq.), Cs₂CO₃ (9.81 g, 30.1 mmol, 3 eq.) and 2-[(3-chloro-4-nitro-pyrazol-1-yl)methoxy]ethyl-trimethyl-silane (Intermediate A1-2) (3.07 g, 11.0 mmol, 1.1 eq.). The mixture was degassed and purged with N₂ for three times, and then stirred at 90° C. for 12 hr under N₂ atmosphere. The reaction mixture was concentrated under reduced pressure, diluted with H₂O (10 mL) and extracted with EtOAc (15 mL×3). The combined organic layers were washed with brine (10 mL), dried over anhydrous Na₂SO₄, filtered and concentrated under reduced pressure. The residue was purified by flash chromatography on silica gel column using 0-30% of EtOAc in petroleum ether as eluent to provide 6-bromo-4-[5-chloro-4-nitro-2-(2-trimethylsilylethoxymethyl)pyrazol-3-yl]pyridin-3-amine as a yellow solid (1.4 g, 29.8%). LCMS (ESI) m/z: 449.9 [M+H]⁺.

### Step 5: 4-[4-amino-5-chloro-2-(2-trimethylsilylethoxymethyl)pyrazol-3-yl]-6-bromo-pyridin-3-amine (B2-3)

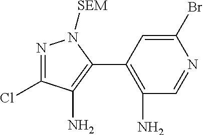 [SMILES: C[Si](C)(C)CCOCn1nc(Cl)c(N)c1-c1cc(Br)ncc1N]

To a solution of 6-bromo-4-[5-chloro-4-nitro-2-(2-trimethylsilylethoxymethyl)pyrazol-3-yl]pyridin-3-amine (1.20 g, 2.67 mmol, 1 eq.) in THF (10 mL) was added NaBH₄ (405 mg, 10.7 mmol, 4 eq.), NiCl₂·6H₂O (2.54 g, 10.7 mmol, 4 eq.) at 0° C. The mixture was degassed and purged with N₂ for three times and then stirred at 0° C. for 1 hr under N₂ atmosphere. The reaction mixture was quenched by addition of ammonium chloride (10 mL) at 0° C. and extracted with EtOAc (10 mL×3). Then the combined organic layers were washed with brine (10 mL), dried over anhydrous Na₂SO₄, filtered and concentrated under reduced pressure. The residue was purified by flash chromatography on silica gel column using 0-30% of EtOAc in petroleum ether as eluent to provide 4-[4-amino-5-chloro-2-(2-trimethylsilylethoxymethyl)pyrazol-3-yl]-6-bromo-pyridin-3-amine as a yellow solid (500 mg, 44.6%). LCMS (ESI) m/z: 420.0 [M+H]⁺.

### Synthesis of 4-[4-amino-5-methyl-2-(2-trimethylsilylethoxymethyl)pyrazol-3-yl]-6-chloro-2-methyl-pyridin-3-amine (Intermediate B2-4)

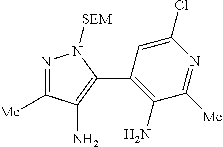 [SMILES: Cc1nc(Cl)cc(-c2c(N)c(C)nn2COCC[Si](C)(C)C)c1N]

### Step 1: 4-bromo-6-chloro-2-methyl-pyridin-3-amine

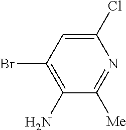

To a solution of 6-chloro-2-methyl-pyridin-3-amine (20 g, 0.140 mol, 1 eq.) in MeCN (300 mL) was added NBS (25.0 g, 0.140 mol, 1 eq.) at 0° C. The mixture was stirred at 25° C. for 16 h under N₂ atmosphere, then concentrated under reduced pressure, diluted with sat. NaHCO₃ aqueous solution (200 mL), extracted with EtOAc (200 mL×2), washed with brine (200 mL), dried over anhydrous Na₂SO₄ and concentrated under reduced pressure. The residue was purified by flash chromatography on silica gel column using 0-30% of EtOAc in petroleum ether as eluent to provide 4-bromo-6-chloro-2-methyl-pyridin-3-amine as a yellow solid (7.5 g, 24.1% yield). ¹H NMR (400 MHz, CDCl₃) δ ppm 2.44 (s, 3H), 3.95-4.27 (m, 2H), 7.28 (s, 1H).

### Step 2: 6-chloro-2-methyl-4-[5-methyl-4-nitro-2-(2-trimethylsilylethoxymethyl)pyrazol-3-yl]pyridin-3-amine

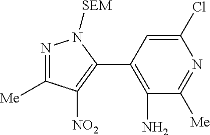 [SMILES: Cc1nc(Cl)cc(-c2c([N+](=O)[O-])c(C)nn2COCC[Si](C)(C)C)c1N]

To a solution of 4-bromo-6-chloro-2-methyl-pyridin-3-amine (11 g, 49.7 mmol, 1 eq.) and trimethyl-[2-[(3-methyl-4-nitro-pyrazol-1-yl)methoxy]ethyl]silane (Intermediate A1-1) (38.4 g, 0.149 mol, 3 eq.) in dioxane (400 mL) was added Cs₂CO₃ (48.6 g, 0.149 mol, 3 eq.), Pd(PPh₃)₄ (5.74 g, 4.97 mmol, 0.1 eq.) and CuI (1.89 g, 9.93 mmol, 0.2 eq.). The mixture was stirred at 100° C. for 16 hr under N₂ atmosphere. The solution was filtered and concentrated. The residue was purified by flash chromatography on silica gel column using 0-30% of EtOAc in petroleum ether as eluent to provide 6-chloro-2-methyl-4-[5-methyl-4-nitro-2-(2-trimethylsilylethoxymethyl)pyrazol-3-yl]pyridin-3-amine as a yellow solid (6 g, 30.4%). ¹H NMR (400 MHz, DMSO-d₆) δ ppm −0.06 (s, 9H), 0.64-0.84 (m, 2H), 2.35 (s, 3H), 2.49-2.51 (m, 3H), 3.36-3.52 (m, 2H), 4.92-5.30 (m, 2H), 5.38 (s, 2H), 7.09 (s, 1H).

### Step 3: 4-[4-amino-5-methyl-2-(2-trimethylsilylethoxymethyl)pyrazol-3-yl]-6-chloro-2-methyl-pyridin-3-amine (B2-4)

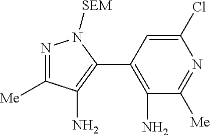 [SMILES: Cc1nc(Cl)cc(-c2c(N)c(C)nn2COCC[Si](C)(C)C)c1N]

To a solution of 6-chloro-2-methyl-4-[5-methyl-4-nitro-2-(2-trimethylsilylethoxymethyl) pyrazol-3-yl]pyridin-3-amine (6 g, 15.1 mmol, 1 eq.) in EtOH/H₂O (3:1, 120 mL) was added Fe (8.42 g, 0.151 mol, 10 eq.) and NH₄Cl (8.07 g, 0.151 mol, 10 eq.). The mixture was stirred at 50° C. for 16 hr under N₂ atmosphere, filtered and extracted with sat NaHCO₃ aqueous solution (200 mL) and EtOAc (200 mL×2), washed with brine (200 mL) and dried over anhydrous Na₂SO₄. The residue was purified by flash chromatography on silica gel column using 0-30% of EtOAc in petroleum ether as eluent to provide 4-[4-amino-5-methyl-2-(2-trimethylsilylethoxymethyl)pyrazol-3-yl]-6-chloro-2-methyl-pyridin-3-amine as a yellow oil (5 g, 90.1%). ¹H NMR (400 MHz, DMSO-d₆) δ ppm −0.07 (s, 9H), 0.76 (br t, J=8.0 Hz, 2H), 2.12 (s, 3H), 2.35 (s, 3H), 3.96 (br s, 2H), 4.97-5.17 (m, 4H), 7.12 (s, 1H).

### Synthesis of 4-[4-amino-2-[(4-methoxyphenyl)methyl]pyrazol-3-yl]-6-chloro-2-methyl-pyridin-3-amine (Intermediate B2-6)

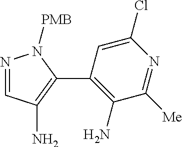

### Step 1: 4-bromo-6-chloro-2-methyl-pyridin-3-amine

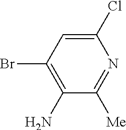

To a solution of 6-chloro-2-methyl-pyridin-3-amine (30 g, 0.210 mol, 1 eq.) in MeOH (200 mL) and AcOH (25.3 g, 0.421 mol, 2 eq.) was added Br₂ (60.5 g, 0.379 mol, 1.8 eq.) at 0° C. The mixture was stirred at 25° C. for 16 hr under N₂ atmosphere, quenched by aqueous NaHSO₃ (50 mL) and NaHCO₃ until pH was adjusted to 7. The solution was extracted with EtOAc (200 mL×2), washed with brine (200 mL) and dried over anhydrous Na₂SO₄ and concentrated under reduced pressure. The residue was purified by flash chromatography on silica gel column using 0-20% of EtOAc in petroleum ether as eluent to provide 4-bromo-6-chloro-2-methyl-pyridin-3-amine as a brown solid (30 g, 64.4%). ¹H NMR (400 MHz, CDCl₃) δ ppm 2.33-2.49 (m, 3H), 7.12-7.41 (m, 1H).

### Step 2: 6-chloro-4-[2-[(4-methoxyphenyl)methyl]-4-nitro-pyrazol-3-yl]-2-methyl-pyridin-3-amine

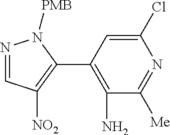

To a solution of 4-bromo-6-chloro-2-methyl-pyridin-3-amine (20 g, 90.3 mmol, 1 eq.) and 1-[(4-methoxyphenyl)methyl]-4-nitro-pyrazole (Intermediate A2-3) (31.6 g, 0.136 mol, 1.5 eq.) in dioxane (400 mL) was added Cs₂CO₃ (88.3 g, 0.271 mol, 3.0 eq.), Pd(PPh₃)₄ (10.4 g, 9.03 mmol, 0.1 eq.) and CuI (3.44 g, 18.1 mmol, 0.2 eq.). The mixture was stirred at 100° C. for 16 hr under N₂ atmosphere, filtered and concentrated. The residue was purified by flash chromatography on silica gel column using 0-30% of EtOAc in petroleum ether as eluent to provide 6-chloro-4-[2-[(4-methoxyphenyl)methyl]-4-nitro-pyrazol-3-yl]-2-methyl-pyridin-3-amine as a yellow oil (20 g, 59.3%). ¹H NMR (400 MHz, CDCl₃) δ ppm 2.36 (s, 3H), 3.49 (s, 2H), 3.69 (s, 3H), 4.85-5.09 (m, 2H), 6.65-6.75 (m, 3H), 6.87 (d, J=8.6 Hz, 2H), 7.18-7.31 (m, 1H), 8.14 (s, 1H).

### Step 3: 4-[4-amino-2-[(4-methoxyphenyl)methyl]pyrazol-3-yl]-6-chloro-2-methyl-pyridin-3-amine (B2-6)

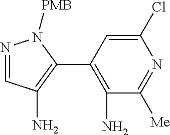

To a solution of 6-chloro-4-[2-[(4-methoxyphenyl)methyl]-4-nitro-pyrazol-3-yl]-2-methyl-pyridin-3-amine (20 g, 53.5 mmol, 1 eq.) in EtOH/H₂O (3:1, 400 mL), Fe (29.9 g, 0.535 mol, 10 eq.), NH₄Cl (28.6 g, 0.535 mol, 10 eq.) was added. The mixture was stirred at 50° C. for 5 hr under N₂ atmosphere, filtered and concentrated under reduced pressure. The residue was extracted with EtOAc (400 mL×2), washed with brine (300 mL) and dried over anhydrous Na₂SO₄ and concentrated under reduced pressure. The residue was purified by flash chromatography on silica gel column using 0-100% of EtOAc in petroleum ether as eluent to provide 4-[4-amino-2-[(4-methoxyphenyl)methyl]pyrazol-3-yl]-6-chloro-2-methyl-pyridin-3-amine as a yellow solid (14 g, 68.5%). ¹H NMR (400 MHz, DMSO-d₆) δ ppm 2.33 (s, 3H), 3.69 (s, 3H), 4.03 (br d, J=6.9 Hz, 2H), 4.82-5.18 (m, 4H), 6.81 (d, J=9.8 Hz, 5H), 7.13-7.23 (m, 1H).

### Synthesis of 3-[4-amino-5-methyl-2-(2-trimethylsilylethoxymethyl)pyrazol-3-yl]-5-(trifluoromethyl)pyridin-2-amine (Intermediate B2-14)

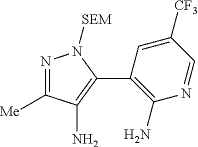 [SMILES: Cc1nn(COCC[Si](C)(C)C)c(-c2cc(C(F)(F)F)cnc2N)c1N]

### Step 1: 3-[5-methyl-4-nitro-2-(2-trimethylsilylethoxymethyl)pyrazol-3-yl]-5-(trifluoromethyl)pyridin-2-amine

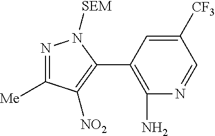 [SMILES: Cc1nn(COCC[Si](C)(C)C)c(-c2cc(C(F)(F)F)cnc2N)c1[N+](=O)[O-]]

A mixture of 3-iodo-5-(trifluoromethyl)pyridin-2-amine (3 g, 10.4 mmol, 1 eq.), trimethyl-[2-[(3-methyl-4-nitro-pyrazol-1-yl)methoxy]ethyl]silane (Intermediate A1-1) (2.68 g, 10.4 mmol, 1 eq.), Pd(PPh₃)₄ (1.20 g, 1.04 mmol, 0.1 eq.), K₂CO₃ (2.16 g, 15.6 mmol, 1.5 eq.) and CuI (595 mg, 3.12 mmol, 0.3 eq.) in DMF (30 mL) was degassed and purged with N₂ for 3 times. The mixture was stirred at 90° C. for 16 hr under N₂ atmosphere, diluted with H₂O (30 mL) and extracted with EtOAc (30 mL×3). The combined organic layers were washed with brine (30 mL×2), dried with anhydrous Na₂SO₄, filtered and concentrated under reduced pressure. The residue was purified by flash chromatography on silica gel column using 0-20% of EtOAc in petroleum ether as eluent to provide 3-[5-methyl-4-nitro-2-(2-trimethylsilylethoxymethyl)pyrazol-3-yl]-5-(trifluoromethyl)pyridin-2-amine as a brown oil (1.7 g, 35.1%). LCMS (ESI) m/z: 418.1 [M+H]⁺.

### Step 2: 3-[4-amino-5-methyl-2-(2-trimethylsilylethoxymethyl)pyrazol-3-yl]-5-(trifluoromethyl)pyridin-2-amine (B2-14)

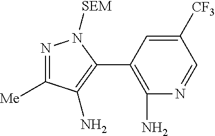 [SMILES: Cc1nn(COCC[Si](C)(C)C)c(-c2cc(C(F)(F)F)cnc2N)c1N]

To a solution of 3-[5-methyl-4-nitro-2-(2-trimethylsilylethoxymethyl)pyrazol-3-yl]-5-(trifluoromethyl)pyridin-2-amine (1 g, 2.40 mmol, 1 eq.) in EtOH/H₂O (2:1, 15 mL) was added NH₄Cl (640 mg, 11.9 mmol, 2 eq.) and Fe (1.34 g, 24.0 mmol, 10 eq.). The mixture was stirred at 70° C. for 4 hr. The reaction mixture was filtered and concentrated under reduced pressure. The residue was purified by flash chromatography on silica gel column using 0-40% of EtOAc in petroleum ether as eluent to provide 3-[4-amino-5-methyl-2-(2-trimethylsilylethoxymethyl)pyrazol-3-yl]-5-(trifluoromethyl)pyridin-2-amine as a brown oil (703 mg, 70.4%). LCMS (ESI) m/z: 388.1 [M+H]⁺.

### Synthesis of 4-[4-amino-2-[(4-methoxyphenyl)methyl]-5-methyl-pyrazol-3-yl]-6-(trifluoromethyl)pyridin-3-amine (Intermediate B2-17)

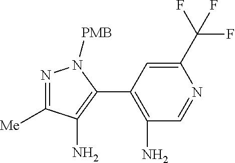

### Step 1: 4-[2-[(4-methoxyphenyl)methyl]-5-methyl-4-nitro-pyrazol-3-yl]-6-(trifluoromethyl)pyridin-3-amine

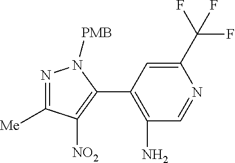

A mixture of 4-iodo-6-(trifluoromethyl)pyridin-3-amine (1 g, 3.47 mmol, 1 eq.), 1-[(4-methoxyphenyl)methyl]-3-methyl-4-nitro-pyrazole (Intermediate A2-1) (858 mg, 3.47 mmol, 1 eq.), Pd(PPh₃)₄ (401 mg, 0.347 mmol, 0.1 eq.), K₂CO₃ (719 mg, 5.21 mmol, 1.5 eq.) and CuI (198 mg, 1.04 mmol, 0.3 eq.) in DMF (10 mL) was degassed and purged with N₂ for 3 times, and then the mixture was stirred at 90° C. for 16 hr under N₂ atmosphere. The reaction mixture was diluted with H₂O (30 mL) and extracted with EtOAc (30 mL×3). The combined organic layers were washed with brine (30 mL×2), dried with anhydrous Na₂SO₄, filtered and concentrated under reduced pressure. The residue was purified by flash chromatography on silica gel column using 0-60% of EtOAc in petroleum ether as eluent to provide 4-[2-[(4-methoxyphenyl)methyl]-5-methyl-4-nitro-pyrazol-3-yl]-6-(trifluoromethyl)pyridin-3-amine as a brown oil (620 mg, 38.5%). No mass signal.

### Step 2: 4-[4-amino-2-[(4-methoxyphenyl)methyl]-5-methyl-pyrazol-3-yl]-6-(trifluoromethyl)pyridin-3-amine (B2-17)

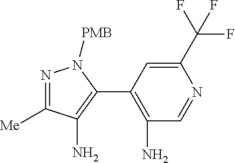

To a solution of 4-[2-[(4-methoxyphenyl)methyl]-5-methyl-4-nitro-pyrazol-3-yl]-6-(trifluoromethyl)pyridin-3-amine (620 mg, 1.52 mmol, 1 eq.) in EtOH/H₂O (3:1, 32 mL) was added NH₄Cl (407 mg, 7.61 mmol) and Fe (850 mg, 15.2 mmol, 10 eq.). The mixture was stirred at 70° C. for 4 hr, diluted with H₂O (30 mL) and extracted with EtOAc (30 mL×3). The combined organic layers were washed with brine (30 mL×2), dried with anhydrous Na₂SO₄, filtered and concentrated under reduced pressure. The residue was purified by flash chromatography on silica gel column using 0-80% of EtOAc in petroleum ether as eluent to provide 4-[4-amino-2-[(4-methoxyphenyl)methyl]-5-methyl-pyrazol-3-yl]-6-(trifluoromethyl)pyridin-3-amine as a brown oil (320 mg, 51.2%). LCMS (ESI) m/z: 378.1 [M+H]⁺.

### Synthesis of 4-[4-amino-2-[(4-methoxyphenyl)methyl]-5-methyl-pyrazol-3-yl]-6-(trifluoromethyl)pyridin-3-amine (Intermediate B2-18)

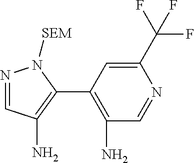 [SMILES: C[Si](C)(C)CCOCn1ncc(N)c1-c1cc(C(F)(F)F)ncc1N]

### Step 1: 4-[2-[(4-methoxyphenyl)methyl]-5-methyl-4-nitro-pyrazol-3-yl]-6-(trifluoromethyl)pyridin-3-amine

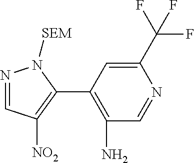 [SMILES: C[Si](C)(C)CCOCn1ncc([N+](=O)[O-])c1-c1cc(C(F)(F)F)ncc1N]

A mixture of 4-iodo-6-(trifluoromethyl)pyridin-3-amine (1.3 g, 4.51 mmol, 1 eq.), 1-[(4-methoxyphenyl)methyl]-3-methyl-4-nitro-pyrazole (Intermediate A1-3) (1.12 g, 4.51 mmol, 1 eq.), Pd(PPh₃)₄ (521 mg, 0.451 mmol, 0.1 eq.), K₂CO₃ (935 mg, 6.77 mmol, 1.5 eq.) and CuI (257 mg, 1.35 mmol, 0.3 eq.) in DMF (10 mL) was degassed and purged with N₂ for 3 times, and then the mixture was stirred at 90° C. for 16 h under N₂ atmosphere. The reaction mixture was diluted with H₂O (30 mL) and extracted with EtOAc (30 mL×3). The combined organic layers were washed with brine (30 mL×2), dried with anhydrous Na₂SO₄, filtered and concentrated under reduced pressure. The residue was purified by flash chromatography on silica gel column using 0-60% of EtOAc in petroleum ether as eluent to provide 4-[2-[(4-methoxyphenyl)methyl]-5-methyl-4-nitro-pyrazol-3-yl]-6-(trifluoromethyl)pyridin-3-amine as a brown oil (1 g, 32.6%). No mass signal.

### Step 2: 4-[4-amino-2-[(4-methoxyphenyl)methyl]-5-methyl-pyrazol-3-yl]-6-(trifluoromethyl)pyridin-3-amine (B2-18)

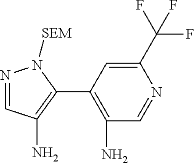 [SMILES: C[Si](C)(C)CCOCn1ncc(N)c1-c1cc(C(F)(F)F)ncc1N]

To a solution of 4-[2-[(4-methoxyphenyl)methyl]-5-methyl-4-nitro-pyrazol-3-yl]-6-(trifluoromethyl)pyridin-3-amine (1 g, 2.45 mmol, 1 eq.) in EtOH/H₂O (5:2, 7 mL) was added NH₄Cl (656 mg, 12.2 mmol, 5 eq.) and Fe (1.37 g, 24.5 mmol, 10 eq.). The mixture was stirred at 70° C. for 4 hr, filtered and concentrated under reduced pressure. The residue was purified by flash chromatography on silica gel column using 0-40% of EtOAc in petroleum ether as eluent to provide 4-[4-amino-2-[(4-methoxyphenyl)methyl]-5-methyl-pyrazol-3-yl]-6-(trifluoromethyl)pyridin-3-amine as a brown oil (300 mg, 27.5%). No mass signal.

### Synthesis of 4-(4-amino-1-(4-methoxybenzyl)-3-(trifluoromethyl)-1H-pyrazol-5-yl)-6-bromopyridin-3-amine (Intermediate B2-19)

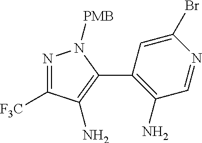

### Step 1: 6-bromo-4-[2-[(4-methoxyphenyl)methyl]-4-nitro-5-(trifluoromethyl)pyrazol-3-yl]pyridin-3-amine

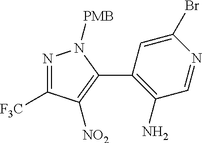

A mixture of 1-[(4-methoxyphenyl)methyl]-4-nitro-3-(trifluoromethyl)pyrazole (Intermediate A2-4) (1 g, 3.32 mmol, 1.0 eq.), 6-bromo-4-iodo-pyridin-3-amine (1.19 g, 3.98 mmol, 1.2 eq.), Cs₂CO₃ (3.24 g, 9.96 mmol, 3.0 eq.), CuI (126 mg, 0.663 mmol, 0.2 eq.) and Pd(PPh₃)₄ (384 mg, 0.331 mmol, 0.1 eq.) in dioxane (20 mL) was degassed and purged with N₂ for 3 times. The mixture was stirred at 70° C. for 16 hr under N₂ atmosphere, diluted with H₂O (10 mL) and extracted with EtOAc (10 mL×3). The combined organic phase was washed with brine (10 mL×2), dried with anhydrous Na₂SO₄, filtered and concentrated under reduced pressure. The residue was purified by flash chromatography on silica gel column using 0-30% of EtOAc in petroleum ether as eluent, to provide 6-bromo-4-[2-[(4-methoxyphenyl)methyl]-4-nitro-5-(trifluoromethyl)pyrazol-3-yl]pyridin-3-amine as a yellow oil (2 g, 52.9%). ¹H NMR (400 MHz, DMSO-d₆) δ ppm 3.72 (s, 3H), 5.05 (d, J=15.1 Hz, 1H), 5.22 (d, J=15.1 Hz, 1H), 5.93 (s, 2H), 6.85 (d, J=8.8 Hz, 2H), 7.02 (d, J=8.6 Hz, 2H), 7.34 (s, 1H), 7.95 (s, 1H).

### Step 2: 4-[4-amino-2-[(4-methoxyphenyl)methyl]-5-(trifluoromethyl)pyrazol-3-yl]-6-bromo-pyridin-3-amine

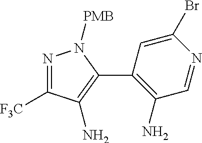

A mixture of 6-bromo-4-[2-[(4-methoxyphenyl)methyl]-4-nitro-5-(trifluoromethyl) pyrazol-3-yl]pyridin-3-amine (2 g, 4.24 mmol, 1.0 eq.) and NiCl₂·6H₂O (6.04 g, 25.4 mmol, 6.0 eq.) in THF (20 mL) was stirred at 25° C. for 10 min, Then NaBH₄ (480 mg, 12.7 mmol, 3.0 eq.) was added to the mixture at 0° C. and the mixture was stirred for 10 min. The reaction mixture was diluted with H₂O (20 mL), filtered to remove the insoluble and extracted with EtOAc (20 mL×3). The combined organic phase was washed with brine (10 mL×2), dried with anhydrous Na₂SO₄, filtered and concentrated in vacuum. The residue was purified by flash chromatography on silica gel column using 0-30% of EtOAc in petroleum ether as eluent, to provide 4-[4-amino-2-[(4-methoxyphenyl)methyl]-5-(trifluoromethyl)pyrazol-3-yl]-6-bromo-pyridin-3-amine as a yellow solid (1.04 g, 53.8%). LCMS (ESI) m/z: 442.0 [M+H]⁺.

### Synthesis of 4-[4-amino-5-(difluoromethyl)-2-[(4-methoxyphenyl)methyl]pyrazol-3-yl]-6-bromo-pyridin-3-amine (Intermediate B2-20)

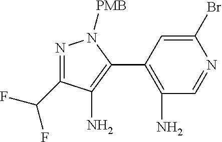

### Step 1: [1-[(4-methoxyphenyl)methyl]-4-nitro-pyrazol-3-yl]methanol

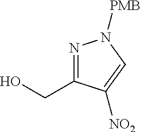

To a solution of methyl 1-[(4-methoxyphenyl)methyl]-4-nitro-pyrazole-3-carboxylate (Intermediate A2-5) (6.5 g, 22.3 mmol, 1 eq.) in THF (100 mL) was added dropwise 1M DIBAL-H in toluene (44.6 mL, 44.6 mmol, 2 eq.) at −78° C. The mixture was stirred at 25° C. for 16 hr, quenched by H₂O (50 mL) and extracted with EtOAc (50 mL×3). The combined organic layers were washed with brine (30 mL), dried with anhydrous Na₂SO₄, filtered and concentrated under reduced pressure to provide the crude product [1-[(4-methoxyphenyl)methyl]-4-nitro-pyrazol-3-yl]methanol as a yellow solid (5.8 g, crude) which was used for the next step without further purification. ¹H NMR (400 MHz, DMSO-d₆) δ ppm 3.73 (s, 3H), 4.63 (d, J=6.02 Hz, 2H), 5.20 (t, J=5.90 Hz, 1H), 5.26 (s, 2H), 6.93 (d, J=8.53 Hz, 2H), 7.31 (d, J=8.53 Hz, 2H), 8.81-9.01 (m, 1H).

### Step 2: 1-[(4-methoxyphenyl)methyl]-4-nitro-pyrazole-3-carbaldehyde

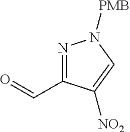

To a solution of [1-[(4-methoxyphenyl)methyl]-4-nitro-pyrazol-3-yl]methanol (5.8 g, 22.0 mmol, 1 eq.) in DCM (100 mL) was added DMP (28.0 g, 66.1 mmol, 3 eq.). The mixture was stirred at 25° C. for 16 hr, diluted with H₂O (40 mL) and extracted with EtOAc (40 mL×3). The combined organic layers were washed with brine (20 mL), dried with anhydrous Na₂SO₄, filtered and concentrated under reduced pressure. The residue was purified by flash chromatography on silica gel column using 0-60% of EtOAc in petroleum ether as eluent, to provide 1-[(4-methoxyphenyl)methyl]-4-nitro-pyrazole-3-carbaldehyde as a white solid (4.95 g, 86.1%). ¹H NMR (400 MHz, DMSO-d₆) δ ppm 3.74 (s, 3H), 5.40 (s, 2H), 6.93-6.95 (m, 2H), 7.34-7.37 (m, 2H), 9.12 (s, 1H), 10.19 (s, 1H).

### Step 3: 3-(difluoromethyl)-1-[(4-methoxyphenyl)methyl]-4-nitro-pyrazole

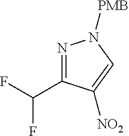

A solution of 1-[(4-methoxyphenyl)methyl]-4-nitro-pyrazole-3-carbaldehyde (6.5 g, 24.8 mmol, 1 eq.) in DCM (100 mL) was added DAST (8.02 g, 49.7 mmol, 2 eq.) at 0° C. The reaction was stirred for 16 hr at 25° C., diluted with H₂O (40 mL) and extracted with DCM (40 mL×3). The combined organic layers were washed with brine (40 mL), dried with anhydrous Na₂SO₄, filtered and concentrated under reduced pressure. The residue was purified by flash chromatography on silica gel column using 0-30% of EtOAc in petroleum ether as eluent, to provide 3-(difluoromethyl)-1-[(4-methoxyphenyl)methyl]-4-nitro-pyrazole as a yellow solid (4.15 g, 58.9%). ¹H NMR (400 MHz, DMSO-d₆) δ ppm 3.74 (s, 3H), 5.36 (s, 2H), 6.92-6.96 (m, 2H), 7.15 (s, 1H), 7.28 (s, 1H), 7.32-7.37 (m, 2H), 7.41 (s, 1H), 9.14 (s, 1H), ¹⁹F NMR (377 MHz, DMSO-d₆) δ ppm −117.49.

### Step 4: 6-bromo-4-[5-(difluoromethyl)-2-[(4-methoxyphenyl)methyl]-4-nitro-pyrazol-3-yl]pyridin-3-amine

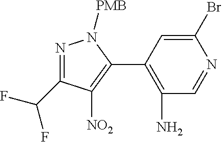

To a solution of 3-(difluoromethyl)-1-[(4-methoxyphenyl)methyl]-4-nitro-pyrazole (2 g, 7.06 mmol) and 6-bromo-4-iodo-pyridin-3-amine (2.11 g, 7.06 mmol) in dioxane (20 mL) was added Pd(PPh₃)₄ (816 mg, 0.706 mmol), Cs₂CO₃ (6.90 g, 21.2 mmol) and CuI (403 mg, 2.12 mmol). The mixture was stirred at 90° C. for 16 hr, filtered and concentrated under reduced pressure. The residue was purified by flash silica gel chromatography (ISCO®; 20 g SepaFlash® Silica Flash Column, petroleum ether/EtOAc with EtOAc from 0˜30%, 35 mL/min, 254 nm) to afford 6-bromo-4-[5-(difluoromethyl)-2-[(4-methoxyphenyl)methyl]-4-nitro-pyrazol-3-yl]pyridin-3-amine (1.61 g, 47.9%) as a yellow solid. LCMS (ESI) m/z: 456.1 [M+H]⁺.

### Step 5: 4-[4-amino-5-(difluoromethyl)-2-[(4-methoxyphenyl)methyl]pyrazol-3-yl]-6-bromo-pyridin-3-amine (B2-20)

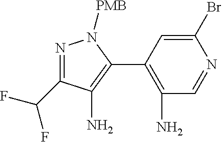

To a solution of 6-bromo-4-[5-(difluoromethyl)-2-[(4-methoxyphenyl)methyl]-4-nitro-pyrazol-3-yl]pyridin-3-amine (1.6 g, 3.52 mmol) in H₂O (7 mL) and EtOH (21 mL) was added Fe (1.97 g, 35.2 mmol) and NH₄Cl (1.88 g, 35.2 mmol). The mixture was stirred at 50° C. for 3 hr, filtered and concentrated under reduced pressure. The residue was purified by flash silica gel chromatography (ISCO®; 4 g SepaFlash® Silica Flash Column, petroleum ether/EtOAc with EtOAc from 0˜50%, 20 mL/min, 254 nm) to give 4-[4-amino-5-(difluoromethyl)-2-[(4-methoxyphenyl)methyl]pyrazol-3-yl]-6-bromo-pyridin-3-amine (1.01 g, 66.8%) as a white solid. LCMS (ESI) m/z: 426.1 [M+H]⁺.

### Synthesis of methyl 4-amino-5-(5-amino-2-chloro-4-pyridyl)-1-(2-trimethylsilylethoxymethyl)pyrazole-3-carboxylate (Intermediate B2-21)

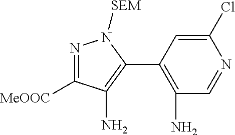 [SMILES: COC(=O)c1nn(COCC[Si](C)(C)C)c(-c2cc(Cl)ncc2N)c1N]

### Step 1: methyl 5-(5-amino-2-chloro-4-pyridyl)-4-nitro-1-(2-trimethylsilylethoxymethyl) pyrazole-3-carboxylate

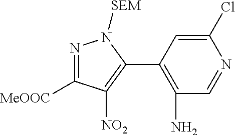 [SMILES: COC(=O)c1nn(COCC[Si](C)(C)C)c(-c2cc(Cl)ncc2N)c1[N+](=O)[O-]]

To a solution of 6-chloro-4-iodo-pyridin-3-amine (5 g, 19.6 mmol) in dioxane (250 mL) was added Cs₂CO₃ (16.0 g, 49.1 mmol) and 2,2-dimethylpropanoic acid (5.02 g, 49.1 mmol) and CuI (4.49 g, 23.5 mmol) and methyl 4-nitro-1-(2-trimethylsilylethoxymethyl)pyrazole-3-carboxylate (Intermediate A1-4) (7.11 g, 23.5 mmol) and Pd(PPh₃)₄ (4.54 g, 3.93 mmol). The mixture was stirred at 100° C. for 16 hr under nitrogen atmosphere, filtered and concentrated under reduced pressure. The residue was purified by purified by flash chromatography (ISCO®; 80 g AgelaFlash® Silica Flash Column, petroleum ether/EtOAc with EtOAc from 0˜50%, flow rate=80 mL/min, 254 nm) to afford methyl 5-(5-amino-2-chloro-4-pyridyl)-4-nitro-1-(2-trimethylsilylethoxymethyl)pyrazole-3-carboxylate (7.26 g, 65.6%) as a yellow oil. LCMS (ESI) m/z: 428.0 [M+H]⁺.

### Step 2: methyl 4-amino-5-(5-amino-2-chloro-4-pyridyl)-1-(2-trimethylsilylethoxymethyl) pyrazole-3-carboxylate (B2-21)

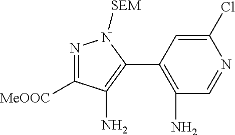 [SMILES: COC(=O)c1nn(COCC[Si](C)(C)C)c(-c2cc(Cl)ncc2N)c1N]

To a solution of methyl 5-(5-amino-2-chloro-4-pyridyl)-4-nitro-1-(2-trimethylsilylethoxy methyl)pyrazole-3-carboxylate (7.26 g, 16.9 mmol) in MeOH (150 mL) and H₂O (50 mL) was added Fe (4.74 g, 84.8 mmol) and NH₄Cl (4.54 g, 84.8 mmol). The mixture was stirred at 80° C. for 3 hr under nitrogen atmosphere, cooled to 20° C., filtered through celite and washed with MeOH (30 mL). The filtrate was concentrated under reduced pressure. The residue was purified by flash chromatography (ISCO®; 80 g AgelaFlash® Silica Flash Column, petroleum ether/EtOAc with EtOAc from 0˜40%, flow rate=80 mL/min, 254 nm) to afford methyl 4-amino-5-(5-amino-2-chloro-4-pyridyl)-1-(2-trimethylsilylethoxymethyl)pyrazole-3-carboxylate (4.8 g, 60.2%) as a yellow solid. ¹H NMR (400 MHz, CDCl₃) δ ppm 0.00 (s, 9H), 0.86-0.95 (m, 2H), 3.57-3.74 (m, 2H), 3.98 (s, 3H), 5.35 (br d, J=13.30 Hz, 2H), 7.41 (s, 1H), 8.01 (s, 1H); LCMS (ESI) m/z: 398.0 [M+H]⁺.

### Synthesis of methyl 4-amino-5-(5-amino-2-chloro-4-pyridyl)-1-(2-trimethylsilylethoxy methyl)pyrazole-3-carboxylate (Intermediate B2-22)

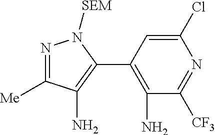 [SMILES: Cc1nn(COCC[Si](C)(C)C)c(-c2cc(Cl)nc(C(F)(F)F)c2N)c1N]

### Step 1: 4-bromo-6-chloro-2-(trifluoromethyl)pyridin-3-amine

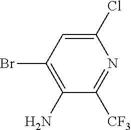 [SMILES: Nc1c(Br)cc(Cl)nc1C(F)(F)F]

To a solution of 6-chloro-2-(trifluoromethyl)pyridin-3-amine (2 g, 10.18 mmol, 1 eq) in MeOH (30 mL) and AcOH (3 mL) was added Br₂ (2.93 g, 18.3 mmol, 1.8 eq) dropwise at 0° C. The mixture was stirred at 20° C. for 16 hr and concentrated in vacuum. The residue was diluted with water (30 mL), quenched by solid NaHCO₃ until pH reached 7 and extracted with a mixture of EtOAc and petroleum ether (2/1, 40 mL×2). The combined organic layers were washed with brine (40 mL), water (40 mL), dried over Na₂SO₄, filtered and concentrated under reduced pressure. The crude product was purified by flash silica gel chromatography (Biotage®; Agela® Flash Column Silica-CS (40 g), Eluent of petroleum ether to 5% EtoAc in petroleum ether, gradient @ 65 mL/min) to afford 4-bromo-6-chloro-2-(trifluoromethyl)pyridin-3-amine (2.48 g, 78.3%) as a yellow solid. LCMS (ESI) m/z: 274.9 [M+H]⁺.

### Step 2: 6-chloro-4-[5-methyl-4-nitro-2-(2-trimethylsilylethoxymethyl)pyrazol-3-yl]-2-(trifluoromethyl)pyridin-3-amine

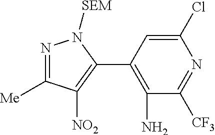 [SMILES: Cc1nn(COCC[Si](C)(C)C)c(-c2cc(Cl)nc(C(F)(F)F)c2N)c1[N+](=O)[O-]]

To a mixture of 4-bromo-6-chloro-2-(trifluoromethyl)pyridin-3-amine (2.6 g, 8.50 mmol, 1 eq) and trimethyl-[2-[(3-methyl-4-nitro-pyrazol-1-yl)methoxy]ethyl]silane (Intermediate A1-1) (6.90 g, 16.1 mmol, 60% purity, 1.89 eq) in dioxane (100 mL) was added Pd(PPh₃)₄ (984 mg, 0.852 mmol, 0.1 eq), CuI (324 mg, 1.70 mmol, 0.2 eq) and Cs₂CO₃ (8.30 g, 25.5 mmol, 3 eq). The reaction mixture was stirred at 100° C. for 16 hr under N₂, filtered and the filtrate was concentrated in vacuum. The residue was purified by flash silica gel chromatography (Biotage®; 220 g Agela Silica Flash Column, Eluent of 0˜25% EtOAc/petroleum ether, gradient @ 100 mL/min) to provide 6-chloro-4-[5-methyl-4-nitro-2-(2-trimethylsilylethoxymethyl)pyrazol-3-yl]-2-(trifluoromethyl)pyridin-3-amine (1.7 g, 39.9%) as a gray oil. LCMS (ESI) m/z: 452.0 [M+H]⁺.

### Step 3: 4-[4-amino-5-methyl-2-(2-trimethylsilylethoxymethyl)pyrazol-3-yl]-6-chloro-2-(trifluoromethyl)pyridin-3-amine (B2-22)

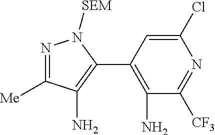 [SMILES: Cc1nn(COCC[Si](C)(C)C)c(-c2cc(Cl)nc(C(F)(F)F)c2N)c1N]

To a mixture of 6-chloro-4-[5-methyl-4-nitro-2-(2-trimethylsilylethoxymethyl)pyrazol-3-yl]-2-(trifluoromethyl)pyridin-3-amine (1.1 g, 2.19 mmol, 90% purity, 1 eq) in EtOH (16 mL) and H₂O (4 mL) was added Fe (1.23 g, 22.0 mmol, 10.04 eq) and NH₄Cl (1.18 g, 22.1 mmol, 10.08 eq). The mixture was stirred at 50° C. for 16 hr under N₂ atmosphere, then filtered. The filtrate was quenched by saturated NaHCO₃ aqueous solution (20 mL) and extracted with EtOAc (30 mL×3). The combined organic layers were washed with brine (30 mL), dried over Na₂SO₄, filtered and concentrated in vacuo. The residue was purified by flash silica gel chromatography (Biotage®; 20 g Agela Silica Flash Column, Eluent of 0˜35% EtOAc/petroleum ether, gradient @ 70 mL/min) to afford 4-[4-amino-5-methyl-2-(2-trimethylsilylethoxymethyl)pyrazol-3-yl]-6-chloro-2-(trifluoromethyl)pyridin-3-amine (970 mg, 64.5%) as a brown solid. LCMS (ESI) m/z: 422.1 [M+H]⁺.

### Synthesis of 5-(2-amino-5-morpholinophenyl)-3-methyl-1-((2-(trimethylsilyl)ethoxy) methyl)-1H-pyrazol-4-amine (Intermediate C1-1)

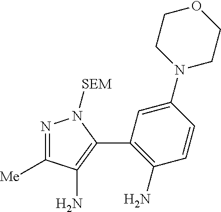 [SMILES: Cc1nn(COCC[Si](C)(C)C)c(-c2cc(N3CCOCC3)ccc2N)c1N]

### Step 1: Synthesis of 4-(3-bromo-4-nitrophenyl)morpholine

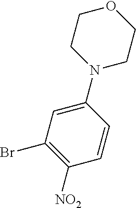 [SMILES: O=[N+]([O-])c1ccc(N2CCOCC2)cc1Br]

A mixture of 2-bromo-4-fluoro-1-nitro-benzene (10 g, 45.5 mmol, 1.0 eq), morpholine (5.1 g, 59.1 mmol, 1.3 eq) and K₂CO₃ (12.6 g, 90.9 mmol, 2.0 eq) in DMF (200 mL) was stirred at 20° C. for 2 hr. Then the reaction mixture was diluted with H₂O (100 mL) and extracted with EtOAc (100 mL×3). The combined organic layers were washed with brine (30 mL×2), dried over anhydrous Na₂SO₄, filtered and concentrated under reduced pressure to give a crude product of 4-(3-bromo-4-nitrophenyl)morpholine as a white solid (14 g, 98% yield). ¹H NMR (400 MHz, DMSO-d₆) δ ppm 3.36-3.47 (m, 4H), 3.64-3.76 (m, 1H), 3.64-3.76 (m, 4H), 7.03 (d, J=2.8 Hz, 1H), 7.27 (d, J=2.8 Hz, 1H), 8.02 (d, J=9.4 Hz, 1H).

### Step 2: Synthesis of 2-bromo-4-morpholinoaniline

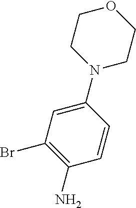 [SMILES: Nc1ccc(N2CCOCC2)cc1Br]

To a solution of 4-(3-bromo-4-nitro-phenyl)morpholine (5 g, 17.41 mmol, 1.0 eq) in EtOH (75 mL) and H₂O (25 mL) was added NH₄Cl (4.66 g, 87.07 mmol, 5.0 eq) and Zn (5.69 g, 87.1 mmol, 5.0 eq). The mixture was stirred at 50° C. for 1 hr. The mixture was filtered to remove insoluble material. The filtrate was collected, dried over sodium sulfate and concentrated. The residue was purified by flash silica gel chromatography (ISCO®; 80 g SepaFlash® Silica Flash Column, petroleum ether/EtOAc with EtOAc from 0˜40%, 70 mL/min, 254 nm), to provide 2-bromo-4-morpholino-aniline as a yellow solid (12 g, 65.6% yield). ¹H NMR (400 MHz, DMSO-d₆) δ ppm 2.78-2.98 (m, 4H), 3.59-3.79 (m, 4H), 3.63-3.79 (m, 1H), 3.63-3.79 (m, 1H), 4.79 (s, 2H), 6.64-6.83 (m, 2H), 6.92 (d, J=2.5 Hz, 1H).

### Step 3: tert-butyl N-(2-bromo-4-morpholino-phenyl)carbamate

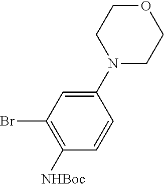 [SMILES: CC(C)(C)OC(=O)Nc1ccc(N2CCOCC2)cc1Br]

To a solution of 2-bromo-4-morpholino-aniline (5.5 g, 21.4 mmol, 1 eq) in THF (200 mL) was added 1M NaHMDS in THF (32 mL, 32 mmol, 1.5 eq) and Boc₂O (5.14 g, 23.5 mmol, 1.1 eq) at 0° C. The mixture was stirred at 20° C. for 16 hr. The reaction mixture was quenched by NH₄Cl/H₂O (20 mL) at 0° C. and then extracted with EtOAc (150 mL×3). The combined organic layers were washed with brine (20 mL), dried over anhydrous Na₂SO₄, filtered and concentrated under reduced pressure. The residue was purified by flash silica gel chromatography (ISCO®; 120 g SepaFlash® Silica Flash Column, petroleum ether/EtOAc with EtOAc from 0˜10%, 85 mL/min, 254 nm), to provide tert-butyl N-(2-bromo-4-morpholino-phenyl)carbamate as a white solid (6.3 g, 41.2%). ¹H NMR (400 MHz, DMSO-d₆) δ ppm 1.42 (s, 9H), 3.00-3.15 (m, 4H), 3.63-3.80 (m, 4H), 6.91 (dd, J=9.0, 2.7 Hz, 1H), 7.11 (d, J=2.7 Hz, 1H), 7.22 (d, J=8.8 Hz, 1H), 8.36 (s, 1H); LCMS (ESI) m/z: 356.9 [M+H]⁺.

### Step 4: tert-butyl N-[2-[5-methyl-4-nitro-2-(2-trimethylsilylethoxymethyl)pyrazol-3-yl]-4-morpholino-phenyl]carbamate

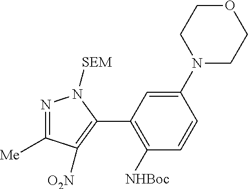 [SMILES: Cc1nn(COCC[Si](C)(C)C)c(-c2cc(N3CCOCC3)ccc2NC(=O)OC(C)(C)C)c1[N+](=O)[O-]]

A mixture of tert-butyl N-(2-bromo-4-morpholino-phenyl)carbamate (1.41 g, 3.95 mmol, 1.0 eq), trimethyl-[2-[(3-methyl-4-nitro-pyrazol-1-yl)methoxy]ethyl]silane (Intermediate A1-1) (1.02 g, 3.95 mmol, 1.0 eq), Cs₂CO₃ (1.93 g, 5.92 mmol, 1.5 eq), cesium(I) 2,2-dimethylpropanoate (1.02 g, 4.34 mmol, 1.1 eq), Pd(OAc)₂ (265.8 mg, 1.18 mmol, 0.3 eq) and XPhos (940.8 mg, 1.97 mmol, 0.5 eq) in toluene (50 mL) was stirred at 100° C. for 16 hr under N₂ atmosphere. The reaction mixture was filtered and concentrated under reduced pressure. The residue was purified by flash silica gel chromatography (ISCO®; 40 g SepaFlash® Silica Flash Column, petroleum ether/EtOAc with EtOAc from 0˜50%, 50 mL/min, 254 nm), to provide tert-butyl N-[2-[5-methyl-4-nitro-2-(2-trimethylsilylethoxymethyl)pyrazol-3-yl]-4-morpholino-phenyl]carbamate as a yellow solid (1.3 g, 49%). LCMS (ESI) m/z: 534.2 [M+H]⁺.

### Step 5: tert-butyl N-[2-[4-amino-5-methyl-2-(2-trimethylsilylethoxymethyl)pyrazol-3-yl]-4-morpholino-phenyl]carbamate

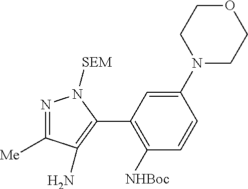 [SMILES: Cc1nn(COCC[Si](C)(C)C)c(-c2cc(N3CCOCC3)ccc2NC(=O)OC(C)(C)C)c1N]

To a solution of tert-butyl N-[2-[5-methyl-4-nitro-2-(2-trimethylsilylethoxymethyl) pyrazol-3-yl]-4-morpholino-phenyl]carbamate (1.3 g, 1.95 mmol, 1.0 eq) in EtOH (3 mL) and H₂O (0.6 mL) was added NH₄Cl (1.04 g, 19.5 mmol, 10.0 eq) and Zn (1.27 g, 19.5 mmol, 10.0 eq). The mixture was stirred at 50° C. for 16 hr under N₂ atmosphere, filtered to remove insoluble material. The filtrate was collected, dried over sodium sulfate and concentrated. The residue was purified by flash silica gel chromatography (ISCO®; 80 g SepaFlash® Silica Flash Column, petroleum ether/EtOAc with EtOAc from 0˜40%, 70 mL/min, 254 nm), to provide tert-butyl N-[2-[4-amino-5-methyl-2-(2-trimethylsilylethoxymethyl)pyrazol-3-yl]-4-morpholino-phenyl]carbamate as a yellow solid (800 mg, 80% yield). ¹H NMR (400 MHz, DMSO-d₆) δ ppm −0.06 (s, 9H), 0.76 (t, J=8.3 Hz, 2H), 1.38 (s, 9H), 2.12 (s, 3H), 3.07 (br d, J=5.0 Hz, 4H), 3.73 (t, J=4.7 Hz, 4H), 3.84 (br s, 2H), 4.83-5.21 (m, 2H), 6.92 (d, J=2.8 Hz, 1H), 7.02 (dd, J=9.1, 2.8 Hz, 1H), 7.55 (br d, J=8.0 Hz, 1H), 8.44 (s, 1H); LCMS (ESI) m/z: 504.2 [M+H]⁺.

### Step 6: 5-(2-amino-5-morpholino-phenyl)-3-methyl-1-(2-trimethylsilylethoxymethyl) pyrazol-4-amine (C1-1)

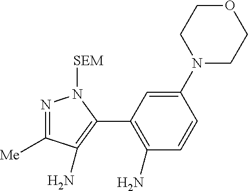 [SMILES: Cc1nn(COCC[Si](C)(C)C)c(-c2cc(N3CCOCC3)ccc2N)c1N]

To a solution of tert-butyl N-[2-[4-amino-5-methyl-2-(2-trimethylsilylethoxymethyl) pyrazol-3-yl]-4-morpholino-phenyl]carbamate (200 mg, 0.397 mmol, 1.0 eq) in DCM (2 mL) was added ZnBr₂ (268 mg, 1.19 mmol, 3.0 eq). The mixture was stirred at 40° C. for 16 hr under N₂ atmosphere, quenched by water (1 mL), extracted with EtOAc (10 mL), dried over Na₂SO₄ and concentrated to provide 5-(2-amino-5-morpholino-phenyl)-3-methyl-1-(2-trimethylsilylethoxymethyl) pyrazol-4-amine as a yellow solid (160 mg), which was used for next step directly without any further purification.

### Synthesis of 5-(2-amino-5-morpholinophenyl)-3-chloro-1-(4-methoxybenzyl)-1H-pyrazol-4-amine (Intermediate C1-2)

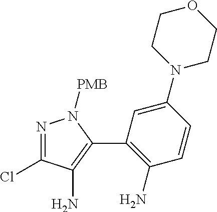

### Step 1: tert-butyl (2-(3-chloro-1-(4-methoxybenzyl)-4-nitro-1H-pyrazol-5-yl)-4-morpholinophenyl)carbamate

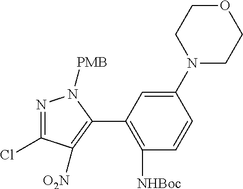

A mixture of tert-butyl N-(2-bromo-4-morpholino-phenyl)carbamate (1.5 g, 4.20 mmol, 1.0 eq), 3-chloro-1-[(4-methoxyphenyl)methyl]-4-nitro-pyrazole (Intermediate A2-2) (1.12 g, 4.20 mmol, 1.0 eq), Cs₂CO₃ (2.05 g, 6.30 mmol, 1.5 eq), cesium; 2,2-dimethylpropanoate (1.08 g, 4.62 mmol, 1.1 eq), Pd(OAc)₂ (282.8 mg, 1.26 mmol, 0.3 eq) and XPhos (1.0 g, 2.10 mmol, 0.5 eq) in dioxane (15 mL) was degassed and purged with N₂ for 3 times. The mixture was stirred at 100° C. for 16 hr, filtered and concentrated under reduced pressure. The residue was purified by flash silica gel chromatography (ISCO®; 12 g SepaFlash® Silica Flash Column, petroleum ether/EtOAc with EtOAc from 0˜30%, 50 mL/min, 254 nm), to provide tert-butyl N-[2-[5-chloro-2-[(4-methoxyphenyl)methyl]-4-nitro-pyrazol-3-yl]-4-morpholino-phenyl]carbamate as a brown oil (2.9 g, 55.2%). LCMS (ESI) m/z: 544.2 [M+H]⁺.

### Step 2: tert-butyl (2-(4-amino-3-chloro-1-(4-methoxybenzyl)-1H-pyrazol-5-yl)-4-morpholinophenyl)carbamate

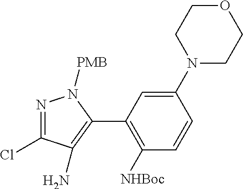

To a solution of tert-butyl N-[2-[5-chloro-2-[(4-methoxyphenyl)methyl]-4-nitro-pyrazol-3-yl]-4-morpholino-phenyl]carbamate (1.4 g, 2.57 mmol, 1.0 eq) in THF (20 mL) was added NaBH₄ (584.2 mg, 15.44 mmol, 7.0 eq) and NiCl₂·6H₂O (1.84 g, 7.72 mmol, 3.0 eq) at 0° C. The mixture was stirred at 25° C. for 1 hr, filtered and concentrated under reduced pressure. The residue was purified by flash silica gel chromatography (ISCO®; 4 g SepaFlash® Silica Flash Column, petroleum ether/EtOAc with EtOAc from 0˜30%, 40 mL/min, 254 nm), to provide tert-butyl N-[2-[4-amino-5-chloro-2-[(4-methoxyphenyl)methyl]pyrazol-3-yl]-4-morpholino-phenyl]carbamate as a white solid (800 mg, 58.6%). LCMS (ESI) m/z: 514.2 [M+H]⁺.

### Step 3: 5-(2-amino-5-morpholinophenyl)-3-chloro-1-(4-methoxybenzyl)-1H-pyrazol-4-amine (C1-2)

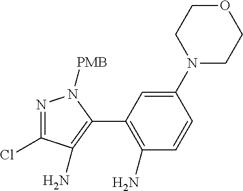

A solution of tert-butyl N-[2-[4-amino-5-chloro-2-[(4-methoxyphenyl)methyl]pyrazol-3-yl]-4-morpholino-phenyl]carbamate (1.5 g, 2.92 mmol, 1.0 eq) in 4N HCl in MeOH (15 mL) was stirred at 25° C. for 2 hr. The reaction mixture was concentrated under reduced pressure to provide 5-(2-amino-5-morpholino-phenyl)-3-chloro-1-[(4-methoxyphenyl)methyl]pyrazol-4-amine as a white solid (1.4 g, 96.3%). LCMS (ESI) m/z: 414.1 [M+H]⁺.

### Synthesis of 5-(2-amino-4-fluoro-5-morpholino-phenyl)-1-[(4-methoxyphenyl)methyl]-3-methyl-pyrazol-4-amine (Intermediate C1-3)

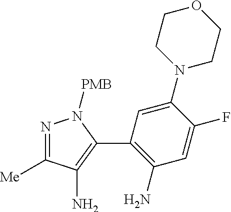

### Step 1: 4-(5-bromo-2-fluoro-4-nitro-phenyl)morpholine

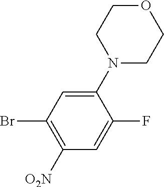 [SMILES: O=[N+]([O-])c1cc(F)c(N2CCOCC2)cc1Br]

To a solution of 1-bromo-4,5-difluoro-2-nitro-benzene (20.0 g, 84.0 mmol, 1 eq.) in DMF (360 mL) was added K₂CO₃ (13.9 g, 0.101 mol, 1.2 eq.) and morpholine (8.05 g, 92.4 mmol, 1.1 eq.). The mixture was stirred at 25° C. for 1 hr and diluted with H₂O (1200 mL). A large number of solids were precipitated out. The resulting mixture was extracted with EtOAc (500 mL×3), washed with brine (500 mL), dried over anhydrous Na₂SO₄ and concentrated under reduced pressure. The residue was purified by flash chromatography on silica gel column using 0-60% of EtOAc in petroleum ether as eluent, to provide 4-(5-bromo-2-fluoro-4-nitro-phenyl)morpholine as a yellow solid (20 g, 78.0%). ¹H NMR (400 MHz, DMSO-d₆) δ ppm 3.23-3.29 (m, 4H), 3.67-3.78 (m, 4H), 7.35 (d, J=8.3 Hz, 1H), 8.05 (d, J=13.4 Hz, 1H).

### Step 2: 2-bromo-5-fluoro-4-morpholino-aniline

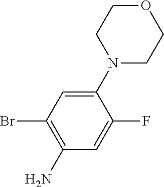 [SMILES: Nc1cc(F)c(N2CCOCC2)cc1Br]

A mixture of 4-(5-bromo-2-fluoro-4-nitro-phenyl)morpholine (20.0 g, 65.6 mmol, 1 eq.), Zn (42.9 g, 0.656 mol, 10 eq.), NH₄Cl (35.1 g, 0.656 mol, 10 eq.) in EtOH/H₂O (5:1, 24 mL) was degassed and purged with N₂ for 3 times, and stirred at 50° C. for 2 hr under N₂ atmosphere. It was filtered and concentrated to give 2-bromo-5-fluoro-4-morpholino-aniline as a white solid (16.0 g, 87.0%). ¹H NMR (400 MHz, DMSO-d₆) δ ppm 2.77-2.89 (m, 4H), 3.62-3.75 (m, 4H), 5.19 (s, 2H), 6.62 (d, J=14.1 Hz, 1H), 7.01 (d, J=8.9 Hz, 1H).

### Step 3: tert-butyl N-(2-bromo-5-fluoro-4-morpholino-phenyl)carbamate

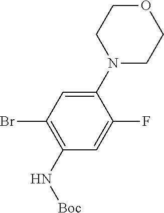 [SMILES: CC(C)(C)OC(=O)Nc1cc(F)c(N2CCOCC2)cc1Br]

To a solution of 2-bromo-5-fluoro-4-morpholino-aniline (16.0 g, 58.2 mmol, 1 eq.) in THF (400 mL) was added dropwise 1M LiHMDS in THF (128 mL, 0.128 mol, 2.2 eq.) at 0° C. After addition, the mixture was stirred at this temperature for 30 min, and then Boc₂O (13.3 g, 61.1 mmol, 1.05 eq.) was added dropwise at 0° C. The resulting mixture was stirred at 20° C. for 16 hr, diluted with H₂O (20 mL), extracted with EtOAc (20 mL×2), washed with brine (20 mL), dried over anhydrous Na₂SO₄ and concentrated under reduced pressure. The residue was purified by flash chromatography on silica gel column using 0-60% of EtOAc in petroleum ether as eluent, to provide tert-butyl N-(2-bromo-5-fluoro-4-morpholino-phenyl)carbamate as a white solid (18.0 g, 80.8%). ¹H NMR (400 MHz, DMSO-d₆) δ ppm 1.32-1.48 (m, 9H), 2.86-2.98 (m, 4H), 3.59-3.71 (m, 4H), 7.11 (d, J=9.0 Hz, 1H), 7.26 (d, J=14.0 Hz, 1H), 8.45 (s, 1H).

### Step 4: tert-butyl N-[5-fluoro-2-[2-[(4-methoxyphenyl)methyl]-5-methyl-4-nitro-pyrazol-3-yl]-4-morpholino-phenyl]carbamate

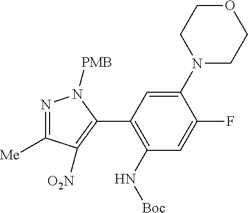

To a solution of tert-butyl N-(2-bromo-5-fluoro-4-morpholino-phenyl)carbamate (1 g, 2.67 mmol, 1 eq), 1-[(4-methoxyphenyl)methyl]-3-methyl-4-nitro-pyrazole (Intermediate A2-1) (725 mg, 2.93 mmol, 1.1 eq.), XPhos (508 mg, 1.07 mmol, 0.4 eq.) and Cs₂CO₃ (1.30 g, 4.00 mmol, 1.5 eq.) and CsOPiv (686 mg, 2.93 mmol, 1.1 eq.) in toluene (10 mL) was de-gassed and added Pd(OAc)₂ (120 mg, 0.533 mmol, 0.2 eq.). The mixture was stirred at 100° C. for 16 hr under N₂ atmosphere, filtered and concentrated under reduced pressure. The residue was purified by flash chromatography on silica gel column using 0-20% of EtOAc in petroleum ether as eluent, to provide tert-butyl N-[5-fluoro-2-[2-[(4-methoxyphenyl)methyl]-5-methyl-4-nitro-pyrazol-3-yl]-4-morpholino-phenyl]carbamate as a yellow oil (680 mg, 47.1%). ¹H NMR (400 MHz, DMSO-d₆) δ ppm 1.35 (s, 9H), 2.48 (s, 3H), 2.62-2.73 (m, 2H), 2.76-2.85 (m, 2H), 3.65 (t, J=4.6 Hz, 4H), 3.72 (s, 3H), 4.80 (d, J=15.4 Hz, 1H), 5.08 (d, J=15.3 Hz, 1H), 6.55 (d, J=9.5 Hz, 1H), 6.80-6.88 (m, 2H), 6.99 (d, J=8.7 Hz, 2H), 7.33 (d, J=14.4 Hz, 1H), 8.90 (s, 1H).

### Step 5: tert-butyl N-[2-[4-amino-2-[(4-methoxyphenyl)methyl]-5-methyl-pyrazol-3-yl]-5-fluoro-4-morpholino-phenyl]carbamate

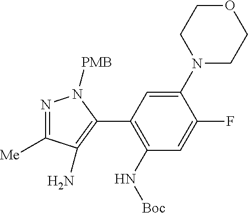

To a solution of tert-butyl N-[5-fluoro-2-[2-[(4-methoxyphenyl)methyl]-5-methyl-4-nitro-pyrazol-3-yl]-4-morpholino-phenyl]carbamate (680 mg, 1.26 mmol, 1 eq.) in EtOH/H₂O (3:1, 12 mL) was added NH₄Cl (672 mg, 12.6 mmol, 10 eq.) and Fe (701 mg, 12.6 mmol, 10 eq.). The mixture was stirred at 50° C. for 16 hr under N₂ atmosphere, filtered and concentrated under reduced pressure. The residue was purified by flash chromatography on silica gel column using 0-100% of EtOAc in petroleum ether as eluent, to provide tert-butyl N-[2-[4-amino-2-[(4-methoxyphenyl)methyl]-5-methyl-pyrazol-3-yl]-5-fluoro-4-morpholino-phenyl]carbamate as a yellow solid (250 mg, 38.9%). ¹H NMR (400 MHz, DMSO-d₆) δ ppm 1.31-1.47 (m, 9H), 2.13 (s, 3H), 2.59-2.84 (m, 4H), 3.64 (t, J=4.5 Hz, 4H), 3.69 (s, 3H), 3.87 (br s, 2H), 4.76-4.87 (m, 1H), 4.99-5.11 (m, 1H), 6.56 (d, J=9.7 Hz, 1H), 6.72-6.89 (m, 4H), 7.40-7.60 (m, 1H), 8.45 (s, 1H); LCMS (ESI) m/z: 312.3 [M+H]⁺.

### Step 6: 5-(2-amino-4-fluoro-5-morpholino-phenyl)-1-[(4-methoxyphenyl)methyl]-3-methyl-pyrazol-4-amine (C1-3)

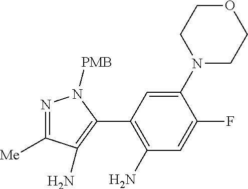

A solution of tert-butyl N-[2-[4-amino-2-[(4-methoxyphenyl)methyl]-5-methyl-pyrazol-3-yl]-5-fluoro-4-morpholinophenyl] carbamate (250 mg, 0.489 mmol, 1 eq.) in 4N HCl in MeOH (10 mL) was stirred at 25° C. for 2 hr. The mixture was then concentrated to provide 5-(2-amino-4-fluoro-5-morpholino-phenyl)-1-[(4-methoxyphenyl)methyl]-3-methyl-pyrazol-4-amine as a yellow solid (220 mg, 92.9%). ¹H NMR (400 MHz, DMSO-d₆) δ ppm 2.27 (s, 3H), 2.73-2.81 (m, 1H), 2.84-2.93 (m, 2H), 3.66-3.75 (m, 7H), 4.85 (br d, J=15.3 Hz, 1H), 5.08 (d, J=15.4 Hz, 1H), 6.52 (br d, J=9.1 Hz, 1H), 6.75-6.94 (m, 5H); LCMS (ESI) m/z: 412.2 [M+H]⁺.

### Synthesis of 5-(2-amino-3-fluoro-5-morpholino-phenyl)-1-[(4-methoxyphenyl)methyl]-3-methyl-pyrazol-4-amine (Intermediate C1-4)

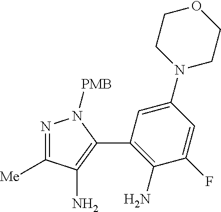

### Step 1: 4-(3-bromo-5-fluoro-4-nitro-phenyl)morpholine

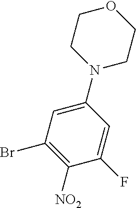 [SMILES: O=[N+]([O-])c1c(F)cc(N2CCOCC2)cc1Br]

A mixture of 1-bromo-3,5-difluoro-2-nitro-benzene (20 g, 84.0 mmol, 1 eq.), morpholine (7.32 g, 84.0 mmol, 1 eq.) in DMF (200 mL) was degassed and purged with N₂ for 3 times. The reaction mixture was stirred at 25° C. for 1 hr under N₂ atmosphere, diluted with H₂O (600 mL), extracted with EtOAc (200 mL×3). The combined organic layers were washed 5 wt % LiCl in H₂O (300 mL×2) and brine (200 mL). The organic layer was dried over anhydrous Na₂SO₄ and concentrated under reduced pressure. The residue was purified by flash chromatography on silica gel column using 0-20% of EtOAc in petroleum ether as eluent, to provide 4-(3-bromo-5-fluoro-4-nitro-phenyl)morpholine as a yellow solid (8.8 g, 34.3%). ¹H NMR (400 MHz, DMSO-d₆) δ ppm 3.35-3.43 (m, 4H), 3.66-3.75 (m, 4H), 7.06 (dd, J=14.6, 2.3 Hz, 1H), 7.14 (d, J=1.0 Hz, 1H).

### Step 2: 2-bromo-6-fluoro-4-morpholino-aniline

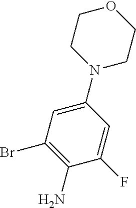 [SMILES: Nc1c(F)cc(N2CCOCC2)cc1Br]

A mixture of 4-(3-bromo-5-fluoro-4-nitro-phenyl)morpholine (8.8 g, 28.8 mmol, 1 eq.), Zn (18.7 g, 0.288 mol, 10 eq.), NH₄Cl (15.4 g, 0.288 mol, 10 eq.) in EtOH/H₂O (3/1, 160 mL) was degassed and purged with N₂ for 3 times, and then the mixture was stirred at 50° C. for 1 hr under N₂ atmosphere. The reaction mixture was filtered and the filtrate was extracted with EtOAc (100 mL×3). The combined organic layers were concentrated under reduced pressure. The residue was purified by flash chromatography on silica gel column using 0-30% of EtOAc in petroleum ether as eluent, to provide 2-bromo-6-fluoro-4-morpholino-aniline as a colorless oil (7.5 g, 94.5%). ¹H NMR (400 MHz, DMSO-d₆) δ ppm 2.88-2.98 (m, 4H), 3.62-3.71 (m, 4H), 4.69 (s, 2H), 6.78-6.84 (m, 2H).

### Step 3: tert-butyl N-(2-bromo-6-fluoro-4-morpholino-phenyl)carbamate

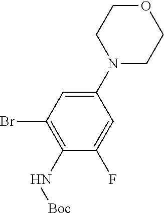 [SMILES: CC(C)(C)OC(=O)Nc1c(F)cc(N2CCOCC2)cc1Br]

To a solution of 2-bromo-6-fluoro-4-morpholino-aniline (6.6 g, 23.9 mmol, 1 eq.) in THF (70 mL) was added dropwise 1M NaHMDS in THF (47.9 mL, 47.9 mmol, 2 eq.) under N₂ at 0° C. for 30 min. Then Boc₂O (7.85 g, 35.9 mmol, 1.5 eq.) in THF (70 mL) was added dropwise at 0° C. The resulting mixture was stirred at 20° C. for 16 hr under N₂ atmosphere. An aqueous solution of NH₄Cl (70 mL) was added at 0° C. to quench the reaction. The mixture was extracted with EtOAc (100 mL×2), dried over anhydrous Na₂SO₄ and concentrated in vacuum. The residue was purified by flash chromatography on silica gel column using 0-20% of EtOAc in petroleum ether as eluent, to provide tert-butyl N-(2-bromo-6-fluoro-4-morpholino-phenyl)carbamate as a white solid (4.1 g, 45.5%). ¹H NMR (400 MHz, DMSO-d₆) δ ppm 1.39-1.48 (m, 9H), 3.12-3.17 (m, 5H), 3.68-3.73 (m, 5H), 6.86 (dd, J=13.0, 2.6 Hz, 1H), 6.99 (s, 1H), 8.48 (br s, 1H).

### Step 4: tert-butyl N-[2-fluoro-6-[2-[(4-methoxyphenyl) methyl]-5-methyl-4-nitro-pyrazol-3-yl]-4-morpholino-phenyl] carbamate

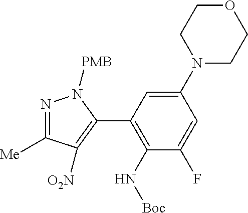

A stirred solution of tert-butyl N-(2-bromo-6-fluoro-4-morpholino-phenyl)carbamate (1 g, 2.67 mmol, 1 eq.), 1-[(4-methoxyphenyl)methyl]-3-methyl-4-nitro-pyrazole (Intermediate A2-1) (658 mg, 2.67 mmol, 1 eq.) in toluene (30 mL) was added with Pd(OAc)₂ (59.8 mg, 0.266 mmol, 0.1 eq.), XPhos (381 mg, 0.799 mmol, 0.3 eq.), CsOPiv (686 mg, 2.93 mmol, 1.1 eq.), Cs₂CO₃ (1.30 g, 4.00 mmol, 1.5 eq.). The mixture was purged and degassed with N₂ for 3 times, then stirred at 100° C. for 16 hr. The mixture was filtered and the filtrate was concentrated in vacuum. The residue was purified by flash chromatography on silica gel column using 0-20% of EtOAc in petroleum ether as eluent, to provide tert-butyl N-[2-fluoro-6-[2-[(4-methoxyphenyl)methyl]-5-methyl-4-nitro-pyrazol-3-yl]-4-morpholino-phenyl]carbamate as a dark brown oil (580 mg, 40.32%). ¹H NMR (400 MHz, CDCl₃) δ ppm 1.36 (s, 11H), 2.61 (s, 3H), 2.80-2.99 (m, 5H), 3.67-3.84 (m, 8H), 4.97-5.24 (m, 2H), 6.12 (d, J=1.8 Hz, 1H), 6.73 (dd, J=12.6, 2.8 Hz, 1H), 6.83 (d, J=8.6 Hz, 2H), 6.99 (s, 1H), 7.09 (br d, J=7.8 Hz, 2H); LCMS (ESI) m/z: 542.3 [M+H]⁺.

### Step 5: tert-butyl N-[2-[4-amino-2-[(4-methoxyphenyl)methyl]-5-methyl-pyrazol-3-yl]-6-fluoro-4-morpholino-phenyl]carbamate

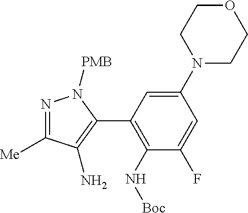

A stirred solution of tert-butyl N-[2-fluoro-6-[2-[(4-methoxyphenyl)methyl]-5-methyl-4-nitro-pyrazol-3-yl]-4-morpholino-phenyl]carbamate (580 mg, 1.07 mmol, 1 eq.), Zn (700 mg, 10.7 mmol, 10 eq.), NH₄Cl (572 mg, 10.7 mmol, 10 eq.) in EtOH/H₂O (1:1, 30 mL) was stirred at 50° C. for 1 hr. The reaction mixture was added with EtOAc (50 mL) and H₂O (50 mL), extracted with EtOAc (50 mL). The combined organic layers were washed with brine (40 mL), dried over anhydrous Na₂SO₄ and concentrated in vacuum. The residue was purified by flash chromatography on silica gel column using 0-60% of EtOAc in petroleum ether as eluent, to provide tert-butyl N-[2-[4-amino-2-[(4-methoxyphenyl)methyl]-5-methyl-pyrazol-3-yl]-6-fluoro-4-morpholino-phenyl]carbamate as a brown solid (280 mg, 51.1%). ¹H NMR (500 MHz, CDCl₃) δ ppm 1.33-1.47 (m, 9H), 2.19-2.31 (m, 3H), 2.84-3.01 (m, 5H), 3.69-3.81 (m, 7H), 4.91-5.01 (m, 1H), 5.13 (d, J=15.7 Hz, 1H), 6.27-6.38 (m, 2H), 6.66 (dd, J=12.7, 2.7 Hz, 1H), 6.73-6.81 (m, 2H), 6.92 (d, J=8.5 Hz, 2H).

### Step 6: 5-(2-amino-3-fluoro-5-morpholino-phenyl)-1-[(4-methoxyphenyl)methyl]-3-methyl-pyrazol-4-amine (C1-4)

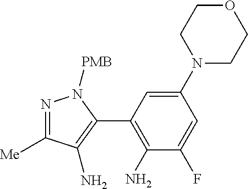

A stirred solution of tert-butyl N-[2-[4-amino-2-[(4-methoxyphenyl) methyl]-5-methyl-pyrazol-3-yl]-6-fluoro-4-morpholino-phenyl]carbamate (280 mg, 0.547 mmol, 1 eq.) in MeOH (1 mL) was added with 4N HCl in MeOH (3.00 mL, 12 mmol, 22 eq.). The mixture was stirred at 25° C. for 1 hr and concentrated in vacuum to provide 5-(2-amino-3-fluoro-5-morpholino-phenyl)-1-[(4-methoxyphenyl)methyl]-3-methyl-pyrazol-4-amine as a dark brown solid (220 mg, 97.6%). ¹H NMR (400 MHz, DMSO-d₆) δ ppm 2.27 (s, 3H), 3.16 (s, 3H), 3.63-3.72 (m, 4H), 3.87 (br s, 6H), 4.78-5.21 (m, 2H), 6.79 (d, J=8.6 Hz, 3H), 6.84-6.95 (m, 2H), 7.28-7.61 (m, 1H).

### Synthesis of 5-(2-amino-5-morpholinophenyl)-1-(4-methoxybenzyl)-1H-pyrazol-4-amine (Intermediate C1-5)

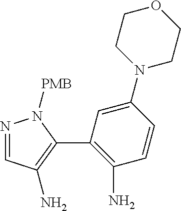

### Step 1: 4-(3-bromo-4-nitrophenyl)morpholine

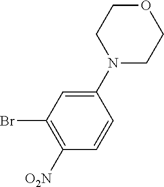 [SMILES: O=[N+]([O-])c1ccc(N2CCOCC2)cc1Br]

To a solution of 2-bromo-4-fluoro-1-nitrobenzene (650 g, 2.87 mol, 1.0 eq.) in DMAc (3.25 L) was added TEA (145 g, 1.43 mol, 0.5 eq.) and then the mixture was added morpholine (274 g, 3.15 mol, 1.1 eq.) at 20° C. The mixture was stirred at 30° C. for 12 hr, poured into water (6 L) at 30° C. and kept stirred at 30° C. for 15 min. The resulting suspension was filtered and the filter cake was collected, washed with water (500 mL) and dissolved in DCM (2.00 L). The combined organic layers were dried over anhydrous Na₂SO₄, filtered and the filtrate was concentrated under reduced pressure. The residue was triturated with petroleum ether (9 L) at 20° C. for 2 hr, then the mixture was filtered and the filter cake was dried under reduced pressure to provide 4-(3-bromo-4-nitrophenyl)morpholine as a yellow solid (2.30 kg, 92.3%). ¹H NMR (400 MHz, DMSO-d₆) δ ppm 3.40 (t, J=4.8 Hz, 4H), 3.72 (t, J=4.8 Hz, 4H), 7.03 (dd, J=2.8, 9.2 Hz, 1H), 7.26 (d, J=2.8 Hz, 1H), 8.00 (d, J=9.2 Hz, 1H).

### Step 2: 2-bromo-4-morpholinoaniline

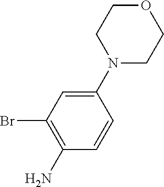 [SMILES: Nc1ccc(N2CCOCC2)cc1Br]

To a solution of 4-(3-bromo-4-nitrophenyl)morpholine (180 g, 0.626 mol, 1.0 eq.) in EtOH/H₂O (2:1, 4.0 L) was added NH₄Cl (168 g, 3.14 mol, 5.0 eq.) at 20° C. Then Fe (175 g, 3.13 mol, 5.0 eq.) was added to mixture under 20° C. and N₂ atmosphere. The mixture was stirred at 40° C. for 3 hr under N₂ atmosphere, cooled to 20° C. and filtered. The filtrate was concentrated under reduced pressure to remove most of EtOH. The residue was extracted with EtOAc (2.00 L) and the organic phase was washed with brine (1.00 L×2), dried over anhydrous Na₂SO₄, filtered and concentrated under reduced pressure to give 2-bromo-4-morpholinoaniline as a dark brown solid (1.75 kg, 89.8%). ¹H NMR (400 MHz, DMSO-d₆) δ ppm 2.89 (t, J=4.8 Hz, 4H), 3.69 (t, J=4.8 Hz, 4H), 4.79 (s, 2H), 6.71-6.82 (m, 2H), 6.92 (d, J=2.4 Hz, 1H).

### Step 3: tert-butyl (2-bromo-4-morpholinophenyl)carbamate

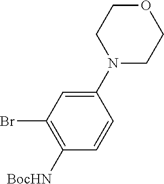 [SMILES: CC(C)(C)OC(=O)Nc1ccc(N2CCOCC2)cc1Br]

To a 10 mL reaction vial was added 2-bromo-4-morpholinoaniline (300 mg, 1.17 mmol, 1.0 eq.), THF (3 mL), NH₄Cl (2 mg, 37.4 μmol, 0.03 eq.) and H₂O (126 mg, 6.99 mmol, 6.0 eq.) at 25° C. Then Boc₂O (382 mg, 1.75 mmol, 1.5 eq.) in THF (1 mL) was added at 60° C. The reaction mixture was stirred at 60° C. for 16 hr, cooled down to 25° C. and concentrated under reduced pressure. The residue was purified by column chromatography (silica gel, petroleum ether/EtOAc=40/1 to 5/1) to give tert-butyl (2-bromo-4-morpholinophenyl)carbamate as a white solid (340 mg, 78.8%). ¹H NMR (400 MHz, CDCl₃) δ ppm 1.53 (s, 9H), 2.97-3.19 (m, 4H), 3.76-3.95 (m, 4H), 6.73 (s, 1H), 6.82-6.91 (m, 1H), 7.05 (d, J=2.8 Hz, 1H), 7.94 (d, J=8.8 Hz, 1H); LCMS (ESI) m/z: 357.1 [M+H]⁺.

### Step 4: tert-butyl (2-(1-(4-methoxybenzyl)-4-nitro-1H-pyrazol-5-yl)-4-morpholinophenyl) carbamate

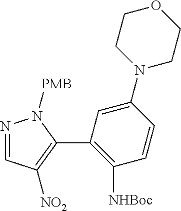

To a 1 L reaction flask was added tert-butyl (2-bromo-4-morpholinophenyl)carbamate (22.0 g, 61.6 mmol, 1 eq.), 1-(4-methoxybenzyl)-4-nitro-1H-pyrazole (Intermediate A2-3) (15.8 g, 67.7 mmol, 1.1 eq.), XPhos (8.81 g, 18.5 mmol, 0.3 eq.), Cs₂CO₃ (30.1 g, 92.4 mmol, 1.5 eq.), CsOPiv (15.8 g, 67.7 mmol, 1.1 eq.) and toluene (440 mL) at 25° C. The reaction mixture was degassed and purged with nitrogen three times. Then Pd(OAc)₂ (1.38 g, 6.16 mmol, 0.1 eq.) was added at 25° C. The reaction mixture was degassed and purged with nitrogen three times and stirred at 100° C. for 16 hr under nitrogen. The reaction mixture was cooled down to 25° C., quenched by water (400 mL) and extracted with EtOAc (450 mL×2). The combined EtOAc phase was washed with brine (300 mL), dried over anhydrous Na₂SO₄ and filtered. The filtrate was concentrated under reduced pressure. The residue was purified by column chromatography (silica gel, petroleum ether/EtOAc=20/1 to 3/1) to provide tert-butyl (2-(1-(4-methoxybenzyl)-4-nitro-1H-pyrazol-5-yl)-4-morpholinophenyl)carbamate as a brown solid (22.0 g, 67.4%). ¹H NMR (400 MHz, DMSO-d₆) δ ppm 1.31 (s, 9H), 2.81-2.97 (m, 4H), 3.60-3.69 (m, 4H), 3.71 (s, 3H), 4.89 (d, J=15.2 Hz, 1H), 5.12 (d, J=15.2 Hz, 1H), 6.54 (d, J=2.8 Hz, 1H), 6.84 (d, J=8.4 Hz, 2H), 7.00 (d, J=8.4 Hz, 2H), 7.04-7.14 (m, 1H), 7.26 (d, J=8.8 Hz, 1H), 8.35 (s, 1H), 8.71 (s, 1H); LCMS (ESI) m/z: 510.3 [M+H]⁺.

### Step 5: tert-butyl (2-(4-amino-1-(4-methoxybenzyl)-1H-pyrazol-5-yl)-4-morpholinophenyl) carbamate

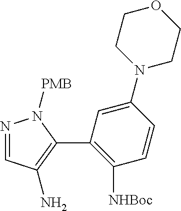

To the solution of tert-butyl (2-(1-(4-methoxybenzyl)-4-nitro-1H-pyrazol-5-yl)-4-morpholinophenyl)carbamate (20.0 g, 39.2 mmol, 1.0 eq.) in EtOH/H₂O (2:1, 450 mL) was added Fe (11.2 g, 0.20 mol, 5.1 eq.) and NH₄Cl (10.6 g, 0.20 mol, 5.1 eq.) at 25° C. After addition, the reaction mixture was stirred at 80° C. for 2 hr under nitrogen, cooled down to 25° C. and filtered through a pad of celite. The filter cake was washed with EtOH (300 mL). The combined organic layers were concentrated under reduced pressure. The residue was diluted with EtOAc (300 mL) and water (200 mL) under stirring. Then the aqueous phase was extracted with EtOAc (200 mL×2). The combined organic phase was washed with brine (200 mL), dried over anhydrous Na₂SO₄, filtered and concentrated under reduced pressure. The residue was purified by column chromatography (silica gel, petroleum ether/EtOAc=5/1 to 1/1) and then triturated in petroleum ether/EtOAc (100 mL, petroleum ether/EtOAc=1/1). The precipitate was collected by filteration, dried under reduced pressure to provide tert-butyl (2-(4-amino-1-(4-methoxybenzyl)-1H-pyrazol-5-yl)-4-morpholinophenyl)carbamate as a light yellow solid (11.9 g, 62.3%). ¹H NMR (400 MHz, DMSO-d₆) δ ppm 1.38 (s, 9H), 2.73-2.83 (m, 2H), 2.84-2.93 (m, 2H), 3.64 (t, J=4.4 Hz, 4H), 3.68 (s, 3H), 4.03 (s, 2H), 4.90 (d, J=15.6 Hz, 1H), 5.15 (d, J=16.0 Hz, 1H), 6.51 (d, J=2.8 Hz, 1H), 6.74-6.89 (m, 4H), 6.92-7.07 (m, 1H), 7.18 (s, 1H), 7.48 (d, J=8.0 Hz, 1H), 8.39 (s, 1H); LCMS (ESI) m/z: 480.3 [M+H]⁺.

### Step 6: 5-(2-amino-5-morpholinophenyl)-1-(4-methoxybenzyl)-1H-pyrazol-4-amine (C1-5)

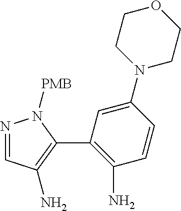

To 250 mL reaction flask was added tert-butyl (2-(4-amino-1-(4-methoxybenzyl)-1H-pyrazol-5-yl)-4-morpholinophenyl)carbamate (11.9 g, 24.8 mmol, 1.0 eq.) and 4N HCl in EtOAc (100 mL) at 25° C. The reaction mixture was stirred at 25° C. for 2 hr. The precipitate was collected, dissolved in water (200 mL), and basified with sodium carbonate solid until the pH was adjusted to 8. White precipitate was collected and dried under reduced pressure to provide 5-(2-amino-5-morpholinophenyl)-1-(4-methoxybenzyl)-1H-pyrazol-4-amine as a light-yellow solid (7.70 g, 79.3%). ¹H NMR (400 MHz, DMSO-d₆) δ ppm 2.64-2.83 (m, 4H), 3.63 (t, J=4.4 Hz, 4H), 3.67-3.84 (m, 5H), 4.49 (s, 2H), 4.88 (d, J=15.6 Hz, 1H), 5.12 (d, J=15.6 Hz, 1H), 6.38 (d, J=2.4 Hz, 1H), 6.69-6.76 (m, 1H), 6.76-6.91 (m, 5H), 7.12 (s, 1H); LCMS (ESI) m/z: 380.2 [M+H]⁺.

### Synthesis of 4-(4-amino-3-methyl-1-((2-(trimethylsilyl)ethoxy)methyl)-1H-pyrazol-5-yl)-6-morpholinopyridin-3-amine (Intermediate C2-1)

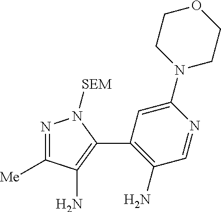 [SMILES: Cc1nn(COCC[Si](C)(C)C)c(-c2cc(N3CCOCC3)ncc2N)c1N]

### Step 1: 4-(5-nitropyridin-2-yl)morpholine

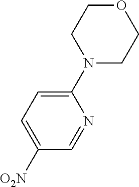 [SMILES: O=[N+]([O-])c1ccc(N2CCOCC2)nc1]

A solution of 2-chloro-5-nitro-pyridine (15 g, 94.6 mmol, 1.0 eq) morpholine (12.36 g, 0.142 mol, 1.5 eq) and Et₃N (28.72 g, 0.284 mol, 3.0 eq) in CH₂Cl₂ (50 mL) was stirred at 20° C. for 12 hr under N₂ atmosphere. The reaction mixture was concentrated under reduced pressure. The residue was diluted with H₂O (50 mL) and extracted with EtOAc (50 mL×3). The combined organic layers were washed with brine (50 mL), dried over Na₂SO₄, filtered and concentrated under reduced pressure to provide 4-(5-nitro-2-pyridyl)morpholine as a yellow solid (130 g, crude), which was used directly without further purification. LCMS (ESI) m/z: 210.1 [M+H]⁺.

### Step 2: 6-morpholinopyridin-3-amine

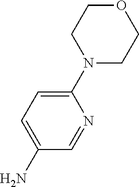 [SMILES: Nc1ccc(N2CCOCC2)nc1]

To a solution of 4-(5-nitro-2-pyridyl)morpholine (10 g, 47.8 mmol, 1.0 eq) in EtOH (100 mL) was added wet Pd/C (5.09 g, 10 wt % Pd with 50 wt % water). The mixture was degassed and purged with H₂ for three times. The mixture was stirred at 20° C. for 12 hr under H₂ atmosphere. The solid was filtered off and washed with MeOH (10 mL×2). The organic layers were combined and concentrated under reduced pressure to provide the crude product 6-morpholinopyridin-3-amine as an airy blue solid (103 g, crude).

### Step 3: 2,2-dimethyl-N-(6-morpholino-3-pyridyl)propanamide

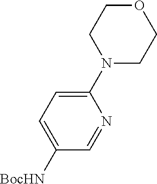 [SMILES: CC(C)(C)OC(=O)Nc1ccc(N2CCOCC2)nc1]

To a solution of 6-morpholinopyridin-3-amine (10 g, 46.4 mmol, 1.0 eq) in THF (150 mL) was added TEA (7.04 g, 69.6 mmol, 1.5 eq) and 2,2-dimethylpropanoyl chloride (8.39 g, 69.6 mmol, 1.5 eq) at 0° C. The mixture was stirred at 20° C. for 2 hr under N₂ atmosphere and concentrated under reduced pressure. The residue was diluted with H₂O (300 mL) and extracted with EtOAc (350 mL×3). The combined organic layers were washed with brine (100 mL), dried over Na₂SO₄, filtered and concentrated under reduced pressure to provide the crude product 2,2-dimethyl-N-(6-morpholino-3-pyridyl)propanamide as a white solid (20 g, 73.7%).

### Step 4: N-(4-iodo-6-morpholinopyridin-3-yl)pivalamide

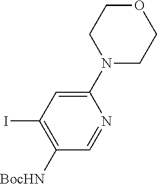 [SMILES: CC(C)(C)OC(=O)Nc1cnc(N2CCOCC2)cc1I]

A solution of 2,2-dimethyl-N-(6-morpholino-3-pyridyl)propanamide (6 g, 22.8 mmol, 1.0 eq) in THF (40 mL) was added TMEDA (7.94 g, 68.4 mmol, 3.0 eq). The mixture was stirred at −78° C. for 1 hr under N₂ atmosphere. Then n-BuLi (2.5 M, 27.3 mL) was added to the above solution and the mixture was stirred at −35° C. for 2 hr. To the mixture was added I₂ (7.52 g, 29.6 mmol, 1.3 eq) in THF (20 mL) and the mixture was stirred at 25° C. for 12 hr. The solution was quenched by sat. NH₄Cl aqueous solution (100 mL) at 0° C. and extracted with EtOAc (100 mL×3). The combined organic layers were washed with brine (100 mL), dried over Na₂SO₄, filtered and concentrated under reduced pressure. The residue was purified by flash chromatography (ISCO®; 40 g SepaFlash® Silica Flash Column, petroleum ether/EtOAc with EtOAc from 0˜30%, 100 mL/min, 254 nm), to provide N-(4-iodo-6-morpholino-3-pyridyl)-2,2-dimethyl-propanamide as a brown solid (4.3 g, 20.6%). LCMS (ESI) m/z: 389.9 [M+H]⁺.

### Step 5: 4-iodo-6-morpholinopyridin-3-amine

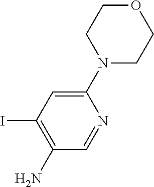 [SMILES: Nc1cnc(N2CCOCC2)cc1I]

To a solution of conc. H₂SO₄ (18.4 g, 0.188 mol) in H₂O (30 mL) was added N-(4-iodo-6-morpholino-3-pyridyl)-2,2-dimethyl-propanamide (4 g, 10.3 mmol, 1 eq) in portions. The mixture was stirred at 100° C. for 4 hr, basified with 2N NaOH aqueous solution until pH reached 8 at 0° C. and extracted with EtOAc (50 mL×3). The combined organic layers were washed with brine (50 mL), dried over Na₂SO₄, filtered and concentrated under reduced pressure. The crude product was purified by preparative HPLC (column: 2_Phenomenex Gemini C18 75×40 mm×3 m; mobile phase: [water (NH₃H₂O+NH₄HCO₃)-MeCN]; B %: 14%-45%, 9.5 min as additive) to provide 4-iodo-6-morpholino-pyridin-3-amine as a purple solid (1.2 g, 37.8%). LCMS (ESI) m/z: 305.9 [M+H]⁺.

### Step 6: tert-butyl (4-iodo-6-morpholinopyridin-3-yl)carbamate

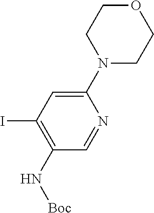 [SMILES: CC(C)(C)OC(=O)Nc1cnc(N2CCOCC2)cc1I]

A solution of 4-iodo-6-morpholino-pyridin-3-amine (1.2 g, 3.93 mmol, 1.0 eq) and Boc₂O (8.58 g, 39.3 mmol, 10.0 eq) in THF (15 mL) was stirred at 70° C. for 8 hr. The reaction mixture was concentrated under reduced pressure to remove the solvent. The residue was purified by flash silica gel chromatography (ISCO®; 12 g SepaFlash® Silica Flash Column, petroleum ether/EtOAc with EtOAc from 0˜30%, 40 mL/min, 254 nm) to provide tert-butyl N-(4-iodo-6-morpholino-3-pyridyl) carbamate as a white solid (1.42 g, 85.5%). LCMS (ESI) m/z: 406.0 [M+H]⁺.

### Step 7: tert-butyl (4-(3-methyl-4-nitro-1-((2-(trimethylsilyl)ethoxy)methyl)-1H-pyrazol-5-yl)-6-morpholinopyridin-3-yl)carbamate

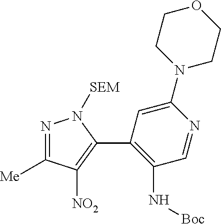 [SMILES: Cc1nn(COCC[Si](C)(C)C)c(-c2cc(N3CCOCC3)ncc2NC(=O)OC(C)(C)C)c1[N+](=O)[O-]]

A mixture of tert-butyl N-(4-iodo-6-morpholino-3-pyridyl)carbamate (1.4 g, 3.45 mmol, 1.0 eq), trimethyl-[2-[(3-methyl-4-nitro-pyrazol-1-yl)methoxy]ethyl]silane (Intermediate A1-1) (1.48 g, 3.45 mmol, 1.0 eq), Cs₂CO₃ (1.69 g, 5.18 mmol, 1.5 eq), XPhos (658.8 mg, 1.38 mmol, 0.3 eq), Pd(OAc)₂ (155.1 mg, 0.691 mmol, 0.2 eq) and cesium(I) 2,2-dimethylpropanoate (889.4 mg, 3.80 mmol, 1.1 eq) in dioxane (15 mL) was stirred at 100° C. for 16 hr under N₂ atmosphere. The reaction mixture was filtered and the filtrate was concentrated under reduced pressure to remove the solvent. The residue was purified by flash chromatography (ISCO®; 20 g SepaFlash® Silica Flash Column, petroleum ether/EtOAc with EtOAc from 0˜25%, 50 mL/min, 254 nm), to provide tert-butyl N-[4-[5-methyl-4-nitro-2-(2-trimethylsilylethoxymethyl)pyrazol-3-yl]-6-morpholino-3-pyridyl]carbamate as a brown oil (880 mg, 31.4%). LCMS (ESI) m/z: 535.2 [M+H]⁺.

### Step 8: tert-butyl (4-(4-amino-3-methyl-1-((2-(trimethylsilyl)ethoxy)methyl)-1H-pyrazol-5-yl)-6-morpholinopyridin-3-yl)carbamate

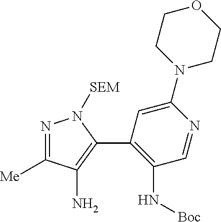 [SMILES: Cc1nn(COCC[Si](C)(C)C)c(-c2cc(N3CCOCC3)ncc2NC(=O)OC(C)(C)C)c1N]

A mixture of tert-butyl N-[4-[5-methyl-4-nitro-2-(2-trimethylsilylethoxymethyl)pyrazol-3-yl]-6-morpholino-3-pyridyl]carbamate (880 mg, 1.65 mmol, 1.0 eq) and wet Pd/C (300 mg, 10 wt % Pd with 50 wt % water) in MeOH (10 mL) was stirred at 25° C. for 16 hr under H₂ (15 psi). The reaction mixture was filtered and concentrated under reduced pressure. The residue was purified by flash chromatography (ISCO®; 12 g SepaFlash® Silica Flash Column, petroleum ether/EtOAc with EtOAc from 0˜70%, 40 mL/min, 254 nm), to provide tert-butyl N-[4-[4-amino-5-methyl-2-(2-trimethylsilylethoxymethyl)pyrazol-3-yl]-6-morpholino-3-pyridyl]carbamate as a white solid (600 mg, 71.5%). LCMS (ESI) m/z: 505.2 [M+H]⁺.

### Step 9: 4-(4-amino-3-methyl-1-((2-(trimethylsilyl)ethoxy)methyl)-1H-pyrazol-5-yl)-6-morpholinopyridin-3-amine (C2-1)

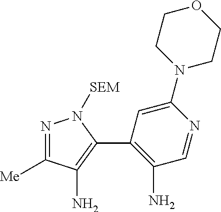 [SMILES: Cc1nn(COCC[Si](C)(C)C)c(-c2cc(N3CCOCC3)ncc2N)c1N]

A mixture of tert-butyl N-[4-[4-amino-5-methyl-2-(2-trimethylsilylethoxymethyl) pyrazol-3-yl]-6-morpholino-3-pyridyl]carbamate (600 mg, 1.19 mmol, 1.0 eq) and ZnBr₂ (1.61 g, 7.13 mmol, 6.0 eq) in DCM (10 mL) was stirred at 40° C. for 16 hr, the diluted with H₂O (10 mL) and extracted with EtOAc (10 mL×3). The combined organic layers were washed with brine (20 mL), dried with anhydrous Na₂SO₄, filtered and concentrated under reduced pressure to provide the tittle compound as a yellow solid (550 mg, 83.4%). ¹H NMR (400 MHz, DMSO-d₆) δ ppm 0.04 (m, 9H), 0.74 (t, J=8.16 Hz, 2H), 2.10 (s, 3H), 3.22 (br d, J=2.76 Hz, 4H), 3.41 (br s, 2H), 3.67-3.73 (m, 6H), 4.67 (br s, 2H), 5.12 (br s, 2H), 6.63-6.75 (m, 1H), 7.85 (s, 1H); LCMS (ESI) m/z: 405.1 [M+H]⁺.

### Synthesis of 3-[4-amino-5-chloro-2-(2-trimethylsilylethoxymethyl)pyrazol-3-yl]-5-morpholino-pyridin-2-amine (Intermediate C2-3)

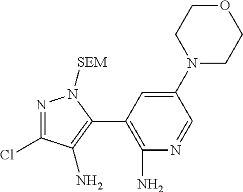 [SMILES: C[Si](C)(C)CCOCn1nc(Cl)c(N)c1-c1cc(N2CCOCC2)cnc1N]

### Step 1: ethyl N-(5-bromo-3-pyridyl)carbamate

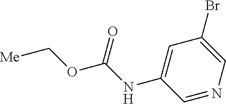 [SMILES: CCOC(=O)Nc1cncc(Br)c1]

To a solution of 5-bromopyridin-3-amine (30 g, 0.173 mmol, 1 eq) in DCM (400 mL) was added pyridine (41.15 g, 0.520 mol, 3 eq) and ethyl carbonochloridate (18.82 g, 0.173 mmol, 1 eq). The mixture was stirred at 25° C. for 2 hr, diluted with DCM (300 mL, quenched by H₂O (300 mL) at 0° C. and extracted with DCM (300 mL×3). Then the combined organic layers were washed with 10 wt % CuSO₄ in H₂O (300 mL), saturated NaHCO₃ aqueous solution (300 mL) and brine (300 mL), dried over anhydrous Na₂SO₄, filtered and concentrated under reduced pressure. The residue was purified by flash chromatography (ISCO®; 120 g SepaFlash® Silica Flash Column, petroleum ether/EtOAc with EtOAc from 0˜50%, 100 mL/min, 254 mn) to afford ethyl N-(5-bromo-3-pyridyl)carbamate (29 g, 68.2%) as a white solid. ¹H NMR (400 MHz, CDCl₃) δ ppm 1.33 (t, J=7.1 Hz, 3H), 4.26 (q, J=7.1 Hz, 2H), 7.48 (br s, 1H), 8.27-8.44 (m, 3H); LCMS (ESI) m/z: 246.7 [M+H]⁺.

### Step 2: ethyl N-(5-bromo-2-nitro-3-pyridyl)carbamate

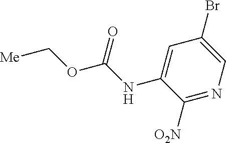 [SMILES: CCOC(=O)Nc1cc(Br)cnc1[N+](=O)[O-]]

To a solution of ethyl N-(5-bromo-3-pyridyl)carbamate (6 g, 24.4 mmol, 1 eq) in H₂SO₄ (18 mL) at 0° C. was added HNO₃ (15.43 g, 0.245 mol, 10 eq) dropwise. After the addition of nitric acid, the reaction mixture was gradually warmed up to 0° C. for 30 min, then stirred at 25° C. for 15.5 hr. The reaction mixture was poured on ice water (500 g), then quenched by 3N NaOH in H₂O to adjust pH to 7. The aqueous phase was extracted with DCM (100 mL×3) to give ethyl N-(5-bromo-2-nitro-3-pyridyl)carbamate (25 g, 87.1%) as a white solid. ¹H NMR (400 MHz, DMSO-d₆) δ ppm 1.21-1.26 (m, 3H), 4.12-4.18 (m, 2H), 8.40-8.49 (m, 2H), 10.10 (s, 1H); LCMS (ESI) m/z: 289.9 [M+H]⁺.

### Step 3: 5-bromo-2-nitro-pyridin-3-amine

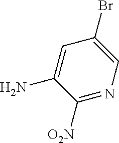 [SMILES: Nc1cc(Br)cnc1[N+](=O)[O-]]

To a solution of ethyl N-(5-bromo-2-nitro-3-pyridyl)carbamate (12.5 g, 43.0 mmol, 1 eq) in EtOH (15 mL) was added KOH (6.47 g, 0.115 mol, 2.6 eq) in H₂O (75 mL). The mixture was stirred at 90° C. for 16 hr. H₂O (200 mL) was added to the mixture and the precipitate was formed and collected by filtration, washed with water, and then dried under reduced pressure to give 5-bromo-2-nitro-pyridin-3-amine (15 g, 76.6%) as a green solid. ¹H NMR (400 MHz, DMSO-d₆) δ ppm 7.45 (br s, 2H), 7.76 (d, J=2.0 Hz, 1H), 7.84 (d, J=2.0 Hz, 1H); LCMS (ESI) m/z: 219.6 [M+H]⁺.

### Step 4: 5-morpholino-2-nitro-pyridin-3-amine

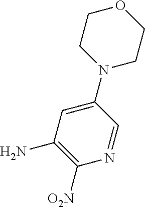 [SMILES: Nc1cc(N2CCOCC2)cnc1[N+](=O)[O-]]

A solution of 5-bromo-2-nitro-pyridin-3-amine (3 g, 13.7 mmol, 1 eq) in morpholine (6.4 g, 73.4 mmol, 5.3 eq) was stirred at 140° C. for 3 hr. The solution was added into H₂O (20 mL), then filtered to collect the solid. The residue was triturated with EtOH (10 ml) to give 5-morpholino-2-nitro-pyridin-3-amine (3 g, 95.2%) as a yellow solid. LCMS (ESI) m/z: 224.9 [M+H]⁺.

### Step 5: 4-(5-iodo-6-nitro-3-pyridyl)morpholine

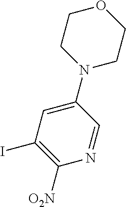 [SMILES: O=[N+]([O-])c1ncc(N2CCOCC2)cc1I]

To a solution of CuI (5.10 g, 26.7 mmol, 2 eq) in MeCN (20 mL) was added dropwise isopentyl nitrite (7.84 g, 66.9 mmol, 5 eq) at 50° C. The reaction mixture stirred at 80° C. for 1 hr, then a solution of 5-morpholino-2-nitro-pyridin-3-amine (3 g, 13.3 mmol, 1 eq) in MeCN (10 mL) was added in portions (evolution of nitrogen gas was observed). The reaction mixture stirred at 80° C. for 15 hr. The mixture was filtered and the filtrate was concentrated under reduced pressure. The residue was purified by flash chromatography (ISCO®; 20 g SepaFlash® Silica Flash Column, petroleum ether/EtOAc with EtOAc from 0˜30%, 50 mL/min, 254 nm) to give 4-(5-iodo-6-nitro-3-pyridyl)morpholine (1.3 g, 27.2%) as a yellow solid. LCMS (ESI) m/z: 335.8 [M+H]⁺.

### Step 6: 3-iodo-5-morpholino-pyridin-2-amine

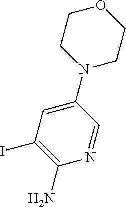 [SMILES: Nc1ncc(N2CCOCC2)cc1I]

To a solution of 4-(5-iodo-6-nitro-3-pyridyl)morpholine (1.3 g, 3.88 mmol, 1 eq) in EtOH (90 mL) under N₂ atmosphere. Tin(II) chloride (5.88 g, 31.0 mmol, 8 eq) and H₂O (1 mL) were added successively. The mixture was degassed and purged with N₂ for three times. The mixture was stirred at 80° C. for 16 hr under N₂ atmosphere. The reaction mixture was concentrated under reduced pressure. The mixture was basified at 0° C. with 2N NaOH in H₂O to adjust pH to 11 and extracted with DCM (50 mL×3). Then the combined organic layers were washed with brine (50 mL), dried by Na₂SO₄, filtered and concentrated under reduced pressure. The residue was purified by flash chromatography (ISCO®; 20 g SepaFlash® Silica Flash Column, petroleum ether/EtOAc with EtOAc from 0˜50%, 50 mL/min, 254 mn) to afford 3-iodo-5-morpholino-pyridin-2-amine (650 mg, 54.9%) as a gray solid. LCMS (ESI) m/z: 305.8 [M+H]⁺.

### Step 7: 3-[5-chloro-4-nitro-2-(2-trimethylsilylethoxymethyl)pyrazol-3-yl]-5-morpholino-pyridin-2-amine

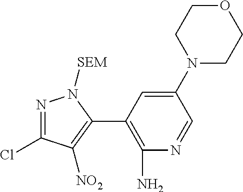 [SMILES: C[Si](C)(C)CCOCn1nc(Cl)c([N+](=O)[O-])c1-c1cc(N2CCOCC2)cnc1N]

To a solution of 3-iodo-5-morpholino-pyridin-2-amine (650 mg, 2.13 mmol, 1 eq) in DMF (8 mL) was added CuI (81.14 mg, 0.426 mmol, 0.2 eq), Pd(PPh₃)₄ (246.17 mg, 0.213 mmol, 0.1 eq), K₂CO₃ (883.3 mg, 6.39 mmol, 3 eq) and 2-[(3-chloro-4-nitro-pyrazol-1-yl)methoxy]ethyl-trimethyl-silane (Intermediate A1-1) (710.1 mg, 2.56 mmol, 1.2 eq). The mixture were degassed and purged with N₂ for three times. The mixture was stirred at 90° C. for 16 hr under N₂ atmosphere. The residue was diluted with H₂O (10 mL) and extracted with EtOAc (15 mL×3). The combined organic layers were washed with brine (10 mL), dried over Na₂SO₄, filtered and concentrated under reduced pressure. The residue was purified by flash chromatography (ISCO®; 12 g SepaFlash® Silica Flash Column, petroleum ether/EtOAc with EtOAc from 0˜50%, 20 mL/min, 254 mn) to afford 3-[5-chloro-4-nitro-2-(2-trimethylsilylethoxymethyl)pyrazol-3-yl]-5-morpholino-pyridin-2-amine (990 mg, 61.2%) as a yellow oil. LCMS (ESI) m/z: 455.1 [M+H]⁺.

### Step 8: 3-[4-amino-5-chloro-2-(2-trimethylsilylethoxymethyl)pyrazol-3-yl]-5-morpholino-pyridin-2-amine (C2-3)

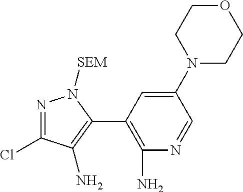 [SMILES: C[Si](C)(C)CCOCn1nc(Cl)c(N)c1-c1cc(N2CCOCC2)cnc1N]

To a solution of 3-[5-chloro-4-nitro-2-(2-trimethylsilylethoxymethyl)pyrazol-3-yl]-5-morpholino-pyridin-2-amine (990 mg, 1.31 mmol, 1 eq) in THF (5 mL) was added NaBH₄ (197.5 mg, 5.22 mmol, 4 eq), NiCl₂·6H₂O (1.24 g, 5.22 mmol, 4 eq) at 0° C. The mixture was degassed and purged with N₂ for three times. The mixture was stirred at 0° C. for 1 hr under N₂ atmosphere. The solution was quenched by addition of ammonium chloride (10 mL) at 0° C. and extracted with EtOAc (10 mL×3). Then the combined organic layers were washed with brine (10 mL), dried by Na₂SO₄, filtered and concentrated under reduced pressure. The residue was purified by flash chromatography (ISCO®; 12 g SepaFlash® Silica Flash Column, petroleum ether/EtOAc with EtOAc from 0˜50%, 20 mL/min, 254 mn) to afford 3-[4-amino-5-chloro-2-(2-trimethylsilylethoxymethyl)pyrazol-3-yl]-5-morpholino-pyridin-2-amine (130 mg, 16.4%) as a black solid. ¹H NMR (400 MHz, DMSO-d₆) δ ppm −0.08 (s, 9H), 0.70-0.75 (m, 2H), 2.98 (br s, 4H), 3.38 (br t, J=8.16 Hz, 2H), 3.68-3.72 (m, 4H), 4.16 (s, 2H), 4.98-5.24 (m, 2H), 6.11 (s, 2H), 7.65 (br d, J=4.02 Hz, 2H); LCMS (ESI) m/z: 425.0 [M+H]⁺.

### Synthesis of 9-bromo-5-(2,6-difluorophenyl)-1-((2-(trimethylsilyl)ethoxy)methyl)-1,6-dihydrobenzo[d]pyrazolo[3,4-f][1,3]diazepine (Intermediate D1-1)

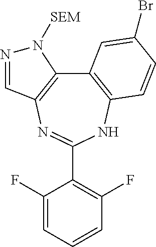 [SMILES: C[Si](C)(C)CCOCn1ncc2c1-c1cc(Br)ccc1NC(c1c(F)cccc1F)=N2]

### Step 1: 9-bromo-5-(2,6-difluorophenyl)-1-((2-(trimethylsilyl)ethoxy)methyl)-1,4,5,6-tetrahydrobenzo[d]pyrazolo[3,4-f][1,3]diazepine

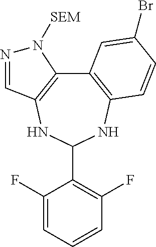 [SMILES: C[Si](C)(C)CCOCn1ncc2c1-c1cc(Br)ccc1NC(c1c(F)cccc1F)N2]

To a solution of 5-(2-amino-5-bromo-phenyl)-1-(2-trimethylsilylethoxymethyl)pyrazol-4-amine (Intermediate B1-10) (6.30 g, 16.4 mmol, 1 eq.) in ^{t}BuOH (60 mL) was added 2,6-difluorobenzaldehyde (2.34 g, 16.4 mmol, 1 eq.), Y(OTf)₃ (440 mg, 0.82 mmol, 0.05 eq.) and K₂CO₃ (6.81 g, 49.3 mmol, 3 eq.). The mixture was stirred at 70° C. for 30 min. The crude 2-[[9-bromo-5-(2,6-difluorophenyl)-5,6-dihydro-4H-pyrazolo[4,3-d][1,3]benzodiazepin-1-yl]methoxy]ethyl-trimethyl-silane (8.3 g, crude) was obtained as a yellow oil, which was used for the next step without further purification.

### Step 2: 9-bromo-5-(2,6-difluorophenyl)-1-((2-(trimethylsilyl)ethoxy)methyl)-1,6-dihydrobenzo[d]pyrazolo[3,4-f][1,3]diazepine (D1-1)

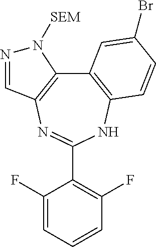 [SMILES: C[Si](C)(C)CCOCn1ncc2c1-c1cc(Br)ccc1NC(c1c(F)cccc1F)=N2]

To a solution of 2-[[9-bromo-5-(2,6-difluorophenyl)-5,6-dihydro-4H-pyrazolo[4,3-d][1,3]benzodiazepin-1-yl]methoxy]ethyl-trimethyl-silane (6.3 g, 12.4 mmol, 1 eq.) in ^{t}BuOH (60 mL) was added DDQ (3.38 g, 14.9 mmol, 1.2 eq.) and the mixture was stirred at 70° C. for 30 min. The reaction mixture was concentrated under reduced pressure. The residue was diluted with H₂O (100 mL) and extracted with EtOAc (150 mL×3). The combined organic layers were washed with brine (150 mL), dried over anhydrous Na₂SO₄, filtered and concentrated under reduced pressure. The residue was purified by flash chromatography on silica gel column using 0-21% of EtOAc in petroleum ether as eluent, to provide compound 2-[[9-bromo-5-(2,6-difluorophenyl)-6H-pyrazolo[4,3-d][1,3]benzodiazepin-1-yl]methoxy]ethyl-trimethyl-silane as a red solid (4.05 g, 64.5%). ¹H NMR (400 MHz, DMSO-d₆) δ ppm −0.01 (s, 9H), 0.80-1.01 (m, 2H), 3.72 (t, J=8.0 Hz, 2H), 5.40 (s, 2H), 6.73 (d, J=8.5 Hz, 1H), 7.19 (br t, J=8.2 Hz, 2H), 7.31 (s, 1H), 7.44 (dd, J=8.5, 2.3 Hz, 1H), 7.49-7.61 (m, 1H), 7.74 (d, J=2.3 Hz, 1H), 8.40 (s, 1H).

### Synthesis of 9-bromo-3-chloro-5-(2,6-difluorophenyl)-1-((2-(trimethylsilyl)ethoxy)methyl)-1,6-dihydrobenzo[d]pyrazolo[3,4-f][1,3]diazepine (Intermediate D1-2)

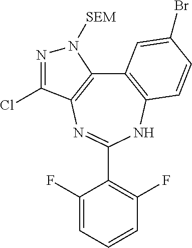 [SMILES: C[Si](C)(C)CCOCn1nc(Cl)c2c1-c1cc(Br)ccc1NC(c1c(F)cccc1F)=N2]

A mixture of 5-(2-amino-5-bromo-phenyl)-3-chloro-1-(2-trimethylsilylethoxymethyl) pyrazol-4-amine (Intermediate B1-2) (4.8 g, 11.5 mmol, 1 eq), 2,6-difluorobenzaldehyde (1.63 g, 11.5 mmol, 1 eq) in t-BuOH (24 mL) and THF (24 mL) was degassed and purged with N₂ for 3 times, and then the mixture was stirred at 25° C. for 16 hr under N₂ atmosphere. Then the mixture was added DDQ (2.76 g, 12.2 mmol, 1.1 eq) and purged with N₂ for 3 times, and then the mixture was stirred at 25° C. for 1 h under N₂ atmosphere. The residue was diluted with NaHCO₃/H₂O (50 mL) and extracted with EtOAc (50 mL×3). The combined organic layers were washed with Brine 50 mL, dried over Na₂SO₄, filtered and concentrated under reduced pressure. The residue was purified by flash silica gel chromatography (ISCO®; 40 g SepaFlash® Silica Flash Column, Eluent of 0˜30% petroleum ether/EtOAc @ 35 mL/min) to give 2-[[9-bromo-3-chloro-5-(2,6-difluorophenyl)-6H-pyrazolo[4,3-d][1,3]benzodiazepin-1-yl]methoxy]ethyl-trimethyl-silane (5 g, 76.9%) as a yellow solid. LCMS (ESI) m/z: 541.2 [M+H]⁺.

### Synthesis of 9-bromo-5-(2,6-difluorophenyl)-3-methyl-1-((2-(trimethylsilyl)ethoxy)methyl)-1,6-dihydrobenzo[d]pyrazolo[3,4-f][1,3]diazepine (Intermediate D1-3)

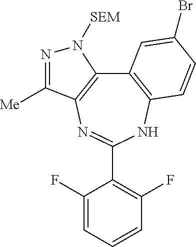 [SMILES: Cc1nn(COCC[Si](C)(C)C)c2c1N=C(c1c(F)cccc1F)Nc1ccc(Br)cc1-2]

To a solution of 5-(2-amino-5-bromo-phenyl)-3-methyl-1-(2-trimethylsilylethoxymethyl) pyrazol-4-amine (Intermediate B1-1) (880 mg, 2.21 mmol, 1 eq) and 2,6-difluorobenzaldehyde (630 mg, 4.43 mmol, 2 eq) in ^{t}BuOH (20 mL) and THF (4 mL) was added K₂CO₃ (1.22 g, 8.86 mmol, 4 eq) at 25° C. for 2 hr, then I₂ (1.12 g, 4.43 mmol, 2 eq) was added. The resulting mixture was stirred at 25° C. for 1 hr under N₂ atmosphere. The reaction mixture was quenched by addition H₂O (50 mL) at 25° C., and then extracted with EtOAc (50 mL×3). The combined organic layers were washed with brine (50 mL×3), dried over anhydrous Na₂SO₄, filtered and concentrated under reduced pressure. The residue was purified by flash chromatography (ISCO®; 40 g SepaFlash® Silica Flash Column, petroleum ether/EtOAc with EtOAc from 0˜10%, flow rate: 40 mL/min, 254 nm) to afford 2-[[9-bromo-5-(2,6-difluorophenyl)-3-methyl-6H-pyrazolo[4,3-d][1,3]benzodiazepin-1-yl]methoxy]ethyl-trimethyl-silane as brown solid (670 mg, 58.2%). ¹H NMR (400 MHz, CDCl₃) δ ppm 0.02 (s, 9H), 0.97-1.04 (m, 2H), 2.25 (s, 3H), 3.77-3.84 (m, 2H), 5.38 (s, 2H), 6.52 (d, J=8.3 Hz, 1H), 6.98 (t, J=8.2 Hz, 2H), 7.32-7.43 (m, 2H), 7.94 (d, J=2.3 Hz, 1H), ¹⁹F NMR (377 MHz, CDCl₃) δ ppm −111.51; LCMS (ESI) m/z: 521.2 [M+H]⁺.

### Synthesis of 2-[[9-bromo-5-(2-chloro-6-fluoro-phenyl)-3-methyl-6H-pyrazolo[4,3-d][1,3]benzodiazepin-1-yl]methoxy]ethyl-trimethyl-silane (Intermediate D1-4)

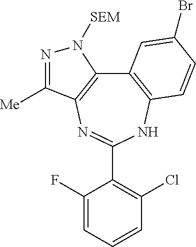 [SMILES: Cc1nn(COCC[Si](C)(C)C)c2c1N=C(c1c(F)cccc1Cl)Nc1ccc(Br)cc1-2]

To a solution of 5-(2-amino-5-bromo-phenyl)-3-methyl-1-(2-trimethylsilylethoxy methyl)pyrazol-4-amine (Intermediate B1-1) (500 mg, 1.26 mmol) and 2-chloro-6-fluoro-benzaldehyde (400 mg, 2.52 mmol) in t-BuOH (15 mL) and THF (3 mL) was added K₂CO₃ (696 mg, 5.04 mmol) at 25° C. for 2 hr, then I₂ (640 mg, 2.52 mmol) was added. The resulting mixture was stirred at 25° C. for 1 hr under N₂ atmosphere. The reaction mixture was quenched by addition H₂O (50 mL) at 25° C., and then extracted with EtOAc (100 mL×3). The combined organic layers were washed with brine (100 mL×3), dried over anhydrous Na₂SO₄, filtered and concentrated under reduced pressure. The residue was purified by flash chromatography (ISCO®; 40 g SepaFlash® Silica Flash Column, petroleum ether/EtOAc with EtOAc from 0˜20%. flow rate: 40 mL/min, 254 nm) to afford 2-[[9-bromo-5-(2-chloro-6-fluoro-phenyl)-3-methyl-6H-pyrazolo[4,3-d][1,3]benzodiazepin-1-yl]methoxy]ethyl-trimethyl-silane (330 mg, 48.9%) as yellow solid. LCMS (ESI) m/z: 537.2 [M+H]⁺.

### Synthesis of 2-[[9-bromo-5-(2,6-difluoro-4-methyl-phenyl)-3-methyl-6H-pyrazolo[4,3-d][1,3]benzodiazepin-1-yl]methoxy]ethyltrimethyl-silane (Intermediate D1-5)

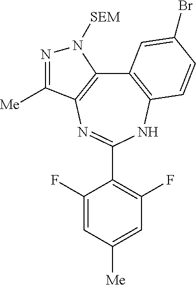 [SMILES: Cc1cc(F)c(C2=Nc3c(C)nn(COCC[Si](C)(C)C)c3-c3cc(Br)ccc3N2)c(F)c1]

To a solution of 5-(2-amino-5-bromo-phenyl)-3-methyl-1-(2-trimethylsilylethoxymethyl) pyrazol-4-amine (Intermediate B1-1) (400 mg, 1.01 mmol, 1 eq), 2,6-difluoro-4-methyl-benzaldehyde (320 mg, 2.05 mmol, 2.04 eq) in t-BuOH (10 mL) and THF (2 mL) was added K₂CO₃ (560 mg, 4.05 mmol, 4.03 eq) at 25° C. for 3 hr, then I₂ (510 mg, 2.01 mmol, 2 eq) was added. The resulting mixture was stirred at 25° C. for 1 hr under N₂ atmosphere. The reaction mixture was quenched by addition H₂O (50 mL) at 25° C., and then extracted with EtOAc (100 mL×3). The combined organic layers were washed with brine (100 mL×3), dried over anhydrous Na₂SO₄, filtered and concentrated under reduced pressure. The residue was purified by flash chromatography (ISCO®; 40 g SepaFlash® Silica Flash Column, petroleum ether/EtOAc with EtOAc from 0˜5%, Flow Rate: 40 mL/min, 254 nm) to afford 2-[[9-bromo-5-(2,6-difluoro-4-methyl-phenyl)-3-methyl-6H-pyrazolo[4,3-d][1,3]benzodiazepin-1-yl]methoxy]ethyltrimethyl-silane (310 mg, 57.7%) as a yellow solid. LCMS (ESI) m/z: 534.1 [M+H]⁺.

### Synthesis of 9-chloro-5-(2,6-difluorophenyl)-1-((2-(trimethylsilyl)ethoxy)methyl)-1,6-dihydropyrazolo[4,3-d]pyrido[4,3-f][1,3]diazepine (Intermediate D2-1)

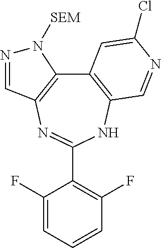 [SMILES: C[Si](C)(C)CCOCn1ncc2c1-c1cc(Cl)ncc1NC(c1c(F)cccc1F)=N2]

To a solution of 4-[4-amino-2-(2-trimethylsilylethoxymethyl)pyrazol-3-yl]-6-chloro-pyridin-3-amine (Intermediate B2-1) (5 g, 14.7 mmol, 1 eq) in ^{t}BuOH (40 mL) and THF (20 mL) was added K₂CO₃ (6.10 g, 44.1 mmol, 3 eq), yttrium(III) trifluoromethanesulfonate (394.3 mg, 0.735 mmol, 0.05 eq) and 2,6-difluorobenzaldehyde (2.09 g, 14.7 mmol, 1 eq). The mixture was stirred at 50° C. for 16 h. The reaction mixture was added filtered. DDQ (3.33 g, 14.7 mmol, 1 eq) was added into the solution. The mixture was stirred at 25° C. for 3 hr. The mixture was diluted with EtOAc (300 mL) and washed with NaHCO₃/H₂O (100 mL). The combined organic layers were washed with brine (100 mL), dried with anhydrous Na₂SO₄, filtered and concentrated under reduced pressure. The residue was purified by flash silica gel chromatography (ISCO®; 80 g SepaFlash® Silica Flash Column, petroleum ether/EtOAc with EtOAc from 0˜30%, 60 mL/min, 254 nm) to provide 2-[[13-chloro-8-(2,6-difluorophenyl)-3,4,7,9,12-pentazatricyclo[8.4.0.02,6]tetradeca-1(10),2(6),4,7,11,13-hexaen-3-yl]methoxy] ethyl-trimethyl-silane as a yellow solid (5.6 g, 82.7%). ¹H NMR (400 MHz, DMSO-d₆) δ ppm 0.03 (s, 9H), 0.90 (t, J=7.91 Hz, 2H), 3.69 (t, J=7.91 Hz, 2H), 5.50 (s, 2H), 7.22 (br t, J=8.03 Hz, 2H), 7.37 (br s, 1H), 7.49-7.65 (m, 2H), 7.73 (br s, 1H), 8.70 (br s, 1H); ¹⁹F NMR (377 MHz, CD₃OD) δ ppm −113.44; LCMS (ESI) m/z: 462.1 [M+H]⁺.

### Synthesis of 9-bromo-3-chloro-5-(2,6-difluorophenyl)-1-((2-(trimethylsilyl)ethoxy)methyl)-1,6-dihydropyrazolo[4,3-d]pyrido[4,3-f][1,3]diazepine (Intermediate D2-2)

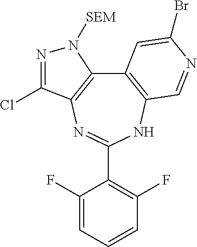 [SMILES: C[Si](C)(C)CCOCn1nc(Cl)c2c1-c1cc(Br)ncc1NC(c1c(F)cccc1F)=N2]

To a solution of 4-[4-amino-5-chloro-2-(2-trimethylsilylethoxymethyl)pyrazol-3-yl]-6-bromo-pyridin-3-amine (Intermediate B2-3) (500 mg, 1.19 mmol, 1 eq) in ^{t}BuOH (5 mL) and THF (1 mL) was added 2,6-difluorobenzaldehyde (169.7 mg, 1.19 mmol, 1 eq). The mixture was stirred at 25° C. for 16 hr under N₂ atmosphere. Then MnO₂ (518.9 mg, 5.97 mmol, 5 eq) was added and the mixture was stirred at 25° C. for 5 hr under N₂ atmosphere. The mixture was filtered and the filtrate was concentrated under reduced pressure. The residue was purified by flash chromatography (ISCO®; 20 g SepaFlash® Silica Flash Column, petroleum ether/EtOAc with EtOAc from 0˜30%, 100 mL/min, 254 mn) to give 2-[[13-bromo-5-chloro-8-(2,6-difluorophenyl)-3,4,7,9,12-pentazatricyclo[8.4.0.0^{2,6}]tetradeca-1(10),2(6),4,7,11,13-hexaen-3-yl]methoxy]ethyl-trimethyl-silane as a yellow solid (250 mg, 32.9%). ¹H NMR (400 MHz, CDCl₃) δ ppm 0.04 (s, 9H), 0.95-1.04 (m, 2H), 3.78-3.85 (m, 2H), 5.43 (s, 2H), 5.77 (s, 1H), 7.02 (t, J=8.19 Hz, 2H), 7.37-7.48 (m, 1H), 7.70 (s, 1H), 7.94 (s, 1H); LCMS (ESI) m/z: 542.0 [M+H]⁺.

### Synthesis of 2-[[13-chloro-8-(2,6-difluorophenyl)-5-methyl-3,4,7,9,12-pentazatricyclo[8.4.0.02,6]tetradeca-1(10),2(6),4,7,11,13-hexaen-3-yl]methoxy]ethyl-trimethyl-silane (Intermediate D2-3)

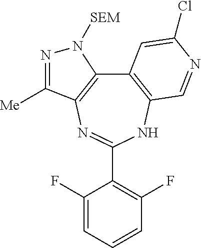 [SMILES: Cc1nn(COCC[Si](C)(C)C)c2c1N=C(c1c(F)cccc1F)Nc1cnc(Cl)cc1-2]

To a solution of 4-[4-amino-5-methyl-2-(2-trimethylsilylethoxymethyl)pyrazol-3-yl]-6-chloro-pyridin-3-amine (Intermediate B2-2) (3.3 g, 9.32 mmol, 1 eq), 2,6-difluorobenzaldehyde (1.99 g, 13.9 mmol, 1.5 eq), K₂CO₃ (3.87 g, 28.0 mmol, 3 eq), I₂ (3.55 g, 13.9 mmol, 1.5 eq) in ^{t}BuOH (50 mL) was degassed and purged with N₂ for 3 times. The mixture was stirred at 60° C. for 1 hr under N₂ atmosphere, filtered and the filtrate was concentrated under reduced pressure. The residue was purified by flash chromatography (ISCO®; 80 g SepaFlash® Silica Flash Column, petroleum ether/EtOAc with EtOAc from 0˜50%, 20 mL/min, 254 mn) to give 2-[[13-chloro-8-(2,6-difluorophenyl)-5-methyl-3,4,7,9,12-pentazatricyclo[8.4.0.02,6]tetradeca-1(10),2(6),4,7,11,13-hexaen-3-yl]methoxy]ethyl-trimethyl-silane (2 g, 45.1%) as a yellow solid. ¹H NMR (400 MHz, DMSO-d₆) δ ppm −0.05-0.00 (m, 9H), 0.83-0.95 (m, 2H), 2.51-2.53 (m, 3H), 3.69 (t, J=7.8 Hz, 2H), 5.43 (s, 2H), 7.23 (t, J=8.1 Hz, 2H), 7.51-7.64 (m, 2H), 7.73 (s, 1H); LCMS (ESI) m/z: 476.1 [M+H]⁺.

### Synthesis of 2-[[13-chloro-8-(2,6-difluorophenyl)-5-methyl-3,4,7,9,12-pentazatricyclo[8.4.0.02,6]tetradeca-1(10),2(6),4,7,11,13-hexaen-3-yl]methoxy]ethyl-trimethyl-silane (Intermediate D2-4)

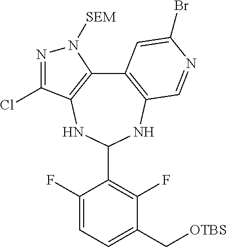 [SMILES: CC(C)(C)[Si](C)(C)OCc1ccc(F)c(C2Nc3cnc(Br)cc3-c3c(c(Cl)nn3COCC[Si](C)(C)C)N2)c1F]

### Step 1: tert-butyl-[(2,4-difluorophenyl)methoxy]-dimethyl-silane

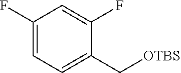 [SMILES: CC(C)(C)[Si](C)(C)OCc1ccc(F)cc1F]

To a solution of (2,4-difluorophenyl)methanol (5 g, 34.7 mmol, 1 eq) in DMF (50 mL) was added TBSCl (6.27 g, 41.6 mmol, 1.2 eq) and imidazole (5.90 g, 86.7 mmol, 2.5 eq). The mixture was stirred at 25° C. for 12 hr. The reaction mixture was diluted with H₂O (50 mL) and extracted with EtOAc (100 mL×3). The combined organic layers were washed with brine (100 mL), dried over Na₂SO₄, filtered and concentrated under reduced pressure. The residue was purified by flash chromatography (ISCO®; 12 g SepaFlash® Silica Flash Column, petroleum ether/EtOAc with EtOAc from 0˜5%, 30 mL/min, 254 mn) to afford tert-butyl-[(2,4-difluorophenyl)methoxy]-dimethyl-silane (7.8 g, 87.0%) as a colorless oil.

### Step 2: tert-butyl-[(2,4-difluorophenyl)methoxy]-dimethyl-silane

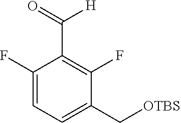 [SMILES: CC(C)(C)[Si](C)(C)OCc1ccc(F)c(C=O)c1F]

To a solution of tert-butyl-[(2,4-difluorophenyl)methoxy]-dimethyl-silane (4 g, 15.5 mmol, 1 eq) in THF (40 mL) at −30° C. was dropwise added 2M LDA in THF (9.3 mL, 18.6 mmol, 1.2 eq) and the mixture was stirred at −30° C. for 30 min. Then DMF (1.36 g, 18.6 mmol, 1.2 eq) in THF (5 mL) was dropwise added to the above solution at 0° C. and the mixture was stirred at −30° C. for 30 min. The solution was quenched by addition of ammonium chloride at 0° C. and extracted with EtOAc (40 mL×3). Then the combined organic layers were washed with brine (30 mL), dried by Na₂SO₄, filtered and concentrated under reduced pressure to afford 3-[[tert-butyl(dimethyl)silyl]oxymethyl]-2,6-difluoro-benzaldehyde (4.01 g, 90.5%) as a yellow oil.

### Step 3: 9-bromo-5-(3-(((tert-butyldimethylsilyl)oxy)methyl)-2,6-difluorophenyl)-3-chloro-1-((2-(trimethylsilyl)ethoxy)methyl)-1,4,5,6-tetrahydropyrazolo[4,3-d]pyrido[4,3-f][1,3]diazepine

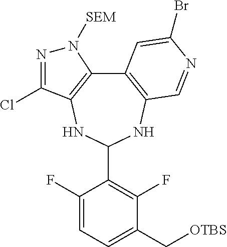 [SMILES: CC(C)(C)[Si](C)(C)OCc1ccc(F)c(C2Nc3cnc(Br)cc3-c3c(c(Cl)nn3COCC[Si](C)(C)C)N2)c1F]

To a solution of 3-[[tert-butyl(dimethyl)silyl]oxymethyl]-2,6-difluoro-benzaldehyde (410.3 mg, 1.43 mmol) in MeOH (8 mL) was added 4-[4-amino-5-chloro-2-(2-trimethylsilyl ethoxymethyl)pyrazol-3-yl]-6-bromo-pyridin-3-amine (Intermediate B2-3) (500 mg, 1.19 mmol) and the mixture was stirred at 40° C. for 30 min, filtered and concentrated under reduced pressure. The residue was triturated with petroleum ether (100 mL) to give 2-[[13-bromo-8-[3-[[tert-butyl(dimethyl)silyl]oxymethyl]-2,6-difluoro-phenyl]-5-chloro-3,4,7,9,12-pentazatricyclo[8.4.0.0^{2,6}]tetradeca-1(10),2(6),4,11,13-pentaen-3-yl]methoxy]ethyl-trimethyl-silane (730 mg, 80.1%) as an off-white solid.

### Step 4: 9-bromo-5-(3-(((tert-butyldimethylsilyl)oxy)methyl)-2,6-difluorophenyl)-3-chloro-1-((2-(trimethylsilyl)ethoxy)methyl)-1,6-dihydropyrazolo[4,3-d]pyrido[4,3-f][1,3]diazepine (D2-4)

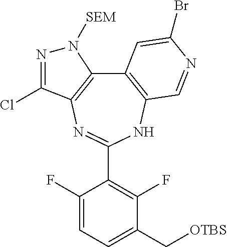 [SMILES: CC(C)(C)[Si](C)(C)OCc1ccc(F)c(C2=Nc3c(Cl)nn(COCC[Si](C)(C)C)c3-c3cc(Br)ncc3N2)c1F]

To a solution of 2-[[13-bromo-8-[3-[[tert-butyl(dimethyl)silyl]oxymethyl]-2,6-difluoro-phenyl]-5-chloro-3,4,7,9,12-pentazatricyclo[8.4.0.0^{2,6}]tetradeca-1(10),2(6),4,11,13-pentaen-3-yl]methoxy]ethyl-trimethyl-silane (730 mg, 1.06 mmol) in DCM (10 mL) was added DDQ (289.4 mg, 1.27 mmol) and the mixture was stirred at 25° C. for 24 hr and then triturated with petroleum ether (20 mL) to give 2-[[13-bromo-8-[3-[[tert-butyl(dimethyl)silyl]oxymethyl]-2,6-difluoro-phenyl]-5-chloro-3,4,7,9,12-pentazatricyclo[8.4.0.0^{2,6}]tetradeca-1(10),2(6),4,7,11,13-hexaen-3-yl]methoxy]ethyl-trimethyl-silane (520 mg, 71.4%) as a yellow solid.

### Synthesis of 2-[[13-bromo-8-[4-[[tert-butyl(dimethyl)silyl]oxymethyl]-2,6-difluoro-phenyl]-5-chloro-3,4,7,9,12-petroleum ethertroleum etherntazatricyclo[8.4.0.02,6]tetradeca-1(10),2(6),4,7,11,13-hexaen-3-yl]methoxy]ethyl-trimethyl-silane (Intermediate D2-5)

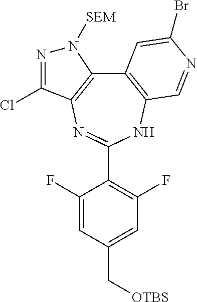 [SMILES: CC(C)(C)[Si](C)(C)OCc1cc(F)c(C2=Nc3c(Cl)nn(COCC[Si](C)(C)C)c3-c3cc(Br)ncc3N2)c(F)c1]

### Step 1: tert-butyl-[(3,5-difluorophenyl)methoxy]-dimethyl-silane

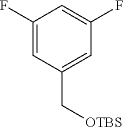 [SMILES: CC(C)(C)[Si](C)(C)OCc1cc(F)cc(F)c1]

A mixture of (3,5-difluorophenyl)methanol (5 g, 34.69 mmol), TBSCl (5.1 mL 41.6 mmol), imidazole (5.90 g, 86.7 mmol) in DMF (50 mL) was degassed and purged with N₂ for 3 times, and then the mixture was stirred at 25° C. for 16 hr under N₂ atmosphere. The reaction mixture was quenched by H₂O (60 mL) and then extracted with EtOAc (40 mL×2). The combined organic phase was washed with H₂O (20 mL×3), brine (20 mL), dried over anhydrous Na₂SO₄, filtered and concentrated in vacuum. The residue was purified by flash silica gel chromatography (ISCO®; 40 g SepaFlash® Silica Flash Column, Eluent of 0˜10% petroleum ether/EtOAc @ 60 mL/min) to give tert-butyl-[(3,5-difluorophenyl)methoxy]-dimethyl-silane (7 g, 78.1%) as a white oil. ¹H NMR (400 MHz, CDCl₃) δ ppm 0.12 (s, 6H) 0.96 (s, 9H) 4.72 (s, 2H) 6.67 (tt, J=8.94, 2.35 Hz, 1H) 6.82-6.89 (m, 2H).

### Step 2: 4-[[tert-butyl(dimethyl)silyl]oxymethyl]-2,6-difluoro-benzaldehyde

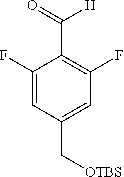 [SMILES: CC(C)(C)[Si](C)(C)OCc1cc(F)c(C=O)c(F)c1]

To a mixture of tert-butyl-[(3,5-difluorophenyl)methoxy]-dimethyl-silane (3 g, 11.61 mmol) in THF (30 mL) was added dropwise 2M LDA in THF (5.8 mL) at −30° C. under N₂. The mixture was stirred at −30° C. for 1 hr, DMF (1.1 mL, 13.9 mmol) was added and stirred for 1 hr at −30° C. The solution was quenched by addition of H₂O (10 mL) at 0° C. and extracted with EtOAc (20 mL×3). Then the combined organic layers were washed with brine (10 mL), dried by Na₂SO₄, filtered and concentrated under reduced pressure. The residue was purified by flash silica gel chromatography (ISCO®; 20 g SepaFlash® Silica Flash Column, Eluent of 0˜10% petroleum ether/EtOAc, flow rate: 60 mL/min) to give 4-[[tert-butyl(dimethyl)silyl]oxymethyl]-2,6-difluoro-benzaldehyde (2 g, 60.2%) as a white solid. ¹H NMR (400 MHz, DMSO-d₆) δ ppm 0.10 (s, 6H), 0.91 (s, 9H), 4.79 (s, 2H), 7.14 (d, J=10.04 Hz, 2H), 10.18 (s, 1H).

### Step 3: 9-bromo-5-(4-(((tert-butyldimethylsilyl)oxy)methyl)-2,6-difluorophenyl)-3-chloro-1-((2-(trimethylsilyl)ethoxy)methyl)-1,4,5,6-tetrahydropyrazolo[4,3-d]pyrido[4,3-f][1,3]diazepine

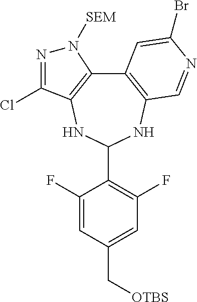 [SMILES: CC(C)(C)[Si](C)(C)OCc1cc(F)c(C2Nc3cnc(Br)cc3-c3c(c(Cl)nn3COCC[Si](C)(C)C)N2)c(F)c1]

A mixture of 4-[[tert-butyl(dimethyl)silyl]oxymethyl]-2,6-difluoro-benzaldehyde (341.9 mg, 1.19 mmol, 1 eq), 4-[4-amino-5-chloro-2-(2-trimethylsilylethoxymethyl)pyrazol-3-yl]-6-bromo-pyridin-3-amine (Intermediate B2-3) (500 mg, 1.19 mmol, 1 eq) in MeOH (20 mL) was degassed and purged with N₂ for 3 times, and stirred at 45° C. for 30 min under N₂ atmosphere. The reaction mixture was concentrated under reduced pressure. The residue was triturated with petroleum ether (5 mL) to give 9-bromo-5-(4-(((tert-butyldimethylsilyl)oxy)methyl)-2,6-difluorophenyl)-3-chloro-1-((2-(trimethylsilyl)ethoxy)methyl)-1,4,5,6-tetrahydropyrazolo[4,3-d]pyrido[4,3-f][1,3]diazepine (580 mg, crude) as a white solid.

### Step 4: 2-[[13-bromo-8-[4-[[tert-butyl(dimethyl)silyl]oxymethyl]-2,6-difluoro-phenyl]-5-chloro-3,4,7,9,12-petroleum ethertroleum etherntazatricyclo[8.4.0.0^{2,6}]tetradeca-1(10),2(6),4,7,11,13-hexaen-3-yl]methoxy]ethyl-trimethyl-silane (D2-5)

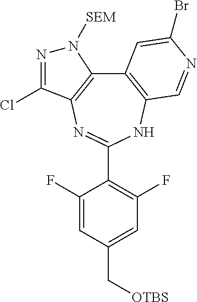 [SMILES: CC(C)(C)[Si](C)(C)OCc1cc(F)c(C2=Nc3c(Cl)nn(COCC[Si](C)(C)C)c3-c3cc(Br)ncc3N2)c(F)c1]

A mixture of 9-bromo-5-(4-(((tert-butyldimethylsilyl)oxy)methyl)-2,6-difluorophenyl)-3-chloro-1-((2-(trimethylsilyl)ethoxy)methyl)-1,4,5,6-tetrahydropyrazolo[4,3-d]pyrido[4,3-f][1,3]diazepine (580 mg, 0.84 mmol), DDQ (191.6 mg, 0.84 mmol) in DCM (1 mL) was degassed and purged with N₂ for 3 times, and then the mixture was stirred at 25° C. for 16 hr under N₂ atmosphere. The residue was diluted with NaHCO₃/H₂O (20 mL) and extracted with DCM (30 mL×3). The combined organic layers were washed with brine (10 mL), dried over Na₂SO₄, filtered and concentrated under reduced pressure. The residue was triturated with petroleum ether (10 mL) to give 2-[[13-bromo-8-[4-[[tert-butyl(dimethyl)silyl]oxymethyl]-2,6-difluoro-phenyl]-5-chloro-3,4,7,9,12-petroleum ethertroleum etherntazatricyclo[8.4.0.0^{2,6}]tetradeca-1(10),2(6),4,7,11,13-hexaen-3-yl]methoxy]ethyl-trimethyl-silane (542 mg, 87.2% yield) as a yellow solid. ¹H NMR (400 MHz, DMSO-d₆) δ ppm −0.02 (s, 9H), 0.11 (s, 6H), 0.87-0.90 (m, 2H), 0.92 (s, 9H), 3.70 (br t, J=7.78 Hz, 2H), 4.77 (s, 2H), 5.46 (s, 2H), 7.12 (br d, J=8.78 Hz, 2H), 7.66-7.74 (m, 2H), 8.87 (br s, 1H); LCMS (ESI) m/z: 686.1 [M+H]⁺.

### Synthesis of 2-[[13-chloro-8-(2-chloro-6-fluoro-phenyl)-3,4,7,9,12-pentazatricyclo[8.4.0.02,6]tetradeca-1(10),2(6),4,7,11,13-hexaen-3-yl]methoxy]ethyl-trimethyl-silane (Intermediate D2-6)

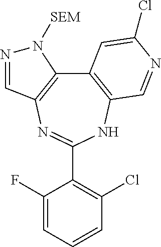 [SMILES: C[Si](C)(C)CCOCn1ncc2c1-c1cc(Cl)ncc1NC(c1c(F)cccc1Cl)=N2]

### Step 1: 2-[[13-chloro-8-(2-chloro-6-fluoro-phenyl)-3,4,7,9,12-pentazatricyclo[8.4.0.02,6]tetradeca-1(10),2(6),4,11,13-pentaen-3-yl]methoxy]ethyl-trimethyl-silane

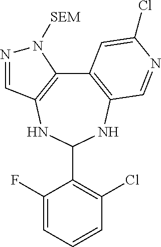 [SMILES: C[Si](C)(C)CCOCn1ncc2c1-c1cc(Cl)ncc1NC(c1c(F)cccc1Cl)N2]

To a solution of 4-[4-amino-2-(2-trimethylsilylethoxymethyl)pyrazol-3-yl]-6-chloro-pyridin-3-amine (Intermediate B2-1) (1 g, 2.94 mmol, 1 eq) and 2-chloro-6-fluoro-benzaldehyde (466.2 mg, 2.94 mmol, 1 eq) in t-BuOH (10 mL) and THF (5 mL) was added yttrium(III) trifluoromethanesulfonate (78.86 mg, 0.14 mmol, 0.05 eq) and K₂CO₃ (1.22 g, 8.82 mmol, 3 eq). The mixture was stirred at 50° C. for 16 hr. The reaction mixture was filtered and concentrated under reduced pressure. The crude 2-[[13-chloro-8-(2-chloro-6-fluoro-phenyl)-3,4,7,9,12-pentazatricyclo[8.4.0.02,6]tetradeca-1(10),2(6),4,11,13-pentaen-3-yl]methoxy]ethyl-trimethyl-silane (1.4 g) was obtained as a dark brown oil, which was used for next step directly.

### Step 2: 2-[[13-chloro-8-(2-chloro-6-fluoro-phenyl)-3,4,7,9,12-pentazatricyclo[8.4.0.02,6]tetradeca-1(10),2(6),4,7,11,13-hexaen-3-yl]methoxy]ethyl-trimethyl-silane (D2-6)

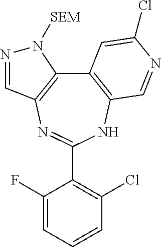 [SMILES: C[Si](C)(C)CCOCn1ncc2c1-c1cc(Cl)ncc1NC(c1c(F)cccc1Cl)=N2]

To a solution of 2-[[13-chloro-8-(2-chloro-6-fluoro-phenyl)-3,4,7,9,12-pentazatricyclo[8.4.0.02,6]tetradeca-1(10),2(6),4,11,13-pentaen-3-yl]methoxy]ethyl-trimethyl-silane (1.4 g, 2.91 mmol) in THF (5 mL) and t-BuOH (10 mL) was added DDQ (661.5 mg, 2.91 mmol). The mixture was stirred at 25° C. for 3 hr. The residue was diluted with EtOAc (30 mL) and washed with NaHCO₃/H₂O. The combined organic layers were washed with brine (20 mL), dried with anhydrous Na₂SO₄, filtered and concentrated under reduced pressure. The residue was purified by flash silica gel chromatography (ISCO®; 20 g SepaFlash® Silica Flash Column, petroleum ether/EtOAc with EtOAc from 0˜30%, 60 mL/min, 254 nm) to give 2-[[13-chloro-8-(2-chloro-6-fluoro-phenyl)-3,4,7,9,12-pentazatricyclo[8.4.0.02,6]tetradeca-1(10),2(6),4,7,11,13-hexaen-3-yl]methoxy]ethyl-trimethyl-silane (1.2 g, 85.1%) as a yellow solid. LCMS (ESI) m/z: 478.0 [M+H]⁺.

### Synthesis of 2-[[13-chloro-8-(2-chloro-6-fluoro-phenyl)-5-methyl-3,4,7,9,12-pentazatricyclo[8.4.0.02,6]tetradeca-1(10),2(6),4,7,11,13-hexaen-3-yl]methoxy]ethyl-trimethyl-silane (Intermediate D2-7)

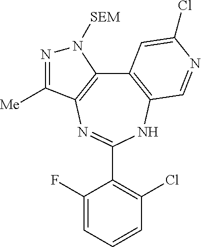 [SMILES: Cc1nn(COCC[Si](C)(C)C)c2c1N=C(c1c(F)cccc1Cl)Nc1cnc(Cl)cc1-2]

### Step 1: 2-[[13-chloro-8-(2-chloro-6-fluoro-phenyl)-5-methyl-3,4,7,9,12-pentazatricyclo[8.4.0.02,6]tetradeca-1(10),2(6),4,11,13-pentaen-3-yl]methoxy]ethyl-trimethyl-silane

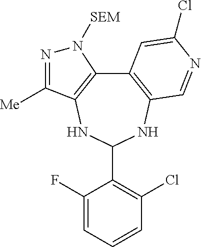 [SMILES: Cc1nn(COCC[Si](C)(C)C)c2c1NC(c1c(F)cccc1Cl)Nc1cnc(Cl)cc1-2]

A mixture of 4-[4-amino-5-methyl-2-(2-trimethylsilylethoxymethyl)pyrazol-3-yl]-6-chloro-pyridin-3-amine (Intermediate B2-2) (1.75 g, 4.94 mmol, 1 eq), 2-chloro-6-fluoro-benzaldehyde (784.0 mg, 4.94 mmol, 1 eq), K₂CO₃ (2.05 g, 14.8 mmol, 3 eq), yttrium(III) trifluoromethanesulfonate (132.5 mg, 0.24 mmol, 0.05 eq) in THF (6 mL) and t-BuOH (14 mL) was degassed and purged with N₂ for 3 times, and then the mixture was stirred at 50° C. for 16 hr under N₂ atmosphere. The reaction mixture was filtered and concentrated under reduced pressure to afford Compound 2-[[13-chloro-8-(2-chloro-6-fluoro-phenyl)-5-methyl-3,4,7,9,12-pentazatricyclo[8.4.0.02,6]tetradeca-1(10),2(6),4,11,13-pentaen-3-yl]methoxy]ethyl-trimethyl-silane (2.2 g, 89.9%) as a yellow solid.

### Step 2: 2-[[13-chloro-8-(2-chloro-6-fluoro-phenyl)-5-methyl-3,4,7,9,12-pentazatricyclo[8.4.0.02,6]tetradeca-1(10),2(6),4,7,11,13-hexaen-3-yl]methoxy]ethyl-trimethyl-silane (D2-7)

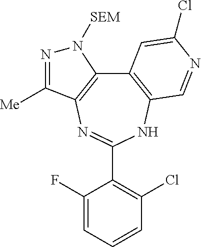 [SMILES: Cc1nn(COCC[Si](C)(C)C)c2c1N=C(c1c(F)cccc1Cl)Nc1cnc(Cl)cc1-2]

To a solution of 2-[[13-chloro-8-(2-chloro-6-fluoro-phenyl)-5-methyl-3,4,7,9,12-pentazatricyclo[8.4.0.02,6]tetradeca-1(10),2(6),4,11,13-pentaen-3-yl]methoxy]ethyl-trimethyl-silane (2.2 g, 4.45 mmol, 1 eq) in THF (12 mL) and t-BuOH (4 mL) was added DDQ (1.01 g, 4.45 mmol, 1 eq). The mixture was stirred at 25° C. for 2 hr. The reaction mixture was added H₂O (16 mL) and extracted with EtOAc (16 mL×3). The combined organic layers were washed with NaHCO₃ aqueous solution (16 mL), dried with anhydrous Na₂SO₄, filtered and concentrated under reduced pressure. The residue was purified by flash silica gel chromatography (ISCO®; 12 g SepaFlash® Silica Flash Column, Eluent of 0˜20% EtOAc/Petroleum ether gradient @ 30 mL/min) to provide 2-[[13-chloro-8-(2-chloro-6-fluoro-phenyl)-5-methyl-3,4,7,9,12-pentazatricyclo[8.4.0.02,6]tetradeca-1(10),2(6),4,7,11,13-hexaen-3-yl]methoxy]ethyl-trimethyl-silane (1.51 g, 66.0%) as a yellow solid. LCMS (ESI) m/z: 492.1 [M+H]⁺.

### Synthesis of 2-[[13-bromo-5-chloro-8-(2-chloro-6-fluoro-phenyl)-3,4,7,9,12-pentazatricyclo[8.4.0.0^{2,6}]tetradeca-1(10),2(6),4,7,11,13-hexaen-3-yl]methoxy]ethyl-trimethyl-silane (Intermediate D2-8)

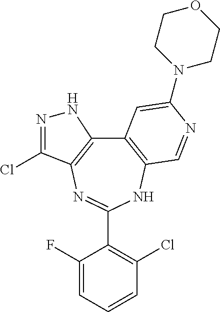 [SMILES: Fc1cccc(Cl)c1C1=Nc2c(Cl)n[nH]c2-c2cc(N3CCOCC3)ncc2N1]

### Step 1: 2-[[13-bromo-5-chloro-8-(2-chloro-6-fluoro-phenyl)-3,4,7,9,12-pentazatricyclo[8.4.0.0^{2,6}]tetradeca-1(10),2(6),4,11,13-pentaen-3-yl]methoxy]ethyl-trimethyl-silane

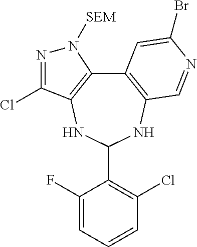 [SMILES: C[Si](C)(C)CCOCn1nc(Cl)c2c1-c1cc(Br)ncc1NC(c1c(F)cccc1Cl)N2]

A mixture of 2-chloro-6-fluoro-benzaldehyde (389 mg, 2.45 mmol, 1 eq), 4-[4-amino-5-chloro-2-(2-trimethylsilylethoxymethyl)pyrazol-3-yl]-6-bromo-pyridin-3-amine (Intermediate B2-3) (1 g, 2.45 mmol, 1 eq) in THF (10 mL) was degassed and purged with N₂ for 3 times, and then the mixture was stirred at 20° C. for 1 hr under N₂ atmosphere. The reaction mixture was concentrated under reduced pressure to give 2-[[13-bromo-5-chloro-8-(2-chloro-6-fluoro-phenyl)-3,4,7,9,12-pentazatricyclo[8.4.0.0^{2,6}]tetradeca-1(10),2(6),4,11,13-pentaen-3-yl]methoxy]ethyl-trimethyl-silane (1.37 g, crude) as a yellow solid. LCMS (ESI) m/z: 560.1 [M+H]⁺.

### Step 2: 2-[[13-bromo-5-chloro-8-(2-chloro-6-fluoro-phenyl)-3,4,7,9,12-pentazatricyclo[8.4.0.0^{2,6}]tetradeca-1(10),2(6),4,7,11,13-hexaen-3-yl]methoxy]ethyl-trimethyl-silane (D2-8)

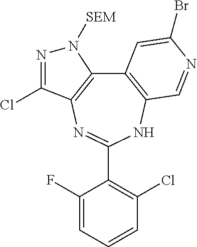 [SMILES: C[Si](C)(C)CCOCn1nc(Cl)c2c1-c1cc(Br)ncc1NC(c1c(F)cccc1Cl)=N2]

A mixture of 2-[[13-bromo-5-chloro-8-(2-chloro-6-fluoro-phenyl)-3,4,7,9,12-pentazatricyclo[8.4.0.0^{2,6}]tetradeca-1(10),2(6),4,11,13-pentaen-3-yl]methoxy]ethyl-trimethyl-silane (1.32 g, 2.36 mmol, 1 eq), DDQ (643 mg, 2.83 mmol, 1.2 eq) in DCM (13 mL) was degassed and purged with N₂ for 3 times, and then the mixture was stirred at 20° C. for 16 hr under N₂ atmosphere. The reaction mixture was diluted with NaHCO₃ aqueous solution (20 mL) and extracted with EtOAc (30 mL×3), dried over Na₂SO₄, concentrated under reduced pressure to give a residue. The residue was purified by column chromatography (ISCO®; 12 g SepaFlash® Silica Flash Column, petroleum ether/EtOAc with EtOAc from 0˜15%, 30 mL/min, 254 nm) to afford 2-[[13-bromo-5-chloro-8-(2-chloro-6-fluoro-phenyl)-3,4,7,9,12-pentazatricyclo[8.4.0.0^{2,6}]tetradeca-1(10),2(6),4,7,11,13-hexaen-3-yl]methoxy]ethyl-trimethyl-silane (1.18 g, 90.0%) as a yellow solid. LCMS (ESI) m/z: 560.1 [M+H]⁺.

### Synthesis of 13-bromo-8-(2,6-difluorophenyl)-3-[(4-methoxyphenyl)methyl]-5-(trifluoromethyl)-3,4,7,9,12-pentazatricyclo[8.4.0.02,6]tetradeca-1(10),2(6),4,7,11,13-hexaene (D3-1)

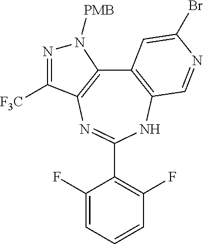

### Step 1: 13-bromo-8-(2,6-difluorophenyl)-3-[(4-methoxyphenyl)methyl]-5-(trifluoromethyl)-3,4,7,9,12-pentazatricyclo[8.4.0.02,6]tetradeca-1(10),2(6),4,11,13-pentaene

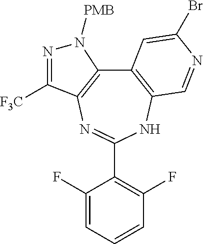

The mixture of 4-[4-amino-2-[(4-methoxyphenyl)methyl]-5-(trifluoromethyl)pyrazol-3-yl]-6-bromo-pyridin-3-amine (Intermediate B2-19) (800 mg, 1.81 mmol, 1 eq) and 2,6-difluorobenzaldehyde (257.0 mg, 1.81 mmol, 1 eq) in THF (1 mL) was stirred at 25° C. for 16 hr. The reaction mixture was concentrated under vacuum. The crude product 13-bromo-8-(2,6-difluorophenyl)-3-[(4-methoxyphenyl)methyl]-5-(trifluoromethyl)-3,4,7,9,12-pentazatricyclo[8.4.0.02,6]tetradeca-1(10),2(6),4,11,13-pentaene (1 g, crude) was obtained as a yellow oil, which was used for the next step without further purification.

### Step 2: 13-bromo-8-(2,6-difluorophenyl)-3-[(4-methoxyphenyl)methyl]-5-(trifluoromethyl)-3,4,7,9,12-pentazatricyclo[8.4.0.02,6]tetradeca-1(10),2(6),4,7,11,13-hexaene (D3-1)

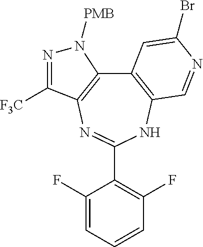

A mixture of 13-bromo-8-(2,6-difluorophenyl)-3-[(4-methoxyphenyl)methyl]-5-(trifluoromethyl)-3,4,7,9,12-pentazatricyclo[8.4.0.02,6]tetradeca-1(10),2(6),4,11,13-pentaene (1 g, 1.77 mmol), MnO₂ (1.54 g, 17.7 mmol, 10 eq) in THF (10 mL) was stirred at 25° C. for 16 hr under N₂ atmosphere. The reaction mixture was filtered. The filter liquor was concentrated in vacuum. The residue was purified by flash chromatography (ISCO®; 24 g SepaFlash® Silica Flash Column, petroleum ether/EtOAc with EtOAc from 0˜30%, 100 mL/min, 254 mn) to provide 13-bromo-8-(2,6-difluorophenyl)-3-[(4-methoxyphenyl)methyl]-5-(trifluoromethyl)-3,4,7,9,12-pentazatricyclo[8.4.0.02,6]tetradeca-1(10),2(6),4,7,11,13-hexaene as a yellow solid (800 mg, 80.2%). LCMS (ESI) m/z: 566.0 [M+H]⁺.

### Synthesis of 13-bromo-5-(difluoromethyl)-8-(2,6-difluorophenyl)-3-[(4-methoxyphenyl)methyl]-3,4,7,9,12-pentazatricyclo[8.4.0.02,6]tetradeca-1(10),2(6),4,11,13-pentaene (Intermediate D3-4)

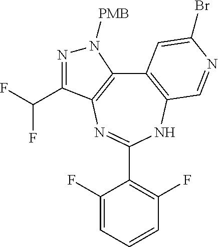

### Step 1: 13-bromo-5-(difluoromethyl)-8-(2,6-difluorophenyl)-3-[(4-methoxyphenyl)methyl]-3,4,7,9,12-pentazatricyclo[8.4.0.02,6]tetradeca-1(10),2(6),4,11,13-pentaene

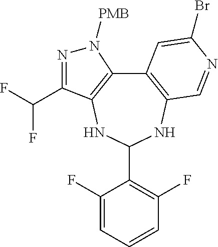

A solution of 4-[4-amino-5-(difluoromethyl)-2-[(4-methoxyphenyl)methyl]pyrazol-3-yl]-6-bromo-pyridin-3-amine (Intermediate B2-20) (500 mg, 1.18 mmol) and 2,6-difluorobenzaldehyde (167 mg, 1.18 mmol) in THF (7 mL) was stirred at 25° C. for 16 hr. The reaction mixture was filtered and concentrated under reduced pressure. The residue was purified by flash silica gel chromatography (ISCO®; 12 g SepaFlash® Silica Flash Column, petroleum ether/EtOAc with EtOAc from 0˜40%, 30 mL/min, 254 nm) to provide 13-bromo-5-(difluoromethyl)-8-(2,6-difluorophenyl)-3-[(4-methoxyphenyl)methyl]-3,4,7,9,12-pentazatricyclo[8.4.0.02,6]tetradeca-1(10),2(6),4,11,13-pentaene (534 mg, 82.1% yield) as a yellow solid. LCMS (ESI) m/z: 550.1 [M+H]⁺.

### Step 2: 13-bromo-5-(difluoromethyl)-8-(2,6-difluorophenyl)-3-[(4-methoxyphenyl)methyl]-3,4,7,9,12-pentazatricyclo[8.4.0.02,6]tetradeca-1(10),2(6),4,7,11,13-hexaene (D3-4)

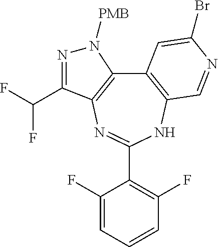

A solution of 13-bromo-5-(difluoromethyl)-8-(2,6-difluorophenyl)-3-[(4-methoxyphenyl) methyl]-3,4,7,9,12-pentazatricyclo[8.4.0.02,6]tetradeca-1(10),2(6),4,11,13-pentaene (Intermediate B2-20) (500 mg, 0.911 mmol) in CH₂Cl₂ (8 mL) was added DDQ (227 mg, 1.00 mmol). The mixture was stirred at 25° C. for 16 hr. The reaction mixture was added NaHCO₃/H₂O (10 mL) and extracted with EtOAc (10 mL×3). The combined organic layers were washed with brine (10 mL), dried with anhydrous Na₂SO₄, filtered and concentrated under reduced pressure. The residue was purified by flash silica gel chromatography (ISCO®; 4 g SepaFlash® Silica Flash Column, petroleum ether/EtOAc with EtOAc from 0˜30%, 20 mL/min, 254 nm) to provide 13-bromo-5-(difluoromethyl)-8-(2,6-difluorophenyl)-3-[(4-methoxyphenyl)methyl]-3,4,7,9,12-pentazatricyclo[8.4.0.02,6]tetradeca-1(10),2(6),4,7,11,13-hexaene (450 mg, 83.4% yield) as a brown solid. ¹H NMR (400 MHz, DMSO-d₆) δ ppm 3.72 (s, 3H), 5.55 (s, 2H), 6.74 (s, 1H), 6.88 (s, 1H), 6.94 (d, J=8.75 Hz, 2H), 7.01 (s, 1H), 7.09 (d, J=8.63 Hz, 2H), 7.24 (t, J=8.13 Hz, 2H), 7.44 (s, 1H), 7.55-7.64 (m, 1H), 7.72 (s, 1H), 8.80 (s, 1H); LCMS (ESI) m/z: 546.1 [M+H]⁺.

### Synthesis of 13-bromo-5-(difluoromethyl)-8-(2,6-difluorophenyl)-3-[(4-methoxyphenyl)methyl]-3,4,7,9,12-pentazatricyclo[8.4.0.02,6]tetradeca-1(10),2(6),4,11,13-pentaene (Intermediate D3-5)

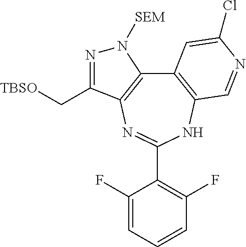 [SMILES: CC(C)(C)[Si](C)(C)OCc1nn(COCC[Si](C)(C)C)c2c1N=C(c1c(F)cccc1F)Nc1cnc(Cl)cc1-2]

### Step 1: methyl 13-chloro-8-(2,6-difluorophenyl)-3-(2-trimethylsilylethoxymethyl)-3,4,7,9,12-pentazatricyclo[8.4.0.02,6]tetradeca-1(10),2(6),4,11,13-pentaene-5-carboxylate

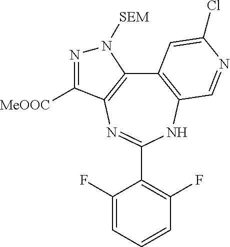 [SMILES: COC(=O)c1nn(COCC[Si](C)(C)C)c2c1N=C(c1c(F)cccc1F)Nc1cnc(Cl)cc1-2]

A mixture of methyl 4-amino-5-(5-amino-2-chloro-4-pyridyl)-1-(2-trimethylsilylethoxy methyl)pyrazole-3-carboxylate (Intermediate B-21) (4.80 g, 12.0 mmol) and 2,6-difluorobenzaldehyde (3.43 g, 24.1 mmol) in t-BuOH (80 mL) and THF (80 mL) was stirred at 20° C. for 16 hr. The mixture was filtered and washed with DCM (200 mL). The filtrate was concentrated under reduced pressure. The residue was purified by flash chromatography (ISCO®; 40 g AgelaFlash® Silica Flash Column, petroleum ether/EtOAc with EtOAc from 0˜35%, flow rate=55 mL/min, 254 nm) to afford methyl 13-chloro-8-(2,6-difluorophenyl)-3-(2-trimethylsilylethoxymethyl)-3,4,7,9,12-pentazatricyclo[8.4.0.02,6]tetradeca-1(10),2(6),4,11,13-pentaene-5-carboxylate (3.13 g, 29.3% yield) as a light yellow solid. LCMS (ESI) m/z: 522.1 [M+H]⁺.

### Step 2: methyl 13-chloro-8-(2,6-difluorophenyl)-3-(2-trimethylsilylethoxymethyl)-3,4,7,9,12-pentazatricyclo[8.4.0.02,6]tetradeca-1(10),2(6),4,7,11,13-hexaene-5-carboxylate

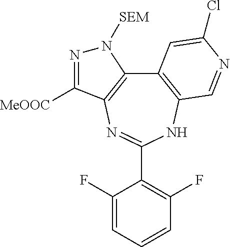 [SMILES: COC(=O)c1nn(COCC[Si](C)(C)C)c2c1N=C(c1c(F)cccc1F)Nc1cnc(Cl)cc1-2]

To a solution of methyl 13-chloro-8-(2,6-difluorophenyl)-3-(2-trimethylsilylethoxy methyl)-3,4,7,9,12-pentazatricyclo[8.4.0.02,6]tetradeca-1(10),2(6),4,11,13-pentaene-5-carboxylate (3.13 g, 6.00 mmol), methyl 13-chloro-8-(2,6-difluorophenyl)-3-(2-trimethylsilylethoxymethyl)-3,4,7,9,12-pentazatricyclo[8.4.0.02,6]tetradeca-1(10),2(6),4,11,13-pentaene-5-carboxylate (3.13 g, 6.00 mmol) in THF (160 mL) was added DDQ (2.72 g, 11.9 mmol) and K₂CO₃ (1.66 g, 11.9 mmol). The mixture was stirred at 20° C. for 16 hr under nitrogen atmosphere, and then concentrated under reduced pressure. The residue was purified by flash chromatography (ISCO®; 40 g AgelaFlash® Silica Flash Column, petroleum ether/EtOAc with EtOAc from 0˜15%, flow rate=50 mL/min, 254 nm) to afford methyl 13-chloro-8-(2,6-difluorophenyl)-3-(2-trimethylsilylethoxymethyl)-3,4,7,9,12-pentazatricyclo[8.4.0.02,6]tetradeca-1(10),2(6),4,7,11,13-hexaene-5-carboxylate (1.4 g, 44.9%) as a yellow solid. LCMS (ESI) m/z: 520.0 [M+H]⁺.

### Step 3: [13-chloro-8-(2,6-difluorophenyl)-3-(2-trimethylsilylethoxymethyl)-3,4,7,9,12-pentazatricyclo[8.4.0.02,6]tetradeca-1(10),2(6),4,7,11,13-hexaen-5-yl]methanol

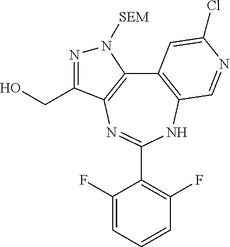 [SMILES: C[Si](C)(C)CCOCn1nc(CO)c2c1-c1cc(Cl)ncc1NC(c1c(F)cccc1F)=N2]

To a solution of methyl 13-chloro-8-(2,6-difluorophenyl)-3-(2-trimethylsilylethoxy methyl)-3,4,7,9,12-pentazatricyclo[8.4.0.02,6]tetradeca-1(10),2(6),4,7,11,13-hexaene-5-carboxylate (300 mg, 0.577 mmol) in EtOH (8 mL) and THF (8 mL) was added NaBH₄ (66.0 mg, 1.74 mmol) and CaCl₂ (192.0 mg, 1.73 mmol) at 0° C. under nitrogen. The mixture was stirred at 20° C. for 2 hr. The reaction was quenched with water (30 mL) at 0° C. slowly and stirred for 15 min. The mixture was extracted with EtOAc (30 mL×2). The combined organic phase was washed with brine (20 mL), dried over anhydrous Na₂SO₄, filtered and concentrated under reduced pressure. The residue was purified by purified by flash chromatography (ISCO®; 4 g AgelaFlash® Silica Flash Column, petroleum ether/EtOAc with EtOAc from 0˜50%, flow rate=35 mL/min, 254 nm) to afford [13-chloro-8-(2,6-difluorophenyl)-3-(2-trimethylsilylethoxy methyl)-3,4,7,9,12-pentazatricyclo[8.4.0.02,6]tetradeca-1(10),2(6),4,7,11,13-hexaen-5-yl]methanol (259 mg, 91.2%) as a yellow solid. LCMS (ESI) m/z: 492.1 [M+H]⁺.

### Step 4: tert-butyl-[[13-chloro-8-(2,6-difluorophenyl)-3-(2-trimethylsilylethoxymethyl)-3,4,7,9,12-pentazatricyclo[8.4.0.02,6]tetradeca-1(10),2(6),4,7,11,13-hexaen-5-yl]methoxy]-dimethyl-silane (D3-5)

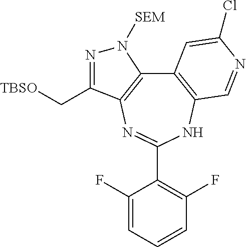 [SMILES: CC(C)(C)[Si](C)(C)OCc1nn(COCC[Si](C)(C)C)c2c1N=C(c1c(F)cccc1F)Nc1cnc(Cl)cc1-2]

To a solution of [13-chloro-8-(2,6-difluorophenyl)-3-(2-trimethylsilylethoxymethyl)-3,4,7,9,12-pentazatricyclo[8.4.0.02,6]tetradeca-1(10),2(6),4,7,11,13-hexaen-5-yl]methanol (259 mg, 0.526 mmol) in DMF (10 mL) was added imidazole (286 mg, 4.20 mmol) then added TBSCl (238 mg, 1.58 mmol) at 0° C. under nitrogen. The mixture was stirred at 60° C. for 3 hr. The reaction was quenched with water (30 mL) at 0° C. slowly and stirred for 15 min. The mixture was extracted with EtOAc (30 mL×2). The combined organic phase was washed with brine (20 mL), dried over anhydrous Na₂SO₄, filtered and concentrated under reduced pressure. The residue was purified by purified by flash chromatography (ISCO®; 4 g AgelaFlash® Silica Flash Column, petroleum ether/EtOAc with EtOAc from 0˜50%, flow rate=35 mL/min, 254 nm) to afford tert-butyl-[[13-chloro-8-(2,6-difluorophenyl)-3-(2-trimethylsilylethoxymethyl)-3,4,7,9,12-pentazatricyclo[8.4.0.02,6]tetradeca-1(10),2(6),4,7,11,13-hexaen-5-yl]methoxy]-dimethyl-silane (227 mg, 69.5%) as a yellow solid. LCMS (ESI) m/z: 606.2 [M+H]⁺.

### Synthesis of 9-chloro-5-(2,6-difluorophenyl)-3,7-dimethyl-1-((2-(trimethylsilyl)ethoxy)methyl)-1,6-dihydropyrazolo[4,3-d]pyrido[4,3-f][1,3]diazepine (Intermediate D4-1)

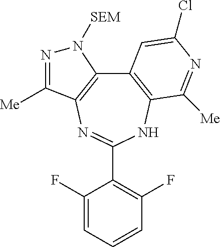 [SMILES: Cc1nn(COCC[Si](C)(C)C)c2c1N=C(c1c(F)cccc1F)Nc1c-2cc(Cl)nc1C]

To a solution of 4-[4-amino-5-methyl-2-(2-trimethylsilylethoxymethyl)pyrazol-3-yl]-6-chloro-2-methyl-pyridin-3-amine (Intermediate B2-4) (3.5 g, 9.51 mmol, 1 eq) and 2,6-difluorobenzaldehyde (1.35 g, 9.51 mmol, 1.02 mL, 1 eq) in ^{t}BuOH (5 mL) and THF (1 mL) was added K₂CO₃ (3.94 g, 28.5 mmol, 3 eq) at 25° C. After addition, the mixture was stirred at this temperature for 30 min, and then DDQ (4.32 g, 19.0 mmol, 2 eq) was added. The resulting mixture was stirred at 25° C. for 4 hr under N₂ atmosphere, then concentrated to give a crude product. The crude was purified by silica gel chromatography (petroleum ether/EtOAc=0%-30%) to give 2-[[13-chloro-8-(2,6-difluorophenyl)-5,11-dimethyl-3,4,7,9,12-pentazatricyclo[8.4.0.02,6]tetradeca-1(10),2(6),4,7,11,13-hexaen-3-yl]methoxy]ethyl-trimethyl-silane as a yellow solid (3 g, 64.4%). ¹H NMR (400 MHz, DMSO-d₆) δ ppm −0.03 (s, 9H), 0.81-0.95 (m, 2H), 2.01-2.08 (m, 3H), 2.26-2.34 (m, 3H), 3.64-3.76 (m, 2H), 5.41 (s, 1H), 7.21 (t, J=8.2 Hz, 2H), 7.47 (s, 1H), 7.52-7.61 (m, 1H), 7.88 (s, 1H); LCMS (ESI) m/z: 490.1 [M+H]⁺.

### Synthesis of 9-chloro-5-(2,6-difluorophenyl)-1-(4-methoxybenzyl)-7-methyl-1,6-dihydropyrazolo[4,3-d]pyrido[4,3-f][1,3]diazepine (Intermediate D4-2)

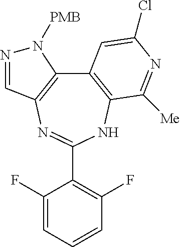

To a solution of 4-[4-amino-2-[(4-methoxyphenyl)methyl]pyrazol-3-yl]-6-chloro-2-methyl-pyridin-3-amine (Intermediate B2-6) (14 g, 40.7 mmol, 1 eq) and 2,6-difluorobenzaldehyde (5.79 g, 40.7 mmol, 1 eq) in ^{t}BuOH (300 mL) was added K₂CO₃ (16.88 g, 0.122 mmol, 3 eq), I₂ (20.67 g, 81.4 mmol, 2 eq) at 25° C. After addition, the mixture was stirred at this temperature for 30 min, and then MnO₂ (35.4 g, 0.407 mol, 10 eq) was added. The resulting mixture was stirred at 60° C. for 2 hr under N₂ atmosphere, then concentrated under reduced pressure. The crude product was purified by silica gel chromatography (petroleum ether:EtOAc=0%-50%) and Pre-HPLC (column: Kromasil Eternity XT 150×30 mm×10 m; mobile phase: [water (NH₃H₂O+NH₄HCO₃)-MeCN]; B %: 18%-58%, 9 min) to afford 13-chloro-8-(2,6-difluorophenyl)-3-[(4-methoxyphenyl) methyl]-11-methyl-3,4,7,9,12-pentazatricyclo[8.4.0.02,6]tetradeca-1(10),2(6),4,7,11,13-hexaene (3.5 g, 18.4%) as a yellow solid. ¹H NMR (400 MHz, DMSO-d₆) δ ppm 2.26 (s, 3H), 3.73 (s, 3H), 5.45 (br s, 2H), 6.92 (d, J=8.5 Hz, 3H), 7.08 (d, J=8.5 Hz, 3H), 7.22 (t, J=8.2 Hz, 2H), 7.57 (quin, J=7.5 Hz, 1H); LCMS (ESI) m/z: 478.0 [M+H]⁺.

### Synthesis of 2-[[13-chloro-8-(2,6-difluorophenyl)-5-methyl-11-(trifluoromethyl)-3,4,7,9,12-pentazatricyclo[8.4.0.02,6]tetradeca-1(10),2(6),4,7,11,13-hexaen-3-yl]methoxy]ethyl-trimethyl-silane (Intermediate D7-1)

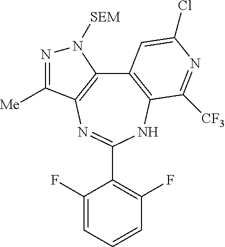 [SMILES: Cc1nn(COCC[Si](C)(C)C)c2c1N=C(c1c(F)cccc1F)Nc1c-2cc(Cl)nc1C(F)(F)F]

To a mixture of 4-[4-amino-5-methyl-2-(2-trimethylsilylethoxymethyl)pyrazol-3-yl]-6-chloro-2-(trifluoromethyl)pyridin-3-amine (Intermediate B2-22) (300 mg, 0.676 μmol, 1 eq) and 2,6-difluorobenzaldehyde (97 mg, 0.683 mmol, 1.01 eq) in t-BuOH (4 mL) and THF (0.8 mL) was added K₂CO₃ (282 mg, 2.04 mmol, 3.02 eq). The mixture was stirred at 25° C. for 30 min. DDQ (308 mg, 1.36 mmol, 2.01 eq) was added to the above mixture. The reaction mixture was stirred at 25° C. for 4 hr under N₂. This mixture was combined with another batch and concentrated in vacuum. The residue was purified by flash silica gel chromatography (Biotage®; 40 g Agela Silica Flash Column, Eluent of 0˜13% EtOAc/petroleum ether, gradient @ 60 mL/min). 2-[[13-chloro-8-(2,6-difluorophenyl)-5-methyl-11-(trifluoromethyl)-3,4,7,9,12-pentazatricyclo[8.4.0.02,6]tetradeca-1(10),2(6),4,7,11,13-hexaen-3-yl]methoxy]ethyl-trimethyl-silane (802 mg, 68.2%) was obtained as a yellow solid. LCMS (ESI) m/z: 544.3 [M+H]⁺.

### Synthesis of 3-chloro-5-(2,6-difluorophenyl)-1,6-dihydrobenzo[d]pyrazolo[3,4-f][1,3]diazepine-9-carboxylic acid (Intermediate E1-1)

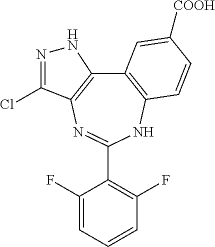 [SMILES: O=C(O)c1ccc2c(c1)-c1[nH]nc(Cl)c1N=C(c1c(F)cccc1F)N2]

### Step 1: methyl 3-chloro-5-(2,6-difluorophenyl)-1-(2-trimethylsilylethoxymethyl)-6H-pyrazolo[4,3-d][1,3]benzodiazepine-9-carboxylate

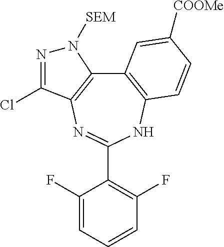 [SMILES: COC(=O)c1ccc2c(c1)-c1c(c(Cl)nn1COCC[Si](C)(C)C)N=C(c1c(F)cccc1F)N2]

A mixture of methyl 3-[4-amino-5-chloro-2-(2-trimethylsilylethoxymethyl)pyrazol-3-yl]-4-(tert-butoxycarbonylamino)benzoate (Intermediate B1-7) (200 mg, 0.402 mmol, 1.0 eq), 2,6-difluorobenzaldehyde (57.1 mg, 0.402 mmol, 1.0 eq), K₂CO₃ (166 mg, 1.21 mmol, 3.0 eq) in t-BuOH/THF (1:1, 3 mL) was stirred for 30 min, and then I₂ (153 mg, 0.603 mmol, 1.5 eq) was added and the mixture was stirred at 25° C. for 2 hr. t-BuOH/THF was evaporated, the residue was purified by column chromatography (ISCO®; 4 g SepaFlash® Silica Flash Column, petroleum ether/EtOAc with EtOAc from 0˜50%, 20 mL/min, 254 mn), to provide methyl 3-chloro-5-(2,6-difluorophenyl)-1-(2-trimethylsilylethoxymethyl)-6H-pyrazolo[4,3-d][1,3]benzodiazepine-9-carboxylate as a white solid (40 mg, 19.1%). LCMS (ESI) m/z: 519.1 [M+H]⁺.

### Step 2: 3-chloro-5-(2,6-difluorophenyl)-1,6-dihydrobenzo[d]pyrazolo[3,4-f][1,3]diazepine-9-carboxylic acid (E1-1)

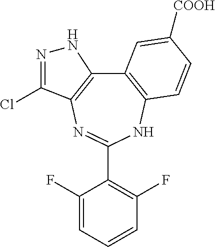 [SMILES: O=C(O)c1ccc2c(c1)-c1[nH]nc(Cl)c1N=C(c1c(F)cccc1F)N2]

A mixture of methyl 3-chloro-5-(2,6-difluorophenyl)-1-(2-trimethylsilylethoxymethyl)-6H-pyrazolo[4,3-d][1,3]benzodiazepine-9-carboxylate (40 mg, 0.077 mmol, 1.0 eq), LiOH H₂O (6.47 mg, 0.154 mmol, 2.0 eq) in THF/H₂O (3:1, 2 mL) was stirred at 25° C. for 16 hr. The reaction mixture was concentrated and neutralized by 1N HCl in H₂O until pH reached 1. The precipitate was formed and collected by filtration and dried over air to provide 3-chloro-5-(2,6-difluorophenyl)-1,6-dihydrobenzo[d]pyrazolo[3,4-f][1,3]diazepine-9-carboxylic acid as a white solid (30 mg), which was used for the next step directly. ¹H NMR (400 MHz, CD₃OD) δ ppm 6.84 (d, J=8.31 Hz, 1H), 7.08-7.25 (m, 2H), 7.44-7.62 (m, 1H), 7.88-8.04 (m, 1H), 8.39 (d, J=1.83 Hz, 1H); LCMS (ESI) m/z: 375.1 [M+H]⁺.

### Synthesis of methyl 5-(2,6-difluorophenyl)-1-[(4-methoxyphenyl)methyl]-6H-pyrazolo[4,3-d][1,3]benzodiazepine-9-carboxylate (Intermediate E2-1)

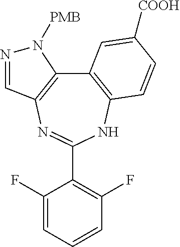

### Step 1: methyl 5-(2,6-difluorophenyl)-1-[(4-methoxyphenyl)methyl]-6H-pyrazolo[4,3-d][1,3]benzodiazepine-9-carboxylate

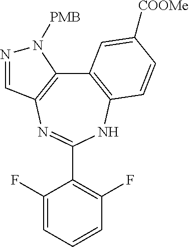

A mixture of methyl 4-amino-3-[4-amino-2-[(4-methoxyphenyl)methyl]pyrazol-3-yl]benzoate (Intermediate B1-8) (230 mg, 0.652 mmol, 1.0 eq), 2,6-difluorobenzaldehyde (231 mg, 1.63 mmol), K₂CO₃ (270 mg, 1.96 mmol) and I₂ (497 mg, 1.96 mmol) in t-BuOH/THF (4:1, 5 mL) was stirred at 20° C. for 2 hr. The mixture was quenched with Na₂S₂O₃/H₂O (30 mL) and extracted with EtOAc (50 mL×2). The extractions were concentrated in the vacuum and purified by flash silica gel chromatography (ISCO®; 4 g SepaFlash® Silica Flash Column, Eluent of 0˜100% EtOAc/Petroleum ether, gradient @ 20 mL/min), to provide methyl 5-(2,6-difluorophenyl)-1-[(4-methoxyphenyl)methyl]-6H-pyrazolo[4,3-d][1,3]benzodiazepine-9-carboxylate as a dark brown oil (210 mg, 67.8%). ¹H NMR (400 MHz, DMSO-d₆) δ ppm 3.74 (d, J=4.63 Hz, 7H) 3.94-4.13 (m, 2H), 5.34 (s, 2H), 6.84 (d, J=8.38 Hz, 1H), 6.94 (d, J=8.76 Hz, 2H), 7.09-7.24 (m, 5H), 7.35 (s, 1H), 7.50-7.59 (m, 1H), 7.64 (d, J=1.88 Hz, 1H), 7.75 (dd, J=8.38, 1.88 Hz, 1H), 8.60 (s, 1H); LCMS (ESI) m/z: 475.1 [M+H]⁺.

### Step 2: methyl 5-(2,6-difluorophenyl)-1-[(4-methoxyphenyl)methyl]-6H-pyrazolo[4,3-d][1,3]benzodiazepine-9-carboxylic acid (E2-1)

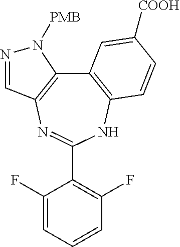

Methyl 5-(2,6-difluorophenyl)-1-[(4-methoxyphenyl)methyl]-6H-pyrazolo[4,3-d][1,3]benzodiazepine-9-carboxylate (210 mg, 0.442 mmol, 1.0 eq) and LiOH—H₂O (185 mg, 4.43 mmol, 10.0 eq) in THF/H₂O (3:1, 4 mL) was stirred at 25° C. for 8 hrs. The mixture was neutralized by 1M HCl (5 mL) until the pH=7. The mixture was extracted with EtOAc (10 mL×3). The extractions were washed with brine (20 mL), dried over anhydrous Na₂SO₄ and concentrated in a vacuum. The residue was purified by flash silica gel chromatography (ISCO®; 4 g SepaFlash® Silica Flash Column, Eluent of 0˜10% MeOH/DCM gradient @ 12 mL/min), to provide the tittle compound as a yellow solid (90.0 mg, 44.2%). ¹H NMR (400 MHz, CDCl₃) δ ppm 3.69-3.88 (m, 4H), 4.13 (q, J=7.17 Hz, 1H), 5.45 (s, 2H), 5.79 (s, 1H), 6.65 (d, J=8.24 Hz, 1H), 6.85-6.92 (m, 2H), 6.99 (t, J=8.16 Hz, 2H), 7.24 (d, J=8.70 Hz, 2H), 7.35-7.43 (m, 1H), 7.53 (s, 1H), 7.86-7.99 (m, 2H); LCMS (ESI) m/z: 461.7 [M+H]⁺.

### Synthesis of 3-chloro-5-(2,6-difluorophenyl)-8-fluoro-1,6-dihydrobenzo[d]pyrazolo[3,4-f][1,3]diazepine-9-carboxylic acid (Intermediate E2-5)

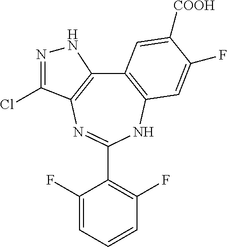 [SMILES: O=C(O)c1cc2c(cc1F)NC(c1c(F)cccc1F)=Nc1c(Cl)n[nH]c1-2]

### Step 1: methyl 3-chloro-5-(2,6-difluorophenyl)-8-fluoro-1-(2-trimethylsilylethoxymethyl)-6H-pyrazolo[4,3-d][1,3]benzodiazepine-9-carboxylate

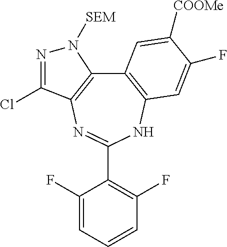 [SMILES: COC(=O)c1cc2c(cc1F)NC(c1c(F)cccc1F)=Nc1c(Cl)nn(COCC[Si](C)(C)C)c1-2]

A mixture of methyl 4-amino-5-[4-amino-5-chloro-2-(2-trimethylsilylethoxymethyl) pyrazol-3-yl]-2-fluoro-benzoate (100 mg, 0.241 mmol, 1.0 eq), 2,6-difluorobenzaldehyde (68.4 mg, 0.482 mmol, 2.0 eq) and K₂CO₃ (66.6 mg, 0.482 mmol, 2.0 eq) in t-BuOH (3 mL) was stirred for 30 min, and then I₂ (91.7 mg, 0.361 mmol, 1.5 eq) was added and the mixture was stirred at 60° C. for 2 hr. The solid was filtered off and washed with MeOH (4 mL×2). The organic layers were combined. Removal of the organic solvents under reduced pressure provided the crude product, which was purified by flash chromatography (ISCO®; 4 g SepaFlash® Silica Flash Column, petroleum ether/EtOAc with EtOAc from 0˜40%, 12 mL/min, 254 mn) to afford methyl 3-chloro-5-(2,6-difluorophenyl)-8-fluoro-1-(2-trimethylsilylethoxymethyl)-6H-pyrazolo[4,3-d][1,3]benzodiazepine-9-carboxylate as a white solid (90 mg, 69.5%). LCMS (ESI) m/z: 537.1, [M+H]⁺.

### Step 2: Synthesis of 3-chloro-5-(2,6-difluorophenyl)-8-fluoro-1,6-dihydrobenzo[d]pyrazolo [3,4-f][1,3]diazepine-9-carboxylic acid-(E2-5)

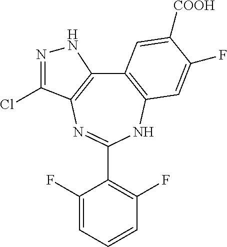 [SMILES: O=C(O)c1cc2c(cc1F)NC(c1c(F)cccc1F)=Nc1c(Cl)n[nH]c1-2]

A mixture of methyl 3-chloro-5-(2,6-difluorophenyl)-8-fluoro-1-(2-trimethylsilylethoxy methyl)-6H-pyrazolo[4,3-d][1,3]benzodiazepine-9-carboxylate (90 mg, 0.167 mmol, 1.0 eq), LiOH—H₂O (21.1 mg, 0.502 mmol, 3.0 eq) in THF/H₂O (3:1, 4 mL) was stirred at 25° C. for 16 hr. THF was evaporated and acidified with 12N HCl in H₂O until pH reached 1. The reaction was extracted with EtOAc (10 mL×3), dried over Na₂SO₄, filtered and concentrated under reduced pressure. The crude product was purified by flash chromatography (ISCO®; 4 g SepaFlash® Silica Flash Column, petroleum ether/EtOAc with EtOAc from 0˜40%, 12 mL/min, 254 mn), to provide the tittle compound as a white solid (30 mg, 45%) as a white solid. LCMS (ESI) m/z: 393.1 [M+H]⁺.

### Example Int-1: Synthesis of 4-[8-(2,6-difluorophenyl)-5-methyl-3,4,7,9,12-pentazatricyclo[8.4.0.02,6] tetradeca-1(10),2(6),4,7,11,13-hexaen-13-yl]morpholine

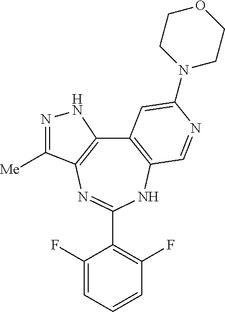 [SMILES: Cc1n[nH]c2c1N=C(c1c(F)cccc1F)Nc1cnc(N3CCOCC3)cc1-2]

### Step 1: 2-[[8-(2,6-difluorophenyl)-5-methyl-13-morpholino-3,4,7,9,12-pentazatricyclo[8.4.0.02,6]tetradeca-1(10),2(6),4,7,11,13-hexaen-3-yl]methoxy]ethyl-trimethyl-silane

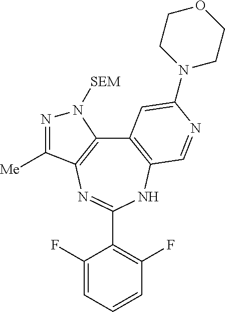 [SMILES: Cc1nn(COCC[Si](C)(C)C)c2c1N=C(c1c(F)cccc1F)Nc1cnc(N3CCOCC3)cc1-2]

A mixture of 2-[[13-chloro-8-(2,6-difluorophenyl)-5-methyl-3,4,7,9,12-pentazatricyclo[8.4.0.02,6] tetradeca-1(10),2(6),4,7,11,13-hexaen-3-yl]methoxy]ethyl-trimethyl-silane (Intermediate D2-3) (160 mg, 0.336 mmol, 1.0 eq), morpholine (29.3 mg, 0.336 mmol, 1.0 eq), DavePhos (132 mg, 0.336 mmol, 1.0 eq), Pd₂(dba)₃ (154 mg, 0.168 mmol, 0.5 eq) and t-BuONa (96.9 mg, 1.01 mmol, 3.0 eq) in dioxane (5 mL) was allowed to stir at 100° C. for 3 hr under N₂ atmosphere. The reaction mixture was filtered and the filtrate was concentrated under reduced pressure. The crude was purified by flash chromatography (ISCO®; 4 g SepaFlash® Silica Flash Column, petroleum ether/EtOAc with EtOAc from 0˜100%, 12 mL/min, 254 nm) to give 2-[[8-(2,6-difluorophenyl)-5-methyl-13-morpholino-3,4,7,9,12-pentazatricyclo[8.4.0.02,6]tetradeca-1(10),2(6),4,7,11,13-hexaen-3-yl]methoxy]ethyl-trimethyl-silane as a red oil (70 mg, 27.7%). LCMS (ESI) m/z: 527.2 [M+H]⁺.

### Step 2: 4-[8-(2,6-difluorophenyl)-5-methyl-3,4,7,9,12-pentazatricyclo[8.4.0.02,6]tetradeca-1(10),2(6),4,7,11,13-hexaen-13-yl]morpholine

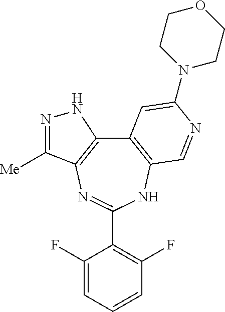 [SMILES: Cc1n[nH]c2c1N=C(c1c(F)cccc1F)Nc1cnc(N3CCOCC3)cc1-2]

A solution of 2-[[8-(2,6-difluorophenyl)-5-methyl-13-morpholino-3,4,7,9,12-pentazatricyclo[8.4.0.02,6]tetradeca-1(10),2(6),4,7,11,13-hexaen-3-yl]methoxy]ethyl-trimethyl-silane (70 mg, 0.133 mmol) in 4N HCl in MeOH (2 mL) was allowed to stir at 25° C. for 2 hr. The resulting mixture was cooled to 0° C. The precipitate was collected by filtration to provide the tittle compound as a yellow solid (30 mg, 54.1%). ¹H NMR (400 MHz, CD₃OD) δ ppm 2.26 (s, 3H), 3.58-3.68 (m, 4H), 3.77-3.87 (m, 4H), 7.31 (t, J=8.6 Hz, 2H), 7.47 (s, 1H), 7.63 (s, 1H), 7.76-7.89 (m, 1H); LCMS (ESI) m/z: 397.2 [M+H]⁺.

### Example Int-2: Synthesis of 9-[1-(difluoromethyl)pyrazol-3-yl]-5-(2,6-difluorophenyl)-3-methyl-1,6-dihydropyrazolo[4,3-d][1,3]benzodiazepine

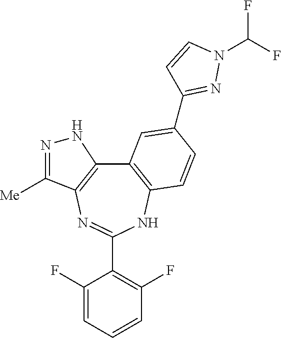 [SMILES: Cc1n[nH]c2c1N=C(c1c(F)cccc1F)Nc1ccc(-c3ccn(C(F)F)n3)cc1-2]

To a solution of 2-[[9-bromo-5-(2,6-difluorophenyl)-3-methyl-6H-pyrazolo[4,3-d][1,3]benzodiazepin-1-yl]methoxy]ethyl-trimethyl-silane (Intermediate D1-3) (200 mg, 0.385 mmol), 1-(difluoromethyl)-3-(4,4,5,5-tetramethyl-1,3,2-dioxaborolan-2-yl)pyrazole (400 mg, 1.64 mmol), K₃PO₄ (160 mg, 0.754 mmol) and XPhos (20.0 mg, 42.0 μmol) in dioxane (10 mL) and H₂O (2 mL) was added Pd₂(dba)₃ (30.0 mg, 52.2 μmol). The mixture was allowed to stir at 100° C. for 12 hr. The residue was purified by flash chromatography (ISCO@; 20 g SepaFlash@ Silica Flash Column, petroleum ether/EtOAc with EtOAc from 0˜20%, flow rate: 30 mL/min, 254 nm) to afford 2-[[9-[1-(difluoromethyl)pyrazol-3-yl]-5-(2,6-difluorophenyl)-3-methyl-6H-pyrazolo[4,3-d][1,3]benzodiazepin-1-yl]methoxy]ethyl-trimethyl-silane (250 mg, 51.3%) as a yellow solid. The resulting product was treated with 4.0 N HCl in MeOH to remove SEM protecting group and purified by reverse phase preparative HPLC (Instrument: Gilson GX-281 Liquid Handler, Gilson 322 Pump, Gilson 156 UV Detector; Column: column: ACE 5 C18-AR 150, 30 mm, 5 m; mobile phase: [water(FA)-ACN]; B %: 35%-65%, 7.8 min, hold 100% B for 2 min; Flow Rate: 25 mL/min; Column Temperature: 30° C.; Wavelength: 220 nm) to provide 9-[1-(Difluoromethyl)pyrazol-3-yl]-5-(2,6-difluorophenyl)-3-methyl-1,6-dihydropyrazolo[4,3-d][1,3]benzodiazepine as a yellow solid (54.4 mg, 31.6%). ¹H NMR (400 MHz, DMSO-d₆) δ ppm 1.96 (s, 3H), 6.62 (d, J=8.25 Hz, 1H), 6.87 (d, J=2.63 Hz, 1H), 7.17 (br t, J=7.94 Hz, 2H), 7.40 (dd, J=8.25, 1.88 Hz, 1H), 7.50 (quin, J=7.47 Hz, 1H), 7.76 (m, 1H), 7.96 (s, 1H), 8.23 (d, J=2.63 Hz, 1H), 8.36 (br s, 1H), 12.33 (br s, 1H); ¹⁹F NMR (377 MHz, DMSO-d₆) δ ppm −94.03, −114.06; LCMS (ESI) m/z: 427.0 [M+H]⁺.

### Example Int-3: Synthesis of 8-(2,6-difluorophenyl)-5-methyl-13-[2-(4-methyltetrahydropyran-4-yl)ethynyl]-3,4,7,9,12-pentazatricyclo[8.4.0.02,6]tetradeca-1(10),2(6),4,7,11,13-hexaene

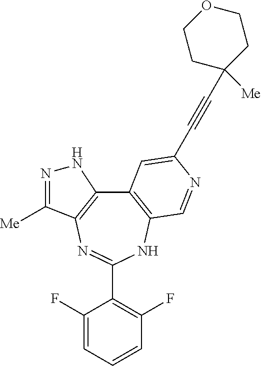 [SMILES: Cc1n[nH]c2c1N=C(c1c(F)cccc1F)Nc1cnc(C#CC3(C)CCOCC3)cc1-2]

### Step 1: 2-[[8-(2,6-difluorophenyl)-5-methyl-13-[2-(4-methyltetrahydropyran-4-yl)ethynyl]-3,4,7,9,12-pentazatricyclo[8.4.0.02,6]tetradeca-1(10),2(6),4,7,11,13-hexaen-3-yl]methoxy]ethyl-trimethyl-silane

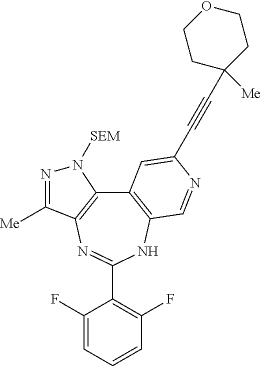 [SMILES: Cc1nn(COCC[Si](C)(C)C)c2c1N=C(c1c(F)cccc1F)Nc1cnc(C#CC3(C)CCOCC3)cc1-2]

A mixture of 2-[[13-chloro-8-(2,6-difluorophenyl)-5-methyl-3,4,7,9,12-pentazatricyclo[8.4.0.02,6] tetradeca-1(10),2(6),4,7,11,13-hexaen-3-yl]methoxy]ethyl-trimethyl-silane (50 mg, 105 umol, 1.0 eq), 4-ethynyl-4-methyl-tetrahydropyran (13.0 mg, 0.105 mmol, 1.0 eq), CuI (4.00 mg, 21.0 μmol, 0.05 eq), Pd(t-Bu₃P)₂ (10.7 mg, 21.0 μmol, 0.05 eq) and Cs₂CO₃ (102 mg, 0.315 mmol, 3.0 eq) in DMF (2 mL) was degassed and purged with N₂ for 3 times, and then stirred at 100° C. for 16 h under N₂ atmosphere. The reaction mixture was then filtered and the filtrate was concentrated under reduced pressure. The crude was purified by flash chromatography (ISCO®; 4 g SepaFlash® Silica Flash Column, petroleum ether/EtOAc with EtOAc from 0˜100%, 12 mL/min, 254 mn) to provide 2-[[8-(2,6-difluorophenyl)-5-methyl-13-[2-(4-methyltetrahydropyran-4-yl)ethynyl]-3,4,7,9,12-pentazatricyclo[8.4.0.02,6]tetradeca-1(10),2(6),4,7,11,13-hexaen-3-yl]methoxy]ethyl-trimethyl-silane as a brown solid (50 mg). LCMS (ESI) m/z: 564.2 [M+H]⁺.

### Step 2: 8-(2,6-difluorophenyl)-5-methyl-13-[2-(4-methyltetrahydropyran-4-yl)ethynyl]-3,4,7,9,12-pentazatricyclo[8.4.0.02,6]tetradeca-1(10),2(6),4,7,11,13-hexaene

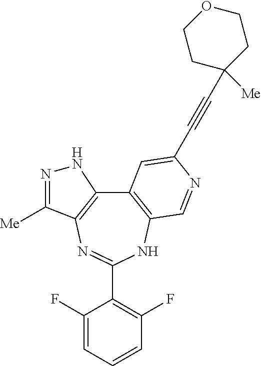 [SMILES: Cc1n[nH]c2c1N=C(c1c(F)cccc1F)Nc1cnc(C#CC3(C)CCOCC3)cc1-2]

A mixture of 2-[[8-(2,6-difluorophenyl)-5-methyl-13-[2-(4-methyltetrahydropyran-4-yl)ethynyl]-3,4,7,9,12-pentazatricyclo[8.4.0.02,6]tetradeca-1(10),2(6),4,7,11,13-hexaen-3-yl]methoxy]ethyl-trimethyl-silane (50 mg, 88.7 μmol) in 4.0 N HCl in MeOH (2 mL) was allowed to stir at 25° C. for 2 h. The reaction was concentrated under reduced pressure to provide the tittle compound as a yellow solid (9.4 mg, 24.4%). ¹H NMR (400 MHz, CD₃OD) δ ppm 1.41 (s, 3H), 1.65-1.73 (m, 2H), 1.78-1.85 (m, 2H), 2.24 (s, 3H), 3.76-3.83 (m, 2H), 3.87-3.93 (m, 2H), 7.33 (t, J=8.5 Hz, 2H), 7.78-7.89 (m, 2H), 7.95 (s, 1H); LCMS (ESI) m/z: 434.5 [M+H]⁺.

### Example Int-4 and Int-5: Synthesis of (R)-5-(2,6-difluorophenyl)-9-(4-(2-fluoroethyl)-2-methylpiperazin-1-yl)-3,7-dimethyl-1,6-dihydropyrazolo[4,3-d]pyrido[4,3-f][1,3]diazepine (Int-4) and (S)-5-(2,6-difluorophenyl)-9-(4-(2-fluoroethyl)-2-methylpiperazin-1-yl)-3,7-dimethyl-1,6-dihydropyrazolo[4,3-d]pyrido[4,3-f][1,3]diazepine (Int-5)

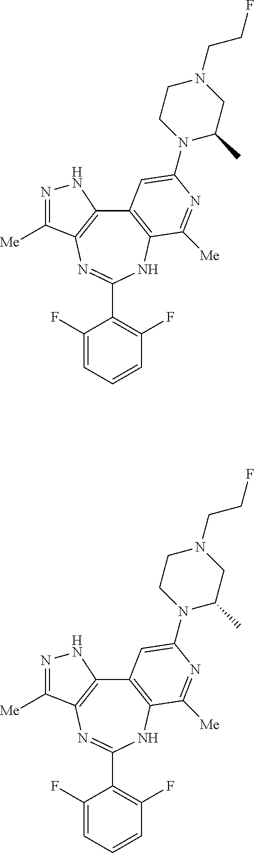 [SMILES: Cc1n[nH]c2c1N=C(c1c(F)cccc1F)Nc1c-2cc(N2CCN(CCF)C[C@H]2C)nc1C ; Cc1n[nH]c2c1N=C(c1c(F)cccc1F)Nc1c-2cc(N2CCN(CCF)C[C@@H]2C)nc1C]

### Step 1: 01-benzyl 04-tert-butyl 2-methylpiperazine-1,4-dicarboxylate

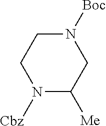 [SMILES: CC1CN(C(=O)OC(C)(C)C)CCN1C(=O)OCc1ccccc1]

To a solution of tert-butyl 3-methylpiperazine-1-carboxylate (5 g, 24.9 mmol) in THF (50 mL) was added CbzCl (5.11 g, 29.9 mmol) and Cs₂CO₃ (24.4 g, 74.9 mmol). The mixture was allowed to stir at 25° C. for 2 hr. The reaction mixture was diluted with H₂O (30 mL) and extracted with EA (30 mL×3). The combined organic layers were washed with brine (30 mL×2), dried with anhydrous Na₂SO₄, filtered and concentrated under reduced pressure. The residue was purified by flash silica gel chromatography (ISCO®; 40 g SepaFlash® Silica Flash Column, Eluent of 0˜40% petroleum ether/EtOAc @ 45 mL/min) to give 1-benzyl 4-tert-butyl 2-methylpiperazine-1,4-dicarboxylate (2.6 g, 30.5%, 98% purity) as a colorless oil. LCMS (ESI) m/z: 335.2 [M+H]⁺.

### Step 2: benzyl 2-methylpiperazine-1-carboxylate

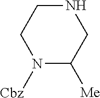 [SMILES: CC1CNCCN1C(=O)OCc1ccccc1]

To a solution of 1-benzyl 4-tert-butyl 2-methylpiperazine-1,4-dicarboxylate (2.6 g, 7.77 mmol) in 4.0 N HCl in MeOH (10 mL). The mixture was allowed to stir at 25° C. for 1 hr. The reaction mixture was concentrated under reduced pressure to give benzyl 2-methylpiperazine-1-carboxylate (2 g, crude) as a white solid.

### Step 3: benzyl 4-(2-fluoroethyl)-2-methyl-piperazine-1-carboxylate

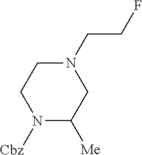 [SMILES: CC1CN(CCF)CCN1C(=O)OCc1ccccc1]

To a solution of benzyl 2-methylpiperazine-1-carboxylate (2 g, 8.54 mmol) and 1-fluoro-2-iodo-ethane (1.63 g, 9.39 mmol) in DMF (26 mL) was added Cs₂CO₃ (8.34 g, 25.6 mmol). The mixture was allowed to stir at 90° C. for 16 hr. The residue was diluted with H₂O (50 mL) and extracted with EtOAc (50 mL×3). The combined organic layers were washed with brine (50 mL), dried over Na₂SO₄, filtered and concentrated under reduced pressure. The residue was purified by flash silica gel chromatography (ISCO®; 12 g SepaFlash® Silica Flash Column, Eluent of 0˜30% Ethyl acetate/Petroleum ether gradient @ 30 mL/min) to give benzyl 4-(2-fluoroethyl)-2-methyl-piperazine-1-carboxylate (1.9 g, 45.3%, 57% purity) as a white oil. LCMS (ESI) m/z: 281.1 [M+H]⁺.

### Step 4: 1-(2-fluoroethyl)-3-methyl-piperazine

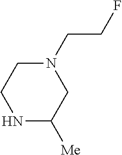

To a solution of benzyl 4-(2-fluoroethyl)-2-methyl-piperazine-1-carboxylate (1.9 g, 6.78 mmol) in MeOH (25 mL) was added Pd/C (0.5 g, 10 wt % Pd with 50 wt % water). The suspension was degassed and purged with H₂ for 3 times. The mixture was allowed to stir under H₂ (15 psi) at 20° C. for 1 hr, filtered and concentrated under reduced pressure to give 1-(2-fluoroethyl)-3-methyl-piperazine (1 g, crude) as a white oil.

### Step 5: 2-[[8-(2,6-difluorophenyl)-13-[4-(2-fluoroethyl)-2-methyl-piperazin-1-yl]-5,11-dimethyl-3,4,7,9,12-pentazatricyclo[8.4.0.0^{2,6}]tetradeca-1(10),2(6),4,7,11,13-hexaen-3-yl]methoxy]ethyl-trimethyl-silane

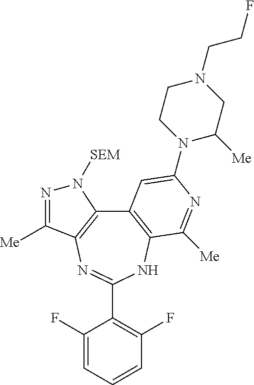 [SMILES: Cc1nn(COCC[Si](C)(C)C)c2c1N=C(c1c(F)cccc1F)Nc1c-2cc(N2CCN(CCF)CC2C)nc1C]

A mixture of 1-(2-fluoroethyl)-3-methyl-piperazine (298 mg, 1.22 mmol), 2-[[13-chloro-8-(2,6-difluorophenyl)-5,11-dimethyl-3,4,7,9,12-pentazatricyclo[8.4.0.0^{2,6}]tetradeca-1(10),2(6),4,7,11,13-hexaen-3-yl]methoxy]ethyl-trimethyl-silane (Intermediate D4-1) (500 mg, 1.02 mmol), RuPhos Pd G3 (427 mg, 0.510 mmol), t-BuONa (294 mg, 3.06 mmol) in dioxane (5 mL) was degassed and purged with N₂ for 3 times, and stirred at 110° C. for 1 hr under N₂ atmosphere. The reaction mixture was concentrated under reduced pressure. The residue was purified by flash silica gel chromatography (ISCO®; 12 g SepaFlash® Silica Flash Column, Eluent of 0˜30% Ethyl acetate/Petroleum ether gradient @30 mL/min) to give 2-[[8-(2,6-difluorophenyl)-13-[4-(2-fluoroethyl)-2-methyl-piperazin-1-yl]-5,11-dimethyl-3,4,7,9,12-pentazatricyclo[8.4.0.0^{2,6}]tetradeca-1(10),2(6),4,7,11,13-hexaen-3-yl]methoxy]ethyl-trimethyl-silane (180 mg, 19.7%, 67% purity) as a yellow solid. LCMS (ESI) m/z: 600.2 [M+H]⁺.

### Step 6: 8-(2,6-difluorophenyl)-13-[4-(2-fluoroethyl)-2-methyl-piperazin-1-yl]-5,11-dimethyl-3,4,7,9,12-pentazatricyclo[8.4.0.0^{2,6}]tetradeca-1(10),2(6),4,7,11,13-hexaene

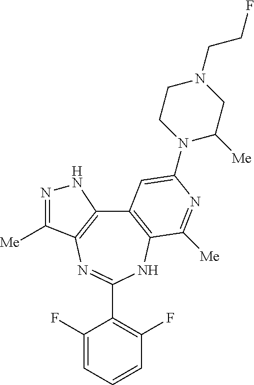 [SMILES: Cc1n[nH]c2c1N=C(c1c(F)cccc1F)Nc1c-2cc(N2CCN(CCF)CC2C)nc1C]

To a solution of 2-[[8-(2,6-difluorophenyl)-13-[4-(2-fluoroethyl)-2-methyl-piperazin-1-yl]-5,11-dimethyl-3,4,7,9,12-pentazatricyclo[8.4.0.0^{2,6}]tetradeca-1(10),2(6),4,7,11,13-hexaen-3-yl]methoxy]ethyl-trimethyl-silane (170 mg, 0.283 mmol) in 4.0 N HCl in MeOH (2 mL) was added Et₃SiH (32.9 mg, 0.283 mmol). The mixture was allowed to stir at 25° C. for 1 h. The reaction mixture was concentrated under reduced pressure. The residue was purified by flash silica gel chromatography (ISCO®; 12 g SepaFlash® Silica Flash Column, Eluent of 0˜30% Ethyl acetate/Petroleum ether gradient @ 30 mL/min) to give 8-(2,6-difluorophenyl)-13-[4-(2-fluoroethyl)-2-methyl-piperazin-1-yl]-5,11-dimethyl-3,4,7,9,12-pentazatricyclo[8.4.0.0^{2,6}]tetradeca-1(10),2(6),4,7,11,13-hexaene (50 mg, 34.2%, 91% purity) as a yellow solid. LCMS (ESI) m/z: 470.2 [M+H]⁺.

### Step 7: (R)-5-(2,6-difluorophenyl)-9-(4-(2-fluoroethyl)-2-methylpiperazin-1-yl)-3,7-dimethyl-1,6-dihydropyrazolo[4,3-d]pyrido[4,3-f][1,3]diazepine (Int-4) And (S)-5-(2,6-difluorophenyl)-9-(4-(2-fluoroethyl)-2-methylpiperazin-1-yl)-3,7-dimethyl-1,6-dihydropyrazolo[4,3-d]pyrido[4,3-f][1,3]diazepine (Int-5)

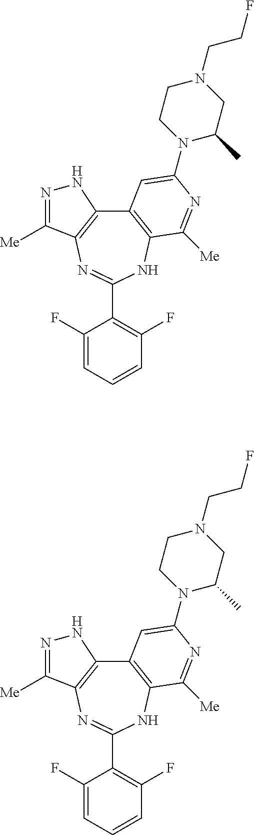 [SMILES: Cc1n[nH]c2c1N=C(c1c(F)cccc1F)Nc1c-2cc(N2CCN(CCF)C[C@H]2C)nc1C ; Cc1n[nH]c2c1N=C(c1c(F)cccc1F)Nc1c-2cc(N2CCN(CCF)C[C@@H]2C)nc1C]

The racemate was dissolved in DMSO and re-subjected to chiral preparative HPLC column: DAICEL CHIRALCEL OJ (250 mm×30 mm×10 um); mobile phase: [0.1% NH₃H₂O ETOH]; B %: 40%-40%, min) to provide the two fractions, which after removal of solvents, provided the following two products respectively:

Fraction A (14 mg, RT=2.87 min) was tentatively assigned as compound Int-4: ¹H NMR (400 MHz, CD₃OD) δ ppm 1.23 (d, J=6.56 Hz, 3H), 2.14-2.22 (m, 3H), 2.23-2.31 (m, 4H), 2.38-2.46 (m, 1H), 2.61-2.82 (m, 2H), 2.87-2.95 (m, 1H), 2.97-3.05 (m, 1H), 3.11-3.20 (m, 1H), 3.84-3.94 (m, 1H), 4.42 (br s, 1H), 4.56 (br d, J=5.60 Hz, 1H), 4.67-4.69 (m, 1H), 6.73-6.87 (m, 1H), 7.06-7.16 (m, 2H), 7.46-7.56 (m, 1H). LCMS (ESI) m/z: 470.2 [M+H]⁺.

Fraction B (14 mg, RT=3.58 min) was tentatively assigned as compound Int-5: ¹H NMR (400 MHz, CD₃OD) δ ppm 1.23 (d, J=8 Hz, 3H), 2.18 (br s, 3H), 2.26 (s, 4H), 2.39-2.46 (m, 1H), 2.62-2.82 (m, 2H), 2.91 (br d, J=12 Hz, 1H), 3.00 (br d, J=12 Hz, 1H), 3.15 (br d, J=4 Hz, 1H), 3.84-3.94 (m, 1H), 4.38-4.47 (m, 1H), 4.56 (dt, J=4, 3.78 Hz, 1H), 4.67-4.70 (m, 1H), 6.81 (br s, 1H), 7.10 (br t, J=8 Hz, 2H), 7.46-7.57 (m, 1H). ¹⁹F NMR (376 MHz, CD₃OD) δ ppm. LCMS (ESI) m/z: 470.2 [M+H]⁺.

### Example Int-6: Synthesis of 4-[8-(2-chloro-6-fluoro-phenyl)-3,4,7,9,12-pentazatricyclo[8.4.0.02,6]tetradeca-1(10),2(6),4,7,11,13-hexaen-13-yl]morpholine

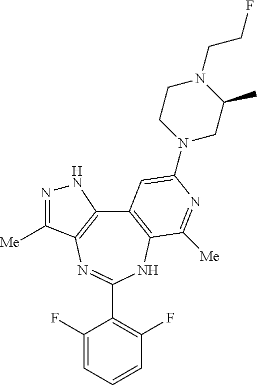 [SMILES: Cc1n[nH]c2c1N=C(c1c(F)cccc1F)Nc1c-2cc(N2CCN(CCF)[C@@H](C)C2)nc1C]

### Step 1: tert-butyl 4-(2-fluoroethyl)-3-methyl-piperazine-1-carboxylate

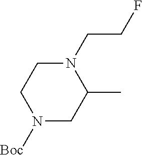 [SMILES: CC1CN(C(=O)OC(C)(C)C)CCN1CCF]

To a solution of tert-butyl 3-methylpiperazine-1-carboxylate (2 g, 9.99 mmol) and 1-bromo-2-fluoro-ethane (1.39 g, 10.9 mmol) in DMF (20 mL) was added Cs₂CO₃ (9.76 g, 29.9 mmol). The mixture was allowed to stir at 90° C. for 16 h. The reaction mixture was diluted with H₂O (30 mL) and extracted with EA (30 mL×3). The combined organic layers were washed with brine (30 mL×2), dried with anhydrous Na₂SO₄, filtered and concentrated under reduced pressure. The residue was purified by flash silica gel chromatography (ISCO®; 20 g SepaFlash® Silica Flash Column, Eluent of 0˜40% petroleum ether/EtOAc @ 45 mL/min) to give tert-butyl 4-(2-fluoroethyl)-3-methyl-piperazine-1-carboxylate as a white oil (1 g, 36.6%).

### Step 2: 1-(2-fluoroethyl)-2-methyl-piperazine

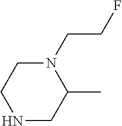

A mixture of tert-butyl 4-(2-fluoroethyl)-3-methyl-piperazine-1-carboxylate (1 g, 4.06 mmol) in 4N HCl in MeOH (10 mL) was stirred at 25° C. for 1 h. The reaction mixture was concentrated under reduced pressure to give 1-(2-fluoroethyl)-2-methyl-piperazine as a white oil (1 g, crude).

### Step 3: 2-[[8-(2,6-difluorophenyl)-13-[4-(2-fluoroethyl)-3-methyl-piperazin-1-yl]-5,11-dimethyl-3,4,7,9,12-pentazatricyclo[8.4.0.0^{2,6}]tetradeca-1(10),2(6),4,7,11,13-hexaen-3-yl]methoxy]ethyl-trimethyl-silane

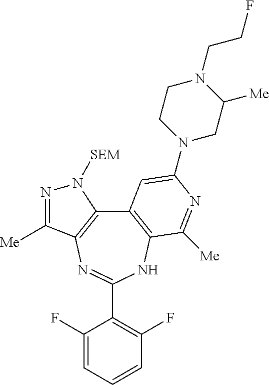 [SMILES: Cc1nn(COCC[Si](C)(C)C)c2c1N=C(c1c(F)cccc1F)Nc1c-2cc(N2CCN(CCF)C(C)C2)nc1C]

A mixture of 1-(2-fluoroethyl)-2-methyl-piperazine (298 mg, 1.22 mmol), 2-[[13-chloro-8-(2,6-difluorophenyl)-5,11-dimethyl-3,4,7,9,12-pentazatricyclo[8.4.0.0^{2,6}]tetradeca-1(10),2(6),4,7,11,13-hexaen-3-yl]methoxy]ethyl-trimethyl-silane (500 mg, 1.02 mmol), SPhos (168 mg, 0.408 mmol,), Pd₂(dba)₃ (187 mg, 0.204 mmol) and t-BuONa (588 mg, 6.12 mmol) in dioxane (5 mL) was degassed and purged with N₂ for 3 times, and then stirred at 110° C. for 1 h under N₂ atmosphere. The reaction mixture was filtered and concentrated under reduced pressure. The residue was purified by flash silica gel chromatography (ISCO®; 12 g SepaFlash® Silica Flash Column, Eluent of 0˜30% Ethyl acetate/Petroleum ether gradient @30 mL/min) to give 2-[[8-(2,6-difluorophenyl)-13-[4-(2-fluoroethyl)-3-methyl-piperazin-1-yl]-5,11-dimethyl-3,4,7,9,12-pentazatricyclo[8.4.0.0^{2,6}]tetradeca-1(10),2(6),4,7,11,13-hexaen-3-yl]methoxy]ethyl-trimethyl-silane as a yellow solid (350 mg, 50.3%). LCMS (ESI) m/z: 600.2 [M+H]⁺.

### Step 4: 8-(2,6-difluorophenyl)-13-[4-(2-fluoroethyl)-3-methyl-piperazin-1-yl]-5,11-dimethyl-3,4,7,9,12-pentazatricyclo[8.4.0.0^{2,6}]tetradeca-1(10),2(6),4,7,11,13-hexaene

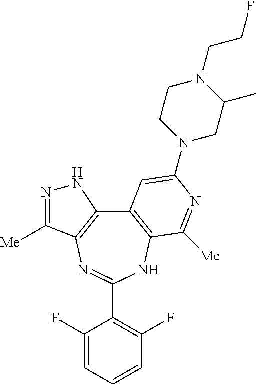 [SMILES: Cc1n[nH]c2c1N=C(c1c(F)cccc1F)Nc1c-2cc(N2CCN(CCF)C(C)C2)nc1C]

To a solution of 2-[[8-(2,6-difluorophenyl)-13-[4-(2-fluoroethyl)-3-methyl-piperazin-1-yl]-5,11-dimethyl-3,4,7,9,12-pentazatricyclo[8.4.0.0^{2,6}]tetradeca-1(10),2(6),4,7,11,13-hexaen-3-yl]methoxy]ethyl-trimethyl-silane (350 mg, 0.584 umol) in 4N HCl in MeOH (4 mL) was added Et₃SiH (67.9 mg, 0.584 mmol). The mixture was allowed to stir at 25° C. for 1 hr, diluted with H₂O (50 mL) and extracted with EtOAc (50 mL×3). The combined organic layers were washed with brine (50 mL), dried over Na₂SO₄, filtered and concentrated under reduced pressure. The residue was purified by flash silica gel chromatography (ISCO®; 12 g SepaFlash® Silica Flash Column, Eluent of 0˜30% Ethyl acetate/petroleum ether gradient @ 30 mL/min) to give 8-(2,6-difluorophenyl)-13-[4-(2-fluoroethyl)-3-methyl-piperazin-1-yl]-5,11-dimethyl-3,4,7,9,12-pentazatricyclo[8.4.0.0^{2,6}]tetradeca-1(10),2(6),4,7,11,13-hexaene (130 mg, 45.1%). LCMS (ESI) m/z: 470.2 [M+H]⁺.

### Step 5: 8-(2,6-difluorophenyl)-13-[(3S)-4-(2-fluoroethyl)-3-methyl-piperazin-1-yl]-5,11-dimethyl-3,4,7,9,12-pentazatricyclo[8.4.0.0^{2,6}]tetradeca-1(10),2(6),4,7,11,13-hexaene

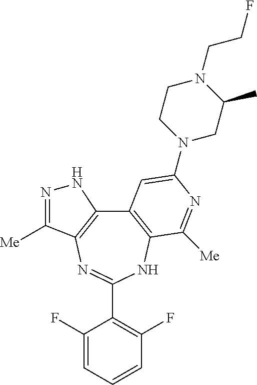 [SMILES: Cc1n[nH]c2c1N=C(c1c(F)cccc1F)Nc1c-2cc(N2CCN(CCF)[C@@H](C)C2)nc1C]

8-(2,6-Difluorophenyl)-13-[4-(2-fluoroethyl)-3-methyl-piperazin-1-yl]-5,11-dimethyl-3,4,7,9,12-pentazatricyclo[8.4.0.0^{2,6}]tetradeca-1(10),2(6),4,7,11,13-hexaene (130 mg, 0.277 mmol). The residue was purified by preparative HPLC to give 8-(2,6-difluorophenyl)-13-[(3S)-4-(2-fluoroethyl)-3-methyl-piperazin-1-yl]-5,11-dimethyl-3,4,7,9,12-pentazatricyclo [8.4.0.0^{2,6}]tetradeca-1(10),2(6),4,7,11,13-hexaene was obtained as a yellow solid. ¹H NMR (400 MHz, CD₃OD) δ ppm 1.17-1.20 (m, 3H), 2.12-2.21 (m, 3H), 2.23-2.30 (m, 3H), 2.49-2.64 (m, 1H), 2.51-2.66 (m, 1H), 2.70 (br dd, J=9.4, 3.4 Hz, 2H), 3.03-3.11 (m, 2H), 3.12-3.25 (m, 1H), 3.33 (s, 14H), 3.90-4.00 (m, 2H), 4.55-4.60 (m, 1H), 4.67-4.71 (m, 1H), 4.91 (s, 15H), 4.98-5.00 (m, 1H), 6.86 (s, 1H), 7.05-7.15 (m, 2H), 7.46-7.57 (m, 1H); LCMS (ESI) m/z: 470.2 [M+H]⁺.

### Example Int-7: Synthesis of 4-[5-cyclopropyl-8-(2,6-difluorophenyl)-3,4,7,9,12-pentazatricyclo[8.4.0.0^{2,6}]tetradeca-1(10),2(6),4,7,11,13-hexaen-13-yl]morpholine

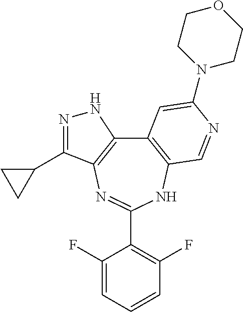 [SMILES: Fc1cccc(F)c1C1=Nc2c(C3CC3)n[nH]c2-c2cc(N3CCOCC3)ncc2N1]

### Step 1: Synthesis of 2-[[5-cyclopropyl-8-(2,6-difluorophenyl)-13-morpholino-3,4,7,9,12-pentazatricyclo[8.4.0.0^{2,6}]tetradeca-1(10),2(6),4,7,11,13-hexaen-3-yl]methoxy]ethyl-trimethyl-silane

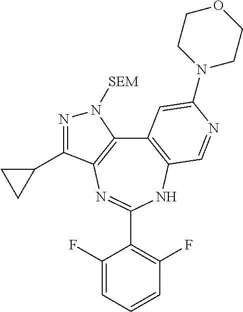 [SMILES: C[Si](C)(C)CCOCn1nc(C2CC2)c2c1-c1cc(N3CCOCC3)ncc1NC(c1c(F)cccc1F)=N2]

A mixture of 2-[[5-chloro-8-(2,6-difluorophenyl)-13-morpholino-3,4,7,9,12-pentazatricyclo[8.4.0.0^{2,6}]tetradeca-1(10),2(6),4,7,11,13-hexaen-3-yl]methoxy]ethyl-trimethyl-silane (Example 23 (described below), 150 mg, 0.213 mmol, 1 eq.), cyclopropylboronic acid (22.0 mg, 0.256 mmol, 1.2 eq.), Cs₂CO₃ (209 mg, 0.641 mmol, 3 eq.), BrettPhos-Pd-G₃ (39.4 mg, 42.7 μmol, 0.2 eq.) in dioxane/H₂O (2:1, 1.5 mL) was degassed and purged with N₂ for 3 times, and then stirred at 100° C. for 16 h under N₂ atmosphere. The reaction mixture was diluted with H₂O (10 mL) and extracted with EtOAc (10 mL×3). The combined organic phase was washed with brine (10 mL×2), dried with anhydrous Na₂SO₄, filtered and concentrated under reduced pressure. The residue was purified by flash chromatography on silica gel column using 0-20% of EtOAc in petroleum ether as eluent, to provide 2-[[5-cyclopropyl-8-(2,6-difluorophenyl)-13-morpholino-3,4,7,9,12-pentazatricyclo [8.4.0.0^{2,6}]tetradeca-1(10),2(6),4,7,11,13-hexaen-3-yl]methoxy]ethyl-trimethyl-silane as a yellow oil (50 mg, 42.3%).

### Step 2: Synthesis of 4-[5-cyclopropyl-8-(2,6-difluorophenyl)-3,4,7,9,12-pentazatricyclo [8.4.0.0^{2,6}]tetradeca-1(10),2(6),4,7,11,13-hexaen-13-yl]morpholine

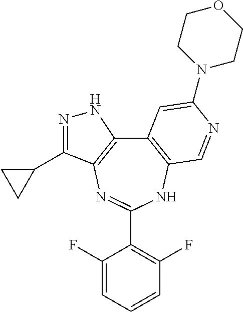 [SMILES: Fc1cccc(F)c1C1=Nc2c(C3CC3)n[nH]c2-c2cc(N3CCOCC3)ncc2N1]

A mixture of 2-[[5-cyclopropyl-8-(2,6-difluorophenyl)-13-morpholino-3,4,7,9,12-pentazatricyclo [8.4.0.0^{2,6}]tetradeca-1(10),2(6),4,7,11,13-hexaen-3-yl]methoxy]ethyl-trimethyl-silane (50 mg, 90.4 μmol, 1 eq.), Et₃SiH (28.9 μL, 0.180 mmol, 2 eq.) in 4N HCl in MeOH (1.0 mL) was allowed to stir at 25° C. for 1 h under N₂ atmosphere. The reaction mixture was concentrated under reduced pressure, diluted with H₂O (2 mL) and quenched by sat. NaHCO₃ aqueous solution to adjust pH to 7 and extracted with EtOAc (10 mL×3). The combined organic phase was washed with brine (10 mL×3), dried with anhydrous Na₂SO₄, filtered and concentrated under reduced pressure. The residue was purified by flash chromatography on silica gel column using 0-50% of EtOAc in petroleum ether as eluent, to provide 4-[5-cyclopropyl-8-(2,6-difluorophenyl)-3,4,7,9,12-pentazatricyclo[8.4.0.0^{2,6}] tetradeca-1(10),2(6),4,7,11,13-hexaen-13-yl]morpholine as a yellow solid (20.2 mg, 52.3%). ¹H NMR (400 MHz, DMSO-d₆) δ ppm 0.87-0.78 (m, 4H), 1.77-1.67 (m, 1H), 3.28-3.24 (m, 4H), 3.69-3.64 (m, 4H), 6.81 (s, 1H), 7.16 (t, J=8.0 Hz, 2H), 7.44 (s, 1H), 7.52-7.45 (m, 1H), 8.24 (s, 1H), 12.25 (s, 1H); ¹⁹F NMR (377 MHz, DMSO-d₆) δ ppm −114.00; LCMS (ESI) m/z: 423.1 [M+H]⁺.

### Example 1

### 8-(2,6-difluorophenyl)-13-[4-(2-fluoroethyl)piperazin-1-yl]-5-methyl-3,4,7,9,12-pentazatricyclo[8.4.0.02,6]tetradeca-1(10),2(6),4,7,11,13-hexaene

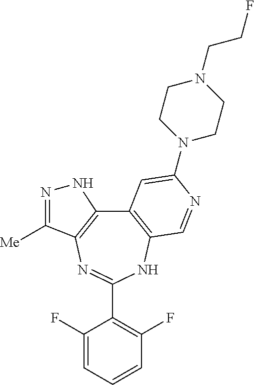 [SMILES: Cc1n[nH]c2c1N=C(c1c(F)cccc1F)Nc1cnc(N3CCN(CCF)CC3)cc1-2]

The title compound was made from intermediate D2-3, following a similar synthetic procedure as described in the synthesis of Int-1 (described above), except that 1-(2-fluoroethyl)piperazine was used in the place of morpholine, and 10% Pd₂(dba)₃ was employed as the catalyst, and the reaction was conducted in dioxane at 100° C. for 12 hr. The obtained crude product was treated with 4N HCl in MeOH to remove the SEM protecting group. The resulting mixture was concentrated under reduced pressure and purified by reverse phase preparative HPLC (Instrument: Gilson GX-281 Liquid Handler, Gilson 322 Pump, Gilson 156 UV Detector; column: ACE 5 C18-AR 150×30 mm×5 m; mobile phase: [water(FA)-ACN]; B %: 19%-49%, 9.5-min, hold 100% B for 2 min; Flow Rate: 25 mL/min; Column Temperature: 30° C.; Wavelength: 220 nm) to provide the title compound as a colorless solid (25.7 mg, 66.6%). ¹H NMR (400 MHz, DMSO-d₆) δ ppm 2.11-2.19 (m, 3H), 3.48-3.63 (m, 8H), 4.27-4.41 (m, 2H), 4.81-5.01 (m, 2H), 7.12 (s, 1H), 7.30-7.44 (m, 2H), 7.70-7.91 (m, 2H); LCMS (ESI) m/z: 442.2 [M+H]⁺.

### Example 2

### 4-[8-(2,6-difluorophenyl)-5-methyl-3,4,7,9,12-pentazatricyclo[8.4.0.02,6]tetradeca-1(10),2(6),4,7,11,13-hexaen-13-yl]-1,4-thiazinane 1,1-dioxide

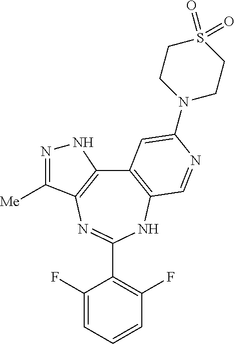 [SMILES: Cc1n[nH]c2c1N=C(c1c(F)cccc1F)Nc1cnc(N3CCS(=O)(=O)CC3)cc1-2]

The title compound was made from intermediate D2-3, following a similar synthetic procedure as described in the synthesis of Int-1 (described above), except that 1,4-thiazinane 1,1-dioxide was used in the place of morpholine, and 10% Pd₂(dba)₃ was employed as the catalyst, and the reaction was conducted in dioxane at 100° C. for 12 hr. The obtained crude product was treated with 4N HCl in MeOH to remove the SEM protecting group. The resulting mixture was concentrated under reduced pressure and purified by reverse phase preparative HPLC (Instrument: Gilson GX-281 Liquid Handler, Gilson 322 Pump, Gilson 156 UV Detector; column: ACE 5 C18-AR 150×30 mm×5 m; mobile phase: [water(FA)-ACN]; B %: 19%-49%, 9.5 min, hold 100% B for 2 min; Flow Rate: 25 mL/min; Column Temperature: 30° C.; Wavelength: 220 nm) to provide the title compound as a yellow solid (28 mg, 30.7%). ¹H NMR (400 MHz, DMSO-d₆) δ ppm 2.15 (br s, 3H), 3.13 (br s, 4H), 4.02 (br s, 3H), 3.97-4.06 (m, 1H), 7.15 (s, 1H), 7.28-7.46 (m, 2H), 7.77 (br s, 2H); LCMS (ESI) m/z: 444.4 [M+H]⁺.

### Example 3 and Example 4

### (5S)-9-[8-(2,6-difluorophenyl)-5-methyl-3,4,7,9,12-pentazatricyclo [8.4.0.02,6]tetradeca-1(10),2(6),4,7,11,13-hexaen-13-yl]-2,6-dioxa-9-azaspiro[4.5]decane (Example 3) and (5R)-9-[8-(2,6-difluorophenyl)-5-methyl-3,4,7,9,12-pentazatricyclo[8.4.0.02,6] tetradeca-1(10),2(6),4,7,11,13-hexaen-13-yl]-2,6-dioxa-9-azaspiro[4.5]decane (Example 4)

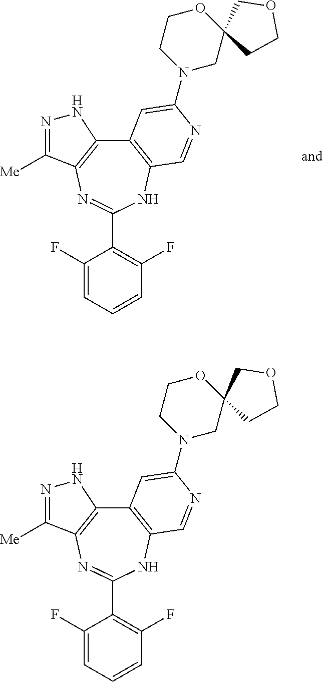 [SMILES: Cc1n[nH]c2c1N=C(c1c(F)cccc1F)Nc1cnc(N3CCO[C@@]4(CCOC4)C3)cc1-2 ; Cc1n[nH]c2c1N=C(c1c(F)cccc1F)Nc1cnc(N3CCO[C@]4(CCOC4)C3)cc1-2]

The title compounds were made from intermediate D2-3, following a similar synthetic procedure as described in the synthesis of Int-1 (described above), except that 2,6-dioxa-9-azaspiro[4.5]decane was used in the place of morpholine, and 10% Pd₂(dba)₃ was employed as the catalyst. The reaction was conducted in dioxane at 100° C. for 12 hr, and then treated with 4N HCl in MeOH to remove the SEM protecting group. The resulting mixture was concentrated under reduced pressure and purified by reverse phase preparative HPLC (Instrument: Gilson GX-281 Liquid Handler, Gilson 322 Pump, Gilson 156 UV Detector; column: ACE 5 C18-AR 150×30 mm×5 m; mobile phase: [water(FA)-ACN]; B %: 19%-49%, 9.5 min, hold 100% B for 2 min; Flow Rate: 25 mL/min; Column Temperature: 30° C.; Wavelength: 220 nm) to provide the racemic compound as a yellow solid (80 mg, 35.5%). The racemate was dissolved in DMSO and re-subjected to chiral preparative HPLC (Column: ChiralPak AS-3 150×4.6 mm I.D., 3 m; Mobile phase: A: CO₂ B:Methanol (0.05% DEA; Gradient: from 5% to 40% of B in 4.5 min, then 5% of B for 1.5 min; Flow rate: 2.5 mL/min; Column temp.: 40° C.; ABPR: 100 bar) to provide the following two products respectively:

Fraction A (34 mg, retention time=3.812 min) was tentatively assigned as compound Example 4: ¹H NMR (400 MHz, DMSO-d₆) δ ppm 1.83-2.01 (m, 5H), 3.27 (br d, J=4.8 Hz, 2H), 3.31 (s, 2H), 3.56-3.84 (m, 6H), 6.86 (s, 1H), 7.17 (t, J=7.9 Hz, 2H), 7.38-7.55 (m, 2H), 8.27 (s, 1H), 12.13-12.75 (m, 1H); LCMS (ESI) m/z: 453.3 [M+H]⁺.

Fraction B (28 mg, retention time=3.916 min) was tentatively assigned as compound Example 3: ¹H NMR (400 MHz, DMSO-d₆) δ ppm 1.87-2.01 (m, 5H), 3.26 (br s, 2H), 3.31 (s, 3H), 3.61 (d, J=9.5 Hz, 1H), 3.68-3.83 (m, 5H), 6.86 (s, 1H), 7.17 (t, J=7.9 Hz, 2H), 7.42-7.53 (m, 2H), 8.28 (s, 1H), 12.03-12.62 (m, 1H); LCMS (ESI) m/z: 453.3 [M+H]⁺.

### Example 5

### 8-[8-(2,6-difluorophenyl)-5-methyl-3,4,7,9,12-pentazatricyclo[8.4.0.02,6]tetradeca-1(10),2(6),4,7,11,13-hexaen-13-yl]-2,5-dioxa-8-azaspiro[3.5]nonane

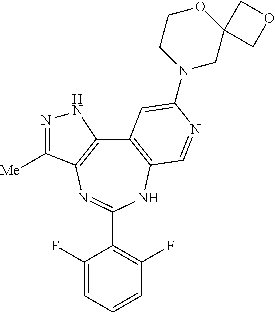 [SMILES: Cc1n[nH]c2c1N=C(c1c(F)cccc1F)Nc1cnc(N3CCOC4(COC4)C3)cc1-2]

The title compound was made from Intermediate D2-3, following a similar synthetic procedure as described in the synthesis of Int-1 (described above), except that 2,5-dioxa-8-azaspiro[3.5]nonane was used in the place of morpholine, and 10% Pd₂(dba)₃ was employed as the catalyst. The reaction was conducted in dioxane at 100° C. for 12 hr, treated with 30% TFA in CH₂Cl₂ for 3 hr, and concentrated under reduced pressure. The resulting residue was purified by reverse phase preparative HPLC (Instrument: Gilson GX-281 Liquid Handler, Gilson 322 Pump, Gilson 156 UV Detector; column: ACE 5 C18-AR 150×30 mm×5 m; mobile phase: [water(FA)-ACN]; B %: 19%-49%, 9.5 min, hold 100% B for 2 min; Flow Rate: 25 mL/min; Column Temperature: 30° C.; Wavelength: 220 nm) to provide the title compound as a yellow solid (20 mg, 28.8%). ¹H NMR (400 MHz, DMSO-d₆) δ ppm 1.96 (s, 3H), 3.23 (br s, 2H), 3.53 (s, 2H), 3.66 (br s, 2H), 4.27-4.65 (m, 5H), 6.90 (s, 1H), 7.17 (t, J=7.9 Hz, 2H), 7.43-7.53 (m, 2H), 8.30 (s, 1H), 12.47 (s, 1H); LCMS (ESI) m/z: 438.4 [M+H]⁺.

### Example 6

### (1S,4S)-5-[8-(2,6-difluorophenyl)-5-methyl-3,4,7,9,12-pentazatricyclo[8.4.0.02,6]tetradeca-1(10),2(6),4,7,11,13-hexaen-13-yl]-2-oxa-5-azabicyclo[2.2.1]heptane

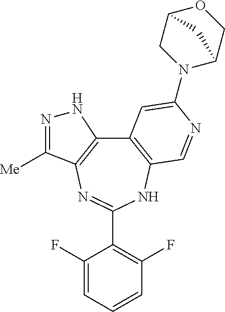 [SMILES: Cc1n[nH]c2c1N=C(c1c(F)cccc1F)Nc1cnc(N3C[C@@H]4C[C@H]3CO4)cc1-2]

The title compound was made from Intermediate D2-3, following a similar synthetic procedure as described in the synthesis of Int-1 (described above), except that (1S,4S)-2-oxa-5-azabicyclo[2.2.1]heptane was used in the place of morpholine, and 10% Pd₂(dba)₃ was employed as the catalyst. The reaction was conducted in dioxane at 100° C. for 12 hr, treated with 4N HCl in MeOH to remove the SEM protecting group. The resulting mixture was concentrated under reduced pressure and the crude product was triturated with EtOAc to provide the title compound as a yellow solid (20 mg, 28.8%). ¹H NMR (400 MHz, CD₃OD) δ ppm 1.96-2.06 (m, 2H), 2.16-2.24 (m, 3H), 3.41 (d, J=10.0 Hz, 1H), 3.60 (d, J=10.5 Hz, 1H), 3.66-3.71 (m, 1H), 3.82-3.87 (m, 1H), 3.88-3.99 (m, 2H), 4.75 (br s, 1H), 7.02 (br d, J=19.8 Hz, 1H), 7.22 (br t, J=8.8 Hz, 2H), 7.49 (br s, 1H), 7.69 (br d, J=3.0 Hz, 1H); LCMS (ESI) m/z: 409.2 [M+H]⁺.

### Example 7

### 8-[8-(2,6-difluorophenyl)-5-methyl-3,4,7,9,12-pentazatricyclo[8.4.0.02,6]tetradeca-1(10),2(6),4,7,11,13-hexaen-13-yl]-2-oxa-5,8-diazaspiro[3.5]nonane

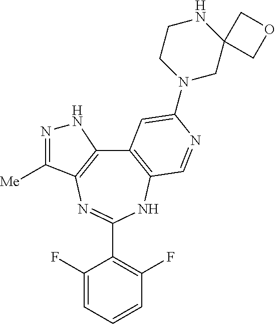 [SMILES: Cc1n[nH]c2c1N=C(c1c(F)cccc1F)Nc1cnc(N3CCNC4(COC4)C3)cc1-2]

The title compound was made from intermediate D2-3, following a similar synthetic procedure as described in the synthesis of Int-1 (described above), except that 2-oxa-5,8-diazaspiro[3.5]nonane was used in the place of morpholine, and 10% Pd₂(dba)₃ was employed as the catalyst. The reaction was conducted in dioxane at 100° C. for 12 hr, treated with 30% TFA in CH₂Cl₂ for 3 hr, and concentrated under reduced pressure. The resulting residue was purified by Pre-HPLC (column: Phenomenex C18 75×30 mm×3 m; mobile phase: [water (ammonia hydroxide v/v)-ACN]; B %: 18%-38%, 9 min) to provide the title compound as a yellow solid (20.1 mg, 37.3%). ¹H NMR (400 MHz, DMSO-d₆) δ ppm 1.96 (s, 3H), 2.68-2.73 (m, 2H), 3.18 (br s, 2H), 3.46-3.51 (m, 2H), 4.29-4.32 (m, 2H), 4.34-4.37 (m, 2H), 6.87 (s, 1H), 7.17 (t, J=7.9 Hz, 2H), 7.43-7.53 (m, 2H), 8.27 (s, 1H), 12.43 (s, 1H); LCMS (ESI) m/z: 437.5 [M+H]⁺.

### Example 8

### 8-(2,6-difluorophenyl)-5-methyl-13-(4-methylpiperazin-1-yl)-3,4,7,9,12-pentazatricyclo[8.4.0.02,6]tetradeca-1(10),2(6),4,7,11,13-hexaene

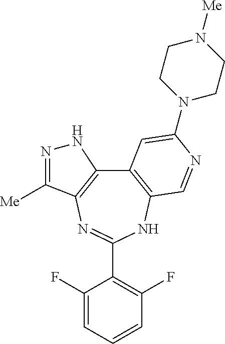 [SMILES: Cc1n[nH]c2c1N=C(c1c(F)cccc1F)Nc1cnc(N3CCN(C)CC3)cc1-2]

The title compound was made from intermediate D2-3, following a similar synthetic procedure as described in the synthesis of Int-1 (described above), except that 1-methylpiperazine was used in the place of morpholine, and 10% Pd₂(dba)₃ was employed as the catalyst. The reaction was conducted in dioxane at 100° C. for 12 hr, purified by prep-HPLC (YMC-Triart Prep C18 150×40 mm×7 m; mobile phase: [water (NH₃H₂O+NH₄HCO₃)-can]; B %: 50%-90%, 9 min), and treated with 4N HCl in MeOH to provide the title compound as a yellow solid (57.6 mg, 75.9%). ¹H NMR (400 MHz, DMSO-d₆) δ ppm 2.20 (s, 3H), 2.77 (br d, J=4.4 Hz, 3H), 3.00-3.11 (m, 2H), 3.31-3.45 (m, 4H), 4.32-4.39 (m, 2H), 7.17 (s, 1H), 7.37 (br t, J=8.5 Hz, 2H), 7.74-7.83 (m, 1H), 7.92 (br s, 1H), 11.37-11.49 (m, 1H); LCMS (ESI) m/z: 410.2 [M+H]⁺.

### Example 9

### 4-[8-(2,6-difluorophenyl)-5-methyl-3,4,7,9,12-pentazatricyclo[8.4.0.02,6] tetradeca-1(10),2(6),4,7,11,13-hexaen-13-yl]-1,4-oxazepane

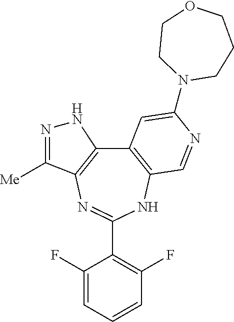 [SMILES: Cc1n[nH]c2c1N=C(c1c(F)cccc1F)Nc1cnc(N3CCCOCC3)cc1-2]

The title compound was made from intermediate D2-3, following a similar synthetic procedure as described in the synthesis of Int-1 (described above), except that 1,4-oxazepane was used in the place of morpholine, and 10% Pd₂(dba)₃ was employed as the catalyst. The reaction was conducted in dioxane at 100° C. for 12 hr, purified by flash chromatography (ISCO®; 4 g SepaFlash® Silica Flash Column, PE/EtOAc with EtOAc from 0˜100%, 20 mL/min, 254 nm) and treated with 4N HCl in MeOH to provide the title compound as a yellow solid (21 mg, 23.0%). ¹H NMR (400 MHz, DMSO-d₆) δ ppm 1.82-1.93 (m, 2H), 2.15 (s, 3H), 3.64 (t, J=5.4 Hz, 2H), 3.69-3.80 (m, 6H), 7.05 (s, 1H), 7.36 (br t, J=8.4 Hz, 2H), 7.56-7.82 (m, 2H); LCMS (ESI) m/z: 411.1 [M+H]⁺.

### Example 10

### 8-(2, 6-difluorophenyl)-5-methyl-13-piperazin-1-yl-3, 4, 7, 9, 12-pentazatricyclo [8. 4. 0. 02, 6] tetradeca-1(10), 2(6), 4, 7, 11, 13-hexaene

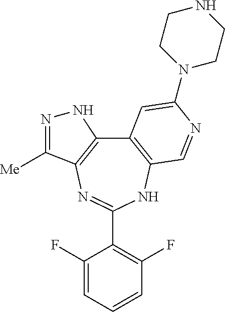 [SMILES: Cc1n[nH]c2c1N=C(c1c(F)cccc1F)Nc1cnc(N3CCNCC3)cc1-2]

The title compound was made from intermediate D2-3, following a similar synthetic procedure as described in the synthesis of Int-1 (described above), except that tert-butyl piperazine-1-carboxylate hydrochloride was used in the place of morpholine, and 10% Pd₂(dba)₃ was employed as the catalyst. The reaction was conducted in dioxane at 100° C. for 12 hr, purified by flash chromatography (ISCO®; 20 g AgelaFlash® Silica Flash Column, petroleum ether/EtOAc with EtOAc from 0˜25%, flow rate=30 mL/min, 254 nm) and treated with 4N HCl in MeOH to remove SEM and Boc protecting groups. The crude product was purified by reverse preparative HPLC (Instrument: Gilson GX-281 Liquid Handler, Gilson 322 Pump, Gilson 156 UV Detector; Column: Welch Xtimate C18 150×25 mm×5 m; Mobile phase A: H₂O with 0.05% NH₃—H₂O (v %); Mobile phase B: ACN; Gradient: B from 20% to 50% in 9.5 min, hold 100% B for 2 min; Flow Rate: 25 mL/min; Column Temperature: 30° C.; Wavelength: 220 nm) to provide the title compound as a yellow solid (26 mg, 41.2%). ¹H NMR (400 MHz, DMSO-d₆) δ ppm 1.96 (s, 3H), 2.08 (s, 2H), 2.81 (br s, 4H), 3.28 (br s, 2H), 6.81 (br s, 1H), 7.17 (br t, J=7.9 Hz, 2H), 7.30-7.62 (m, 2H), 8.23 (br s, 1H), 12.46 (br s, 1H); LCMS (ESI) m/z: 396.3 [M+H]⁺.

### Example 11

### 8-(2, 6-difluorophenyl)-13-(4-ethylsulfonylpiperazin-1-yl)-5-methyl-3, 4, 7, 9, 12-pentazatricyclo [8. 4. 0. 02, 6] tetradeca-1(10), 2(6), 4, 7, 11, 13-hexaene

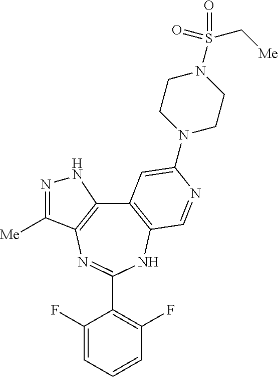 [SMILES: CCS(=O)(=O)N1CCN(c2cc3c(cn2)NC(c2c(F)cccc2F)=Nc2c(C)n[nH]c2-3)CC1]

The title compound was made from intermediate D2-3, following a similar synthetic procedure as described in the synthesis of Int-1 (described above), except that 1-(ethylsulfonyl)piperazine was used in the place of morpholine, and 10% Pd₂(dba)₃ was employed as the catalyst. The reaction was conducted in dioxane at 100° C. for 12 hr, purified by flash chromatography (ISCO®; 20 g AgelaFlash® Silica Flash Column, petroleum ether/EtOAc with EtOAc from 0˜25%, flow rate=30 mL/min, 254 nm) and treated with 4N HCl in MeOH to remove SEM and Boc protecting groups. The crude product was purified by reverse preparative HPLC (Instrument: Gilson GX-281 Liquid Handler, Gilson 322 Pump, Gilson 156 UV Detector; Column: Welch Xtimate C18 150×25 mm×5 m; Mobile phase A: H₂O with 0.05% NH₃—H₂O (v %); Mobile phase B: ACN; Gradient: B from 20% to 50% in 9.5 min, hold 100% B for 2 min; Flow Rate: 25 mL/min; Column Temperature: 30° C.; Wavelength: 220 nm) to provide the title compound as a yellow solid (25.5 mg, 11.5%). ¹H NMR (400 MHz, DMSO-d₆) δ ppm 1.23 (t, J=7.3 Hz, 3H), 1.95 (s, 3H), 3.08 (q, J=7.5 Hz, 2H), 3.16-3.35 (m, 8H), 6.87 (s, 1H), 7.16 (t, J=7.9 Hz, 2H), 7.40-7.60 (m, 2H), 8.26 (br s, 1H), 12.05-12.77 (m, 1H), 12.47 (br s, 1H); LCMS (ESI) m/z: 488.3 [M+H]⁺.

### Example 12 and 13

### (R)-1-(5-(2,6-difluorophenyl)-3-methyl-1,6-dihydropyrazolo[4,3-d]pyrido[4,3-f][1,3]diazepin-9-yl)-3-methylpiperidine-3-carbonitrile (Example 12) and (S)-1-(5-(2,6-difluorophenyl)-3-methyl-1,6-dihydropyrazolo[4,3-d]pyrido[4,3-f][1,3]diazepin-9-yl)-3-methylpiperidine-3-carbonitrile (Example 13)

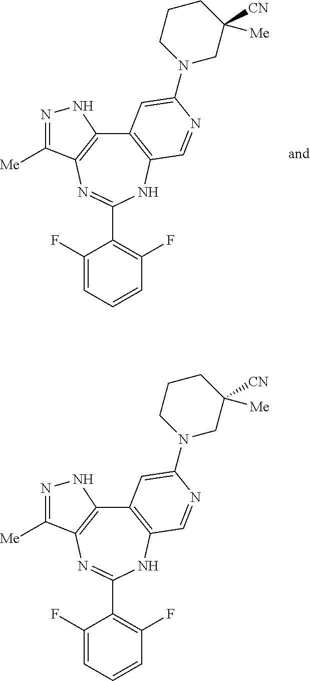 [SMILES: Cc1n[nH]c2c1N=C(c1c(F)cccc1F)Nc1cnc(N3CCC[C@@](C)(C#N)C3)cc1-2 ; Cc1n[nH]c2c1N=C(c1c(F)cccc1F)Nc1cnc(N3CCC[C@](C)(C#N)C3)cc1-2]

The title compounds were made from intermediate D2-3, following a similar synthetic procedure as described in the synthesis of Int-1 (described above), except that 3-methylpiperidine-3-carbonitrile was used in the place of morpholine, and 10% Pd₂(dba)₃ was employed as the catalyst. The reaction was conducted in dioxane at 100° C. for 12 hr and treated with 4N HCl in MeOH to remove the SEM protecting group. The resulting mixture was concentrated under reduced pressure and purified by reverse phase preparative HPLC (column: YMC-Triart Prep C18 150×40 mm×7 m; mobile phase: [water (NH₃H₂O+NH₄HCO₃)-ACN]; B %: 16%-56%, 9 min) to provide the racemic compound as a yellow solid (80 mg, 35.5%). The racemate was dissolved in DMSO and re-subjected to chiral preparative HPLC column: DAICEL CHIRALPAK AS (250 mm×30 mm×10 m); mobile phase: [0.1% NH₃H₂O EtOH]; B %: 35%-35%, 6 min) to provide the following two products respectively:

Fraction A (29.8 mg, retention time=3.710 min) was tentatively assigned as compound Example 12: ¹H NMR (400 MHz, CD₃OD) δ ppm 1.40 (s, 3H), 1.58 (td, J=12.7, 4.0 Hz, 1H), 1.77-1.93 (m, 2H), 2.08 (br s, 1H), 2.12 (s, 3H), 2.72-2.90 (m, 2H), 4.06 (br d, J=13.1 Hz, 1H), 4.36 (br d, J=13.1 Hz, 1H), 7.02-7.09 (m, 3H), 7.39 (s, 1H), 7.43-7.52 (m, 1H); LCMS (ESI) m/z: 434.2 [M+H]⁺.

Fraction B (27.5 mg, retention time=4.030 min) was tentatively assigned as compound Example 13: ¹H NMR (400 MHz, CD₃OD) δ ppm 1.40 (s, 4H), 1.54-1.61 (m, 1H), 1.80-1.92 (m, 2H), 2.08 (br s, 1H), 2.11 (s, 3H), 2.74-2.87 (m, 2H), 4.06 (br d, J=12.8 Hz, 1H), 4.36 (br d, J=13.1 Hz, 1H), 7.02-7.08 (m, 3H), 7.39 (s, 1H), 7.43-7.52 (m, 1H); LCMS (ESI) m/z: 434.2 [M+H]⁺.

### Example 14

### 2,2,3,3,5,5,6,6-octadeuterio-4-[8-(2,6-difluorophenyl)-5-methyl-3,4,7,9,12-pentazatricyclo [8.4.0.02,6]tetradeca-1(10),2(6),4,7,11,13-hexaen-13-yl]morpholine

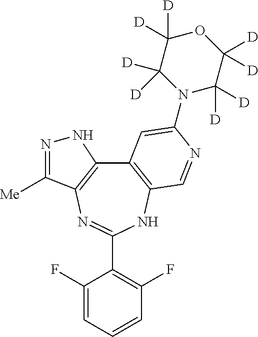 [SMILES: [2H]C1([2H])OC([2H])([2H])C([2H])([2H])N(c2cc3c(cn2)NC(c2c(F)cccc2F)=Nc2c(C)n[nH]c2-3)C1([2H])[2H]]

The title compound was made from intermediate D2-3, following a similar synthetic procedure as described in the synthesis of Int-1 (described above), except that 2,2,3,3,5,5,6,6-octadeuteriomorpholine was used in the place of morpholine, and 10% Pd₂(dba)₃ was employed as the catalyst. The reaction was conducted in dioxane at 100° C. for 12 hr, purified by flash chromatography (ISCO®; 20 g SepaFlash® Silica Flash Column, petroleum ether/EtOAc with EtOAc from 0˜55%, 30 mL/min, 254 nm), treated with 4N HCl in MeOH to provide the title compound as a yellow solid (82.8 mg, 60.8%). ¹H NMR (400 MHz, DMSO-d₆) δ ppm 1.95 (s, 3H), 6.77-6.90 (m, 1H), 7.16 (t, J=7.94 Hz, 2H), 7.40-7.45 (m, 1H), 7.46-7.52 (m, 1H), 8.25 (s, 1H), 12.42 (s, 1H); LCMS (ESI) m/z: 405.0 [M+H]⁺.

### Example 15 and Example 16

### (2R)-4-[8-(2,6-difluorophenyl)-5-methyl-3,4,7,9,12-pentazatricyclo[8.4.0.02,6]tetradeca-1(10),2(6),4,7,11,13-hexaen-13-yl]-2-(methoxymethyl)morpholine (Example 15) and (2S)-4-[8-(2,6-difluorophenyl)-5-methyl-3,4,7,9,12-pentazatricyclo[8.4.0.02,6]tetradeca-1(10),2(6),4,7,11,13-hexaen-13-yl]-2-(methoxymethyl)morpholine (Example 16)

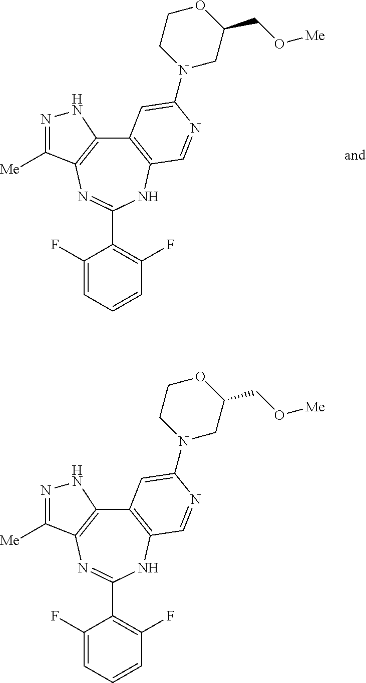 [SMILES: COC[C@H]1CN(c2cc3c(cn2)NC(c2c(F)cccc2F)=Nc2c(C)n[nH]c2-3)CCO1 ; COC[C@@H]1CN(c2cc3c(cn2)NC(c2c(F)cccc2F)=Nc2c(C)n[nH]c2-3)CCO1]

The title compounds were made from intermediate D2-3, following a similar synthetic procedure as described in the synthesis of Int-1 (described above), except that 2-(methoxymethyl)morpholine was used in the place of morpholine, and 10% RuPhos-Pd-G₃ was employed as the catalyst. The reaction was conducted in dioxane at 100° C. for 12 hr and treated with 4N HCl in MeOH to remove the SEM protecting group. The resulting mixture was concentrated under reduced pressure and purified by flash silica gel chromatography (ISCO®; 12 g SepaFlash® Silica Flash Column, Eluent of 0˜80% petroleum ether/EtOAc @ 35 mL/min) to provide the racemic compound (200 mg, 83.8%). The racemate was dissolved in DMSO and re-subjected to chiral preparative HPLC column: DAICEL CHIRALPAK AS (250 mm×30 mm×m); mobile phase: [0.1% NH₃H₂O EtOH]; B %: 35%-35%, 6 min) to provide the following two products respectively:

Fraction A (17.9 mg, retention time=5.120 min) was tentatively assigned as compound Example 15: ¹H NMR (400 MHz, DMSO-d₆) δ ppm 1.95 (s, 3H), 2.72 (br dd, J=3.1, 11.9 Hz, 1H), 3.28 (s, 3H), 3.46-3.35 (m, 3H), 3.53 (dt, J=2.5, 11.5 Hz, 1H), 3.64-3.58 (m, 1H), 3.78 (br d, J=12.5 Hz, 1H), 3.89 (br d, J=11.5 Hz, 2H), 6.82 (s, 1H), 7.16 (t, J=7.9 Hz, 2H), 7.43 (s, 1H), 7.53-7.44 (m, 1H), 8.26 (s, 1H), 12.42 (s, 1H); LCMS (ESI) m/z: 441.2 [M+H]⁺.

Fraction B (25.8 mg, retention time=5.240 min) was tentatively assigned as compound Example 16: ¹H NMR (400 MHz, DMSO-d₆) δ ppm 1.95 (s, 3H), 2.72 (br dd, J=3.1, 11.9 Hz, 1H), 3.28 (s, 3H), 3.46-3.35 (m, 3H), 3.53 (dt, J=2.5, 11.5 Hz, 1H), 3.64-3.58 (m, 1H), 3.78 (br d, J=12.5 Hz, 1H), 3.89 (br d, J=11.5 Hz, 2H), 6.82 (s, 1H), 7.16 (t, J=7.9 Hz, 2H), 7.43 (s, 1H), 7.53-7.44 (m, 1H), 8.26 (s, 1H), 12.42 (s, 1H); LCMS (ESI) m/z: 441.2 [M+H]⁺.

### Example 17

### 1-[8-(2,6-difluorophenyl)-5-methyl-3,4,7,9,12-pentazatricyclo[8.4.0.02,6]tetradeca-1(10),2(6),4,7,11,13-hexaen-13-yl]-4-methyl-piperidine-4-carbonitrile

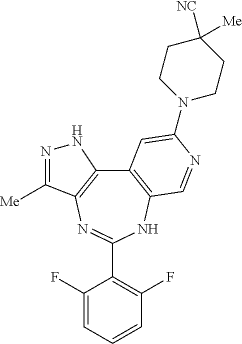 [SMILES: Cc1n[nH]c2c1N=C(c1c(F)cccc1F)Nc1cnc(N3CCC(C)(C#N)CC3)cc1-2]

The title compound was made from intermediate D2-3, following a similar synthetic procedure as described in the synthesis of Int-1 (described above), except that 4-methylpiperidine-4-carbonitrile was used in the place of morpholine, and 10% of Pd-PEPPSI-IPent was employed as the catalyst. The reaction was conducted in dioxane at 100° C. for 12 hr, purified by flash chromatography (ISCO®; 12 g SepaFlash® Silica Flash Column, petroleum ether/EtOAc with EtOAc from 0˜100%, 30 mL/min, 254 nm) and treated with 4N HCl in MeOH to provide the title compound as a yellow solid (300 mg, 48.8%). ¹H NMR (400 MHz, DMSO-d₆) δ ppm 1.36 (s, 3H), 1.48-1.57 (m, 2H), 1.85-1.98 (m, 5H), 2.81-2.93 (m, 2H), 4.00-4.11 (m, 2H), 6.84-6.91 (m, 1H), 7.14-7.20 (m, 2H), 7.41-7.54 (m, 2H), 8.21-8.31 (m, 1H); LCMS (ESI) m/z: 434.1 [M+H]⁺.

### Example 18

### (3R)-1-[8-(2,6-difluorophenyl)-5-methyl-3,4,7,9,12-pentazatricyclo[8.4.0.02,6]tetradeca-1(10),2(6),4,7,11,13-hexaen-13-yl]piperidine-3-carbonitrile

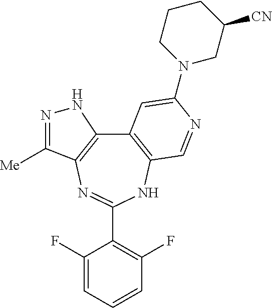 [SMILES: Cc1n[nH]c2c1N=C(c1c(F)cccc1F)Nc1cnc(N3CCC[C@@H](C#N)C3)cc1-2]

The title compound was made from intermediate D2-3, following a similar synthetic procedure as described in the synthesis of Int-1 (described above), except that (3R)-piperidine-3-carbonitrile was used in the place of morpholine, and 10% Pd₂(dba)₃ was employed as the catalyst. The reaction was conducted in dioxane at 100° C. for 12 hr, purified by flash chromatography (ISCO®; 20 g AgelaFlash® Silica Flash Column, petroleum ether/EtOAc with EtOAc from 0˜100%, flow rate=30 mL/min, 254 nm) and treated with 4N HCl in MeOH to provide the title compound as a yellow solid (36.0 mg, 27.7%). ¹H NMR (400 MHz, DMSO-d₆) δ ppm 1.49-1.69 (m, 2H), 1.77-1.93 (m, 3H), 1.95 (s, 3H), 3.01 (br s, 2H), 3.55-3.82 (m, 2H), 6.88 (s, 1H), 7.16 (t, J=7.9 Hz, 2H), 7.42 (s, 1H), 7.48 (td, J=7.4, 15.0 Hz, 1H), 8.27 (s, 1H), 12.45 (s, 1H); LCMS (ESI) m/z: 420.2 [M+H]⁺.

### Example 19

### (3S)-1-[8-(2,6-difluorophenyl)-5-methyl-3,4,7,9,12-pentazatricyclo [8.4.0.02,6]tetradeca-1(10),2(6),4,7,11,13-hexaen-13-yl]piperidine-3-carbonitrile

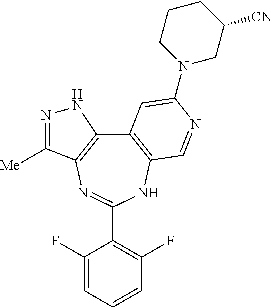 [SMILES: Cc1n[nH]c2c1N=C(c1c(F)cccc1F)Nc1cnc(N3CCC[C@H](C#N)C3)cc1-2]

The title compound was made from intermediate D2-3, following a similar synthetic procedure as described in the synthesis of Int-1 (described above), except that (3S)-piperidine-3-carbonitrile was used in the place of morpholine, and 10% Pd₂(dba)₃ was employed as the catalyst. The reaction was conducted in dioxane at 100° C. for 12 hr, purified by flash chromatography (ISCO®; 20 g AgelaFlash® Silica Flash Column, petroleum ether/EtOAc with EtOAc from 0˜100%, flow rate=30 mL/min, 254 nm) and treated with 4N HCl in MeOH to provide the title compound as a yellow solid (11.1 mg, 14.6%). ¹H NMR (400 MHz, DMSO-d₆) δ ppm 1.49-1.59 (m, 1H), 1.62-1.69 (m, 1H), 1.83-1.91 (m, 2H), 1.95-1.98 (m, 3H), 2.99-3.04 (m, 1H), 3.39-3.48 (m, 2H), 3.62-3.68 (m, 2H), 6.87-6.90 (m, 1H), 7.14-7.18 (m, 2H), 7.38-7.45 (m, 1H), 7.46-7.53 (m, 1H), 8.15-8.38 (m, 1H), 12.39-12.49 (m, 1H); LCMS (ESI) m/z: 420.2 [M+H]⁺.

### Example 20

### Synthesis of 2,2,3,3,5,5,6,6-octadeuterio-4-[8-(2,6-difluorophenyl)-3,4,7,9,12-pentazatricyclo[8.4.0.02,6]tetradeca-1(10),2(6),4,7,11,13-hexaen-13-yl]morpholine

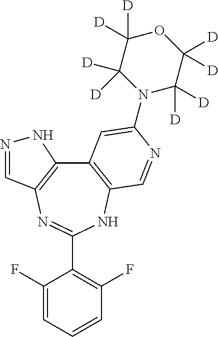 [SMILES: [2H]C1([2H])OC([2H])([2H])C([2H])([2H])N(c2cc3c(cn2)NC(c2c(F)cccc2F)=Nc2cn[nH]c2-3)C1([2H])[2H]]

The title compound was made from intermediate D2-1, following a similar synthetic procedure as described in the synthesis of Int-1 (described above), except that 2,2,3,3,5,5,6,6-octadeuteriomorpholine was used in the place of morpholine. The reaction was conducted in dioxane at 110° C. for 30 min and treated with 4N HCl in MeOH. The residue was purified by reverse HPLC (column: 2_Phenomenex Gemini C18 75×40 mm×3 μm; mobile phase: [water(NH₃—H₂O+NH₄HCO₃)-ACN]; B %: 10%-50%, 9.5 min) to provide 2,2,3,3,5,5,6,6-octadeuterio-4-[8-(2,6-difluorophenyl)-3,4,7,9,12-pentazatricyclo[8.4.0.02,6]tetradeca-1(10),2(6),4,7,11,13-hexaen-13-yl]morpholine as a yellow solid (25.5 mg, 28.5%). ¹H NMR (400 MHz, DMSO-d₆) δ ppm 6.80-6.90 (m, 1H), 7.16 (t, J=8.03 Hz, 2H), 7.28-7.33 (m, 1H), 7.42-7.54 (m, 2H), 8.34 (s, 1H), 12.64 (s, 1H); ¹⁹F NMR (377 MHz, DMSO-d₆) δ ppm −114.25; LCMS (ESI) m/z: 391.0 [M+H]⁺.

### Example 21

### Synthesis of (3-(3-chloro-9-morpholino-1,6-dihydropyrazolo[4,3-d]pyrido[4,3-f][1,3]diazepin-5-yl)-2,4-difluorophenyl)methanol

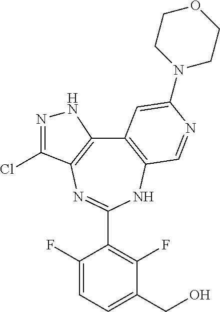 [SMILES: OCc1ccc(F)c(C2=Nc3c(Cl)n[nH]c3-c3cc(N4CCOCC4)ncc3N2)c1F]

The title compound was made from Intermediate D2-4, following a similar synthetic procedure as described in the synthesis of Int-1 (described above). The reaction was conducted in dioxane at 100° C. for 30 min and treated with 4N HCl in MeOH. The residue was purified by reverse HPLC (column: 2_Phenomenex Gemini C18 75×40 mm×3 μm; mobile phase: [water(NH₄HCO₃)-ACN]; B %: 15%-45%, 9.5 min) to provide [3-(5-chloro-13-morpholino-3,4,7,9,12-pentazatricyclo[8.4.0.0^{2,6}]tetradeca-1(10),2(6),4,7,11,13-hexaen-8-yl)-2,4-difluoro-phenyl]methanol as a yellow solid (24.1 mg, 12.4%). ¹H NMR (400 MHz, DMSO-d₆) δ ppm 3.24-3.31 (m, 4H), 3.67 (br s, 4H), 4.54 (br s, 2H), 5.03-5.58 (m, 1H), 6.73 (br s, 1H), 7.08-7.27 (m, 1H), 7.35 (br s, 1H), 7.53 (br d, J=7.28 Hz, 1H), 8.15-8.94 (m, 1H), 13.27 (br s, 1H); ¹⁹F NMR (377 MHz, DMSO-d₆) δ ppm −116.38, −119.97; LCMS (ESI) m/z: 447.0 [M+H]⁺.

### Example 22

### Synthesis of (4-(3-chloro-9-morpholino-1,6-dihydropyrazolo[4,3-d]pyrido[4,3-f][1,3]diazepin-5-yl)-3,5-difluorophenyl)methanol

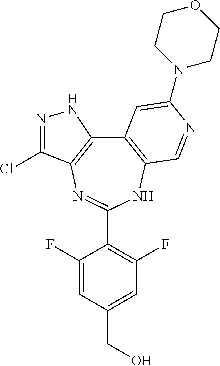 [SMILES: OCc1cc(F)c(C2=Nc3c(Cl)n[nH]c3-c3cc(N4CCOCC4)ncc3N2)c(F)c1]

The title compound was made from intermediate D2-5, following a similar synthetic procedure as described in the synthesis of Int-1 (described above). The reaction was conducted in dioxane at 100° C. for 30 min. and treated with 4N HCl in MeOH. The residue was purified by reverse HPLC (column: Welch Xtimate C18 100×40 mm×3 μm; mobile phase: [water (HCl)-ACN]; B %: 10%-40%, 8.5 min) to provide (4-(3-chloro-9-morpholino-1,6-dihydropyrazolo[4,3-d]pyrido[4,3-f][1,3]diazepin-5-yl)-3,5-difluorophenyl)methanol as a yellow solid (33.9 mg, 18.7%). ¹H NMR (400 MHz, DMSO-d₆) δ ppm 3.52 (br s, 4H) 3.71 (br s, 5H) 4.56 (s, 2H) 7.16 (br d, J=9.0 Hz, 3H) 7.41 (br s, 1H); ¹⁹F NMR (377 MHz, DMSO-d₆) δ ppm −113.28; LCMS (ESI) m/z: 447.0 [M+H]⁺.

### Example 23

### Synthesis of 4-(3-chloro-5-(2,6-difluorophenyl)-1,6-dihydropyrazolo[4,3-d]pyrido[4,3-f][1,3]diazepin-9-yl)morpholine

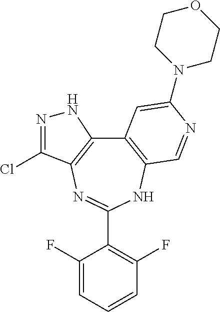 [SMILES: Fc1cccc(F)c1C1=Nc2c(Cl)n[nH]c2-c2cc(N3CCOCC3)ncc2N1]

### Step 1: 2-[[5-chloro-8-(2,6-difluorophenyl)-13-morpholino-3,4,7,9,12-pentazatricyclo[8.4.0.0^{2,6}]tetradeca-1(10),2(6),4,7,11,13-hexaen-3-yl]methoxy]ethyl-trimethyl-silane

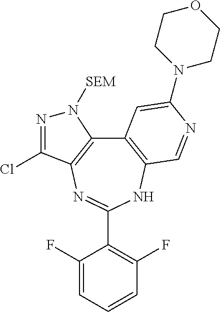 [SMILES: C[Si](C)(C)CCOCn1nc(Cl)c2c1-c1cc(N3CCOCC3)ncc1NC(c1c(F)cccc1F)=N2]

The title compound was made from Intermediate D2-2, following a similar synthetic procedure as described in the synthesis of Int-1 (described above). The reaction was conducted in dioxane at 100° C. for 12 hr and purified by preparative TLC (silica, petroleum ether/EtOAc=3/1, 254 nm) to give 2-[[5-chloro-8-(2,6-difluorophenyl)-13-morpholino-3,4,7,9,12-pentazatricyclo[8.4.0.0^{2,6}]tetradeca-1(10),2(6),4,7,11,13-hexaen-3-yl]methoxy]ethyl-trimethyl-silane as a yellow solid (30 mg, 17.4%). LCMS (ESI) m/z: 547.1 [M+H]⁺.

### Step 2: 4-[5-chloro-8-(2,6-difluorophenyl)-3,4,7,9,12-pentazatricyclo[8.4.0.0^{2,6}]tetradeca-1(10),2(6),4,7,11,13-hexaen-13-yl]morpholine

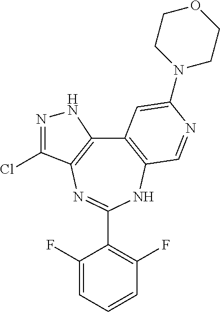 [SMILES: Fc1cccc(F)c1C1=Nc2c(Cl)n[nH]c2-c2cc(N3CCOCC3)ncc2N1]

To a solution of 2-[[5-chloro-8-(2,6-difluorophenyl)-13-morpholino-3,4,7,9,12-pentazatricyclo[8.4.0.0^{2,6}]tetradeca-1(10),2(6),4,7,11,13-hexaen-3-yl]methoxy]ethyl-trimethyl-silane (30 mg, 0.05 mmol, 1 eq) in 4N HCl in MeOH (1 mL) was added triethylsilane (19.1 mg, 0.16 mmol, 3 eq). The mixture was allowed to stir at 25° C. for 30 min. and concentrated under reduced pressure. The residue was diluted with NaHCO₃ (2 mL) and extracted with EtOAc (5 mL×3). The combined organic layers were washed with brine (5 mL), dried over Na₂SO₄, filtered and concentrated under reduced pressure. The residue was purified by preparative TLC (silica, petroleum ether/EtOAc=1/1, 254 nm) to give compound 4-[5-chloro-8-(2,6-difluorophenyl)-3,4,7,9,12-pentazatricyclo[8.4.0.0^{2,6}]tetradeca-1(10),2(6),4,7,11,13-hexaen-13-yl]morpholine as a yellow solid (10.3 mg, 45.0%). ¹H NMR (400 MHz, DMSO-d₆) δ ppm 3.32 (br d, J=5.77 Hz, 4H), 3.65-3.70 (m, 4H), 6.71 (br s, 1H), 7.18 (t, J=8.00 Hz, 2H), 7.35 (br s, 1H), 7.46-7.58 (m, 1H), 8.40 (br s, 1H), 13.25 (br s, 1H); LCMS (ESI) m/z: 417.0 [M+H]⁺.

### Example 24

### Synthesis of (4-[5-chloro-8-(2-chloro-6-fluoro-phenyl)-3,4,7,9,12-pentazatricyclo[8.4.0.02,6]tetradeca-1(10),2(6),4,7,11,13-hexaen-13-yl]morpholine)

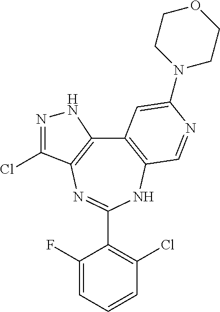 [SMILES: Fc1cccc(Cl)c1C1=Nc2c(Cl)n[nH]c2-c2cc(N3CCOCC3)ncc2N1]

The title compound was made from Intermediate D2-8, following a similar synthetic procedure as described in the synthesis of Int-1 (described above). The reaction was conducted in dioxane at 80° C. for 1 hr and treated with 4N HCl in MeOH to remove the SEM protecting group. The resulting mixture was concentrated under reduced pressure and purified by column chromatography (silica, petroleum ether/Ethyl acetate=1:1, 254 nm) to afford 4-[5-chloro-8-(2-chloro-6-fluoro-phenyl)-3,4,7,9,12-pentazatricyclo[8.4.0.0^{2,6}]tetradeca-1(10),2(6),4,7,11,13-hexaen-13-yl]morpholine as a yellow solid (10 mg, 53.1%). ¹H NMR (400 MHz, CD₃OD) δ ppm 3.37-3.44 (m, 4H), 3.73-3.81 (m, 4H), 6.68 (br s, 1H), 7.19 (t, J=8.7 Hz, 1H), 7.30-7.40 (m, 2H), 7.45 (dt, J=6.0, 8.3 Hz, 1H); LCMS (ESI) m/z: 433.3 [M+H]⁺.

### Example 25

### Synthesis of 4-[5-chloro-8-(2,6-difluorophenyl)-3,4,7,9,12-pentazatricyclo[8.4.0.02,6]tetradeca-1(10),2(6),4,7,11,13-hexaen-13-yl]-2,2,3,3,5,5,6,6-octadeuterio-morpholine

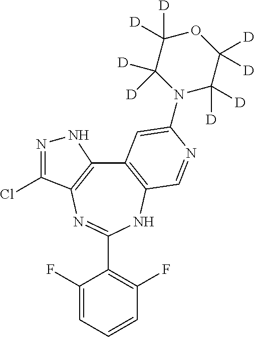 [SMILES: [2H]C1([2H])OC([2H])([2H])C([2H])([2H])N(c2cc3c(cn2)NC(c2c(F)cccc2F)=Nc2c(Cl)n[nH]c2-3)C1([2H])[2H]]

The title compound was made from Intermediate D2-2, following a similar synthetic procedure as described in the synthesis of Int-1 (described above), except that 2,2,3,3,5,5,6,6-octadeuteriomorpholine was used in the place of morpholine. The reaction was conducted in dioxane at 100° C. for 30 min. and treated with 4N HCl in MeOH to remove the SEM protecting group. The resulting mixture was concentrated under reduced pressure and purified by preparative HPLC to give 4-[5-chloro-8-(2,6-difluorophenyl)-3,4,7,9,12-pentazatricyclo[8.4.0.02,6]tetradeca-1(10),2(6),4,7,11,13-hexaen-13-yl]-2,2,3,3,5,5,6,6-octadeuterio-morpholine as a yellow solid (101.2 mg, 71.6%, HCl salt). ¹H NMR (400 MHz, DMSO-d₆) δ ppm 7.22 (br t, J=8.1 Hz, 3H), 7.27-7.49 (m, 2H), 7.51-7.62 (m, 1H), 9.19 (br s, 1H), 13.03-14.44 (m, 1H); LCMS (ESI) m/z: 425.0 [M+H]⁺.

### Example 26

### Synthesis of (2S,6S)-4-(3-chloro-5-(2,6-difluorophenyl)-1,6-dihydropyrazolo[4,3-d]pyrido[4,3-f][1,3]diazepin-9-yl)-2,6-dimethylmorpholine

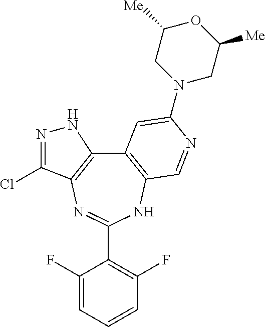 [SMILES: C[C@H]1CN(c2cc3c(cn2)NC(c2c(F)cccc2F)=Nc2c(Cl)n[nH]c2-3)C[C@H](C)O1]

The title compound was made from Intermediate D2-2, following a similar synthetic procedure as described in the synthesis of Int-1 (described above), except that (2S,6S)-2,6-dimethylmorpholine was used in the place of morpholine. The reaction was conducted in dioxane at 100° C. for 3 hr. and treated with 4N HCl in MeOH to remove the SEM protecting group. The resulting mixture was concentrated under reduced pressure and purified by preparative HPLC to give (2S,6S)-4-(3-chloro-5-(2,6-difluorophenyl)-1,6-dihydropyrazolo[4,3-d]pyrido[4,3-f][1,3]diazepin-9-yl)-2,6-dimethylmorpholine as a white solid (4.8 mg, 31.0%). ¹H NMR (400 MHz, CD₃OD) δ ppm 1.26 (d, J=6.5 Hz, 6H), 3.39 (dd, J=12.9, 6.7 Hz, 2H), 3.73 (dd, J=13.1, 3.3 Hz, 2H), 4.16 (td, J=6.5, 3.3 Hz, 2H), 7.10 (br s, 1H), 7.27 (t, J=8.4 Hz, 2H), 7.53 (s, 1H), 7.69-7.83 (m, 1H); LCMS (ESI) m/z: 445.1 [M+H]⁺.

### Example 27

### Synthesis of (2R)-4-[8-(2,6-difluorophenyl)-5-(trideuteriomethyl)-3,4,7,9,12-pentazatricyclo[8.4.0.02,6]tetradeca-1(10),2(6),4,7,11,13-hexaen-13-yl]-2-methyl-morpholine

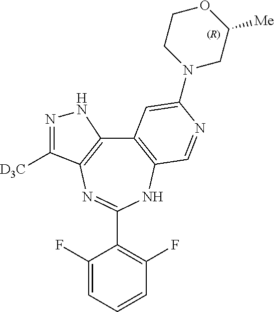

### Step 1: 2-[[5-chloro-8-(2,6-difluorophenyl)-13-[(2R)-2-methylmorpholin-4-yl]-3,4,7,9,12-pentazatricyclo[8.4.0.0^{2,6}]tetradeca-1(10),2(6),4,7,11,13-hexaen-3-yl]methoxy]ethyl-trimethyl-silane

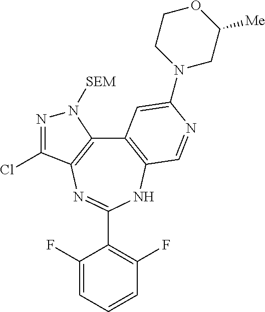 [SMILES: C[C@@H]1CN(c2cc3c(cn2)NC(c2c(F)cccc2F)=Nc2c(Cl)nn(COCC[Si](C)(C)C)c2-3)CCO1]

The title compound was made from Intermediate D2-2, following a similar synthetic procedure as described in the synthesis of Int-1 (described above), except that (2R)-2-methylmorpholine was used in the place of morpholine. The reaction was conducted in dioxane at 100° C. for 1 hr and purified by flash silica gel chromatography (ISCO®; 40 g SepaFlash® Silica Flash Column, Eluent of 0˜30% petroleum ether/EtOAc, flow rate: 40 mL/min) to give 2-[[5-chloro-8-(2,6-difluorophenyl)-13-[(2R)-2-methylmorpholin-4-yl]-3,4,7,9,12-pentazatricyclo[8.4.0.0^{2,6}]tetradeca-1(10),2(6),4,7,11,13-hexaen-3-yl]methoxy]ethyl-trimethyl-silane as a red solid (500 mg, 35.1%). LCMS (ESI) m/z: 561.1 [M+H]⁺.

### Step 2: 2-[[8-(2,6-difluorophenyl)-13-[(2R)-2-methylmorpholin-4-yl]-5-(trideuteriomethyl)-3,4,7,9,12-pentazatricyclo[8.4.0.0^{2,6}]tetradeca-1(10),2(6),4,7,11,13-hexaen-3-yl]methoxy]ethyl-trimethyl-silane

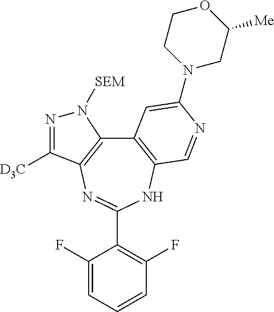 [SMILES: [2H]C([2H])([2H])c1nn(COCC[Si](C)(C)C)c2c1N=C(c1c(F)cccc1F)Nc1cnc(N3CCO[C@H](C)C3)cc1-2]

The title compound was made from 2-[[5-chloro-8-(2,6-difluorophenyl)-13-[(2R)-2-methylmorpholin-4-yl]-3,4,7,9,12-pentazatricyclo[8.4.0.0^{2,6}]tetradeca-1(10),2(6),4,7,11,13-hexaen-3-yl]methoxy]ethyl-trimethyl-silane, following a similar synthetic procedure as described in the synthesis of Int-7 (described above), except that trideuteriomethylboronic acid was used in the place of cyclopropylboronic acid. The reaction was conducted in dioxane and water at 100° C. for 16 hr and treated with 4N HCl in MeOH to remove the SEM protecting group. The resulting mixture was concentrated under reduced pressure and the crude product was purified by preparative HPLC (column: 2_Phenomenex Gemini C18 75×40 mm×3 m; mobile phase: [water (NH₃H₂O+NH₄HCO₃)-ACN]; B %: 30%-60%, 9.5 min) to give (2R)-4-[8-(2,6-difluorophenyl)-5-(trideuteriomethyl)-3,4,7,9,12-pentazatricyclo[8.4.0.0^{2,6}]tetradeca-1(10),2(6),4,7,11,13-hexaen-13-yl]-2-methyl-morpholine as a yellow solid (96.7 mg, 39.7%). ¹H NMR (400 MHz, DMSO-d₆) δ ppm 1.13 (d, J=6.3 Hz, 3H), 2.29-2.41 (m, 1H), 2.61-2.77 (m, 1H), 3.47-3.60 (m, 2H), 3.79 (br d, J=12.3 Hz, 1H), 3.87 (br d, J=11.5 Hz, 2H), 6.82 (br s, 1H), 7.16 (t, J=8.0 Hz, 2H), 7.33-7.58 (m, 2H), 8.24 (br s, 1H), 12.43 (br s, 1H); LCMS (ESI) m/z: 414.0 [M+H]⁺.

### Example 28

### 13-[1-(2,2-difluoroethyl)pyrazol-4-yl]-8-(2,6-difluorophenyl)-5-methyl-3,4,7,9,12-pentazatricyclo[8.4.0.02,6]tetradeca-1(10),2(6),4,7,11,13-hexaene

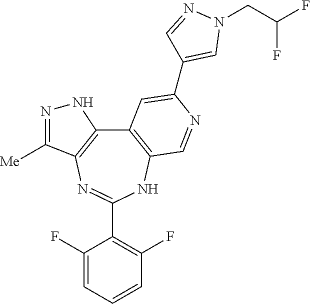 [SMILES: Cc1n[nH]c2c1N=C(c1c(F)cccc1F)Nc1cnc(-c3cnn(CC(F)F)c3)cc1-2]

### Step 1: 2-[[13-[1-(2,2-difluoroethyl)pyrazol-4-yl]-8-(2,6-difluorophenyl)-5-methyl-3,4,7,9,12-pentazatricyclo[8.4.0.02,6] tetradeca-1(10),2(6),4,7,11,13-hexaen-3-yl]methoxy]ethyl-trimethyl-silane

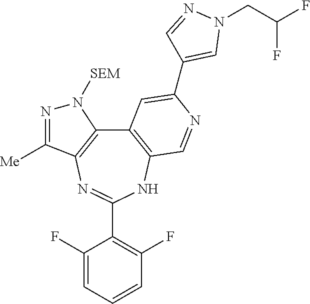 [SMILES: Cc1nn(COCC[Si](C)(C)C)c2c1N=C(c1c(F)cccc1F)Nc1cnc(-c3cnn(CC(F)F)c3)cc1-2]

A mixture of 2-[[13-chloro-8-(2,6-difluorophenyl)-5-methyl-3,4,7,9,12-pentazatricyclo[8.4.0.02,6]tetradeca-1(10),2(6),4,7,11,13-hexaen-3-yl]methoxy]ethyl-trimethyl-silane (Intermediate D2-3, 200 mg, 0.420 mmol, 1.0 eq), 1-(2,2-difluoroethyl)-4-(4,4,5,5-tetramethyl-1,3,2-dioxaborolan-2-yl)pyrazole (108 mg, 0.420 mmol, 1.0 eq), Na₂CO₃ (134 mg, 1.26 mmol, 3.0 eq) and Pd(dppf)Cl₂ (92.2 mg, 0.126 mmol, 0.3 eq) in dioxane/H₂O (5:1, 12 mL) was allowed to stir at 100° C. for 16 hr under N₂ atmosphere. The reaction mixture was filtered and the filtrate was concentrated under reduced pressure. The crude was purified by flash chromatography (ISCO®; 4 g SepaFlash® Silica Flash Column, petroleum ether/EtOAc with EtOAc from 0˜50%, 12 mL/min, 254 nm) to give 2-[[13-[1-(2,2-difluoroethyl)pyrazol-4-yl]-8-(2,6-difluorophenyl)-5-methyl-3,4,7,9,12-pentazatricyclo[8.4.0.02,6] tetradeca-1(10),2(6),4,7,11,13-hexaen-3-yl]methoxy]ethyl-trimethyl-silane as a brown oil (100 mg, 41.6%). ¹H NMR (400 MHz, DMSO-d₆) δ ppm −0.03 (s, 9H), 0.90-0.97 (m, 2H), 2.04 (s, 3H), 3.66-3.77 (m, 2H), 4.66-4.75 (m, 2H), 5.49 (s, 2H), 6.22-6.57 (m, 1H), 7.23 (t, J=8.2 Hz, 2H), 7.53-7.63 (m, 1H), 7.74-7.77 (m, 1H), 7.90 (d, J=2.5 Hz, 2H), 8.18 (s, 1H), 8.52 (s, 1H).

### Step 2: 13-[1-(2,2-difluoroethyl)pyrazol-4-yl]-8-(2,6-difluorophenyl)-5-methyl-3,4,7,9,12-pentazatricyclo[8.4.0.02,6]tetradeca-1(10),2(6),4,7,11,13-hexaene

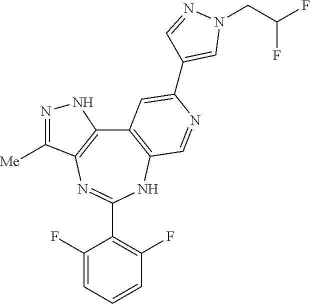 [SMILES: Cc1n[nH]c2c1N=C(c1c(F)cccc1F)Nc1cnc(-c3cnn(CC(F)F)c3)cc1-2]

A solution of 2-[[13-[1-(2,2-difluoroethyl)pyrazol-4-yl]-8-(2,6-difluorophenyl)-5-methyl-3,4,7,9,12-pentazatricyclo[8.4.0.02,6]tetradeca-1(10),2(6),4,7,11,13-hexaen-3-yl]methoxy]ethyl-trimethyl-silane (100 mg, 0.175 mmol) in 4N HCl in MeOH (2 mL) was allowed to stir at 20° C. for 5 hr. The resulting mixture was cooled to 0° C. The precipitate was collected by filtration to provide the tittle compound as a yellow solid (43.0 mg, 55.5%). ¹H NMR (400 MHz, DMSO-d₆) δ ppm 2.08 (s, 3H), 4.71 (td, J=15.1, 3.1 Hz, 2H), 6.09-6.62 (m, 1H), 7.24-7.37 (m, 2H), 7.66 (br s, 1H), 7.80 (s, 1H), 7.89 (br s, 1H), 8.23 (s, 1H), 8.57 (br s, 1H); LCMS (ESI) m/z: 441.1 [M+H]⁺.

### Example 29

### 8-(2,6-difluorophenyl)-5-methyl-13-(1H-pyrazol-4-yl)-3,4,7,9,12-pentazatricyclo[8.4.0.02,6]tetradeca-1(10),2(6),4,7,11,13-hexaene

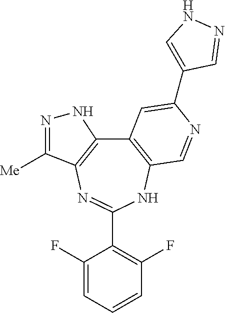 [SMILES: Cc1n[nH]c2c1N=C(c1c(F)cccc1F)Nc1cnc(-c3cn[nH]c3)cc1-2]

The title compound was made from intermediate D2-3, following a similar synthetic procedure as described in the synthesis of Example 28 (described above), except that 4-(4,4,5,5-tetramethyl-1,3,2-dioxaborolan-2-yl)-1H-pyrazole was used in the place of 1-(2,2-difluoroethyl)-4-(4,4,5,5-tetramethyl-1,3,2-dioxaborolan-2-yl)pyrazole, and 10% Pd(dppf)Cl₂ was employed as the catalyst. The reaction was conducted in dioxane/H₂O (5:1) solvent at 100° C. for 12 hr. The reaction was purified by flash chromatography ((ISCO®; 4 g SepaFlash® Silica Flash Column, Eluent of 0˜100% Ethyl acetate/petroleum ether gradient @ 15 mL/min, 254 nm) and treated with 4N HCl in MeOH and triturated with EtOAc to provide the title compound as a yellow solid (40 mg, 44.8%). ¹H NMR (400 MHz, DMSO-d₆) δ ppm 2.03 (s, 3H), 7.22-7.31 (m, 2H), 7.61 (br s, 1H), 7.73-7.83 (m, 2H), 8.34 (br s, 2H); LCMS (ESI) m/z: 378.1 [M+H]⁺.

### Example 30

### Synthesis of (3-chloro-9-(1-(2,2-difluoroethyl)-1H-pyrazol-3-yl)-5-(2,6-difluorophenyl)-1,6-dihydropyrazolo[4,3-d]pyrido[4,3-f][1,3]diazepine)

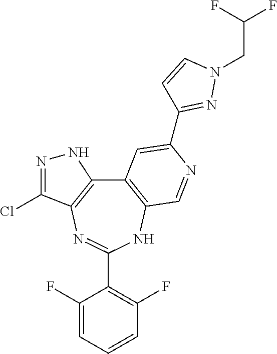 [SMILES: Fc1cccc(F)c1C1=Nc2c(Cl)n[nH]c2-c2cc(-c3ccn(CC(F)F)n3)ncc2N1]

The title compound was made from intermediate D2-2, following a similar synthetic procedure as described in the synthesis of Int-2 (described above) except that 1-(2,2-difluoroethyl)-3-(4,4,5,5-tetramethyl-1,3,2-dioxaborolan-2-yl)pyrazole was used in the place of 1-(difluoromethyl)-3-(4,4,5,5-tetramethyl-1,3,2-dioxaborolan-2-yl)pyrazole. The reaction was conducted in dioxane and water at 90° C. for 16 hr and treated with 4N HCl in MeOH to remove the SEM protecting group. The resulting mixture was concentrated under reduced pressure and the residue was purified by preparative HPLC (column: 2_Phenomenex Gemini C18 75×40 mm×3 m; mobile phase: [water (NH₄HCO₃)-ACN]; B %: 32%-62%, 7.8 min) to give 5-chloro-13-[1-(2,2-difluoroethyl)pyrazol-3-yl]-8-(2,6-difluorophenyl)-3,4,7,9,12-pentazatricyclo[8.4.0.02,6]tetradeca-1(10),2(6),4,7,11,13-hexaene (49.35 mg, 48.6%) as a yellow solid. ¹H NMR (400 MHz, DMSO-d₆) δ ppm 4.69 (td, J=15.1, 3.9 Hz, 2H), 6.19-6.60 (m, 1H), 6.72 (d, J=2.3 Hz, 1H), 7.18-7.25 (m, 2H), 7.50-7.59 (m, 1H), 7.63-7.88 (m, 3H), 8.50-8.92 (m, 1H), 13.54 (br s, 1H); LCMS (ESI) m/z: 461.0 [M+H]⁺.

### Example 31

### 8-(2,6-difluorophenyl)-13-[2-(4-methoxytetrahydropyran-4-yl)ethynyl]-5-methyl-3,4,7,9,12-pentazatricyclo[8.4.0.02,6]tetradeca-1(10),2(6),4,7,11,13-hexaene (2044)

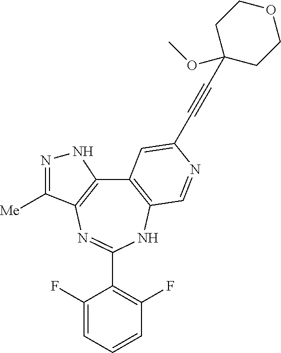 [SMILES: COC1(C#Cc2cc3c(cn2)NC(c2c(F)cccc2F)=Nc2c(C)n[nH]c2-3)CCOCC1]

The title compound was made from Intermediate D2-2, following a similar synthetic procedure as described in the synthesis of Int-3 (described above), except that 4-ethynyl-4-methoxy-tetrahydropyran was used in the place of 4-ethynyl-4-methyl-tetrahydropyran, and 10% of Pd(dppf)Cl₂ was employed as the catalyst. The reaction was conducted in CH₃CN at 110° C. for 16 h, purified by flash chromatography (ISCO®; 4 g SepaFlash® Silica Flash Column, Eluent of 0˜50% Ethyl acetate/Petroleum ether gradient @ 12 mL/min) and treated with 4N HCl in MeOH to provide the title compound as a yellow solid (77.4 mg, 65.2%). ¹H NMR (400 MHz, DMSO-d₆) δ ppm 1.74 (ddd, J=12.9, 9.0, 3.9 Hz, 2H), 1.96 (br d, J=13.4 Hz, 2H), 2.02 (s, 3H), 3.34 (s, 3H), 3.51-3.61 (m, 2H), 3.75-3.84 (m, 2H), 5.56-8.10 (m, 1H), 7.27 (t, J=8.2 Hz, 2H), 7.45 (s, 1H), 7.58-7.68 (m, 1H), 7.85 (s, 1H); LCMS (ESI) m/z: 450.1 [M+H]⁺.

### Example 32

### Synthesis of 8-(2,6-difluorophenyl)-13-[4-(3-fluoropropyl)piperazin-1-yl]-5,11-dimethyl-3,4,7,9,12-pentazatricyclo[8.4.0.02,6]tetradeca-1(10),2(6),4,7,11,13-hexaene

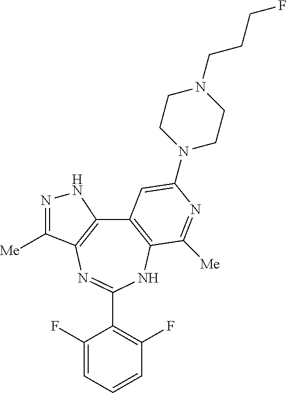 [SMILES: Cc1n[nH]c2c1N=C(c1c(F)cccc1F)Nc1c-2cc(N2CCN(CCCF)CC2)nc1C]

### Step 1: benzyl 4-(3-fluoropropyl)piperazine-1-carboxylate

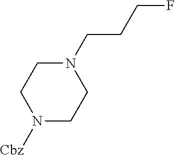 [SMILES: O=C(OCc1ccccc1)N1CCN(CCCF)CC1]

To a solution of benzyl piperazine-1-carboxylate (2 g, 9.08 mmol) in DMF (20 mL) was added 1-fluoro-3-iodo-propane (1.88 g, 9.99 mmol) and Cs₂CO₃ (8.88 g, 27.2 mmol). The mixture was allowed to stir at 90° C. for 16 h. The reaction mixture was diluted with H₂O (30 mL) and extracted with EtOAc (30 mL×3). The combined organic layers were washed with brine (30 mL×2), dried with anhydrous Na₂SO₄, filtered and concentrated under reduced pressure. The residue was purified by flash chromatography (ISCO®; 12 g SepaFlash® Silica Flash Column, petroleum ether/EtOAc with EtOAc from 0˜30%, 35 mL/min, 254 mn) to give benzyl 4-(3-fluoropropyl)piperazine-1-carboxylate (1.9 g, 70.1%) as a yellow oil. LCMS (ESI) m/z: 281.1 [M+H]⁺.

### Step 2: 1-(3-fluoropropyl)piperazine

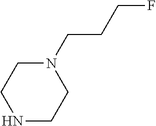

To a solution of benzyl 4-(3-fluoropropyl)piperazine-1-carboxylate (1 g, 3.57 mmol) in MeOH (10 mL) was added Pd/C (379 mg, 10 wt % Pd with 50 wt % water) under N₂ atmosphere. The suspension was degassed and purged with H₂ for 3 times. The mixture was allowed to stir under H₂ (15 psi) at 25° C. for 3 h. The reaction mixture was filtered and concentrated under reduced pressure to give 1-(3-fluoropropyl)piperazine (500 mg, crude) as a white solid.

### Step 3: 8-(2,6-difluorophenyl)-13-[4-(3-fluoropropyl)piperazin-1-yl]-5,11-dimethyl-3,4,7,9,12-pentazatricyclo[8.4.0.02,6]tetradeca-1(10),2(6),4,7,11,13-hexaene

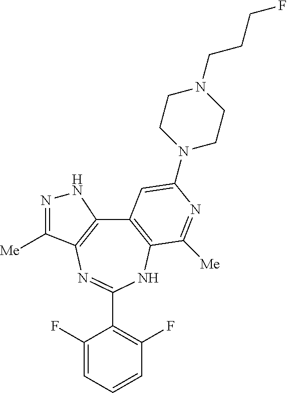 [SMILES: Cc1n[nH]c2c1N=C(c1c(F)cccc1F)Nc1c-2cc(N2CCN(CCCF)CC2)nc1C]

The title compound was made from intermediate D4-1, following a similar synthetic procedure as described in the synthesis of Int-4 (described above), except that 1-(3-fluoropropyl)piperazin was used in the place of 1-(2-fluoroethyl)-3-methyl-piperazine. The reaction was conducted in dioxane at 100° C. for 12 h. and treated with 4N HCl in MeOH to remove the SEM protecting group. The resulting mixture was concentrated under reduced pressure and then triturated with MeCN at 25° C. for 30 min to give 8-(2,6-difluorophenyl)-13-[4-(3-fluoropropyl)piperazin-1-yl]-5,11-dimethyl-3,4,7,9,12-pentazatricyclo[8.4.0.02,6]tetradeca-1(10),2(6),4,7,11,13-hexaene (77.28 mg, 98.4%) as a yellow solid. ¹H NMR (400 MHz, CD₃OD) δ ppm 2.14-2.25 (m, 1H), 2.29 (s, 4H), 2.45 (s, 3H), 3.08-3.26 (m, 2H), 3.35-3.45 (m, 4H), 3.70 (br s, 2H), 4.55 (t, J=5.4 Hz, 3H), 4.66 (t, J=5.5 Hz, 1H), 7.15 (s, 1H), 7.35 (t, J=8.8 Hz, 2H), 7.83-7.92 (m, 1H); LCMS (ESI) m/z: 470.3 [M+H]⁺.

### Example 33

### Synthesis of 4-[8-(2,6-difluorophenyl)-3,4,7,9,12-pentazatricyclo[8.4.0.02,6]tetradeca-1(10),2(6),4,7,11,13-hexaen-13-yl]morpholine

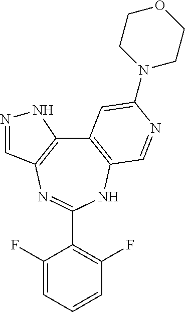 [SMILES: Fc1cccc(F)c1C1=Nc2cn[nH]c2-c2cc(N3CCOCC3)ncc2N1]

The title compound was made from intermediate D2-1, following a similar synthetic procedure as described in the synthesis of Int-4 (described above). The reaction was conducted in dioxane at 100° C. for 16 h. and treated with 4N HCl in MeOH to remove the SEM protecting group. The resulting mixture was concentrated under reduced pressure and then purified by flash chromatography on silica gel column using 0-80% of EtOAc in petroleum ether as eluent, to provide 4-[8-(2,6-difluorophenyl)-3,4,7,9,12-pentazatricyclo[8.4.0.02,6]tetradeca-1(10),2(6),4,7,11,13-hexaen-13-yl]morpholine as a yellow solid (100 mg, 51.5%). ¹H NMR (400 MHz, DMSO-d₆) δ ppm 3.27 (br dd, J=3.9, 8.9 Hz, 4H), 3.64-3.69 (m, 5H), 6.85 (br d, J=17.6 Hz, 1H), 6.85 (s, 1H), 7.15 (t, J=7.9 Hz, 3H), 7.30 (s, 1H), 7.44 (s, 1H), 7.45-7.52 (m, 1H), 8.33 (s, 1H), 12.63 (s, 1H), 12.61-12.66 (m, 1H); LCMS (ESI) m/z: 383.0 [M+H]⁺.

### Example 34 and Example 35

### Synthesis of 13-[(3R)-3-(difluoromethyl)pyrrolidin-1-yl]-8-(2,6-difluorophenyl)-3,4,7,9,12-pentazatricyclo[8.4.0.02,6]tetradeca-1(10),2(6),4,7,11,13-hexaene (Example 34) and 13-[(3S)-3-(difluoromethyl)pyrrolidin-1-yl]-8-(2,6-difluorophenyl)-3,4,7,9,12-pentazatricyclo[8.4.0.02,6]tetradeca-1(10),2(6),4,7,11,13-hexaene (Example 35)

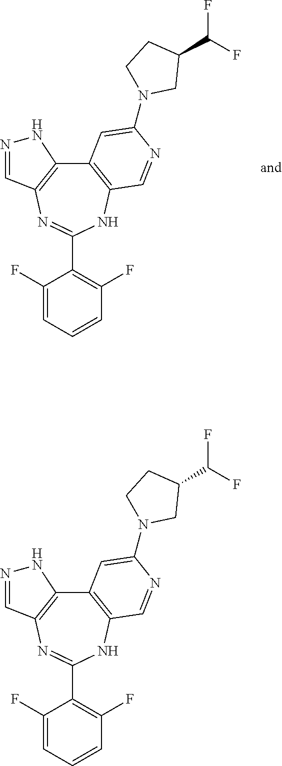 [SMILES: Fc1cccc(F)c1C1=Nc2cn[nH]c2-c2cc(N3CC[C@@H](C(F)F)C3)ncc2N1 ; Fc1cccc(F)c1C1=Nc2cn[nH]c2-c2cc(N3CC[C@H](C(F)F)C3)ncc2N1]

The title compound was made from intermediate D2-1, following a similar synthetic procedure as described in the synthesis of Int-4 (described above), except that 3-(difluoromethyl)pyrrolidine was used in the place of 1-(2-fluoroethyl)-3-methyl-piperazine. The reaction was conducted in dioxane at 110° C. for 30 min and treated by 4N HCl in MeOH. The residue was purified by flash chromatography on silica gel column using 0-60% of EtOAc in petroleum ether as eluent, to provide the racemic product as a yellow solid (72 mg, 32.2%). The crude product was dissolved in DMSO and re-subjected to chiral preparative HPLC (column: Daicel Chiralpak AD (250 mm×30 mm, 10 μm); mobile phase: [0.1% NH₃—H₂O EtOH]; B %: 30%-30%, 9 min) to provide the following two products respectively:

Fraction A (15.8 mg, RT=5.289 min, @ xxx condition) was tentatively assigned as compound Example 34: ¹H NMR (400 MHz, DMSO-d₆) δ ppm 1.91-2.00 (m, 1H), 2.04-2.16 (m, 1H), 2.79 (br d, J=11.5 Hz, 1H), 3.28-3.31 (m, 2H), 3.41-3.55 (m, 2H), 6.31-5.96 (m, 1H), 6.53-6.59 (m, 1H), 7.15 (br t, J=8.0 Hz, 2H), 7.29 (s, 1H), 7.40 (s, 1H), 7.44-7.53 (m, 1H), 8.28 (s, 1H), 12.61 (s, 1H); LCMS (ESI) m/z: 417.2 [M+H]⁺.

Fraction B (16.1 mg, RT=5.526 min, @ xxx condition) was tentatively assigned as compound Example 35: ¹H NMR (400 MHz, DMSO-d₆) δ ppm 1.91-2.00 (m, 1H), 2.04-2.16 (m, 1H), 2.79 (br d, J=11.5 Hz, 1H), 3.28-3.31 (m, 2H), 3.41-3.55 (m, 2H), 6.31-5.96 (m, 1H), 6.53-6.59 (m, 1H), 7.15 (br t, J=8.0 Hz, 2H), 7.29 (s, 1H), 7.40 (s, 1H), 7.44-7.53 (m, 1H), 8.28 (s, 1H), 12.61 (s, 1H); LCMS (ESI) m/z: 417.2 [M+H]⁺.

### Example 36

### Synthesis of 2-[(2S)-4-[5-(2,6-difluorophenyl)-3-methyl-1,6-dihydropyrazolo[4,3-d][1,3]benzodiazepin-9-yl]morpholin-2-yl]propan-2-ol

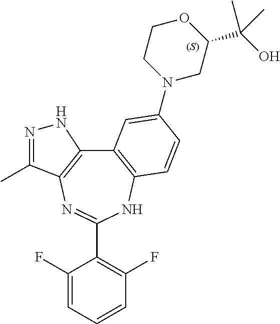

### Step 1: Synthesis of tert-butyl 4-(2-fluoroethyl)-3-methyl-piperazine-1-carboxylate

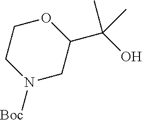 [SMILES: CC(C)(C)OC(=O)N1CCOC(C(C)(C)O)C1]

A mixture of 4-(tert-butyl) 2-ethyl morpholine-2,4-dicarboxylate (1 g, 3.86 mmol, 1 eq) and bromo(methyl)magnesium (1.84 g, 15.43 mL, 4 eq) in THF (20 mL) was degassed and purged with N₂ for 3 times. The mixture was stirred at 0-20° C. for 6 hr under N₂ atmosphere, diluted with H₂O (30 mL) and extracted with EA (90 mL). The combined organic layers were washed with brine (60 mL), dried with anhydrous Na₂SO₄, filtered and concentrated under reduced pressure. The residue was purified by flash silica gel chromatography (ISCO®; 12 g SepaFlash® Silica Flash Column, Eluent of 0˜40% petroleum ether/EtOAc @ 45 mL/min) to give tert-butyl 2-(1-hydroxy-1-methyl-ethyl)morpholine-4-carboxylate as a white oil (600 mg, 63.42%).

### Step 2: Synthesis of 1-(2-fluoroethyl)-2-methyl-piperazine

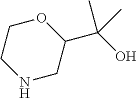

A mixture of tert-butyl 2-(1-hydroxy-1-methyl-ethyl)morpholine-4-carboxylate (600 mg, 2.45 mmol, 1 eq) in 4N HCl in MeOH (10 mL) was stirred at 20° C. for 1 hr. The reaction mixture was concentrated under reduced pressure to provide 2-morpholin-2-ylpropan-2-ol as a white oil (300 mg, 84.48%).

### Step 3: Synthesis of 2-[4-[5-(2,6-difluorophenyl)-3-methyl-1-(2-trimethylsilylethoxymethyl)-6H-pyrazolo[4,3-d][1,3]benzodiazepin-9-yl]morpholin-2-yl]propan-2-ol

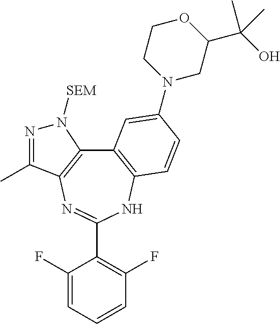 [SMILES: Cc1nn(COCC[Si](C)(C)C)c2c1N=C(c1c(F)cccc1F)Nc1ccc(N3CCOC(C(C)(C)O)C3)cc1-2]

A mixture of 2-morpholin-2-ylpropan-2-ol (185 mg, 1.27 mmol, 1.1 eq), 2-[[9-bromo-5-(2,6-difluorophenyl)-3-methyl-6H-pyrazolo[4,3-d][1,3]benzodiazepin-1-yl]methoxy]ethyl-trimethyl-silane (600 mg, 1.16 mmol, 1 eq), t-BuONa (666 mg, 6.93 mmol, 6 eq) and RuPhos Pd G3 (386 mg, 0.46 mmol, 0.4 eq) in dioxane (6 mL) was degassed and purged with N₂ for 3 times. The mixture was stirred at 110° C. for 1 hr under N₂ atmosphere, filtered and concentrated under reduced pressure. The residue was purified by flash silica gel chromatography (ISCO®; 12 g SepaFlash® Silica Flash Column, Eluent of 0˜30% Ethyl acetate/Petroleum ether gradient @ 30 mL/min) to give 2-[4-[5-(2,6-difluorophenyl)-3-methyl-1-(2-trimethylsilylethoxymethyl)-6H-pyrazolo[4,3-d][1,3]benzodiazepin-9-yl]morpholin-2-yl]propan-2-ol as a green solid (200 mg, 22.5%). LCMS (ESI) m/z: 584.3 [M+H]⁺.

### Step 4: Synthesis of 2-[4-[5-(2,6-difluorophenyl)-3-methyl-1,6-dihydropyrazolo[4,3-d][1,3]benzodiazepin-9-yl]morpholin-2-yl]propan-2-ol

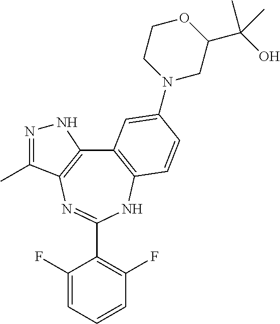 [SMILES: Cc1n[nH]c2c1N=C(c1c(F)cccc1F)Nc1ccc(N3CCOC(C(C)(C)O)C3)cc1-2]

To a solution of 2-[4-[5-(2,6-difluorophenyl)-3-methyl-1-(2-trimethylsilylethoxymethyl)-6H-pyrazolo[4,3-d][1,3]benzodiazepin-9-yl]morpholin-2-yl]propan-2-ol (200 mg, 0.34 mmol, 1 eq) in 4N HCl in MeOH (2 mL) was added Et₃SiH (39.8 mg, 0.34 mmol, 1 eq). The mixture was stirred at 20° C. for 1 hr, diluted with H₂O (10 mL) and extracted with EtOAc (20 mL). The combined organic layers were washed with brine (10 mL), dried over Na₂SO₄, filtered and concentrated under reduced pressure. The residue was purified by flash silica gel chromatography (ISCO®; 4 g SepaFlash® Silica Flash Column, Eluent of 0˜30% Ethyl acetate/Petroleum ether gradient @ 30 mL/min) to give 2-[4-[5-(2,6-difluorophenyl)-3-methyl-1,6-dihydropyrazolo[4,3-d][1,3]benzodiazepin-9-yl]morpholin-2-yl]propan-2-ol as a green solid (120 mg, 70.28%). LCMS (ESI) m/z: 454.2 [M+H]⁺.

### Step 5: Synthesis of 2-[(2S)-4-[5-(2,6-difluorophenyl)-3-methyl-1,6-dihydropyrazolo[4,3-d][1,3]benzodiazepin-9-yl]morpholin-2-yl]propan-2-ol

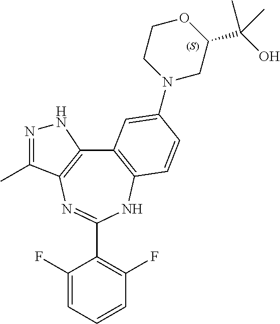

The racemate was purified by SFC (column: Phenomenex-Cellulose-2 (250 mm×30 mm×10 m); mobile phase: [0.1% NH₃H₂O MEOH]; B %: 45%-45%, 12 min) to provide the following two products respectively:

Fraction A (33.59 mg, RT=4.43 min, @ (column: DAICEL CHIRALPAK AD (250 mm×30 mm×10 m); mobile phase: [0.1% NH₃H₂O EtOH]; B %: 40%-40%, min as additive) was the desired compound Example 36: ¹H NMR (400 MHz, CD₃OD) δ ppm 1.26 (d, J=7.63 Hz, 6H), 2.12 (s, 3H), 2.56 (s, 1H), 2.67-2.77 (m, 1H), 3.40 (br d, J=1.91 Hz, 2H), 3.58 (br d, J=11.56 Hz, 1H), 3.75 (br d, J=2.38 Hz, 1H), 4.00-4.10 (m, 1H), 6.50 (d, J=8.70 Hz, 1H), 6.64-6.76 (m, 1H), 7.07 (s, 2H), 7.12-7.26 (m, 1H), 7.42-7.55 (m, 1H); ¹⁹F NMR (376 MHz, CD₃OD) δ ppm −112.27; LCMS (ESI) m/z: 454.2 [M+H]⁺.

### Example 37

### Synthesis of (R)-(4-(5-(2,6-difluorophenyl)-3,7-dimethyl-1,6-dihydropyrazolo[4,3-d]pyrido[4,3-f][1,3]diazepin-9-yl)morpholin-2-yl)methanol

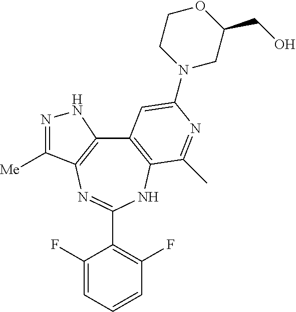 [SMILES: Cc1n[nH]c2c1N=C(c1c(F)cccc1F)Nc1c-2cc(N2CCO[C@@H](CO)C2)nc1C]

The title compound was made from Intermediate D4-1, following a similar synthetic procedure as described in the synthesis of Int-1 (described above), except that [(2R)-morpholin-2-yl]methanol was used in the place of morpholine. The reaction was conducted in dioxane at 100° C. for 2 hr. and treated with 4.0 N HCl in MeOH to remove the SEM protecting group. The resulting mixture was concentrated under reduced pressure and purified by reverse phase preparative HPLC (column: Welch Xtimate C18 100×40 mm×3 m; mobile phase: [water (HCl)-ACN]; B %: 0%-30%, 8 min) to provide the title compound as a yellow solid (45.5 mg, 84.1%). ¹H NMR (400 MHz, CD₃OD) δ ppm 2.32 (s, 3H), 2.56 (s, 3H), 3.07-3.19 (m, 1H), 3.27 (br d, J=3.81 Hz, 1H), 3.64-3.70 (m, 3H), 3.71-3.78 (m, 1H), 4.06-4.14 (m, 2H), 4.19 (br d, J=12.87 Hz, 1H), 7.35 (t, J=8.82 Hz, 2H), 7.42 (s, 1H), 7.87 (tt, J=8.57, 6.45 Hz, 1H); ¹⁹F NMR (376 MHz, CD₃OD) δ ppm −112.27; LCMS (ESI) m/z: 441.2 [M+H]⁺.

### Example 38

### 8-[8-(2,6-difluorophenyl)-5-methyl-3,4,7,9,12-pentazatricyclo[8.4.0.02,6] tetradeca-1(10),2(6),4,7,11,13-hexaen-13-yl]-2-oxa-5,8-diazaspiro[3.5]nonane

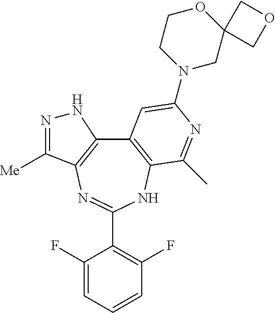 [SMILES: Cc1n[nH]c2c1N=C(c1c(F)cccc1F)Nc1c-2cc(N2CCOC3(COC3)C2)nc1C]

The title compound was made from Intermediate D4-1, following a similar synthetic procedure as described in the synthesis of Int-1 (described above), except that 2,5-dioxa-8-azaspiro[3.5]nonane was used in the place of morpholine. The reaction was conducted in dioxane at 100° C. for 3 hr and treated with 4.0 N HCl in MeOH to remove the SEM protecting group. The resulting mixture was concentrated under reduced pressure and purified by filtration to provide the title compound as a yellow solid (14.6 mg, 34.59%). ¹H NMR (400 MHz, DMSO-d₆) δ ppm 2.13 (br s, 3H), 2.27-2.33 (m, 3H), 3.39 (br s, 2H), 3.68 (br s, 2H), 3.71 (br s, 2H), 4.40 (br d, J=6.75 Hz, 2H), 4.47 (br d, J=6.88 Hz, 2H), 6.93 (s, 1H), 7.24-7.43 (m, 2H), 7.58-7.87 (m, 1H). LCMS (ESI) m/z: 453.4 [M+H]⁺.

### Example 39

### 2-[(2R)-4-[8-(2,6-difluorophenyl)-5,11-dimethyl-3,4,7,9,12-pentazatricyclo[8.4.0.0^{2,6}]tetradeca-1(10),2(6),4,7,11,13-hexaen-13-yl]morpholin-2-yl]propan-2-ol

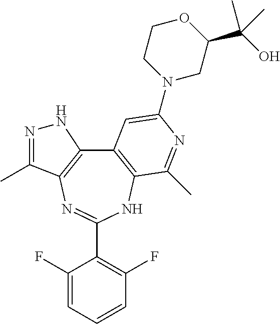 [SMILES: Cc1n[nH]c2c1N=C(c1c(F)cccc1F)Nc1c-2cc(N2CCO[C@@H](C(C)(C)O)C2)nc1C]

The title compound was made from Intermediate D4-1, following a similar synthetic procedure as described in the synthesis of Int-1 (described above), except that 2-morpholin-2-ylpropan-2-ol was used in the place of morpholine. The reaction was conducted in dioxane at 100° C. for 2 hr. and treated with 4N HCl in MeOH to remove the SEM protecting group. The resulting mixture was concentrated under reduced pressure and purified by filtration to provide the racemic compound as a yellow solid (1.2 g, 93.04%). The racemate was purified by SFC (column: DAICEL CHIRALPAK IC (250 mm×30 mm×10 m); mobile phase: [0.1% NH₃H₂O MEOH]; B %: 40%-40%, 12 min) to provide the following two products respectively:

Fraction A (49.78 mg, RT=1.468 min, @ (column: DAICEL CHIRALPAK AD (250 mm×30 mm×10 m); mobile phase: [0.1% NH₃H₂O EtOH]; B %: 40%-40%, 12 min) was the desired compound Example 39: ¹H NMR (400 MHz, CD₃OD) δ ppm 1.2-1.25 (m, 3H), 1.25-1.28 (m, 3 H), 2.16 (br s, 3H), 2.21-2.29 (m, 3H), 2.62-2.71 (m, 1H), 2.76-2.86 (m, 1H), 3.32-3.36 (m, 1H), 3.61-3.70 (m, 1H), 3.92-4.05 (m, 2H), 4.15 (br d, J=12.01 Hz, 1H), 6.79-6.90 (m, 1H), 7.03-7.12 (m, 2H), 7.44-7.54 (m, 1H); ¹⁹F NMR (377 MHz, CD₃OD) δ ppm −115.16; LCMS (ESI) m/z 469.2 [M+H]⁺.

### Example 40

### 1-(5-(2,6-difluorophenyl)-3,7-dimethyl-1,6-dihydropyrazolo[4,3-d]pyrido[4,3-f][1,3]diazepin-9-yl)piperidin-4-ol

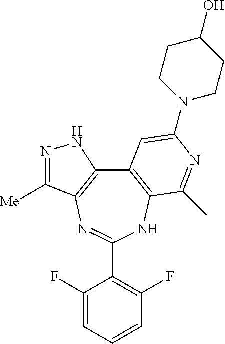 [SMILES: Cc1n[nH]c2c1N=C(c1c(F)cccc1F)Nc1c-2cc(N2CCC(O)CC2)nc1C]

The title compound was made from Intermediate D4-1, following a similar synthetic procedure as described in the synthesis of Int-1 (described above), except that piperidin-4-ol was used in the place of morpholine. The reaction was conducted in dioxane at 100° C. for 2 hrs and treated with 4N HCl in MeOH to remove the SEM protecting group. The resulting mixture was concentrated under reduced pressure and purified by prep-HPLC (column: Boston Prime C18 150×30 mm×5 m; mobile phase: [water (ammonia hydroxide v/v)-ACN]; B %: 15%-45%, 16 min) to provide 1-[8-(2,6-difluorophenyl)-5,11-dimethyl-3,4,7,9,12-pentazatricyclo[8.4.0.02,6]tetradeca-1(10),2(6),4,7,11,13-hexaen-13-yl]piperidin-4-ol as a yellow solid (14 mg, 11.4%). ¹H NMR (400 MHz, CD₃OD) δ ppm 1.46-1.61 (m, 2H), 1.87-2.00 (m, 2H), 2.10 (s, 3H), 2.26 (s, 3H), 2.97-3.13 (m, 2H), 3.74-3.88 (m, 1H), 3.94-4.10 (m, 2H), 6.74-6.96 (m, 1H), 6.99-7.22 (m, 2H), 7.41-7.60 (m, 1H); LCMS (ESI) m/z: 425.1 [M+H]⁺.

### Example 41

### 1-[8-(2,6-difluorophenyl)-5,11-dimethyl-3,4,7,9,12-pentazatricyclo[8.4.0.02,6]tetradeca-1(10),2(6),4,7,11,13-hexaen-13-yl]-4-methyl-piperidin-4-ol

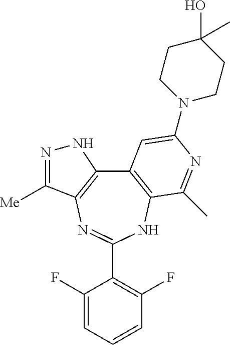 [SMILES: Cc1n[nH]c2c1N=C(c1c(F)cccc1F)Nc1c-2cc(N2CCC(C)(O)CC2)nc1C]

The title compound was made from Intermediate D4-1, following a similar synthetic procedure as described in the synthesis of Int-1 (described above), except that 4-methylpiperidin-4-ol was used in the place of morpholine. The reaction was conducted in dioxane at 100° C. for 2 hr. and treated with 4.0 N HCl in MeOH to remove the SEM protecting group. The resulting mixture was concentrated under reduced pressure and purified by filtration to provide the title compound as a yellow solid (19.69 mg, 38.29%). 1H NMR (400 MHz, CD₃OD) δ ppm 1.25 (s, 3H), 1.59-1.67 (m, 4H), 2.16 (br s, 3H), 2.24 (s, 3H), 3.33-3.41 (m, 2H), 3.74 (br d, J=12.80 Hz, 2H), 6.86 (br s, 1H), 7.08 (t, J=8.03 Hz, 2H), 7.49 (q, J=7.40 Hz, 1H). LCMS (ESI) m/z: 439.2 [M+H]⁺.

### Example 42

### 7-[8-(2,6-difluorophenyl)-5-methyl-3,4,7,9,12-pentazatricyclo[8.4.0.0^{2,6}]tetradeca-1(10),2(6),4,7,11,13-hexaen-13-yl]-2-oxa-7-azaspiro[3.5]nonane

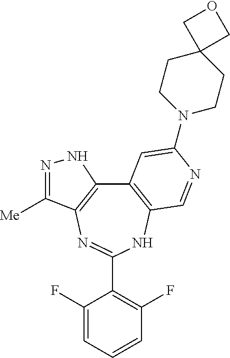 [SMILES: Cc1n[nH]c2c1N=C(c1c(F)cccc1F)Nc1cnc(N3CCC4(CC3)COC4)cc1-2]

The title compound was made from Intermediate D2-3, following a similar synthetic procedure as described in the synthesis of Int-1 (described above), except that 2-[[8-(2,6-difluorophenyl)-5,13-dimethyl-3,4,7,9,12-pentazatricyclo[8.4.0.0^{2,6}]tetradeca-1(10),2(6),4,7,11, 13-hexaen-3-yl]methoxy]ethyl-trimethyl-silane was used in the place of morpholine. The reaction was conducted in dioxane at 100° C. for 2 hrs. and treated with TFA in DCM to remove the SEM protecting group. The crude residue was purified by prep-HPLC (Boston Prime C18 150×30 mm×5 m; mobile phase: [water (NH₃H₂O+NH₄HCO₃)-ACN]; B %: 25%-65%, 10 min) to provide the title compound as a yellow solid (27 mg, 35.1%). ¹H NMR (400 MHz, CD₃OD) δ ppm 1.91-1.97 (m, 4H), 2.09-2.17 (m, 3H), 3.39-3.45 (m, 4H), 4.49-4.53 (m, 4H), 6.99-7.12 (m, 3H), 7.36-7.54 (m, 2H); ¹⁹F NMR (376 MHz, CD₃OD) δ ppm −115.55; LCMS (ESI) m/z: 437.1 [M+H]⁺.

### Example 43

### 6-[8-(2,6-difluorophenyl)-11-methyl-3,4,7,9,12-pentazatricyclo[8.4.0.02,6]tetradeca-1(10),2(6),4,7,11,13-hexaen-13-yl]-2-oxa-6-azaspiro[3.3]heptane

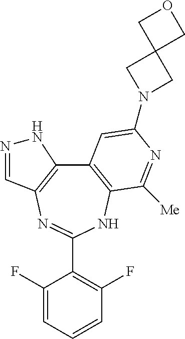 [SMILES: Cc1nc(N2CC3(COC3)C2)cc2c1NC(c1c(F)cccc1F)=Nc1cn[nH]c1-2]

The title compound was made from Intermediate D4-2, following a similar synthetic procedure as described in the synthesis of Int-6 (described above), except that 2-oxa-6-azaspiro[3.3]heptane was used in the place of morpholine. The reaction was conducted in dioxane at 100° C. for 2 hrs. and treated with TFA to remove the PMB protecting group. The crude was purified by Prep-HPLC (column: Boston Prime C18 150×30 mm×5 m; mobile phase: [water (ammonia hydroxide v/v)-ACN]; B %: 12%-42%, 9 min) to provide the title compound as a yellow solid (20.8 mg, 29.9% yield). ¹H NMR (400 MHz, CD₃OD) δ ppm 2.24 (s, 3H), 4.16 (m, 4H), 4.86 (m, 4H), 6.56 (br s, 1H), 7.10 (br t, J=7.8 Hz, 2H), 7.33 (br s, 1H), 7.50 (br d, J=6.3 Hz, 1H); ¹⁹F NMR (376 MHz, CD₃OD) δ ppm −114.35; LCMS (ESI) m/z: 449.9 [M+H]⁺.

### Example 44

### 8-(2,6-difluorophenyl)-5,11-dimethyl-13-(4-methylsulfonylpiperazin-1-yl)-3,4,7,9,12-pentazatricyclo[8.4.0.0^{2,6}]tetradeca-1(10),2(6),4,7,11,13-hexaene

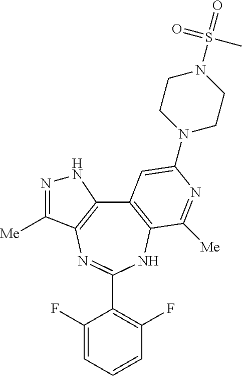 [SMILES: Cc1n[nH]c2c1N=C(c1c(F)cccc1F)Nc1c-2cc(N2CCN(S(C)(=O)=O)CC2)nc1C]

### Step 1: Synthesis of 2-[[8-(2,6-difluorophenyl)-5,11-dimethyl-13-(4-methylsulfonylpiperazin-1-yl)-3,4,7,9,12-pentazatricyclo[8.4.0.0^{2,6}]tetradeca-1(10),2(6),4,7,11,13-hexaen-3-yl]methoxy]ethyl-trimethyl-silane

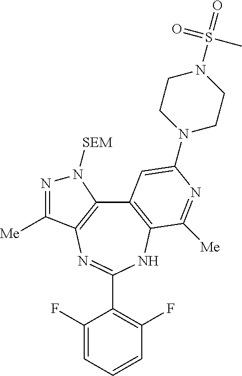 [SMILES: Cc1nn(COCC[Si](C)(C)C)c2c1N=C(c1c(F)cccc1F)Nc1c-2cc(N2CCN(S(C)(=O)=O)CC2)nc1C]

To a solution of 2-[[8-(2,6-difluorophenyl)-5,11-dimethyl-13-piperazin-1-yl-3,4,7,9,12-pentazatricyclo[8.4.0.0^{2,6}]tetradeca-1(10),2(6),4,7,11,13-hexaen-3-yl]methoxy]ethyl-trimethyl-silane (100 mg, 0.18 mmol, 1 eq) in DCM (2 mL) was added MsCl (21.2 mg, 0.18 mmol, 1 eq) and TEA (37.5 mg, 0.37 mmol, 2 eq). The mixture was stirred at 0° C. for 1 hr., then quenched by H₂O (5 mL×1) and extracted with EtOAc (10 mL×3). The combined organic layers were washed with Brine (10 mL), dried over Na₂SO₄, filtered and concentrated under reduced pressure. The residue was purified by column chromatography (ISCO®; 4 g SepaFlash® Silica Flash Column, petroleum ether/EtOAc with EtOAc from 0˜39%, 50 mL/min, 254 nm) to afford 2-[[8-(2,6-difluorophenyl)-5,11-dimethyl-13-(4-methylsulfonylpiperazin-1-yl)-3,4,7,9,12-pentazatricyclo[8.4.0.0^{2,6}]tetradeca-1(10),2(6),4,7,11,13-hexaen-3-yl]methoxy]ethyl-trimethyl-silane as a yellow solid (77.8 mg, 65.2%); LCMS (ESI) m/z: 618.2 [M+H]⁺.

### Step 2: Synthesis of 8-(2,6-difluorophenyl)-5,11-dimethyl-13-(4-methylsulfonylpiperazin-1-yl)-3,4,7,9,12-pentazatricyclo[8.4.0.0^{2,6}]tetradeca-1(10),2(6),4,7,11,13-hexaene

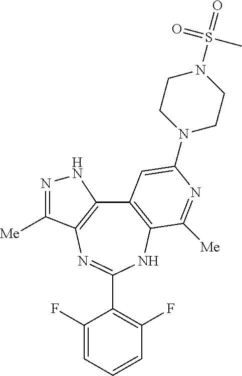 [SMILES: Cc1n[nH]c2c1N=C(c1c(F)cccc1F)Nc1c-2cc(N2CCN(S(C)(=O)=O)CC2)nc1C]

To a solution of 2-[[8-(2,6-difluorophenyl)-5,11-dimethyl-13-(4-methylsulfonylpiperazin-1-yl)-3,4,7,9,12-pentazatricyclo[8.4.0.0^{2,6}]tetradeca-1(10),2(6),4,7,11,13-hexaen-3-yl]methoxy]ethyl-trimethyl-silane (77.8 mg, 0.12 mmol, 1 eq) in 4N HCl in MeOH (2 mL) was added Et₃SiH (0.14 mg, 1.26 umol, 0.01 eq). The mixture was stirred at 25° C. for 1 hr, diluted with NaHCO₃ aqueous solution (10 mL), extracted with EtOAc (10 mL×3), dried over Na₂SO₄ and concentrated under reduced pressure. The residue was purified by column chromatography (ISCO®; 4 g SepaFlash® Silica Flash Column, petroleum ether/EtOAc with EtOAc from 0˜70%, 50 mL/min, 254 nm) to afford the title compound as a yellow solid (32 mg, 50.99%). ¹H NMR (400 MHz, CD₃OD) δ ppm 2.16 (br s, 3H), 2.25 (s, 3H), 2.86 (s, 3H), 3.29 (br s, 2H), 3.64-3.56 (m, 4H), 4.63 (s, 2H), 6.88 (br s, 1H), 7.08 (br t, J=8.0 Hz, 1H), 7.08-7.08 (m, 1H), 7.54-7.46 (m, 1H); ¹⁹F NMR (377 MHz, CD₃OD) δ ppm −115.21; LCMS (ESI) m/z: 488.2 [M+H]⁺.

### Example 45

### (R)-1-((S)-4-(5-(2,6-difluorophenyl)-3,7-dimethyl-1,6-dihydropyrazolo[4,3-d]pyrido[4,3-f][1,3]diazepin-9-yl)morpholin-2-yl)ethan-1-ol

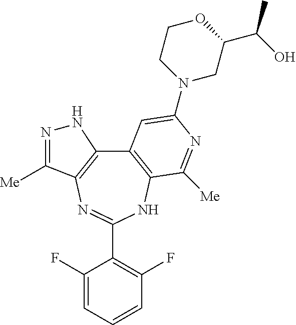 [SMILES: Cc1n[nH]c2c1N=C(c1c(F)cccc1F)Nc1c-2cc(N2CCO[C@H]([C@@H](C)O)C2)nc1C]

The title compound was made from Intermediate D4-1, following a similar synthetic procedure as described in the synthesis of Int-1 (described above), except that 1-(morpholin-2-yl)ethan-1-ol was used in the place of morpholine. The reaction was conducted in dioxane at 100° C. for 2 hrs and treated with 4N HCl in MeOH to remove the SEM protecting group. The crude product was triturated with EtOAc at 25° C. for 60 mins, filtered, contracted and purified by chiral separation (column: Phenomenex-Cellulose-2 (250 mm×30 mm×10 m); mobile phase: [0.1% NH₃H₂O:EtOH]; B %: 30%-30%, min) to provide the title compound as a yellow solid (18 mg, 18.9%). ¹H NMR (400 MHz, CD₃OD) δ ppm 1.26 (d, J=6.53 Hz, 3H), 1.31 (s, 1H), 2.18 (s, 3H), 2.29 (s, 3H), 2.68-2.76 (m, 1H), 2.83-2.94 (m, 1H), 3.39 (ddd, J=10.67, 5.40, 2.51 Hz, 1H), 3.63-3.84 (m, 2H), 3.91-4.08 (m, 3H), 6.88 (br s, 1H), 7.12 (t, J=8.16 Hz, 2H), 7.45-7.64 (m, 1H); ¹⁹F NMR (377 MHz, CD₃OD) δ ppm −114.25; LCMS (ESI) m/z: 455.2 [M+H]⁺.

### Example 46

### 4-[8-(2,6-difluorophenyl)-5,11-dimethyl-3,4,7,9,12-pentazatricyclo[8.4.0.02,6]tetradeca-1(10),2(6),4,7,11,13-hexaen-13-yl]-1,4-thiazinane 1,1-dioxide

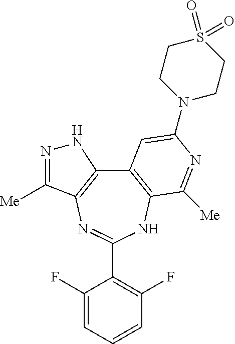 [SMILES: Cc1n[nH]c2c1N=C(c1c(F)cccc1F)Nc1c-2cc(N2CCS(=O)(=O)CC2)nc1C]

The title compound was made from Intermediate D4-1, following a similar synthetic procedure as described in the synthesis of Int-1 (described above), except that 1,4-thiazinane 1,1-dioxide was used in the place of morpholine, and 10% Pd₂(dba)₃ was employed as the catalyst. The reaction was conducted in dioxane at 100° C. for 2 hr. and treated with 4N HCl in MeOH to remove the SEM protecting group. The resulting residue was purified by prep-HPLC (column: Welch Xtimate C18 150×30 mm×5 um; mobile phase: [water(0.05% HCl)-ACN]) to provide the title compound as a yellow solid (7.1 mg, 7.45%). ¹H NMR (400 MHz, DMSO-d₆) δ ppm 2.20 (s, 3H), 2.36 (s, 3H), 3.15 (br s, 4H), 4.05 (br s, 4H), 7.04 (s, 1H), 7.41 (br t, J=8.82 Hz, 2H), 7.76-7.90 (m, 1H); ¹⁹F NMR (377 MHz, DMSO-d₆) δ ppm −115.55; LCMS (ESI) m/z: 459.4 [M+H]⁺.

### Example 47

### (3R)-1-[8-(2,6-difluorophenyl)-11-methyl-3,4,7,9,12-pentazatricyclo[8.4.0.02,6] tetradeca-1(10),2(6),4,7,11,13-hexaen-13-yl]piperidine-3-carbonitrile

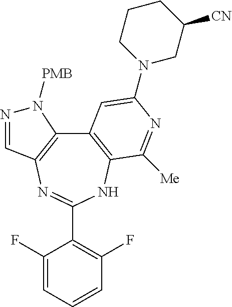

The title compound was made from Intermediate D4-2, following a similar synthetic procedure as described in the synthesis of Int-6 (described above), except that (3R)-piperidine-3-carbonitrile was used in the place of morpholine. The reaction was conducted in dioxane at 100° C. for 2 hrs and treated with TFA to remove the PMB protecting group. The reaction was purified by Pre-HPLC (column: YMC-Triart Prep C18 150×40 mm×7 m; mobile phase: [water (NH₃H₂O+NH₄HCO₃)-ACN]; B %: 24%-64%, 9 min) and then chiral separation (column: DAICEL CHIRALPAK AS (250 mm×30 mm, 10 m); mobile phase: [0.1% NH₃H₂O EtOH]; B %: 30%-30%, min) to provide the title compound as a yellow solid (70.6 mg, 60.6%). ¹H NMR (400 MHz, CD₃OD) δ ppm 1.60-1.74 (m, 1H), 1.78-1.97 (m, 2H), 2.07 (br s, 1H), 1.99-2.11 (m, 1H), 2.26 (s, 3H), 2.97 (dt, J=7.7, 4.0 Hz, 1H), 3.42-3.51 (m, 1H), 3.62 (br d, J=7.9 Hz, 2H), 3.96 (br d, J=10.5 Hz, 1H), 4.63 (s, 1H), 6.94 (br s, 1H), 7.10 (br t, J=8.2 Hz, 2H), 7.32 (s, 1H), 7.52 (br t, J=6.9 Hz, 1H); ¹⁹F NMR (377 MHz, CD₃OD) δ ppm −114.81; LCMS (ESI) m/z: 420.1 [M+H]⁺.

### Example 48

### 1-[8-(2,6-difluorophenyl)-5-methyl-3,4,7,9,12-pentazatricyclo[8.4.0.02,6] tetradeca-1(10),2(6),4,7,11,13-hexaen-13-yl]piperidine-4-carbonitrile

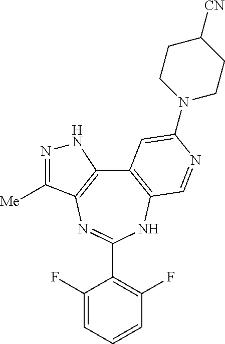 [SMILES: Cc1n[nH]c2c1N=C(c1c(F)cccc1F)Nc1cnc(N3CCC(C#N)CC3)cc1-2]

The title compound was made from Intermediate D2-3, following a similar synthetic procedure as described in the synthesis of Int-1 (described above), except that piperidine-4-carbonitrile was used in the place of morpholine. The reaction was conducted in dioxane at 100° C. for 2 hrs and treated with TFA/DCM to remove the SEM protecting group. The residue was purified by prep-HPLC (column: YMC-Triart Prep C18 150×40 mm×7 m; mobile phase: [water(NH₃H₂O+NH₄HCO₃)-ACN]; B %: 23%-63%, 9 min) to provide the title compound as a yellow solid (120 mg, 19.6%). ¹H NMR (400 MHz, DMSO-d₆) δ ppm 1.69-1.76 (m, 2H), 1.90 (br dd, J=6.65, 2.89 Hz, 2H), 1.96 (s, 3H), 3.05-3.11 (m, 1H), 3.18-3.25 (m, 2H), 3.58-3.65 (m, 2H), 6.83-6.89 (m, 1H), 7.16 (t, J=8.03 Hz, 2H), 7.39-7.45 (m, 1H), 7.45-7.51 (m, 1H), 8.25 (s, 1H), 12.43 (s, 1H); ¹⁹F NMR (376 MHz, DMSO-d₆) δ ppm −115.56; LCMS (ESI) m/z: 420.5 [M+H]⁺.

### Example 49

### (3R)-1-[8-(2,6-difluorophenyl)-5-methyl-3,4,7,9,12-pentazatricyclo[8.4.0.0^{2,6}] tetradeca-1(10),2(6),4,7,11,13-hexaen-13-yl]pyrrolidine-3-carbonitrile

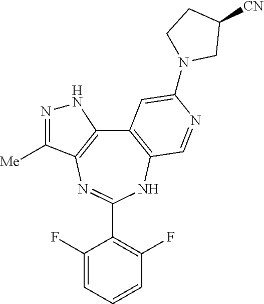 [SMILES: Cc1n[nH]c2c1N=C(c1c(F)cccc1F)Nc1cnc(N3CC[C@@H](C#N)C3)cc1-2]

The title compound was made from Intermediate D2-3, following a similar synthetic procedure as described in the synthesis of Int-1 (described above), except that (3R)-pyrrolidine-3-carbonitrile was used in the place of morpholine. The reaction was conducted in dioxane at 100° C. for 2 hrs and treated with TFA/DCM to remove the SEM protecting group. The residue was purified by prep-HPLC (column: Boston Prime C18 150×30 mm×5 m; mobile phase: [water (ammonia hydroxide v/v)-ACN]; B %: 15%-45%, 9 min) to provide the title compound as a yellow solid (13 mg, 24.5%). ¹H NMR (400 MHz, CD₃OD) δ ppm 2.14 (s, 3H), 2.28-2.49 (m, 2H), 3.45-3.56 (m, 2H), 3.60-3.70 (m, 2H), 3.75-3.82 (m, 1H), 6.76 (s, 1H), 7.08 (t, J=8.01 Hz, 2H), 7.40 (s, 1H), 7.46-7.55 (m, 1H); ¹⁹F NMR (376 MHz, CD₃OD) δ ppm −115.30; LCMS (ESI) m/z: 406.1 [M+H]⁺.

### Example 50

### [2,4-difluoro-3-(5-methyl-13-morpholino-3,4,7,9,12-pentazatricyclo[8.4.0.02,6]tetradeca-1(10),2(6),4,7,11,13-hexaen-8-yl)phenyl]methanol

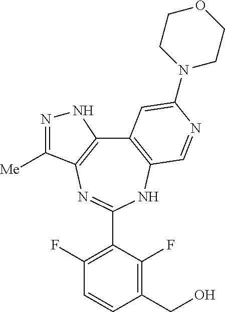 [SMILES: Cc1n[nH]c2c1N=C(c1c(F)ccc(CO)c1F)Nc1cnc(N3CCOCC3)cc1-2]

The title compound was made from Intermediate D2-4, following a similar synthetic procedure as described in the synthesis of Int-1 (described above). The resulting mixture was treated with 4N HCl in MeOH, concentrated under reduced pressure and purified by preparative HPLC (Instrument: FD: Gilson GX-215, Gilson 322 Pump, Gilson 156 UV Detector; 30° C.; Column: Durashell 150×30 mm×5 m; Mobile phase A: H₂O with 0.25% FA (v %); Mobile phase B: MeCN; Gradient: B from 10% to 40% in 9.5 min, hold 100% B for 2 min; Flow Rate: 25 mL/min; Column Temperature: 30° C.; Wavelength: 220 nm, 254 nm) to provide the title compound as a yellow solid (21.0 mg, 12.1%). ¹H NMR (400 MHz, DMSO-d₆) δ ppm 1.95 (s, 3H), 3.24-3.28 (m, 4H), 3.66 (br s, 4H), 4.53 (d, J=5.52 Hz, 2H), 5.36 (t, J=5.65 Hz, 1H), 6.83 (s, 1H), 7.13 (t, J=8.66 Hz, 1H), 7.43 (s, 1H), 7.46-7.55 (m, 1H), 8.24 (s, 1H), 12.41 (s, 1H); ¹⁹F NMR (376 MHz, DMSO-d₆) δ ppm −120.14, −116.52; LCMS (ESI) m/z: 427.0 [M+H]⁺.

### Example 51

### 4-(5-(2,6-difluoro-4-methylphenyl)-1,6-dihydropyrazolo[4,3-d]pyrido[4,3-f][1,3]diazepin-9-yl)morpholine

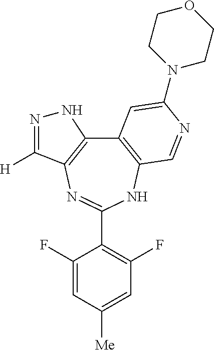 [SMILES: Cc1cc(F)c(C2=Nc3cn[nH]c3-c3cc(N4CCOCC4)ncc3N2)c(F)c1]

The title compound was made from Intermediate D2-1, following a similar synthetic procedure as described in the synthesis of Int-1 (described above), except that the reaction was conducted in dioxane at 100° C. for 2 hr. The reaction mixture was treated with 4N HCl in MeOH to remove the SEM protecting group. The resulting residue was purified by reverse phase preparative HPLC (column: Phenomenex C18 150 by prep-HPLC (column: Welch Xtimate C18 150×30 mm×5 um; mobile phase: [water(0.05% HCl)-ACN]) 40 mm×5 m; mobile phase: [water(HCl)-ACN]; B %: 1%-30%, 10 min) to provide the title compound as a yellow solid (20 mg, 28.8%). ¹H NMR (400 MHz, CD₃OD) δ ppm 2.49 (s, 3H), 3.64-3.74 (m, 4H), 3.80-3.91 (m, 4H), 7.17 (d, J=9.79 Hz, 2H), 7.58-7.64 (m, 2H), 7.66 (s, 1H); ¹⁹F NMR (376 MHz, CD₃OD) δ ppm −113.55; LCMS (ESI) m/z: 397.0 [M+H]⁺.

### Example 52

### (R)-1-(3-chloro-5-(2,6-difluorophenyl)-1,6-dihydropyrazolo[4,3-d]pyrido[4,3-f][1,3]diazepin-9-yl)piperidine-3-carbonitrile

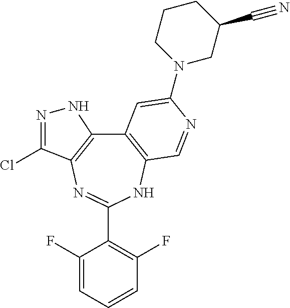 [SMILES: N#C[C@@H]1CCCN(c2cc3c(cn2)NC(c2c(F)cccc2F)=Nc2c(Cl)n[nH]c2-3)C1]

The title compound was made from Intermediate D2-2, following a similar synthetic procedure as described in the synthesis of Int-1 (described above), except that (3R)-piperidine-3-carbonitrile was used in the place of morpholine, and 10% Pd₂(dba)₃ was employed as the catalyst. The reaction was conducted in dioxane at 100° C. for 0.5 hr and treated with 4N HCl in MeOH to remove the SEM protecting group. The resulting mixture was purified by flash chromatography (ISCO®; 12 g SepaFlash® Silica Flash Column, petroleum ether/EtOAc with EtOAc from 0˜60%, 30 mL/min, 254 mn) to give the title compound as a yellow solid (38.40 mg, 48.2%). ¹H NMR (400 MHz, DMSO-d₆) δ ppm 1.50-1.66 (m, 2H), 1.79-1.98 (m, 1H), 3.01 (dt, J=7.28, 3.64 Hz, 1H), 3.37-3.51 (m, 2H), 3.61-3.73 (m, 2H), 6.75 (s, 1H), 7.18 (t, J=7.91 Hz, 2H), 7.32 (s, 1H), 7.47-7.55 (m, 1H), 8.39 (s, 1H), 13.21 (s, 1H); ¹⁹F NMR (377 MHz, DMSO-d₆) δ ppm −114.02; LCMS (ESI) m/z: 440.0 [M+H]⁺.

### Example 53

### 3-[5-chloro-8-(2,6-difluorophenyl)-3,4,7,9,12-pentazatricyclo[8.4.0.02,6]tetradeca-1(10),2(6),4,7,11,13-hexaen-13-yl]-8-oxa-3-azabicyclo[3.2.1]octane

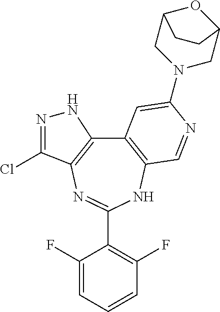 [SMILES: Fc1cccc(F)c1C1=Nc2c(Cl)n[nH]c2-c2cc(N3CC4CCC(C3)O4)ncc2N1]

The title compound was made from Intermediate D2-2, following a similar synthetic procedure as described in the synthesis of Int-1 (described above), except that 8-oxa-3-azabicyclo [3.2.1] octane was used in the place of morpholine. The reaction was conducted in dioxane at 100° C. for 2 hrs and was treated with 4N HCl in MeOH to remove the SEM protecting group. The crude product was triturated with petroleum ether (2 mL) to provide the title compound as a white solid (10.8 mg, 86%). ¹H NMR (400 MHz, CD₃OD) δ ppm 1.82-2.19 (m, 4H), 3.83 (br d, J=11.54 Hz, 2H), 4.55 (br s, 2H), 4.96 (br s, 2H), 7.16-7.41 (m, 3H), 7.57 (s, 1H), 7.79 (br t, J=6.65 Hz, 1H); LCMS (ESI) m/z: 443.1 [M+H]⁺.

### Example 54

### 3-chloro-5-(2,6-difluorophenyl)-9-(1-methyl-1H-pyrazol-4-yl)-1,6-dihydropyrazolo[4,3-d]pyrido[4,3-f][1,3]diazepine

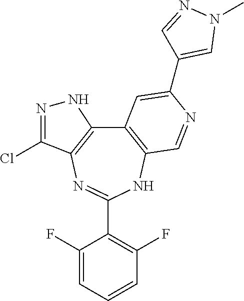 [SMILES: Cn1cc(-c2cc3c(cn2)NC(c2c(F)cccc2F)=Nc2c(Cl)n[nH]c2-3)cn1]

### Step 1: Synthesis of 3-chloro-5-(2,6-difluorophenyl)-9-(1-methyl-1H-pyrazol-4-yl)-1-((2-(trimethylsilyl)ethoxy)methyl)-1,6-dihydropyrazolo[4,3-d]pyrido[4,3-f][1,3]diazepine

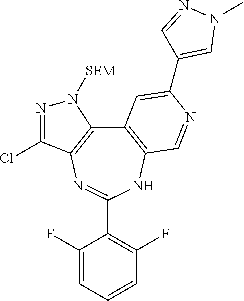 [SMILES: Cn1cc(-c2cc3c(cn2)NC(c2c(F)cccc2F)=Nc2c(Cl)nn(COCC[Si](C)(C)C)c2-3)cn1]

To a solution of 2-[[13-bromo-5-chloro-8-(2,6-difluorophenyl)-3,4,7,9,12-pentazatricyclo[8.4.0.02,6]tetradeca-1(10),2(6),4,7,11,13-hexaen-3-yl]methoxy]ethyl-trimethyl-silane (Intermediate D2-2, 200 mg, 0.36 mmol, 1 eq) in dioxane (2 mL) and H₂O (0.1 mL) was added 1-methyl-4-(4,4,5,5-tetramethyl-1,3,2-dioxaborolan-2-yl)pyrazole (115 mg, 0.55 mmol, 1.5 eq), XPhos (35.2 mg, 0.073 mmol, 0.2 eq), K₃PO₄ (235 mg, 0.01 mmol, 0.3 eq) and Pd₂(dba)₃ (33.8 mg, 0.036 mmol, 0.1 eq). The reaction mixture was degassed and purged with N₂ for three times, stirred at 90° C. for 16 hr under N₂ atmosphere and concentrated under reduced pressure. The residue was diluted with H₂O (10 mL) and extracted with EtOAc (15 mL×3). The combined organic layers were washed with brine (10 mL), dried over Na₂SO₄, filtered and concentrated under reduced pressure. The residue was purified by prep-TLC (silica, PE/EtOAc=1/1) to provide 2-[[5-chloro-8-(2,6-difluorophenyl)-13-(1-methylpyrazol-4-yl)-3,4,7,9,12-pentazatricyclo[8.4.0.0^{2,6}]tetradeca-1(10),2(6),4,7,11,13-hexaen-3-yl]methoxy]ethyl-trimethyl-silane as a yellow solid (130 mg, 44.7%). LCMS (ESI) m/z: 542.1 [M+H]⁺.

### Step 2: Synthesis of 3-chloro-5-(2,6-difluorophenyl)-9-(1-methyl-1H-pyrazol-4-yl)-1,6-dihydropyrazolo[4,3-d]pyrido[4,3-f][1,3]diazepine

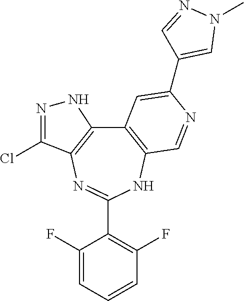 [SMILES: Cn1cc(-c2cc3c(cn2)NC(c2c(F)cccc2F)=Nc2c(Cl)n[nH]c2-3)cn1]

To a solution of 2-[[5-chloro-8-(2,6-difluorophenyl)-13-(1-methylpyrazol-4-yl)-3,4,7,9,12-pentazatricyclo[8.4.0.0^{2,6}]tetradeca-1(10),2(6),4,7,11,13-hexaen-3-yl]methoxy]ethyl-trimethyl-silane (130 mg, 0.165 mmol, 1 eq) in 4N HCl in MeOH (3 mL, 4 M) was added Et₃SiH (57.73 mg, 0.496 mmol, 3 eq). The reaction mixture was stirred at 20° C. for 2 hrs and concentrated under reduced pressure. The residue was diluted with saturated Na₂HCO₃ aqueous solution (10 mL) and extracted with EtOAc (15 mL×3). The combined organic layers were washed with brine (10 mL), dried over Na₂SO₄, filtered and concentrated under reduced pressure. The crude product was purified by prep-HPLC (column: 2_Phenomenex Gemini C18 75×40 mm×3 m; mobile phase: [water(NH₄HCO₃)-ACN]; B %: 25%-55%, 9.5 min) to give the title compound (3.68 mg, 5.4%) as a yellow solid. ¹H NMR (400 MHz, DMSO-d₆) δ ppm 3.88 (s, 3H), 7.16 (t, J=8.03 Hz, 2H), 7.41 (br s, 1H), 7.47-7.53 (m, 1H), 7.67 (br s, 1H), 7.78 (s, 1H), 7.98 (br s, 1H), 8.38 (br s, 1H), 13.14 (br s, 1H); ¹⁹F NMR (376 MHz, DMSO-d₆) δ ppm −113.96; LCMS (ESI) m/z: 412.0 [M+H]⁺.

### Example 55

The compounds disclosed herein were tested for their inhibitory effect on LRRK2 kinase according to the following procedures.

The protocol for basic TR-FRET LanthaScreen Tb Kinase Activity Assay inhibitor studies were as follows. LanthaScreen Kinase Activity Assays (ThermoFisher/USA) to evaluate inhibitors were performed by addition of 100 nL of test compound in corresponding DMSO dilutions/5 μL of kinase/fluorescein-ERM (LRRKtide) peptide mixture, Compound & kinase were pre-incubated for 15 minutes, then 5 μl of ATP into 384 well small volume plates. After incubation for 120 minutes at room temperature, the detection reagents containing Tb-anti-pLRRKtide antibody were added to monitor phosphorylation level of peptide. Then, after 60 min minutes incubation at room temperature plates were read in Envision. Data analysis of emission ratios was according to LanthaScreen Tb Kinase Activity Assay protocol.

Kinase and assay components were adjusted to final concentrations according to the kit protocol. For LRRK2: 2 nM wt human LRRK2, catalytic site, catalytic site (ThermoFisher/USA), 400 nM peptide, 38 μM ATP in 1× Kinase Buffer A.

Basic protocol for TR-FRET LanthaScreen Tb Kinase Activity Assay inhibitor studies involved two steps:

Enzymatic step: Addition of 100 nl of test compound in corresponding DMSO dilutions, 5 μl of kinase/substrate mixture, 5 μl of ATP into 384 well small volume plates. Incubation for 120 minutes at room temperature.

Detection step: Addition of 10 μl EDTA & antibody, read plate after 60 minutes. Data analysis of emission ratios according to KinEASE assay protocol.

Kinase and assay components were adjusted to final concentrations according to the kit protocol. The inhibitory effect of the these compounds are provided in Table 2.

### Example 56

The compounds disclosed herein were tested on CD-1 mice for brain penetration (Kp, uu) according to the following procedures.

1 Materials and Methods

1.1 Study Design

Nine male CD-1 mice were administered TEST COMPOUND by single intravenous bolus administration at 1 mg/kg. The vehicle used for IV study was 5% DMSO, 5% solutol, 20% PEG300 and 70% water. The detailed dosing and sampling regimens are described in the following tables.

| | | | | | | | Dose | |
|---|---|---|---|---|---|---|---|---|
| Group | | No. of | | Dose | Dose | Conc | Volume | |
| No. | Test Article | animals | Sex | Route | (mg/kg) | (mg/mL) | (mL/kg) | Vehicle |
| 01 | Test | 9 | Male | IV | 1 | 0.5 | 2 | 5% DMSO, 5% |
| | compound | | | bolus | | | | solutol, 20% |
| | | | | | | | | PEG300 and 70% |
| | | | | | | | | water |

| | Dosage | | | | Time Point | | |
|---|---|---|---|---|---|---|---|
| Group | (mg/kg) | Animal Number | | | (hr) | Brain | Plasma |
| 01 | 1 | M01 | M02 | M03 | 0.25 | 1 | 1 |
| | | M04 | M05 | M06 | 0.5 | 1 | 1 |
| | | M07 | M08 | M09 | 1.5 | 1 | 1 |

1.2 Intravenous (IV) Dose

1.0 mg of test compound was accurately weighed into a glass vial, and slowly added with 104 μL of DMSO and vortexed for 1 min. Then added with 104 μL of solutol and vortexed for 1 min. Then added with 415 μL of PEG300 and vortexed for 1 min. Then added with 1000 μL of water and vortexed for 1 min. Finally, added with 451 μL of water and vortexed for 1 min to get a clear solution.

1.3 Formulation Analysis

Dose formulations were analyzed in duplicates for each dose using HPLC-UV methodologies with a six-point calibration curve. Two aliquots were taken from the middle region of IV dosing solution.

Acceptance criteria: The measured concentrations of test article in each dose formulation must fall within 80% to 120% of the nominal concentrations.

1.4 In-Life Work

2 Animal Husbandry

Nine male CD-1 mice supplied by Shanghai Institute of Planned Parenthood Research were used in this study. The animals were confirmed to be healthy before being assigned to the study. Each animal was given a unique identification number which was marked on the tail and written on the cage card.

The room (s) was controlled and monitored for relative humidity (targeted mean range 40% to 70%) and temperature (targeted mean range 20° to 26° C.) with 10 to 20 air changes/hour. The room was on a 12-hour light/dark cycle except when interruptions were necessitated by study activities.

3 Feeding

Fresh drinking water (reverses osmosis) was available to all animals, ad libitum.

All animals were fasted at least 12 hours prior to the administration. Certified rodent diet food was withheld until 4 hours post-dose. The fasting time would not exceed 20 hours.

4 Dose Administration

Animals were weighed prior to dose administration on each day of dosing to calculate the actual dose volume.

The body weights were in the range from 28.81 to 31.25 g for males on the first dosing day. All animals received a single intravenous bolus administration of TEST COMPOUND from the tail vein.

5 Clinical Observation

Cage-side observations for the general health condition and appearance of the animal were performed before and after dosing and at each time point of sample collection. Unusual observations were recorded throughout the duration of the study.

6 Sample Collection and Processing

Each blood collection (about 0.06 mL per time point) was performed from saphenous vein of each animal into pre-chilled commercial K2-EDTA tubes as anti-coagulant and placed on wet ice until centrifugation.

Plasma samples were then prepared by centrifuging the blood samples at approximately 4° C., 3200×g for 10 minutes. All plasma samples were then quickly frozen over dry ice and kept at −60° C. or lower until LC-MS/MS analysis.

Brain tissue was homogenized using homogenizing buffer (15 mM PBS (pH7.4): MeOH=2:1) at the ratio of 1:9 (1 g tissue with 9 mL buffer, the dilution ratio is 10). The tissue homogenate was kept at −60° C. or lower until LC-MS/MS analysis.

6.1 Bioanalytical Analysis

The concentrations of TEST COMPOUND in plasma and brain were determined by using an LC-MS/MS method.

6.2 Pharmacokinetics Data Analysis

The plasma and brain concentration of TEST COMPOUND in study animals was subjected to a non-compartmental pharmacokinetic analysis by using the Phoenix WinNonlin software program (version 6.3 or above, Pharsight). The linear/log trapezoidal rule was applied in obtaining the PK parameters.

Individual plasma and brain concentration values that were below the lower limit of quantitation (LLOQ) were excluded from the PK parameter calculation. The nominal dose levels and nominal sampling times were used in the calculation of all pharmacokinetic parameters.

Mouse Kp, uu=(% free in mouse brain*concentration in mouse brain)/(% free in mouse plasma*concentration in mouse plasma)

Plasma Protein Binding of Test Article Assessed In Vitro by Equilibrium Dialysis

1. Preparation of 100 mM Sodium Phosphate and 150 mM NaCl Buffer (PBS)

A basic solution was prepared by dissolving 14.2 g/L Na₂HPO₄ and 8.77 g/L NaCl in deionized water, and an acidic solution was prepared by dissolving 12.0 g/L NaH₂PO₄ and 8.77 g/L NaCl in deionized water. The basic solution was titrated with the acidic solution to pH 7.4. All solutions were stored at 4° C. for up to 7 days. The pH was checked on the day of experiment and was adjusted if outside specification of 7.4±0.1.

2. Thawing of the Frozen Plasma (Stored at −80° C.)

Frozen plasma was thawed at room temperature. The plasma was centrifuged at 3,220 g for 10 minutes to remove clots and the supernatant was collected into a fresh tube. The pH of the plasma was checked and recorded. Plasma was only used if it was within the range of pH 7 to pH 8 and been thawed no more than twice since arrival.

3. Preparation of Working Solutions

The working solutions of test compounds and control compound ketoconazole were prepared in dimethyl sulfoxide (DMSO) at the concentration of 200 μM. 3 μL of working solution was removed and mixed with 597 μL of plasma to achieve a final concentration of 1 μM (0.5% DMSO). Plasma samples were vortexed thoroughly.

4. Preparation of Dialysis Membranes

The dialysis membranes (HTD 96a/b Dialysis Membrane Strips MWCO 12-14K, Cat. #1101, Batch #3763 (11-21)) were soaked in ultrapure water for 60 minutes to separate the strips, then in 20% ethanol for 20 minutes, and lastly in dialysis buffer for 20 minutes.

5. Procedure for Equilibrium Dialysis

The dialysis apparatus was assembled according to the manufacturer's instruction. Each Cell was filled with 120 μL of spiked plasma sample and dialyzed against equal volume of dialysis buffer (PBS). The assay was performed in duplicate. The dialysis plates were sealed and incubated in an incubator at 37° C. with 5% CO₂ and rotated at 100 rpm for 6 hours. At the end of incubation, the seals were removed and 50 μL of samples from both buffer and plasma chambers were transferred to a 96-well plate.

6. Procedure for Sample Preparation

50 μL of blank plasma was added to each buffer sample and an equal volume of PBS was added to the collected plasma sample. 300 μL of room temperature quench solution (acetonitrile containing internal standards (IS, 100 nM alprazolam, 500 nM labetalol and 2 μM ketoprofen)) was added to precipitate protein. The plates were vortexed for 5 minutes and centrifuged at 3,220 g for 30 minutes at 4° C. 100 μL of the supernatants was combined with 100 μL of water in a new 96-well plate for LC-MS/MS analysis.

All calculations were carried out using Microsoft Excel. The concentrations of test compound and control compound in the buffer and plasma chambers were determined from peak area ratios (PAR) of analyte to internal standard. Protein binding (% unbound and % bound) and recovery (% recovery) of test and control compounds was calculated as follows:

$$\% Fu = ( Peak Area {Ratio}_{buffer chamber} / Peak Area {Ratio}_{plasma chamber} ) * 100$$

$$\% Bound = 100 - \% Fu$$

$$\% Recovery = ( Peak Area {Ratio}_{buffer chamber} + Peak Area {Ratio}_{plasma chamber} ) \text{} / Peak Area {Ratio}_{total sample} * 100$$

$$Log K = Log ( \frac{\% Bound}{100 - \% Bound} )$$

Where:
Fu=fraction unbound
Peak Area Ratio_{buffer chamber}=concentration of free fraction
Peak Area Ratio_{plasma chamber}=concentration of both the free and bound fractions
Peak Area Ratio_{total sample}=concentration of starting sample before incubation

Binding of Test Article in Brain Tissue Assessed by Equilibrium Dialysis

1. Thawing of the Frozen Brain (Stored at −80° C.)

Frozen brain tissue was thawed at room temperature in a bath. Only brain tissue was used that was thawed no more than once.

2. Preparation of Brain Tissue Homogenate

Brain tissue homogenate was prepared by diluting one quantity (g) of the whole brain tissue with three volumes (mL) of buffer (PBS, pH 7.4) and homogenizing the mixture using a tissue homogenizer. Brain tissue homogenate was frozen at −80° C. for later use/comparison.

3. Preparation of Working Solutions

The working solutions of test compound and the control compound propranolol were prepared in dimethyl sulfoxide (DMSO) at the concentration of 200 μM. 4 μL of working solution was removed and mixed with 796 μL of brain tissue homogenate to achieve a final concentration of 1 μM (0.5% DMSO). Brain tissue homogenate samples were vortexed thoroughly.

4. Preparation of Dialysis Membranes

The dialysis membranes (HTD 96a/b Dialysis Membrane Strips MWCO 12-14K, Cat. #1101, Batch #3763 (11-21)) were soaked in ultrapure water for 60 minutes to separate the strips, then in 20% ethanol for 20 minutes, and lastly in dialysis buffer for 20 minutes.

5. Procedure for Equilibrium Dialysis

The dialysis apparatus was assembled according to the manufacturer's instruction. Each Cell was filled with 150 μL of brain homogenate sample and dialyzed against equal volume of dialysis buffer (PBS). The assay was performed in duplicate. The dialysis plates were sealed and incubated in an incubator at 37° C. with 5% CO₂ and rotated at 100 rpm for 6 hours. At the end of incubation, the seals were removed and 50 μL of samples from both buffer and brain homogenate chambers were transferred into separate tubes on a plate.

6. Procedure for Sample Preparation

50 μL of blank brain homogenate was added to each buffer sample and an equal volume of PBS was added to the collected brain homogenate sample. 400 μL of room temperature quenching solution (acetonitrile containing internal standards (IS, 100 nM alprazolam, 500 nM labetalol and 2 μM ketoprofen)) was added to precipitate protein. The plates were vortexed for 5 minutes and centrifuged at 3,220 g for 30 minutes at room temperature. 100 μL of the supernatants was combined with 100 μL of water in a new 96-well plate for LC-MS/MS analysis.

All calculations were carried out using Microsoft Excel. The concentrations of test compound and control compound in the buffer and brain homogenate chambers were determined from peak area ratios (PAR) of analyte to internal standard. Protein binding (% unbound and % bound) and recovery (% recovery) of test and control compounds was calculated as follows:

$$\% {Fu}_{meas} = ( Peak Area {Ratio}_{buffer chamber} / Peak Area {Ratio}_{brain homogenate chamber} ) * 100$$

$$Undiluted fu = \frac{1 / D}{( ( 1 / {Fu}_{meas} ) - 1 ) + 1 / D}$$

$$\% Bound = ( 1 - Undiluted fu ) * 100$$

$$\% Recovery = ( {PAR}_{buffer chamber} + {PAR}_{brain homogenate chamber} ) / {PAR}_{total sample} * 100$$

$$Log K = Log ( \frac{\% Bound}{100 - \% Bound} )$$

Where:
Fuₘₑₐₛ=measured fraction unbound in brain homogenate
D=dilution factor of brain tissue
PAR_{buffer chamber}=concentration of free fraction
PAR_{brain homogenate chamber}=concentration of both the free and bound fractions
PAR_{total sample}=concentration of starting sample before incubation
% Bound=fraction bound in brain homogenate

### Example 57

The compounds disclosed herein were tested in vitro on CaCo2 cell penetration according to the following procedures.

Test compounds were prepared in dimethyl sulfoxide (DMSO).

Caco-2 cells purchased from ATCC were seeded onto polyethylene membranes (PET) in 96-well Corning Insert plates at 1×10⁵ cells/cm². The media were refreshed every 4 to 5 days until the 21st to 28th day for confluent cell monolayer formation.

Experiments were run in duplicate using a HBSS transport buffer containing 10 mM HEPES at pH 7.4. The test article TEST COMPOUND was tested bi-directionally at 2 and 10 M concentrations, the control compound digoxin was tested bi-directionally at 10 μM, and nadolol and metoprolol were tested at 2.0 μM in the A to B direction. Final DMSO concentrations were adjusted to less than 1%. The plates were incubated for 2 hours in a CO₂ incubator at 37±1° C. with 5% CO₂ at saturated humidity without shaking. Following incubation, samples were mixed with acetonitrile containing internal standard and centrifuged at 3200×g for 10 minutes. Then 200 μL of supernatant was taken from all samples. For TEST COMPOUND and digoxin, the supernatant was diluted with 200 μL of ultra-pure water and for nadolol and metoprolol, the supernatant was diluted with 600 μL of ultra-pure water. All samples were analyzed by LC-MS/MS. Concentrations of TEST COMPOUND and control compounds were determined in starting solutions, donor well solutions, and receiver well solutions using peak area ratios (PAR) of analyte to internal standard. After the transport assay, a lucifer yellow rejection assay was run to determine the Caco-2 cell monolayer integrity.

Bidirectional Permeability Assessment

The apparent permeability (Pₐₚₚ), the % recovery and the efflux ratio were calculated as follows:

Pₐₚₚ=(dCᵣ/dt)×Vᵣ/(A×C₀) 1.

% Recovery=100×[(Vᵣ×Cᵣ)+(V_{d}×C_{d})]/(V_{d}×C₀) 2.

Efflux Ratio=Pₐₚₚ(BA)/Pₐₚₚ(AB) 3.
Where:
dCᵣ/dt is the slope of the cumulative concentration in the receiver compartment versus time (mM/s).
Vᵣ and V_{d} is the volume in the receiver and donor compartments (0.075 mL on the apical side, 0.25 mL on the basolateral side), respectively.
A is the surface area of the cell monolayer (0.0804 cm²).
C₀ is the measured donor concentrations (μM) at the beginning of the experiment.
C_{d} and Cᵣ are the final measured concentrations in the donor and receiver compartments, respectively.

The cell monolayer tight junction integrity assay was conducted after completion of the transport assay by adding Lucifer Yellow (LY) to the apical wells at 100 μM and incubating the cells for 30 minutes. Following incubation, tight junction integrity was assessed from LY concentrations in the basolateral compartment (receiver well) and the apical compartment (donor well) using fluorescence detection as follows:

$$\% Lucifer Yellow = \frac{{V}_{Basolateral} \times {RFU}_{B a solateral}}{{V}_{Apical} \times {RFU}_{Apical} + {V}_{Basolateral} \times RF {U}_{B a s o l a t e r a l}} \times 100$$

Where:
   RFU_{Apical} and RFU_{Basolateral} are the relative fluorescence unit values of LY in the apical and basolateral wells, respectively.
   V_{Apical} and V_{Basolateral} are the volume of apical and basolateral wells (0.075 mL and 0.25 mL), respectively.

### Example 58

The compounds disclosed herein were tested in vitro on human hepatocyte for in vitro h-Hept CL predict according to the following procedures.
1. Compound sock solutions were prepared by dissolving test compound and positive control in dimethyl sulfoxide (DMSO) at a concentration of 10 mM. Stock solutions were then diluted to 100 μM solutions by combining 198 μL of 50% acetonitrile/50% water with 2 μL of the 10 mM stock in separate conical tubes.
2. Cryopreserved hepatocytes were prepared as follows: Incubation medium (William's E Medium supplemented with GlutaMAX) and hepatocyte thawing medium were placed in a 37° C. water bath and allowed to warm for at least 15 minutes prior to use. Cryopreserved hepatocytes were removed from storage, ensuring that vials remained at cryogenic temperatures until thawed. Cells were thawed by placing the vials in a 37° C. water bath and gently shaking the vials for 2 minutes. After thawing was completed, vials were sprayed with 70% ethanol and transferred to a biosafety cabinet. Hepatocytes were transferred into 50 mL conical tube containing thawing medium using wide-bore pipette tips. Tubes were placed into a centrifuge and spun at 100 g for 10 minutes. Then thawing medium was aspirated and hepatocytes were resuspended in enough incubation medium to yield ˜1.5×10⁶ cells/mL. Cells were counted using acridine orange and propidium iodide (AO/PI) staining, and the viable cell density was determined. Cells were diluted with incubation medium to a working cell density of 0.5×10⁶ viable cells/mL.
3. To determine hepatocyte stability, 198 μL of hepatocytes were transferred into each well of a 96-well non-coated plate. Plates were placed in an incubator to allow the hepatocytes to warm for 10 minutes.
4. The reaction was started by transferring 2 μL of the 100 μM test compound or positive control solution into respective wells of the 96-well non-coated plates. Plates were returned to the incubator until 25 μL aliquots of well contents were sampled at time points of 0, 15, 30, 60, 90 and 120 minutes. The aliquots were then mixed with 6 volumes (150 μL) of acetonitrile containing internal standard, IS (100 nM alprazolam, 200 nM caffeine and 100 nM tolbutamide) to terminate the reaction and vortexed for 5 minutes. After centrifuging the samples for 45 minutes at 3,220 g, aliquots of 100 μL of the supernatant were taken and diluted with 100 μL of ultra-pure water. The mixture was used for LC/MS/MS analysis. All incubations were performed in duplicate.

All calculations were carried out using Microsoft Excel. Peak area ratios (PAR) were determined from extracted ion chromatograms. The in vitro half-life (t_{1/2}) of parent compound was determined by regression analysis of the percent parent disappearance vs. time curve as follows:

The in vitro half-life (in vitro t_{1/2}) was determined from the slope value:

In vitro t_{1/2}=0.693/k

Conversion of the in vitro t_{1/2} (in minutes) into the in vitro intrinsic clearance (in vitro CLᵢₙₜ, in μL/min/1×10⁶ cells) was done using the following equation (mean of duplicate determinations):

In vitro CLᵢₙₜ=kV/N

The scaled-up intrinsic clearance (CLᵢₙₜ, mL/min/kg), predicted hepatic clearance (CL_{H}, mL/min/kg) and hepatic extraction ratio (ER) were calculated using the following equations:

CLᵢₙₜ=(0.693/t_{1/2})*(1/(hepatocyte concentration (0.5*10⁶ cells/mL)))*scaling factors (Table (A) Appendix Table 1)

CL_{Hept-predict}=(Q_{H}*Scaled-up CLᵢₙₜ*fub)/(Q_{H}+Scaled-up CLᵢₙₜ*fub)

ER=CL_{H}/Q_{H}
Where:
V=incubation volume (0.2 mL)
N=number of hepatocytes per well (0.1*10⁶ cells)
Q_{H}=hepatic blood flow (mL/min/kg) (Table (A) Appendix Table 1)
fub=fraction of unbound drug in plasma, assumed to be 1

Values used for calculation of the predicted hepatic clearance parameters data processing rules are outlined in the following tables (A) (B)

TABLE (A) APPENDIX TABLE 1
Scaling Factors for Clearance Prediction in Human and Rat

| | | Liver | Scaling | |
|---|---|---|---|---|
| | Hepatocellularity | weight | factors^{a} | |
| | (10⁶ | (g/kg of | (10⁶ | Liver |
| | cells/g | body | cells/ | blood flow |
| Species | liver) | weight) | kg) | (ml/min/kg) |
| Human | 99 | 25.7 | 2544.3 | 21 |
| Rat | 108 | 40 | 4320 | 70 |

Scaling factor = (microsomal protein per gram of liver) × (liver weight per kilogram of body weight)

TABLE (B)

| Remaining % | Processing Rules |
|---|---|
| ≥80% at | If T-test with p < 0.05 is obtained, report the calculated Clᵢₙₜ value; |
| 120 min | when the calculated Clᵢₙₜ value <2.72, then report <2.72 instead |
| | of calculated value. |
| | If T-test with p < 0.05 is not obtained, then report <2.72 for Clᵢₙₜ |
| | value when all the other data points fall in the range of |
| | 80%~120% (one data point within the range of 70%~130% is |
| | accepted); otherwise, the experiment should be repeated. |
| <80% at | Always remove all points from the calculation if <10% left of |
| 120 min | 0.5-minute sample but leave at least 2 points. |
| | If T-test with p < 0.05 is obtained, report the calculated CLᵢₙₜ |
| | value. |
| | If T-test with p < 0.05 is not obtained, the experiment must be |
| | repeated. |

### Example 59

Compounds disclosed in Table 2 were tested according to methods described in Examples 55-58.

In Table 2, the compounds were found to exhibit an LRRK2 IC₅₀ as indicated as the following: “++”: IC₅₀<1 nM; “+”: 1 nM≤IC₅₀≤100 nM.

In Table 2, the compounds were found to exhibit Kp,uu in mouse as: “++”: Kp,uu in mouse≥0.1; “+”: Kp,uu in mouse<0.1.

In Table 2, the compounds were found to exhibit Caco-2 permeability as followed: “L (Low permeability)”: A2B (B2A)<1; “M (Moderate permeability)”: 1≤A2B (B2A)≤5; “H (High permeability)”: A2B (B2A)>5; “N (Non-substrate)”: ER≤2.5; “L (Likely)”: ER≥2.5.

In Table 2, the compounds were found to exhibit human Hep Cl (mL/min/kg) in the following range: “L (Low Cl)”: Hep Cl (mL/min/kg)<6.3; “I (Intermediate Cl)”: 6.3≤Hep Cl (mL/min/kg)≤14.7; “H (High Cl)”: Hep Cl (mL/min/kg)>14.7; considering human hepatic blood flow is 21 mL/min/kg.

TABLE 2

| | | | | | | hHep Cl |
|---|---|---|---|---|---|---|
| Ex- | LRRK2 | | Caco-2 | | | or hLM Cl |
| ample | IC₅₀ | K_{p, uu} | A2B | B2A | | (mL/min/ |
| No. | (nM) | mouse | (10⁻⁶ cm/s) | (10⁻⁶ cm/s) | ER | kg) |
| 1 | ++ | | M | H | L | I |
| 2 | ++ | + | L | H | L | L |
| 3 | ++ | | M | H | L | L |
| 4 | ++ | | M | H | L | L |
| 5 | ++ | | L | H | L | L |
| 6 | + | + | M | H | L | L |
| 7 | ++ | | L | H | L | L |
| 8 | ++ | | M | H | L | L |
| 9 | + | | M | H | L | L |
| 10 | + | | L | H | L | L |
| 11 | ++ | | L | H | L | I |
| 12 | ++ | | M | H | L | L* |
| 13 | ++ | | M | H | L | I |
| 14 | ++ | + | H | H | L | L |
| 15 | + | | M | H | L | L |
| 16 | + | | M | H | L | L |
| 17 | + | | H | H | L | I* |
| 18 | ++ | + | M | H | L | L |
| 19 | + | | M | H | L | L |
| 20 | ++ | + | M | H | L | L |
| 21 | ++ | | L | H | L | L |
| 22 | ++ | | L | H | L | I |
| 23 | ++ | + | M | H | L | L |
| 24 | ++ | | M | H | L | I |
| 25 | ++ | | M | H | L | L |
| 26 | ++ | + | H | H | L | I |
| 27 | ++ | + | H | H | L | L |
| 28 | ++ | ++ | M | H | L | L |
| 29 | ++ | | L | H | L | L |
| 30 | ++ | | M | H | L | I |
| 31 | ++ | | M | H | L | I |
| 32 | ++ | | M | H | L | L* |
| 33 | ++ | + | M | H | L | L |
| 34 | ++ | | M | H | L | I* |
| 35 | ++ | + | H | H | L | L |
| 36 | + | + | M | H | L | L |
| 37 | ++ | + | M | H | L | L |
| 38 | ++ | | M | H | L | L* |
| 39 | ++ | + | M | H | L | L |
| 40 | ++ | | M | H | L | I |
| 41 | + | + | M | H | L | I |
| 42 | + | | M | H | L | L* |
| 43 | + | | M | H | L | L* |
| 44 | ++ | | L | H | L | L |
| 45 | + | | L | H | L | L* |
| 46 | ++ | | L | H | L | L* |
| 47 | ++ | | M | H | L | H |
| 48 | + | | M | H | L | I* |
| 49 | + | | M | H | L | I |
| 50 | + | | L | H | L | L |
| 51 | + | | H | H | L | I |
| 52 | ++ | | M | H | L | I |
| 53 | ++ | | M | H | L | I |
| 54 | ++ | + | M | H | L | I |

*hLM Cl (human liver microsomal Clearance)

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification, and guidance that can be adapted to the practice of this invention in its various embodiments and equivalents thereof.

1. A compound of Formula (I): 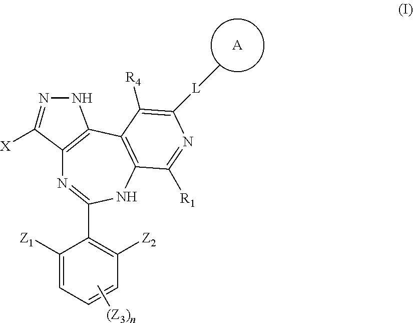
or an enantiomer, mixture of enantiomers, tautomer, or pharmaceutically acceptable salt thereof,
wherein:
n is 1, 2, or 3;
Y is N or CR₂;
Z₁, Z₂, and Z₃ are independently selected from H, —OH, halo, cyano, amino, C₁-C₆ alkyl, C₁-C₆ heteroalkyl, haloalkyl, alkoxy, haloalkxoxy, —CH(OH)-alkyl, hydroxyalkyl, or hydroxyalkoxy;
X is H, halo, cyano, cycloalkyl, C₁-C₆ alkyl, optionally deuterated C₁-C₆ alkyl, C₁-C₆ heteroalkyl, haloalkyl, alkoxy, haloalkxoxy, —CH(OH)-alkyl, hydroxyalkyl, or hydroxyalkoxy;
R₁, R₂, and R₄ are independently selected from H, halo, cyano, C₁-C₆ alkyl, optionally deuterated C₁-C₆ alkyl, C₁-C₆ heteroalkyl, haloalkyl, alkoxy, haloalkxoxy, —CH(OH)-alkyl, hydroxyalkyl, or hydroxyalkoxy;
L is a linker, wherein L is a single bond, substituted or unsubstituted C₁-C₄ alkyl, substituted or unsubstituted C₁-C₄ heteroalkyl, wherein the one or more heteroatoms are selected from O, S, or N;
A is 4-8 membered substituted or unsubstituted heterocycloalkyl, spiroheterocycloalkyl, heteroaryl, wherein one or more heteroatoms are selected from a group consisting of O, S, or N; and
wherein the substituents may be selected from a group consisting of substituted or unsubstituted 3-7 membered heterocycle, ═O, —CH₂-cycloalkyl, —CF₂-cycloalky, —C(═O)—O-alkyl, halo, deuterium, cyano, cyanoalkyl, —CF₃, mono-, di-, or tri-halo alkyl, CH(CH₃)-cycloalkyl, —CH₂-aryl, —CF₂-aryl, —CH(—CH₃)-aryl, C(═O)-alkyl, —C(═O)cycloalkyl, —C(═O)—NH-alkyl, —COOH (and esters and carboxamides thereof), —C(═O)-morpholine, —C(═O)-heterocycles, —C(—CH₃)₂—OH, —CH₂—C(═O)—NH₂; -hydroxy, alkylhydroxy, alkyl-COOH (and esters and carboxamides thereof), amino, NHC(═O)alkyl, —N(alkyl)C(═O)alkyl, —NHC(═O)aryl, —N(alkyl)C(═O)aryl, —N(H)S(═O)₂alkyl, —N(H)S(═O)₂aryl, —N(alkyl)S(═O)₂alkyl, cyanoalkyl, haloalkyl, ═S═(O)₂-alkyl, ═S═(O)₂-cycloalkyl, ═S═(O)₂-aryl, —S(═O)₂N(H)alkyl, substituted or unsubstituted morpholine, 3-7 membered heterocycle, any of which may have one or more substituents, 3-7 membered cycloalkyl or heterocycle, wherein the 3-7 membered cycloalkyl or heterocycle is optionally fused with another 3-7 cycloalkyl or heterocycle, wherein the rings are spiro, bridged bicyclic, or spiro, wherein the at least one heteroatom in the heterocycle rings are independently selected from O, S, and N; and
wherein one or more hydrogen atoms are optionally deuterium.

2. The compound of claim 1, wherein Y is N.

3. The compound of claim 1, wherein Y is CR₂.

4. The compound of claim 1, wherein Z₁ and Z₂ are F.

5. The compound of claim 1, wherein Z₁ and Z₂ are independently selected from Cl or F.

6. The compound of claim 1, wherein Z₃ is H.

7. The compound of claim 1, wherein Z₃ is F.

8. The compound of claim 1, wherein Z₃ is —CH₂—OH.

9. The compound of claim 1, wherein X is selected from a group consisting of methyl, H, Cl, or —CD₃.

10. The compound of claim 1, wherein X is methyl.

11. The compound of claim 1, wherein R₁ is selected from H or methyl.

12. The compound of claim 1, wherein R₂ is H.

13. The compound of claim 1, wherein R₄ is H.

14. The compound of claim 1, wherein A is selected from:

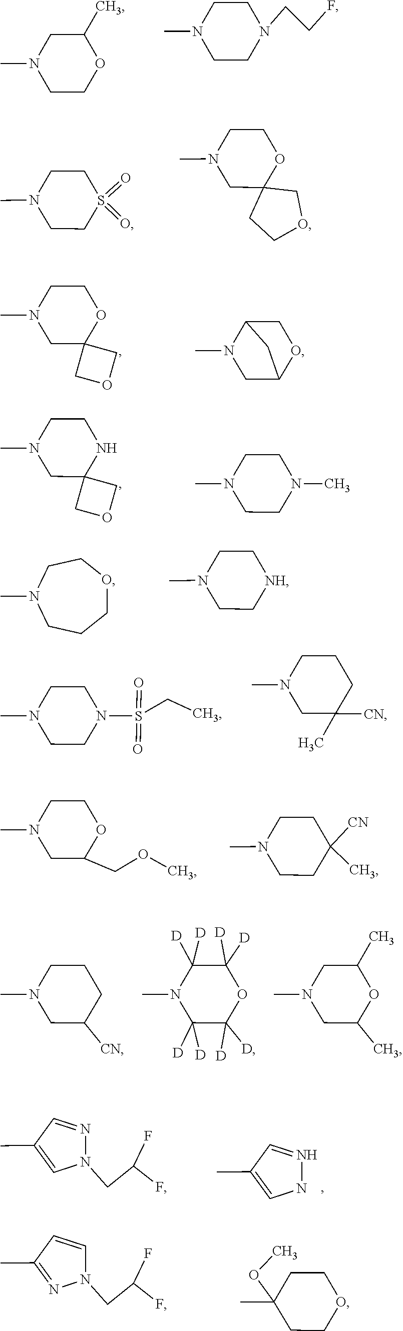

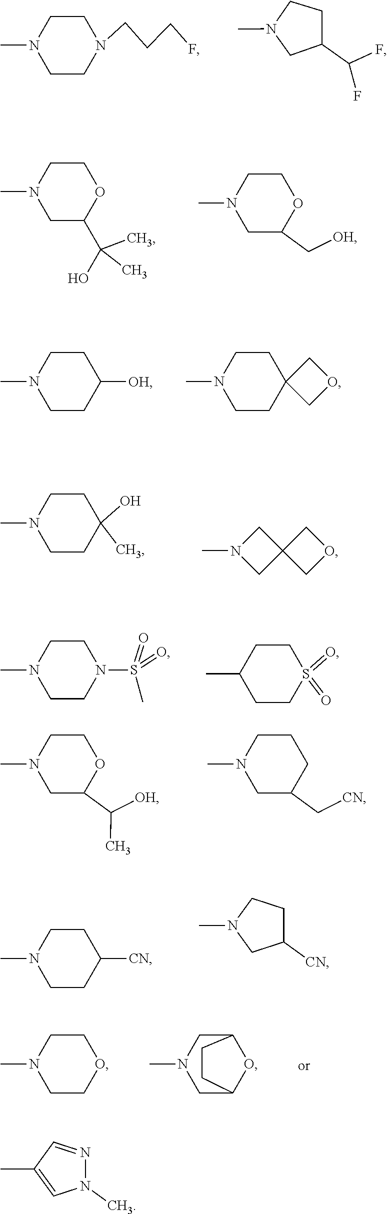

15. The compound of claim 1, wherein the compound of Formula (I) is selected from:

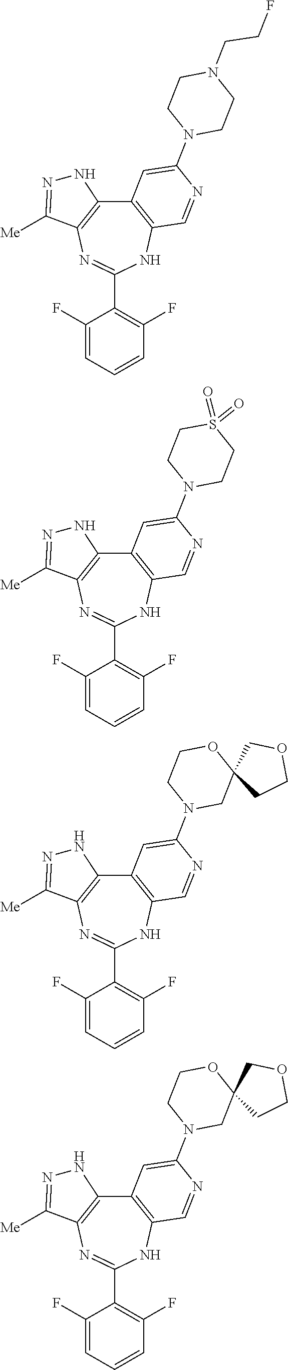 [SMILES: Cc1n[nH]c2c1N=C(c1c(F)cccc1F)Nc1cnc(N3CCN(CCF)CC3)cc1-2 ; Cc1n[nH]c2c1N=C(c1c(F)cccc1F)Nc1cnc(N3CCS(=O)(=O)CC3)cc1-2 ; Cc1n[nH]c2c1N=C(c1c(F)cccc1F)Nc1cnc(N3CCO[C@@]4(CCOC4)C3)cc1-2 ; Cc1n[nH]c2c1N=C(c1c(F)cccc1F)Nc1cnc(N3CCO[C@]4(CCOC4)C3)cc1-2]

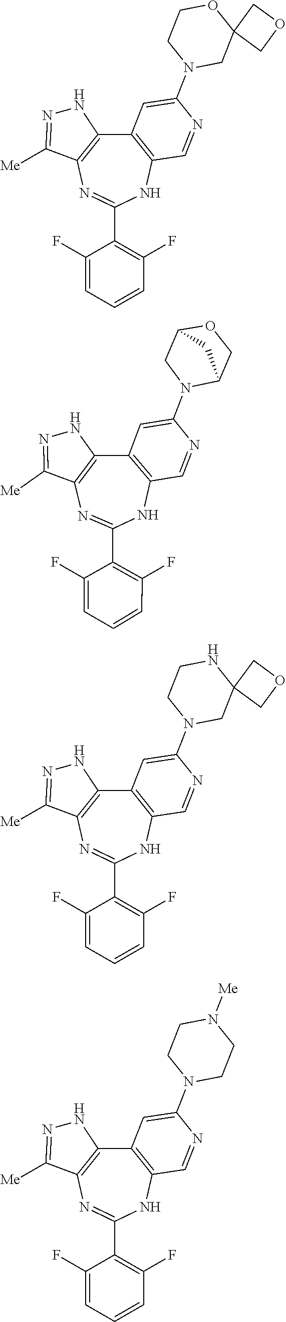 [SMILES: Cc1n[nH]c2c1N=C(c1c(F)cccc1F)Nc1cnc(N3CCOC4(COC4)C3)cc1-2 ; Cc1n[nH]c2c1N=C(c1c(F)cccc1F)Nc1cnc(N3C[C@@H]4C[C@H]3CO4)cc1-2 ; Cc1n[nH]c2c1N=C(c1c(F)cccc1F)Nc1cnc(N3CCNC4(COC4)C3)cc1-2 ; Cc1n[nH]c2c1N=C(c1c(F)cccc1F)Nc1cnc(N3CCN(C)CC3)cc1-2]

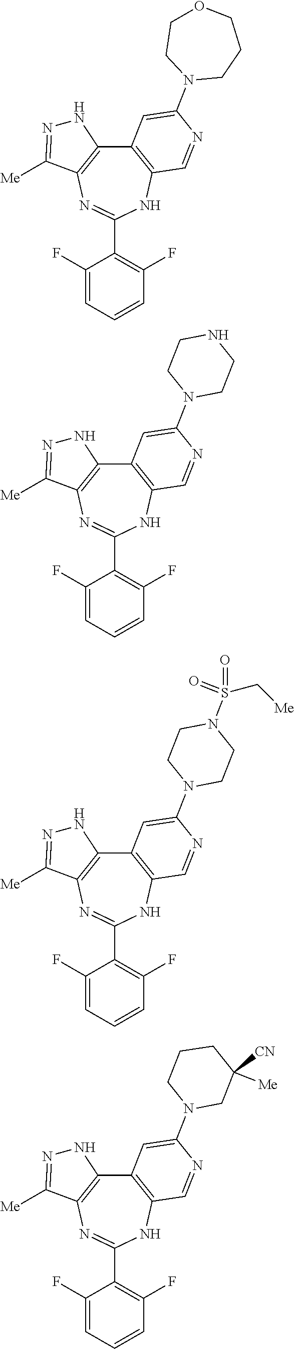 [SMILES: Cc1n[nH]c2c1N=C(c1c(F)cccc1F)Nc1cnc(N3CCCOCC3)cc1-2 ; Cc1n[nH]c2c1N=C(c1c(F)cccc1F)Nc1cnc(N3CCNCC3)cc1-2 ; CCS(=O)(=O)N1CCN(c2cc3c(cn2)NC(c2c(F)cccc2F)=Nc2c(C)n[nH]c2-3)CC1 ; Cc1n[nH]c2c1N=C(c1c(F)cccc1F)Nc1cnc(N3CCC[C@@](C)(C#N)C3)cc1-2]

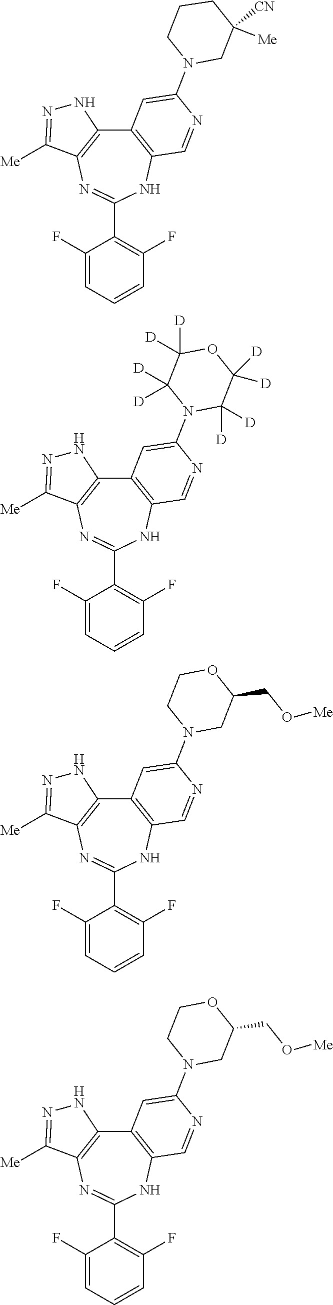 [SMILES: Cc1n[nH]c2c1N=C(c1c(F)cccc1F)Nc1cnc(N3CCC[C@](C)(C#N)C3)cc1-2 ; [2H]C1([2H])OC([2H])([2H])C([2H])([2H])N(c2cc3c(cn2)NC(c2c(F)cccc2F)=Nc2c(C)n[nH]c2-3)C1([2H])[2H] ; COC[C@H]1CN(c2cc3c(cn2)NC(c2c(F)cccc2F)=Nc2c(C)n[nH]c2-3)CCO1 ; COC[C@@H]1CN(c2cc3c(cn2)NC(c2c(F)cccc2F)=Nc2c(C)n[nH]c2-3)CCO1]

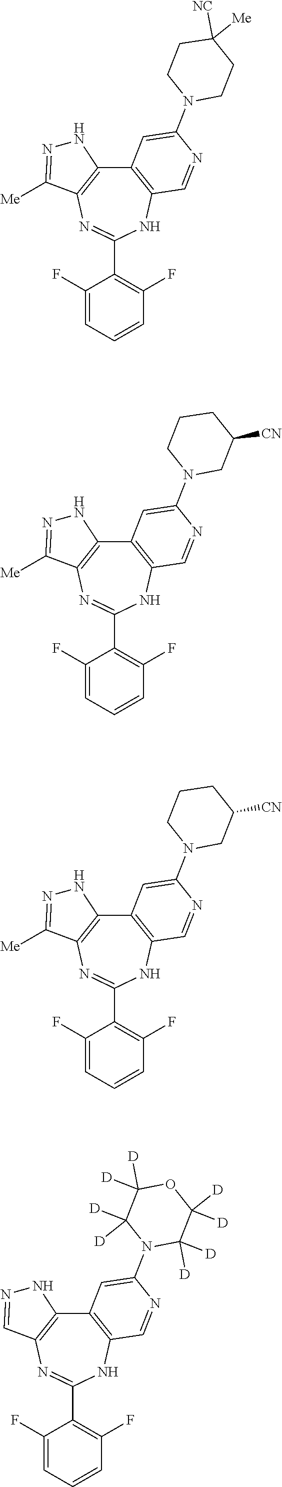 [SMILES: Cc1n[nH]c2c1N=C(c1c(F)cccc1F)Nc1cnc(N3CCC(C)(C#N)CC3)cc1-2 ; Cc1n[nH]c2c1N=C(c1c(F)cccc1F)Nc1cnc(N3CCC[C@@H](C#N)C3)cc1-2 ; Cc1n[nH]c2c1N=C(c1c(F)cccc1F)Nc1cnc(N3CCC[C@H](C#N)C3)cc1-2 ; [2H]C1([2H])OC([2H])([2H])C([2H])([2H])N(c2cc3c(cn2)NC(c2c(F)cccc2F)=Nc2cn[nH]c2-3)C1([2H])[2H]]

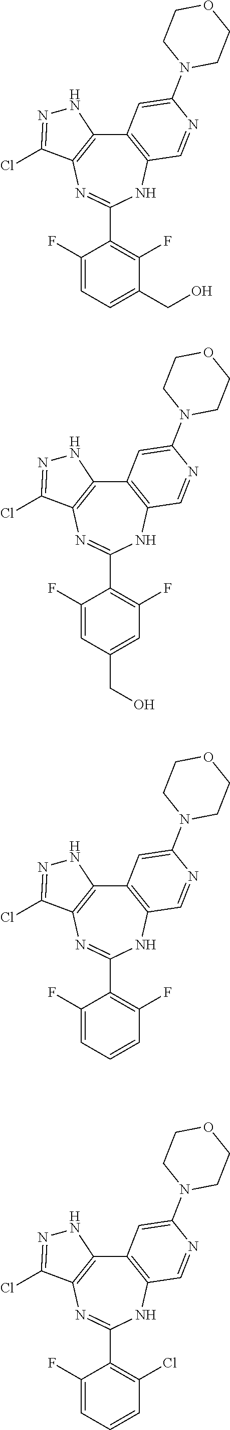 [SMILES: OCc1ccc(F)c(C2=Nc3c(Cl)n[nH]c3-c3cc(N4CCOCC4)ncc3N2)c1F ; OCc1cc(F)c(C2=Nc3c(Cl)n[nH]c3-c3cc(N4CCOCC4)ncc3N2)c(F)c1 ; Fc1cccc(F)c1C1=Nc2c(Cl)n[nH]c2-c2cc(N3CCOCC3)ncc2N1 ; Fc1cccc(Cl)c1C1=Nc2c(Cl)n[nH]c2-c2cc(N3CCOCC3)ncc2N1]

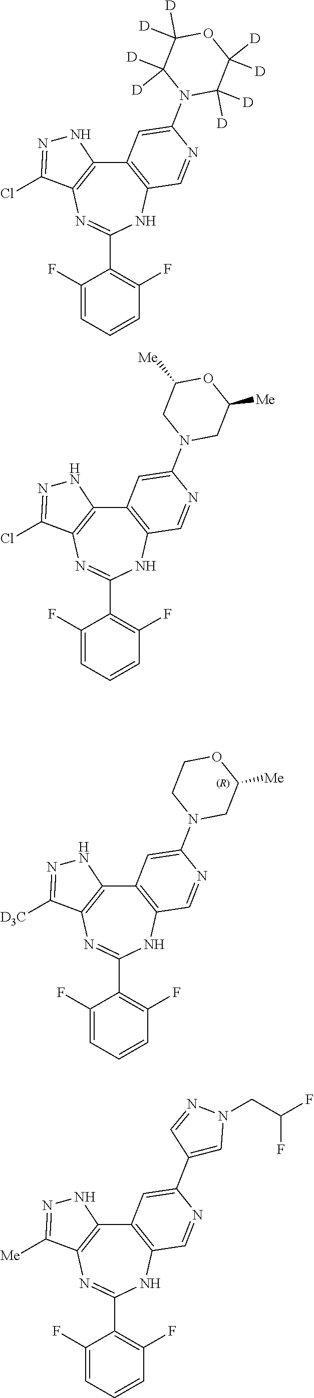

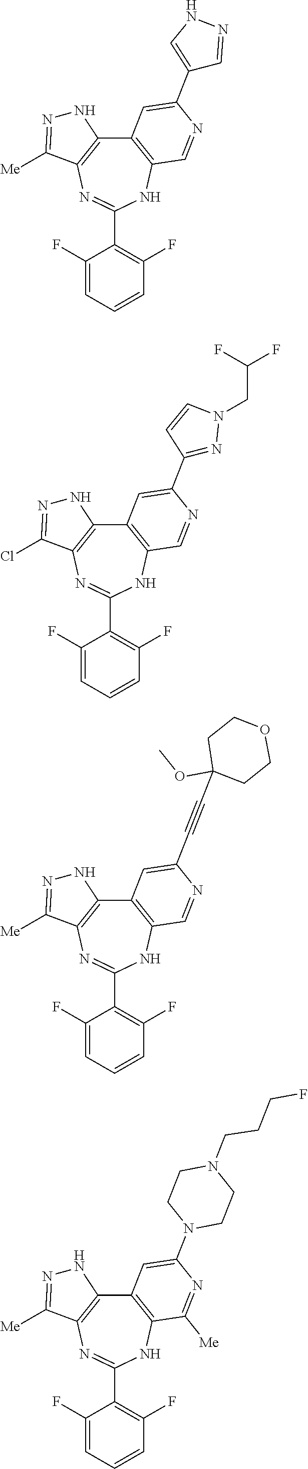 [SMILES: Cc1n[nH]c2c1N=C(c1c(F)cccc1F)Nc1cnc(-c3cn[nH]c3)cc1-2 ; Fc1cccc(F)c1C1=Nc2c(Cl)n[nH]c2-c2cc(-c3ccn(CC(F)F)n3)ncc2N1 ; COC1(C#Cc2cc3c(cn2)NC(c2c(F)cccc2F)=Nc2c(C)n[nH]c2-3)CCOCC1 ; Cc1n[nH]c2c1N=C(c1c(F)cccc1F)Nc1c-2cc(N2CCN(CCCF)CC2)nc1C]

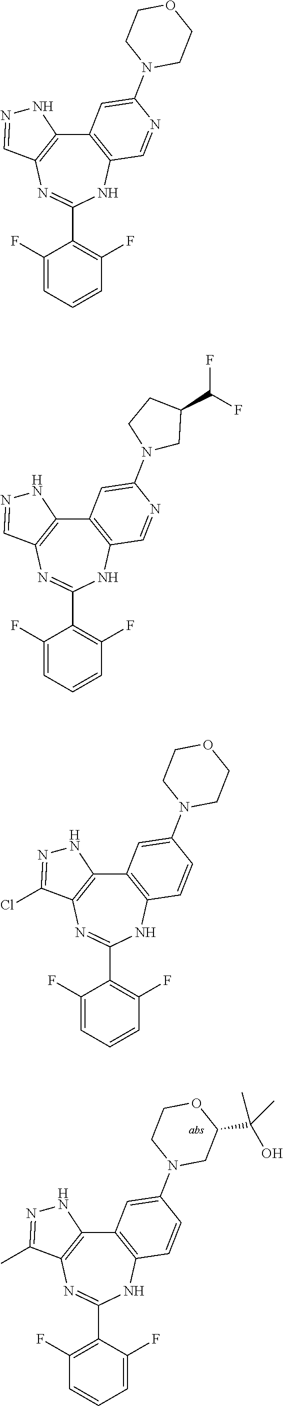 [SMILES: Fc1cccc(F)c1C1=Nc2cn[nH]c2-c2cc(N3CCOCC3)ncc2N1 ; Fc1cccc(F)c1C1=Nc2cn[nH]c2-c2cc(N3CC[C@@H](C(F)F)C3)ncc2N1 ; Fc1cccc(F)c1C1=Nc2c(Cl)n[nH]c2-c2cc(N3CCOCC3)ccc2N1 ; Cc1n[nH]c2c1N=C(c1c(F)cccc1F)Nc1ccc(N3CCO[C@H](C(C)(C)O)C3)cc1-2]

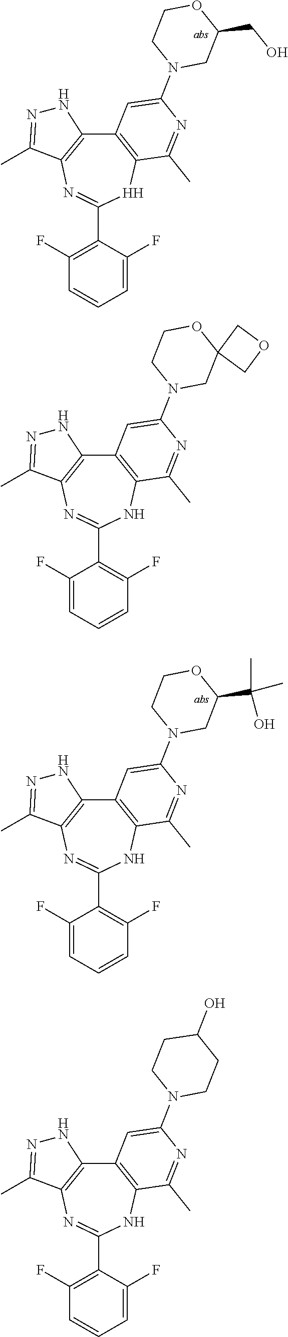

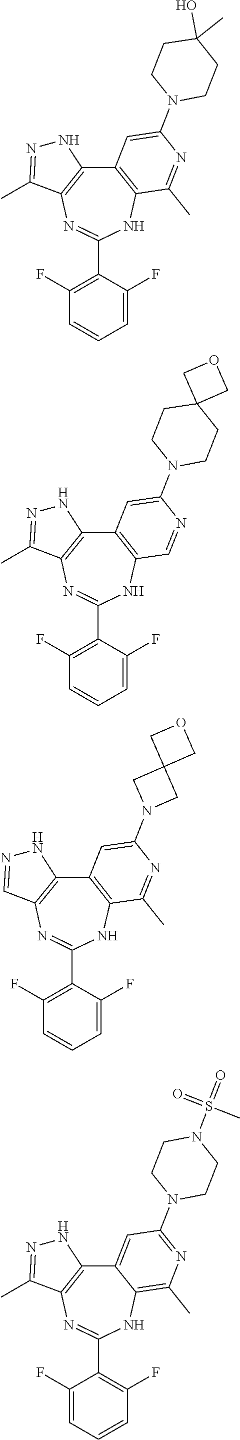

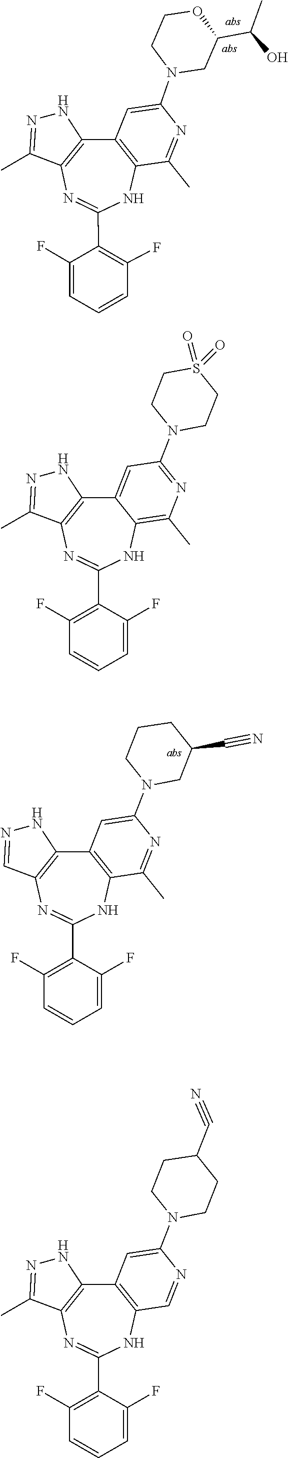 [SMILES: Cc1n[nH]c2c1N=C(c1c(F)cccc1F)Nc1c-2cc(N2CCO[C@H]([C@@H](C)O)C2)nc1C ; Cc1n[nH]c2c1N=C(c1c(F)cccc1F)Nc1c-2cc(N2CCS(=O)(=O)CC2)nc1C ; Cc1nc(N2CCC[C@@H](C#N)C2)cc2c1NC(c1c(F)cccc1F)=Nc1cn[nH]c1-2 ; Cc1n[nH]c2c1N=C(c1c(F)cccc1F)Nc1cnc(N3CCC(C#N)CC3)cc1-2]

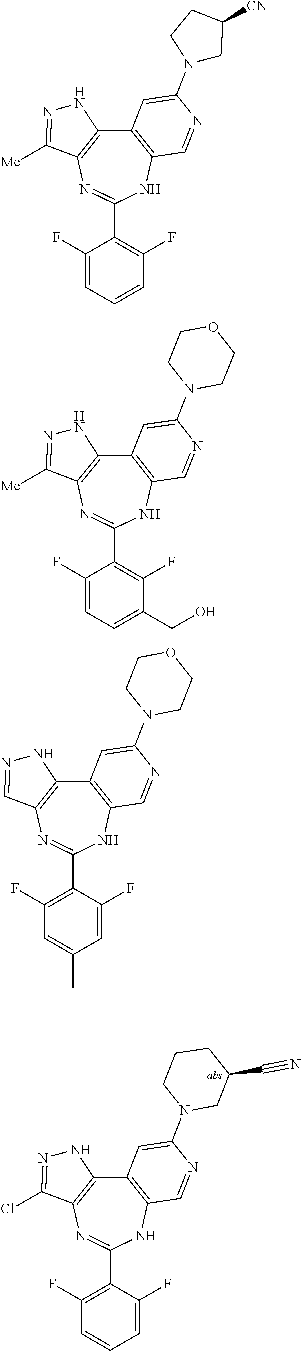

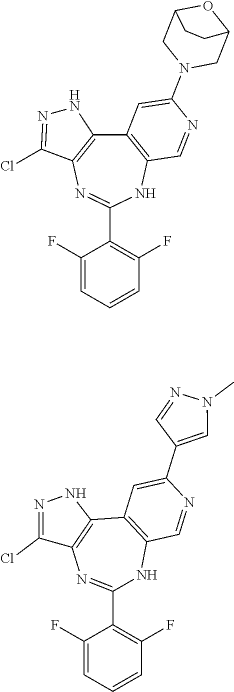 [SMILES: Fc1cccc(F)c1C1=Nc2c(Cl)n[nH]c2-c2cc(N3CC4CCC(C3)O4)ncc2N1 ; Cn1cc(-c2cc3c(cn2)NC(c2c(F)cccc2F)=Nc2c(Cl)n[nH]c2-3)cn1]

16. A method of treatment of a patient suffering from an inflammatory condition, wherein the compound comprises administering a therapeutically effective amount of compound of Formula (I): 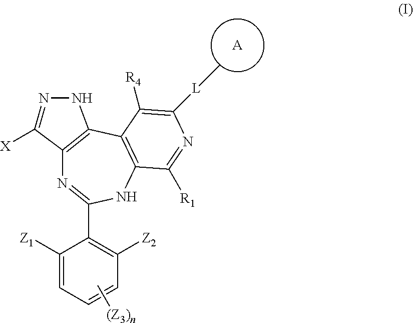
or an enantiomer, mixture of enantiomers, tautomer, or pharmaceutically acceptable salt thereof,
wherein:
n is 1, 2, or 3;
Y is N or CR₂;
Z₁, Z₂, and Z₃ are independently selected from H, —OH, halo, cyano, amino, C₁-C₆ alkyl, C₁-C₆ heteroalkyl, haloalkyl, alkoxy, haloalkxoxy, —CH(OH)-alkyl, hydroxyalkyl, or hydroxyalkoxy;
X is H, halo, cyano, cycloalkyl, C₁-C₆ alkyl, optionally deuterated C₁-C₆ alkyl, C₁-C₆ heteroalkyl, haloalkyl, alkoxy, haloalkxoxy, —CH(OH)-alkyl, hydroxyalkyl, or hydroxyalkoxy;
R₁, R₂, and R₄ are independently selected from H, halo, cyano, C₁-C₆ alkyl, optionally deuterated C₁-C₆ alkyl, C₁-C₆ heteroalkyl, haloalkyl, alkoxy, haloalkxoxy, —CH(OH)-alkyl, hydroxyalkyl, or hydroxyalkoxy;
L is a linker, wherein L is a single bond, substituted or unsubstituted C₁-C₄ alkyl, substituted or unsubstituted C₁-C₄ heteroalkyl, wherein the one or more heteroatoms are selected from O, S, or N;
A is 4-8 membered substituted or unsubstituted heterocycloalkyl, spiroheterocycloalkyl, heteroaryl, wherein one or more heteroatoms are selected from a group consisting of O, S, or N; and
wherein the substituents may be selected from a group consisting of substituted or unsubstituted 3-7 membered heterocycle, ═O, —CH₂-cycloalkyl, —CF₂-cycloalky, —C(═O)—O-alkyl, halo, deuterium, cyano, cyanoalkyl, —CF₃, mono-, di-, or tri-halo alkyl, CH(CH₃)-cycloalkyl, —CH₂-aryl, —CF₂-aryl, —CH(—CH₃)-aryl, C(═O)-alkyl, —C(═O)cycloalkyl, —C(═O)—NH-alkyl, —COOH (and esters and carboxamides thereof), —C(═O)-morpholine, —C(═O)-heterocycles, —C(—CH₃)₂—OH, —CH₂—C(═O)—NH₂; -hydroxy, alkylhydroxy, alkyl-COOH (and esters and carboxamides thereof), amino, NHC(═O)alkyl, —N(alkyl)C(═O)alkyl, —NHC(═O)aryl, —N(alkyl)C(═O)aryl, —N(H)S(═O)₂alkyl, —N(H)S(═O)₂aryl, —N(alkyl)S(═O)₂alkyl, cyanoalkyl, haloalkyl, ═S═(O)₂-alkyl, ═S═(O)₂-cycloalkyl, ═S═(O)₂-aryl, —S(═O)₂N(H)alkyl, substituted or unsubstituted morpholine, 3-7 membered heterocycle, any of which may have one or more substituents, 3-7 membered cycloalkyl or heterocycle, wherein the 3-7 membered cycloalkyl or heterocycle is optionally fused with another 3-7 cycloalkyl or heterocycle, wherein the rings are spiro, bridged bicyclic, or spiro, wherein the at least one heteroatom in the heterocycle rings are independently selected from O, S, and N; and
wherein one or more hydrogen atoms are optionally deuterium.

17. The method of claim 16, wherein Y is N.

18. The method of claim 16, wherein Y is CR₂.

19. The method of claim 16, wherein Z₁ and Z₂ are F.

20. The method of claim 16, wherein Z₁ and Z₂ are independently selected from Cl or F.

21. The method of claim 16, wherein Z₃ is H.

22. The method of claim 16, wherein Z₃ is F.

23. The method of claim 16, wherein Z₃ is —CH₂—OH.

24. The method of claim 16, wherein X is selected from a group consisting of methyl, H, Cl, or —CD₃.

25. The method of claim 16, wherein X is methyl.

26. The method of claim 16, wherein R₁ is selected from H or methyl.

27. The method of claim 16, wherein R₂ is H.

28. The method of claim 16, wherein R₄ is H.

29. The method of claim 16, wherein A is selected from:

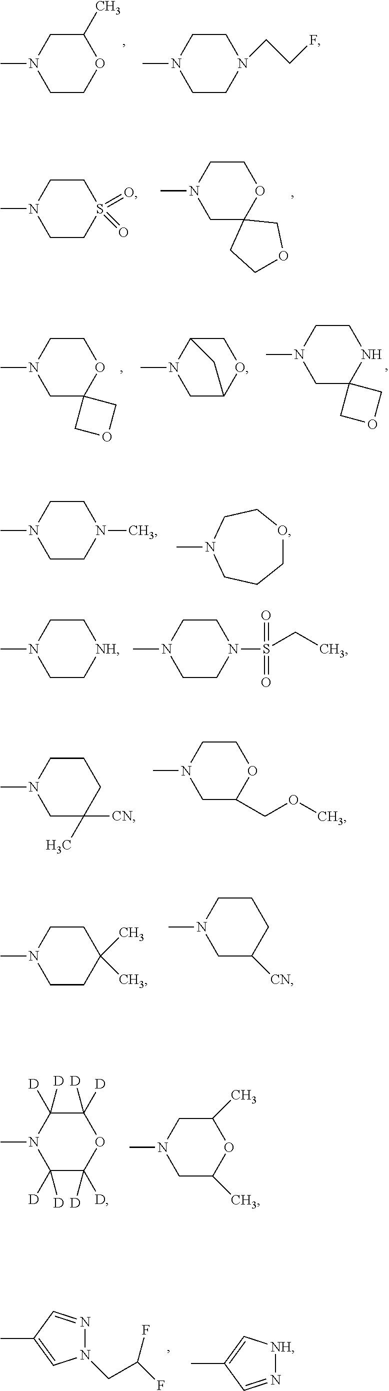

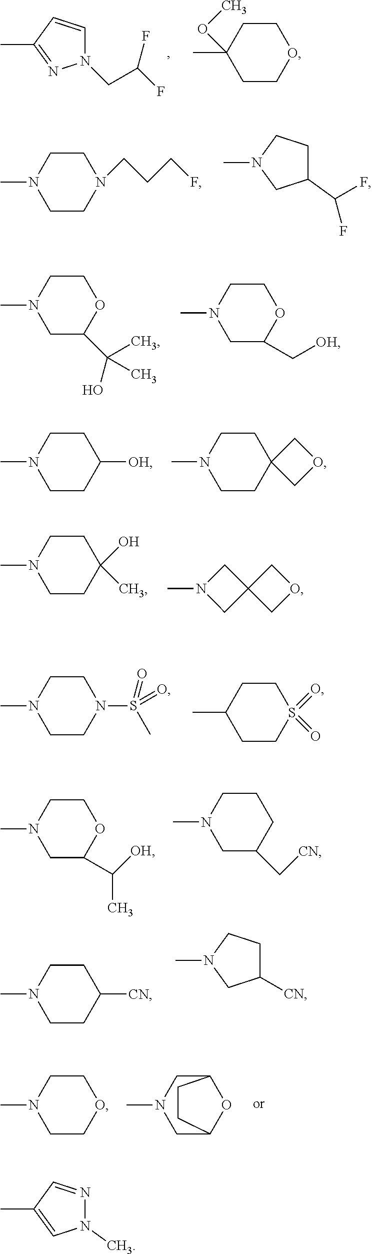

30. The method of claim 16, wherein the compound of Formula (I) is selected from:

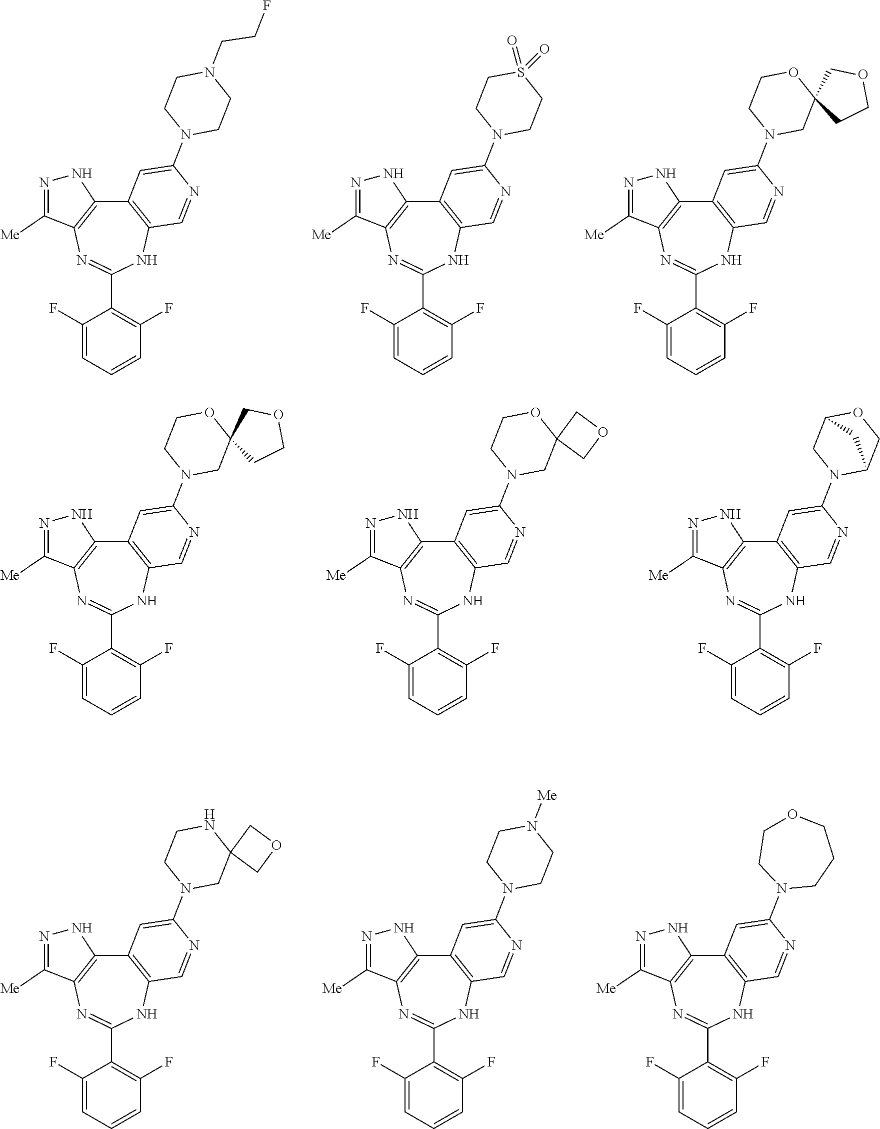

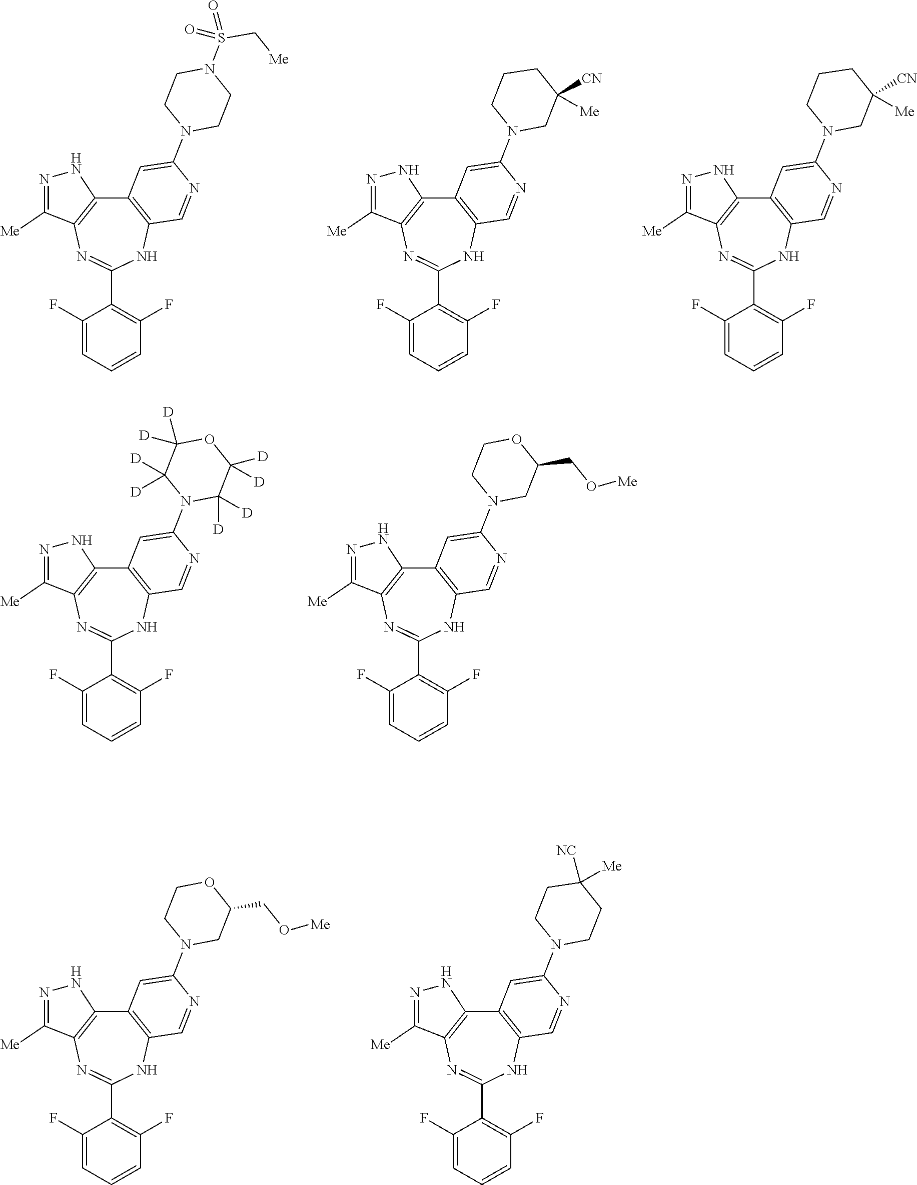

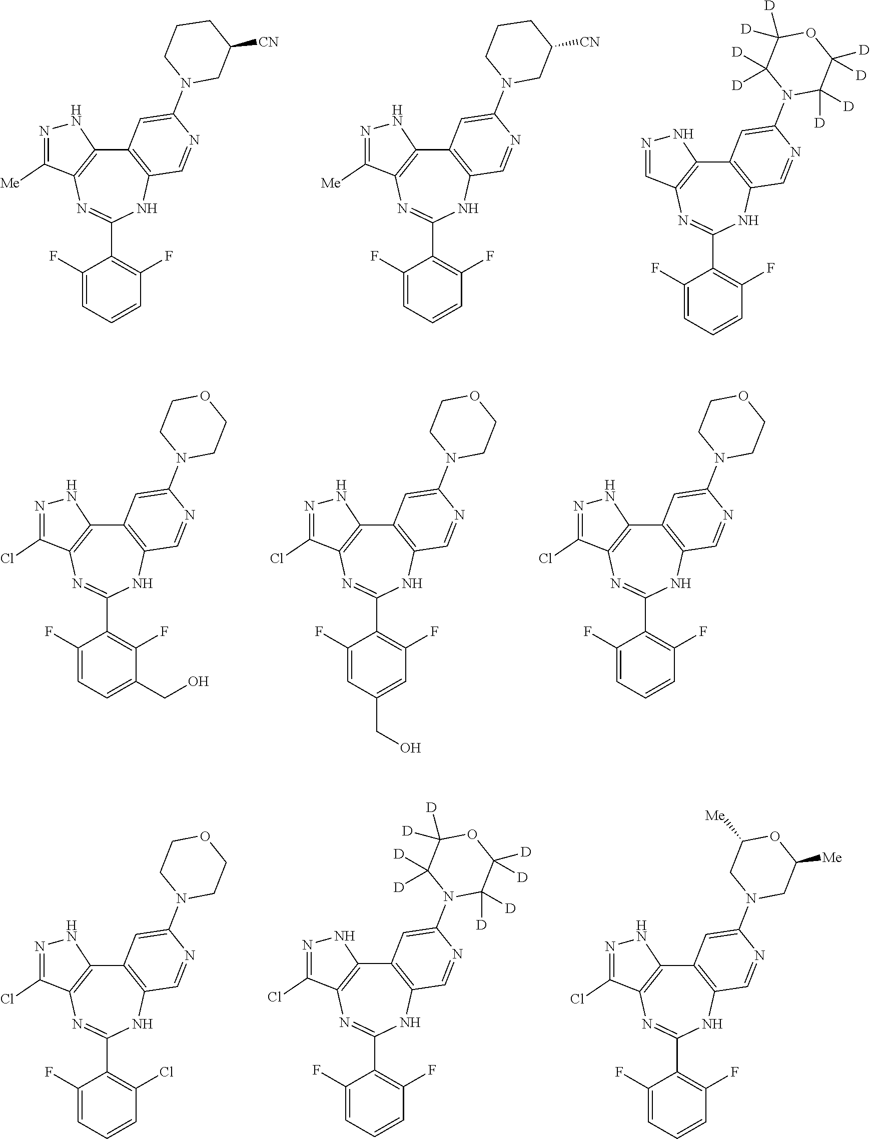

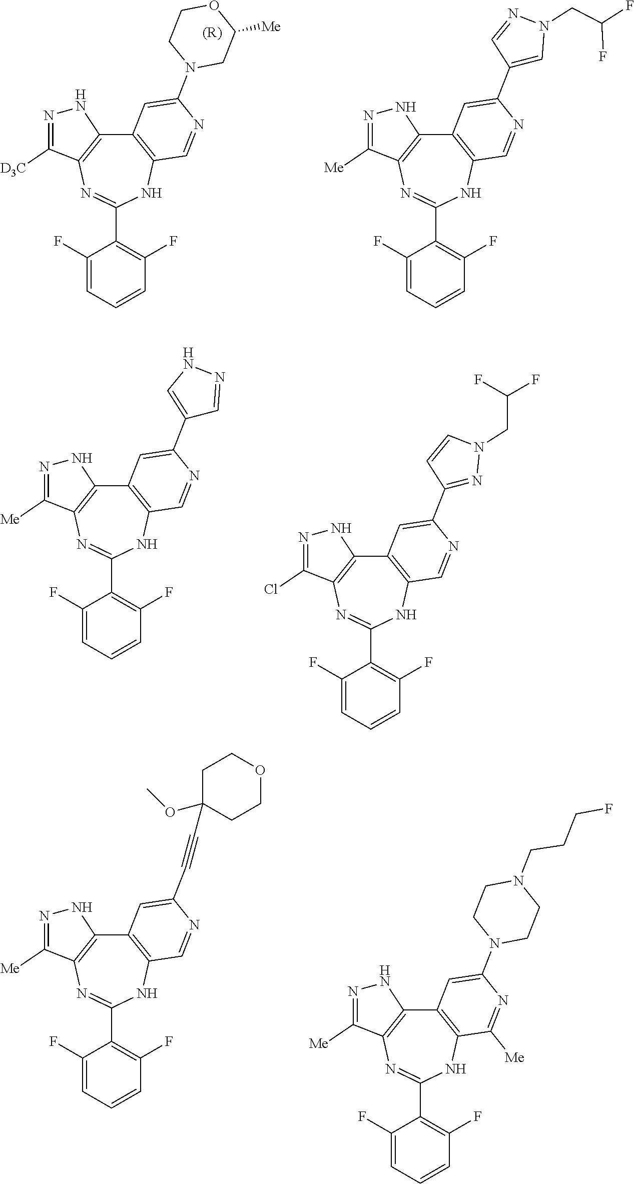

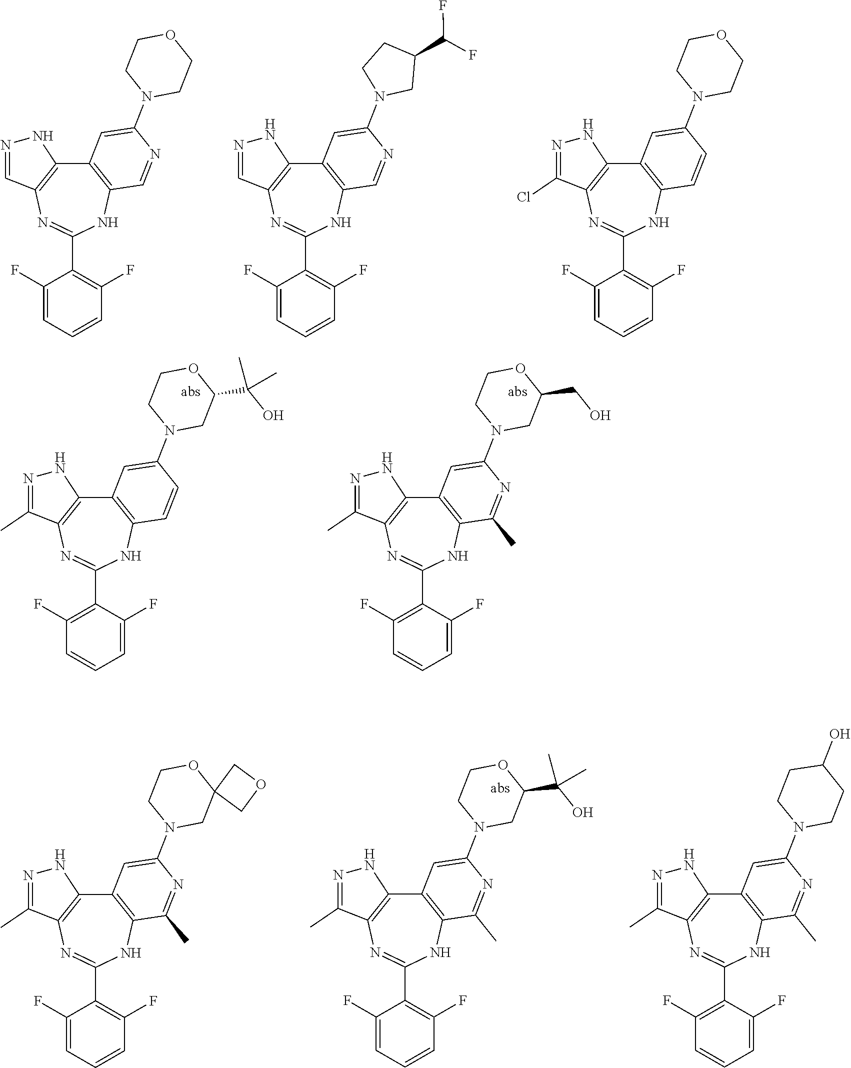

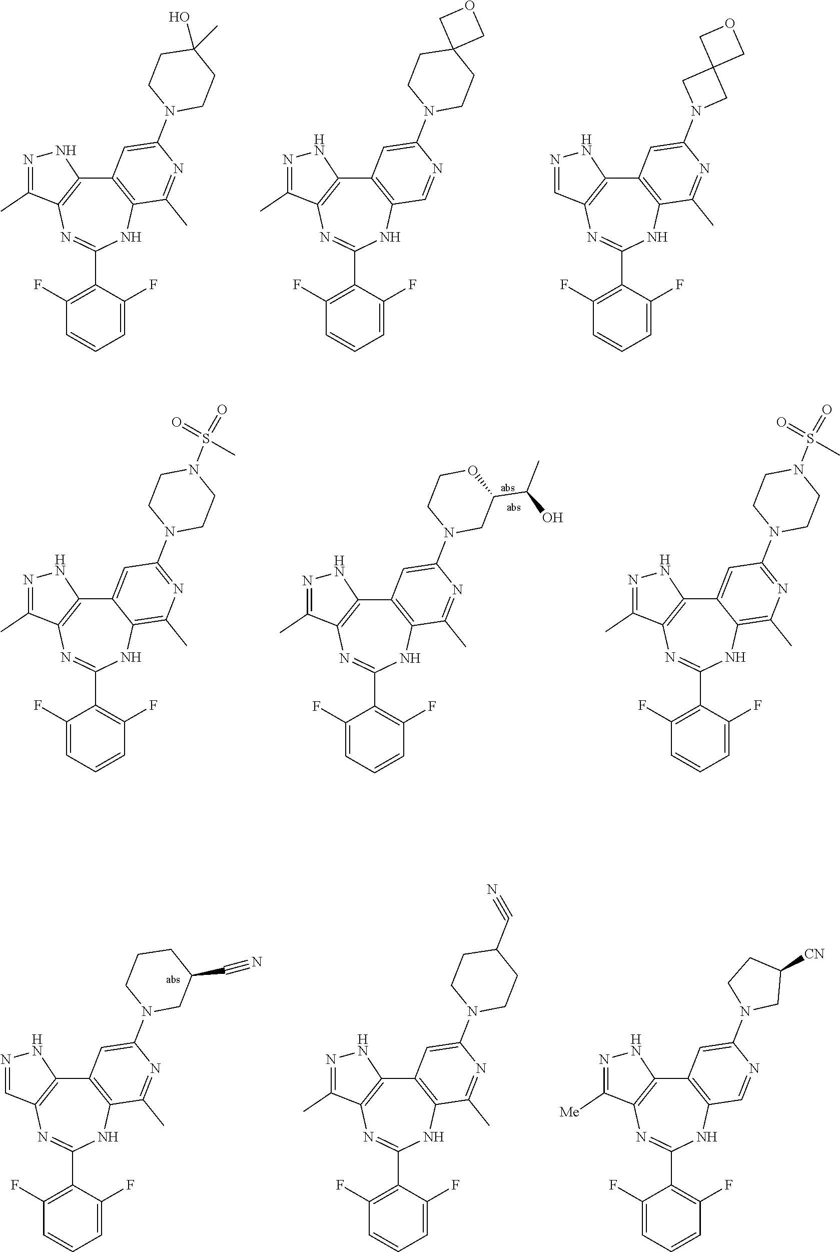

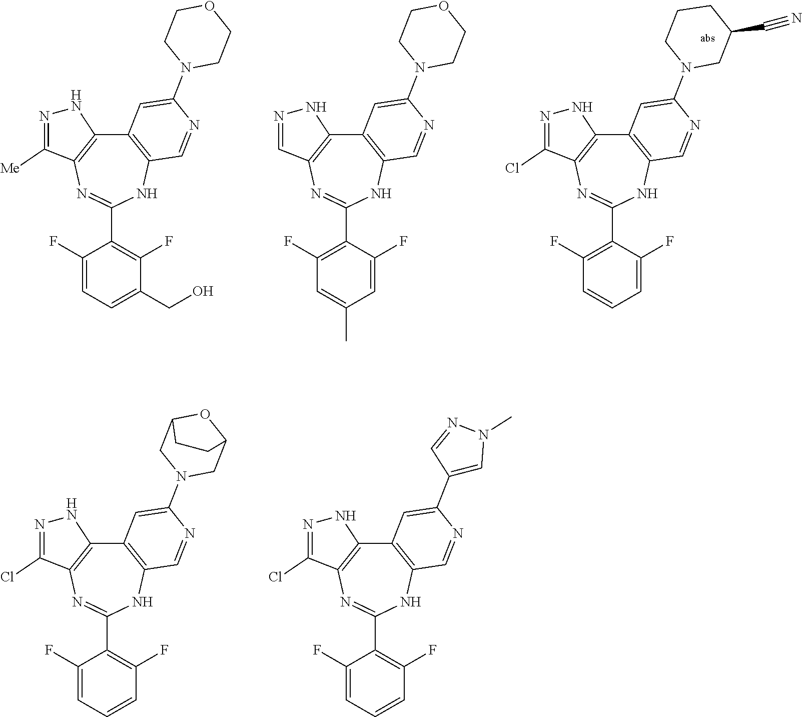 [SMILES: Cc1n[nH]c2c1N=C(c1c(F)ccc(CO)c1F)Nc1cnc(N3CCOCC3)cc1-2 ; Cc1cc(F)c(C2=Nc3cn[nH]c3-c3cc(N4CCOCC4)ncc3N2)c(F)c1 ; N#C[C@@H]1CCCN(c2cc3c(cn2)NC(c2c(F)cccc2F)=Nc2c(Cl)n[nH]c2-3)C1 ; Fc1cccc(F)c1C1=Nc2c(Cl)n[nH]c2-c2cc(N3CC4CCC(C3)O4)ncc2N1 ; Cn1cc(-c2cc3c(cn2)NC(c2c(F)cccc2F)=Nc2c(Cl)n[nH]c2-3)cn1]

31. The method of claim 16, wherein the inflammatory condition is inflammatory bowel disease.
